(19) United States
(12) Patent Application Publication
(10) Pub. No.: US 2023/0203214 A1
(43) Pub. Date: Jun. 29, 2023

(54) AZACOUMARIN AND AZATHIOCOUMARIN DERIVATIVES FOR USE IN OPTICALLY ACTIVE DEVICES

(71) Applicant: MERCK PATENT GMBH, Darmstadt (DE)

(72) Inventors: Stefan RIEDMUELLER, Darmstadt (DE); Stefan GERSTENECKER, Darmstadt (DE)

(21) Appl. No.: 17/999,382

(22) PCT Filed: May 17, 2021
(86) PCT No.: PCT/EP2021/062919

§ 371 Date: Nov. 18, 2022

(30) Foreign Application Priority Data
May 20, 2020 (EP) 20175709.3

(51) Int. Cl. C08F 20/36; A61F 2/16

(52) U.S. Cl. CPC C08F 20/36; A61F 2/16; A61F 2002/16965

(57) ABSTRACT

The present invention relates to novel ophthalmic devices comprising polymerized compounds comprising a photoactive chromophore, said polymerized compounds, and special monomer compounds being particularly suitable for compositions and ophthalmic devices. The present invention is also directed to a process of changing the optical properties of said ophthalmic device or a precursor article for manufacturing said ophthalmic device.

### FIELD OF THE INVENTION

The present invention relates to novel ophthalmic devices comprising polymerized compounds comprising a photoactive chromophore, said polymerized compounds, and special monomer compounds being particularly suitable for compositions and ophthalmic devices. The present invention is also directed to a process of changing the optical properties of said ophthalmic device or a precursor article for manufacturing said ophthalmic device.

### BACKGROUND OF THE INVENTION

Cataract is a general term for an affection of the eye that leads to a loss of vision and in the extreme to blindness by clouding of the normally clear lens of the eye. It is the major cause of blindness in the world, affecting more than 100 million people. Due to the fact that its major cause is age and the population's average age is increasing, it is expected that the number of cataracts will continue to increase substantially in the future.

Effective treatment of cataract is only possible by surgical intervention, whereby the natural lens of the eye is removed through an incision in the cornea and replaced with an ophthalmic device, often also referred to as “intraocular lens”. In preparation of surgery current state-of-the-art surgical methods employ eye mapping so as to approximate the refractive power best suited to the respective patient.

Even though cataract surgery is one of the most widely used and safest surgical procedures it is not without specific post-surgery problems. It frequently happens that the refractive power of the implanted intraocular lens (IOL) is insufficient for restoring good vision. Such problems may, for example, be caused by changes in eye geometry as consequence of the surgery as well as irregular wound healing and positioning errors that result in the ophthalmic device not having the optimal optical properties. As a result the patient will still require corrective vision aids, e.g. glasses, to be able to see correctly. In some cases the resulting refractive power of the implanted ophthalmic device is so far removed from the required refractive power that further surgery will be required. Particularly for aged persons this is not desirable because the body's capability for healing is reduced with increasing age. Furthermore, there is the risk of attracting endophthalmitis, an inflammation of the eye, which can even lead to a complete loss of vision or worse, loss of the eye.

There is therefore a need in the health sector for optically active ophthalmic devices, and particularly for artificial intraocular lenses, that would allow for non-invasive adjustment of refractive power after implantation of the lens, thereby preferably further reducing the need for post-surgery vision aids.

Some developments in this sense have already been made, as for example evidenced by WO 2007/033831, WO 2009/074520, US 2010/0324165, WO 2017/032442, WO 2017/032443, WO 2017/032444, WO 2018/149850, WO 2018/149852, WO 2018/149853, WO 2018/149855, WO 2018/149856 or WO 2018/149857.

M. Schraub et al, European Polymer Journal 51 (2014) 21-27 describes the photochemistry of 3-phenyl-coumarin containing polymethacrylates.

The synthesis of azacoumarines is known from literature e.g. R. B. Moffett, J. Org. Chem. 1970, 35(11), 3596-3600, D. Bonnetaud et al, J. Heterocycl. Chem. 1972, 9(1), 165-166D, F. Trécourt et al, J. Chem. Soc. Perkin Trans I, 1990, 2409-2415, Billeret et al, J. Heterocycl. Chem 1993, 30(3), 671-674, G. Brufola et al, Heterocycles, 1997, 45, 9, 1715-1721 and D. Wang et al, Org. Lett. 2017, 19, 984-987 as well as from patent literature e.g. CN106810559.

The synthesis of pyranopyidones is known from e.g. O. S. Wolfbeis, Monatshefte for Chemie, 1982, 113, 365-370.

U.S. Pat. No. 4,103,256 describes a dye laser comprising a laser dye solution of azacoumarin compounds.

JP8301849, JP8337583 and U.S. Pat. No. 5,585,385 describe heterocyclic compounds having tachykinin receptor antagonistic action.

JP2004203751 describes 6,6-heterobicyclic derivatives as corticotropin releasing factor (hormone) CRF (CRH) antagonists useful for Alzheimer*s disease and obesity.

CN106810560 describes a synthesis method of azacoumarin derivatives and their application in antitumor drugs.

CN106810559 describes selective inhibitors of fibroblast growth factor receptor comprising a nitrogen comprising heterocyclic six-membered ring where the double bond comprising group is attached to the bicycle via a N-phenyl-N group.

WO2007136125 and WO2007132948 describe compositions for inhibiting the extracellular matrix gene transcription.

WO2007082178 describes prostaglandin reductase inhibitors.

WO2008094476 describes substituted pyrano[2,3-B]pyridine derivatives as cannabinoid-1 receptor modulators.

WO2010049269, WO2010049270, WO2011117195 describe substituted pyridines and their use as herbicides. WO2011057942 describes substituted pyridines for insecticidal use and purposes in agriculture and in the veterinary field. WO2012150550 describes amino pyranones as pesticidal compounds.

WO2018171688 describes compounds for treating and/or preventing obesity and obesity-related disorders.

WO2000008026 describes the synthesis of fungicidal fused bicyclic heterocycles.

US20070053831 describes a method for labeling structures, such as p-amyloid plaques and neurofibrillary tangles, in vivo or in vitro comprising contacting brain tissue with specific azacoumarin compounds.

WO2009032754 describes compounds and methods useful as modulators of CB2 for the treatment or prevention of disease states.

US20110021522 describes azacoumarin compounds as activators of procaspases 3, 6 and/or 7 and related derivatives and pharmaceutical compositions thereof.

WO2013130689 describes compounds for treating spinal muscular atrophy.

WO2016146583 describes the synthesis of KV1.3 inhibitors and the relevant starting materials.

However, there is still a need to provide alternative or improved ophthalmic devices e.g. contact lenses or lenses to be implanted by state of the art cataract surgical methods and there is still a need to provide special compounds for the manufacture of ophthalmic devices e.g. of intraocular lenses to be implanted by state of the art cataract surgical methods, particularly by state of the art micro-incision cataract surgical methods.

Consequently, it is an objective of the present application to provide for alternative or improved ophthalmic devices and suitable compounds for the manufacture of such ophthalmic devices.

It is also an objective of the present application to provide for compounds, the optical properties of which may be changed, preferably by non-invasive techniques.

It is a further objective of the present application to provide alternative compounds or compounds having advantages over currently known compounds, preferably in combination with being suitable for ophthalmic devices.

An advantage for the monomers of formula (I) to be used for the preparation of the ophthalmic device according to the invention is the better handling through low melting points by using them in compositions and or polymers/copolymers. A further advantage is that the liquid to low melting monomers of formula (I) to be used for the preparation of the ophthalmic device according to the invention enable a higher flexibility in the choice of initiators for a thermally activated polymerization.

Advantages for polymers or copolymers comprising polymerized monomers of formula (I) according to the invention are good flexibilities and low glass transition temperatures. The polymers or copolymers according to the invention preferably show a significant polarizability change or refractive index change after irradiation and partially increased higher refractive starting indices. The total value of refractive index change per mmol photoactive chromophore is much higher compared to prior art materials. This enables a higher flexibility in adjusting the polarizability or refractive index of the ophthalmic device according to the invention. Based on this advantage of the polymers or copolymers of the invention, the ophthalmic device comprising said materials can be manufactured thinner compared to ophthalmic devices comprising prior art materials.

### SUMMARY OF THE INVENTION

The present inventors have now found that the above objects may be attained either individually or in any combination by ophthalmic devices and the compounds of the present application.

The invention relates to an ophthalmic device or a precursor article for manufacturing an ophthalmic device comprising at least one polymerized compound of formula (I),

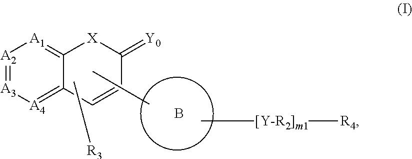

wherein

the divalent group

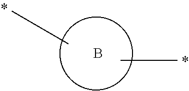

is selected from the group of formulae (B-1), (B-2), (B-3), (B-4) or (B-5)

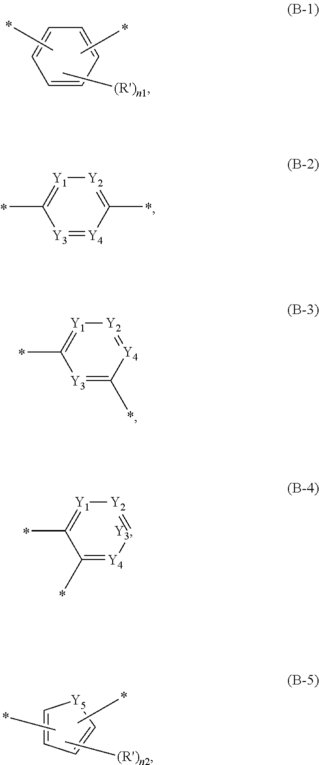

the asterisk * indicates the linkage to the remainder of formula (I);
Y₁, Y₂, Y₃, Y₄ are each independently of each other CR′ or N, provided that only one of Y₁, Y₂, Y₃ and Y₄ is N and the others are CR′;
Y₅ is O, S or NR_{B};
R_{B} is at each occurrence independently selected from a linear or branched alkyl group having 1 to 10 C atoms or a linear or branched partially or fully fluorinated alkyl group having 1 to 10 C atoms;
X is O or S;
Y₀ is O or S;
A₁, A₂, A₃, A₄ are each independently of each other N, CR″ or C—Y—R₂—R₁, provided that in case m1 is 1 only one of A₁, A₂, A₃ and A₄ is N and the others are CR″ and provided that in case m1 is 0, only one of A₁, A₂, A₃ and A₄ is N, only one of A₁, A₂, A₃ and A₄ is C—Y—R₂—R₁ and the others are each independently CR″; or
   in case of m1 is 0, adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″;
Y is independently of each other O, S, SO₂, or a bond;
m1 is 0 or 1;
n1 is 4;
n2 is 2;
R′ is at each occurrence independently selected from the group consisting of H, F, SF₅, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated, partially or completely halogenated thioalkyl group having 1 to 20 C atoms;
R″ is at each occurrence independently selected from the group consisting of H, F, Cl, Br, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated and partially or completely halogenated thioalkyl group having 1 to 20 C atoms;
R₁ is a trialkoxysilyl group or a dialkoxyalkylsilyl group where the alkyl and/or alkoxy groups are each independently linear or branched having 1 to 6 C atoms, or a silyl group of formula (1), (2) or (3) or a polymerizable group of formula (4),

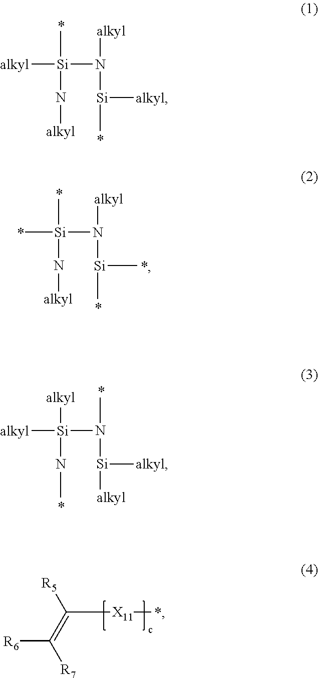

where alkyl means at each occurrence independently of each other a linear or branched alkyl group having 1 to 6 C atoms and the asterisk “*” denotes at each occurrence independently of each other a linkage to the linker —R₂—, —R₂—Y or [Y—R₂—]ₘ₁; and wherein
X₁₁ is selected from the group consisting of O, S, O—SO₂, SO₂—O, C(═O), OC(═O), C(═O)O, S(C═O) and (C═O)S, R₅, R₆, R₇ are at each occurrence independently of each other selected from the group consisting of H, F, a linear or branched, non-fluorinated, partially or completely fluorinated alkyl group having 1 to 20 C atoms and aryl with 6 to 14 C atoms and
c is 0 or 1;
—R₂— is —(C(R)₂)ₒ—, or —(C(R)₂)ₚ—X₈—(C(R)₂)_{q}—(X₉)ₛ—(C(R)₂)ᵣ—(X₁₀)ₜ—(C(R)₂)ᵤ—;
   R is at each occurrence independently selected from the group consisting of H, F, a linear or branched alkyl group having 1 to 4 C atoms or a linear or branched partially or fully fluorinated alkyl group having 1 to 4 C atoms;
   is selected from the group consisting of 0 to 20,
   X₈, X₉, X₁₀ are at each occurrence independently O, S, SO₂, or NR₀,
   s, t is 0 or 1,
   p, q are at each occurrence independently selected from the group consisting of 1 to 10,
   r, u are at each occurrence independently selected from the group consisting of 0 to 10, wherein the overall number of atoms for —(C(R)₂)ₚ—X₈—(C(R)₂)_{q}—(X₉)ₛ—(C(R)₂)ᵣ—(X₁₀)ₜ—(C(R)₂)ᵤ— is up to 20 atoms,
R₀ is at each occurrence independently selected from the group consisting of a linear or branched alkyl group having 1 to 4 C atoms and a linear or branched partially or fully fluorinated alkyl group having 1 to 4 C atoms;
R₃ is H, F, Cl, Br, CN or a linear or branched, non-halogenated, partially or completely halogenated alkyl group having 1 to 20 C atoms;
R₄ is R′ in case m1 is 0 and
R₄ is R₁ in case m1 is 1.

The invention relates further to a process of forming an ophthalmic device or a precursor article for manufacturing an ophthalmic device as described before or preferably described below, said process comprising the steps of
providing a composition comprising at least one compound of formula (I) as described before or preferably described below and/or an oligomer or polymer derived from a compound of formula (I) as described below or preferably described below but having at least one reactive group left for polymerization and optionally further monomers different from compounds of formula (I) and/or crosslinking agents and/or UV absorbers and/or radical initiators;
subsequently forming the ophthalmic device or the precursor article of said composition.

The invention relates further to a process of changing the optical properties of an ophthalmic device or a precursor article for manufacturing an ophthalmic device as described before or preferably described below said process comprising the steps of
providing an ophthalmic device or a precursor article with the process as described before or preferably described below, and
subsequently exposing said ophthalmic device or precursor article to irradiation having a wavelength of at least 200 nm and at most 1500 nm.

The invention relates further to an ophthalmic device or precursor article for manufacturing an ophthalmic device obtainable by said process of changing the optical properties described before or preferably described below.

The invention relates further to oligomers, polymers or copolymers comprising at least one polymerized compound of formula (I) as described before or preferably described below.

The invention relates further to compositions for polymerization comprising at least one compound of formula (I) as described before or preferably described below and/or an oligomer or polymer derived from compounds of formula (I) as described before or preferably described below having at least one reactive group left for polymerization and/or a crosslinking agent and/or a UV absorber and/or a radical initiator and optionally further monomers different from compounds of formula (I).

The invention relates further to compounds of formula (I),

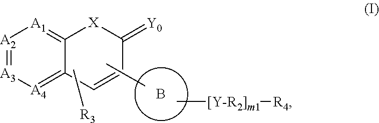

wherein

the divalent group

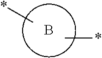

is selected from the group of formulae (B-1), (B-2), (B-3), (B-4) or (B-5)

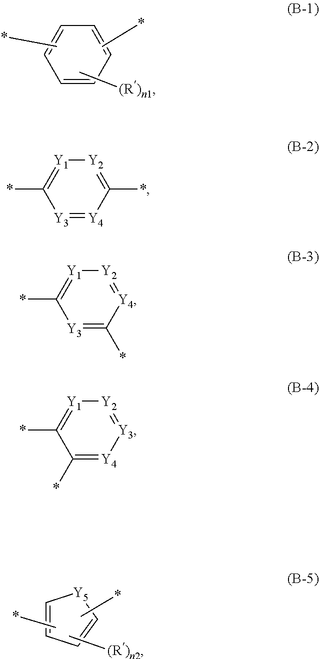

the asterisk * indicates the linkage to the remainder of formula (I);
Y₁, Y₂, Y₃, Y₄ are each independently of each other CR′ or N, provided that only one of Y₁, Y₂, Y₃ and Y₄ is N and the others are CR′;
   Y₅ is O, S or NR_{B};
R_{B} is at each occurrence independently selected from a linear or branched alkyl group having 1 to 10 C atoms or a linear or branched partially or fully fluorinated alkyl group having 1 to 10 C atoms;
A₁, A₂, A₃, A₄ are each independently of each other N, CR″ or C—Y—R₂—R₁, provided that in case m1 is 1 only one of A₁, A₂ and A₃ is N and the others and A₄ are CR″ and provided that in case m1 is 0, only one of A₁, A₂ and A₃ is N, only one of A₁, A₂, A₃ and A₄ is C—Y—R₂—R₁ and the others are each independently CR″; or
   in case of m1 is 0, adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″;
Y is independently of each other O, S, SO₂, or a bond;
m1 is 0 or 1;
n1 is 4;
n2 is 2;
R′ is at each occurrence independently selected from the group consisting of H, F, SF₅, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated, partially or completely halogenated thioalkyl group having 1 to 20 C atoms;
R″ is at each occurrence independently selected from the group consisting of H, F, Cl, Br, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated and partially or completely halogenated thioalkyl group having 1 to 20 C atoms;
R₁ is a trialkoxysilyl group or a dialkoxyalkylsilyl group where the alkyl and/or alkoxy groups are each independently linear or branched having 1 to 6 C atoms, or a silyl group of formula (1), (2) or (3) or a polymerizable group of formula (4),

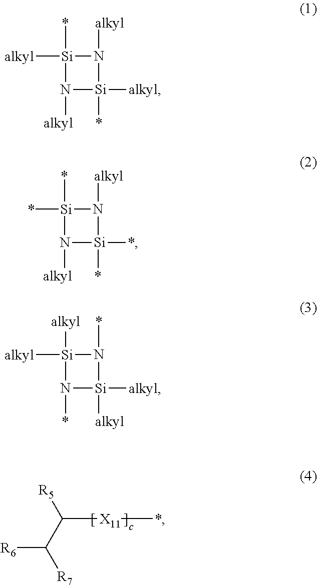

where alkyl means at each occurrence independently of each other a linear or branched alkyl group having 1 to 6 C atoms and the asterisk “*” denotes at each occurrence independently of each other a linkage to the linker —R₂—, —R₂—Y or [Y—R₂—]ₘ₁; and wherein
X₁₁ is selected from the group consisting of O, S, O—SO₂, SO₂—O, C(═O), OC(═O), C(═O)O, S(C═O) and (C═O)S, R₅, R₆, R₇ are at each occurrence independently of each other selected from the group consisting of H, F, a linear or branched, non-fluorinated, partially or completely fluorinated alkyl group having 1 to 20 C atoms and aryl with 6 to 14 C atoms and
c is 0 or 1;
—R₂— is —(C(R)₂)ₒ—, or —(C(R)₂)ₚ—X₈—(C(R)₂)_{q}—(X₉)ₛ—(C(R)₂)ᵣ—(X₁₀)ₜ—(C(R)₂)ᵤ—;
   R is at each occurrence independently selected from the group consisting of H, F, a linear or branched alkyl group having 1 to 4 C atoms or a linear or branched partially or fully fluorinated alkyl group having 1 to 4 C atoms;
   is 0 to 20,
   X₈, X₉, X₁₀ are at each occurrence independently O, S, SO₂, or NR₀,
   s, t is 0 or 1,
   p, q are at each occurrence independently selected from the group consisting of 1 to 10,
   r, u are at each occurrence independently selected from the group consisting of 0 to 10, wherein the overall number of atoms for —(C(R)₂)ₚ—X₈—(C(R)₂)_{q}—(X₉)ₛ—(C(R)₂)ᵣ—(X₁₀)ₜ—(C(R)₂)ᵤ— is up to 20 atoms,
R₀ is at each occurrence independently selected from the group consisting of a linear or branched alkyl group having 1 to 4 C atoms and a linear or branched partially or fully fluorinated alkyl group having 1 to 4 C atoms;
R₃ is H, F, Cl, Br, CN or a linear or branched, non-halogenated, partially or completely halogenated alkyl group having 1 to 20 C atoms;
R₄ is R′ in case m1 is 0 and
R₄ is R₁ in case m1 is 1;

provided that in case of m1 is 0, Y is O or S, A₂ is C—Y—R₂—R₁,

the divalent group

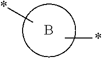

is in position 3 and is selected from formulae (B-3) and (B-4) where Y₄ within formula (B-3) is N and where Y₃ in formula (B-4) is N, c is 1;

provided that in case of m1 is 1, A₂ is CR″ and R″ is a linear or branched, non-halogenated, partially or completely halogenated alkoxy group having 1 to 20 C atoms and a linear or branched, non-halogenated and partially or completely halogenated thioalkyl group having 1 to 20 C atoms,

the divalent group

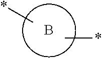

is in position 3 and is selected from formula (B-3), Y₂ within formula (B-3) is CR′ and R′ is H, Y₄ within formula (B-3) is N, and Y is a bond, O or S, c is 1;

provided that in case of m1 is 1, Y is a bond, c is 0, R₃ is C and

the divalent group

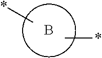

is in position 3, o is 5 to 20; provided that in case of m1 is 0, Y is a bond or O, c is 0, R₃ is Cl and

the divalent group

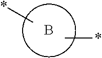

is in position 3, o is 7 to 20;

provided that in case of m1 is 0, X is O, Y₀ is O and Y is a bond, c is 1 and X₁₁ is O, S, O—SO₂, SO₂—O, OC(═O), C(═O)O, S(C═O) and (C═O)S; provided that in case of m1 is 1, X is O and Y₀ is O, o is 5 to 20; provided that in case of m1 is 1, c is 0, Y is a bond or O and

the divalent group

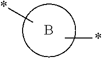

is in position 3, o is 11 to 20;

provided that in case of m1 is 1, A₁ or A₃ is N, Y is a bond, c is 0,

the divalent group

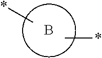

is in position 3, o is 7 to 20 and provided that in case of m1 is 0, X is O, Y₀ is O, Y is O or S, c is 0, A₂ is C—Y—R₂—R₁, A₃ is Br, A₁ is N,

the divalent group

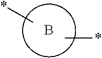

is in position 4 and is selected from formulae (B-1), (B-3) and (B-4), o is 2 to 20.

### DETAILED DESCRIPTION OF THE INVENTION

Compounds of formula (I) as described before or preferably described below can be preferably used as monomers for the preparation of a precursor article such as a blank which may be transformed to an ophthalmic device such as an eye-implant or specifically an intraocular lens or can be preferably used for the preparation of the ophthalmic device as such as described before or preferably described below.

The compounds of formula (I) and all preferred embodiments of compounds of formula (I) including any monomeric units according to the present invention include all stereoisomers or racemic mixtures.

The compounds of formula (I) provide several advantages over prior art materials for the preparation of ophthalmic devices or precursor articles for manufacturing an ophthalmic device as described before. Moreover, the addition of the nitrogen atom to the left aromatic part of the central chromophore in the compounds of formula (I) or oligomers, polymers and copolymers comprising polymerized compounds of formula (I) has a significant impact on the optical properties over prior art compounds as described before.

Polymers that are foldable at room temperature generally exhibit glass transition temperatures (T_{g}) lower than room temperature (ca. 21° C.). They are easily deformable at this temperature without causing physical damage to the polymer, for example by inducing creep, stress or fissures. For polymers in intraocular lenses, T_{g}s of less than or equal to 15° C. are preferred.

Polymers used in ophthalmic device manufacturing, preferably in intraocular lens manufacturing, have preferably relatively high refractive indices, which enable the fabrication of thinner ophthalmic devices such as intraocular lenses. Preferably, the polymer used in an ophthalmic device, preferably an intraocular lens, will have a refractive index greater than about 1.5 and presently most preferably greater than about 1.55.

In case an asterisk (“*”) is used within the description of the present invention, it denotes a linkage to an adjacent unit or group or, in case of a polymer, to an adjacent repeating unit or any other group whenever it is not specifically defined.

A linear or branched alkyl group having 1 to 10 C atoms denotes an alkyl group having 1, 2, 3, 4, 5, 6, 7, 8, 9 or 10 C atoms, for example methyl, ethyl, iso-propyl, n-propyl, iso-butyl, n-butyl, tert-butyl, n-pentyl, 1-, 2- or 3-methylbutyl, 1,1-, 1,2- or 2,2-dimethylpropyl, 1-ethylpropyl, n-hexyl, n-heptyl, n-octyl, ethylhexyl, n-nonyl or n-decyl. A linear or branched alkyl group having 1 to 20 C atoms include all examples for a linear or branched alkyl group having 1 to 10 C atoms including any alkyl group having 11, 12, 13, 14, 15, 16, 17, 18, 19 and 20 C atoms such as n-undecyl, n-dodecyl, n-tridecyl, n-tetradecyl, n-pentadecyl, n-hexadecyl, n-heptadecyl, n-octadecyl, n-nonadecyl and n-eicosyl.

The term partially halogenated alkyl group denotes that at least one H atom of the alkyl group is replaced by F, Cl, Br or I. Preferably, the alkyl group is partially fluorinated meaning that at least one H atom of the alkyl group is replaced by F. A preferred partially halogenated alkyl group is CH₂CF₃.

The term completely halogenated alkyl group denotes that all H atoms of the alkyl group are replaced by F, Cl, Br and/or I. Preferably, the alkyl group is completely fluorinated meaning that all H atoms of the alkyl group are replaced by F. A preferred completely fluorinated alkyl group is trifluoromethyl or pentafluoroethyl.

The term halogenated or preferably fluorinated corresponds additionally to other groups such as a halogenated cycloalkyl group, a halogenated alkoxy group or a halogenated thioalkyl group.

A cycloalkyl group having 3 to 6 C atoms includes cyclopropyl, cyclobutyl, cyclopentyl and cyclohexyl which may be partially or completely halogenated or fluorinated as explained before. Preferably, the cycloalkyl group is cyclopropyl.

A linear or branched alkoxy group having 1 to 20 C atoms denotes an O-alkyl group having 1, 2, 3, 4, 5, 6, 7, 8, 9, 10, 11, 12, 13, 14, 15, 16, 17, 18, 19 or 20 C atoms, for example methoxy, ethoxy, iso-propoxy, n-propoxy, iso-butoxy, n-butoxy, tert-butoxy, n-pentyloxy, 1-, 2- or 3-methylbutyloxy, 1,1-, 1,2- or 2,2-dimethylpropoxy, 1-ethylpropoxy, n-hexyloxy, n-heptyloxy, n-octyloxy, ethylhexyloxy, n-nonyloxy, n-decyloxy, n-undecyloxy, n-dodecyloxy, n-tridecyloxy, n-tetradecyloxy, n-pentadecyloxy, n-hexadecyloxy, n-heptadecyloxy, n-octadecyloxy, n-nonadecyloxy and n-eicosyloxy which may be partially or completely halogenated or preferably may be partially or completely fluorinated. A preferred completely fluorinated alkoxy group is trifluoromethoxy.

A linear or branched thioalkyl group having 1 to 20 C atoms denotes a S-alkyl group having 1, 2, 3, 4, 5, 6, 7, 8, 9, 10, 11, 12, 13, 14, 15, 16, 17, 18, 19 or 20 C atoms, for example thiomethyl, 1-thioethyl, 1-thio-iso-propyl, 1-thio-n-propoyl, 1-thio-iso-butyl, 1-thio-n-butyl, 1-thio-tert-butyl, 1-thio-n-pentyl, 1-thio-1-, -2- or -3-methylbutyl, 1-thio-1,1-, -1,2- or -2,2-dimethylpropyl, 1-thio-1-ethylpropyl, 1-thio-n-hexyl, 1-thio-n-heptyl, 1-thio-n-octyl, 1-thio-ethylhexyl, 1-thio-n-nonyl, 1-thio-n-decyl, 1-thio-n-undecyl, 1-thio-n-dodecyl, 1-thio-n-tridecyl, 1-thio-n-tetradecyl, 1-thio-n-pentadecyl, 1-thio-n-hexadecyl, 1-thio-n-heptadecyl, 1-thio-n-octadecyl, 1-thio-n-nonadecyl and 1-thio-n-eicosyl which may be partially or completely halogenated or preferably may be partially or completely fluorinated. A preferred completely fluorinated thioether group is trifluoromethyl thioether.

Preferred alkyl and alkoxy radicals have 1, 2, 3, 4, 5, 6, 7, 8, 9 or 10 C atoms.

An aryl group in the context of this invention contains 6 to 40 ring atoms and a heteroaryl group in the context of this invention contains 5 to 40 ring atoms comprising at least one heteroatom. The heteroatoms are preferably selected from N, O and/or S. An aryl group or heteroaryl group is understood here to mean either a simple aromatic cycle, i.e. phenyl, or a simple heteroaromatic cycle, for example pyridinyl, pyrimidinyl, thiophenyl, etc., or a fused (annelated) aryl or heteroaryl group, for example naphthyl, anthracenyl, phenanthrenyl, quinolinyl or isoquinolinyl.

An aryl group or heteroaryl group is preferably derived from benzene, naphthalene, anthracene, phenanthrene, pyrene, benzanthracene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

A polymerizable group is a group which can be subject to or can undergo polymerization thus forming an oligomer or a polymer.

Polymerization is the process of taking individual monomers and chaining them together to make longer units. These longer units are called polymers. The compounds of formula (1) as described before and preferably described below are suitable monomers for the preparation of an ophthalmic device or a precursor article for manufacturing an ophthalmic device.

Within the gist of the invention, the polymerizable group R₁ once oligomerized or polymerized thus forms or is part of the backbone of the oligomer, polymer or copolymer comprising polymerized compounds of formula (I). Suitable polymerizable groups are defined to be a trialkoxysilyl group or a dialkoxyalkylsilyl group where the alkyl and/or alkoxy groups are each independently linear or branched having 1 to 6 C atoms, or a silyl group of formula (1), (2) or (3) or a polymerizable group of formula (4),

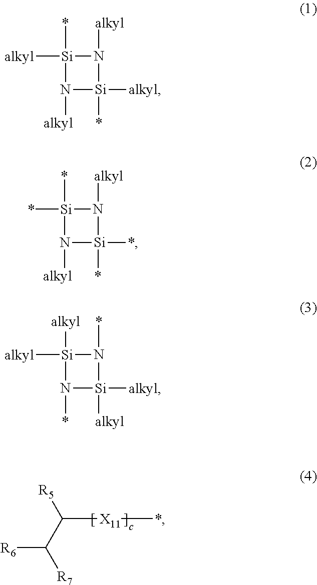

where alkyl means at each occurrence independently of each other a linear or branched alkyl group having 1 to 6 C atoms and the asterisk “*” denotes at each occurrence independently of each other a linkage to the linker —R₂—, —R₂—Y or [Y—R₂—]ₘ₁; and wherein

X₁₁ is selected from the group consisting of O, S, O—SO₂, SO₂—O, C(═O), OC(═O), C(═O)O, S(C═O) and (C═O)S,

R₅, R₆, R₇ are at each occurrence independently of each other selected from the group consisting of H, F, a linear or branched, non-fluorinated, partially or completely fluorinated alkyl group having 1 to 20 C atoms and aryl with 6 to 14 C atoms and

c is 0 or 1.

Particularly preferred polymerizable groups are described below. Particularly preferred polymerized groups are described below.

Aryl with 6 to 14 C atoms is an aryl group preferably selected from the group consisting of phenyl, naphthyl or anthryl, particularly preferably phenyl.

In one preferred embodiment, the compounds of formula (I) acting as monomers for the preparation of the ophthalmic device or the precursor article for manufacturing an ophthalmic device as described before or for the preparation of an oligomer, polymer or copolymer according to the invention or as compound according to the invention contain the polymerizable group R₁ attached via —R₂— and Y to the photoactive ring system. This is the case for compounds of formula (I) in which m1 is 0 which can be described accordingly in formula (I′).

The invention is therefore additionally directed to an ophthalmic device or a precursor article for manufacturing an ophthalmic device comprising at least one polymerized compound of formula (I′),

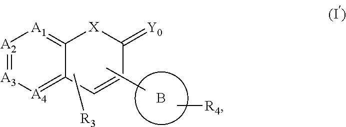

wherein R₁, —R₂—, Y, R₃, X, Y₀, R′, R″ and

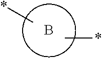

have a meaning as described before or preferably described before or below and

A₁, A₂, A₃, A₄ are each independently of each other N, CR″ or C—Y—R₂—R₁, provided that only one of A₁, A₂, A₃ and A₄ is N, only one of A₁, A₂, A₃ and A₄ is C—Y—R₂—R₁ and the others are each independently CR″; or

adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″ and R₄ is R′.

The invention is therefore additionally directed to compounds of formula (I) wherein m1 is 0 which can preferably be described according to formula (I′),

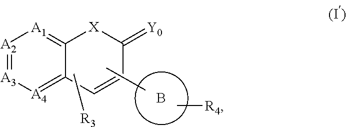

wherein R₁, —R₂—, Y, R₃, X, Y₀, R′, R″ and

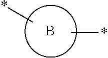

have a meaning as described before or preferably described before or below and

A₁, A₂, A₃, A₄ are each independently of each other N, CR″ or C—Y—R₂—R₁, provided that only one of A₁, A₂ and A₃ is N, only one of A₁, A₂, A₃ and A₄ is C—Y—R₂—R₁ and the others are each independently CR″; or

adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″ and R₄ is R′,

provided that in case Y is O or S, A₂ is C—Y—R₂—R₁ and

the divalent group

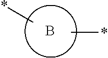

is in position 3 and is selected from formulae (B-3) and (B-4) where Y₄ within formula (B-3) is N and where Y₃ in formula (B-4) is N, c is 1;

provided that in case Y is a bond or O, c is 0, R₃ is Cl and

the divalent group

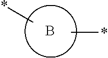

is in position 3, o is 7 to 20; provided that in case of X is O, Y₀ is O and Y is a bond, c is 1 and X₁₁ is O, S, O—SO₂, SO₂—O, OC(═O), C(═O)O, S(C═O) and (C═O)S and

provided that in case of X is O, Y₀ is O, Y is O or S, c is 0, A₂ is C—Y—R₂—R₁, A₃ is Br, A₁ is N, and

the divalent group

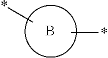

is in position 4 and is selected from formulae (B-1), (B-3) and (B-4), o is 2 to 20.

The position of substituent R₁—R₂—Y or R₁—R₂— in formulae (I) or (I′) is determined through the position of A₁, A₂, A₃, A₄, A₁-A₂, A₂-A₃ and A₃-A₄.

In compounds of formula (I′-a), the substituent A₁ is C—Y—R₂—R₁ which is in position 8 of the photoactive chromophore and A₂, A₃ and A₄ have a meaning as described for compounds of formulae (I) or (I′) before. In compounds of formula (I′-a), A₂ is preferably N.

In compounds of formula (I′-b), the substituent A₂ is C—Y—R₂—R₁ which is in position 7 of the photoactive chromophore and A₁, A₃ and A₄ have a meaning as described for compounds of formulae (I) or (I′) before. In compounds of formula (I′-b), A₁ or A₃ are preferably N.

In compounds of formula (I′-c), the substituent A₃ is C—Y—R₂—R₁ which is in position 6 of the photoactive chromophore and A₁, A₂ and A₄ have a meaning as described for compounds of formulae (I) or (I′) before. In compounds of formula (I′-c), A₁ or A₂ are preferably N.

In compounds of formula (I′-d), the substituent A₄ is C—Y—R₂—R₁ which is in position 5 of the photoactive chromophore and A₁, A₂ and A₃ have a meaning as described for compounds of formulae (I) or (I′) before. In compounds of formula (I′-d), A₁ or A₂ are preferably N.

In compounds of formula (I′-e), the substituent A₁-A₂ is —N(R₂—R₁)—CO— and A₃ and A₄ have a meaning as described for compounds of formulae (I) or (I′) before.

In compounds of formula (I′-f), the substituent A₁-A₂ is —CO—N(R₂—R₁)— and A₃ and A₄ have a meaning as described for compounds of formulae (I) or (I′) before.

In compounds of formula (I′-g), the substituent A₂-A₃ is —N(R₂—R₁)—CO— and A₁ and A₄ have a meaning as described for compounds of formulae (I) or (I′) before.

In compounds of formula (I′-h), the substituent A₂-A₃ is —CO—N(R₂—R₁)— and A₁ and A₄ have a meaning as described for compounds of formulae (I) or (I′) before.

In compounds of formula (I′-i), the substituent A₃-A₄ is —N(R₂—R₁)—CO— and A₁ and A₂ have a meaning as described for compounds of formulae (I) or (I′) before.

The invention is therefore additionally directed to an ophthalmic device or a precursor article for manufacturing an ophthalmic device comprising at least one polymerized compound of formula (I) wherein m1 is 0 which can preferably be described according to formulae (I′-a), (I′-b), (I′-c), (I′-d), (I′-e), (I′-f), (I′-g), (I′-h) and (I′-i),

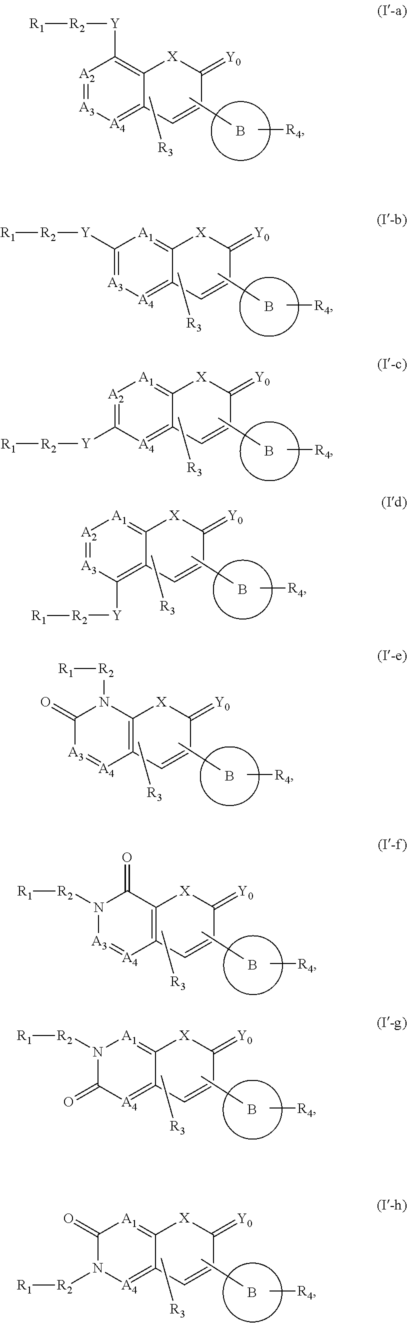

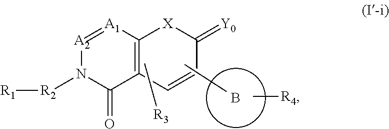

wherein R₁, —R₂—, X, Y₀, Y, A₁, A₂, A₃, A₄, R₃, R₄ and

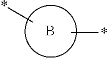

have a meaning as described before or preferably described before or below.

The invention is therefore additionally directed to compounds of formula (I) wherein n is 1 and m1 is 0 which can preferably be described according to formulae (I′-a), (I′-b), (I′-c), (I′-d), (I′-e), (I′-f), (I′-g), (I′-h) and (I′-i) as described before wherein R₁, —R₂—, X, Y₀, Y, A₁, A₂, A₃, A₄, R₃, R₄ and

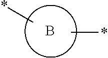

have a meaning as described before or preferably described before or below and where the given disclaimer have to be used as disclosed.

Preferred positions of R₁—R₂—Y—C or R₁—R₂—N are position 8 and/or 7 of the photoactive chromophore e.g. visualized in formulae (I′-a), (I′-b), (I′-e), (I′-g) and (I′-f).

In another preferred embodiment of the invention, the compounds of formula (I) acting as monomers for the preparation of the ophthalmic device or the precursor article for manufacturing of an ophthalmic device as described before or for the preparation of an oligomer, polymer or copolymer according to the invention or as compound according to the invention as described before contain the polymerizable group R₁ attached via —R₂— and Y to the divalent group linked to the photoactive chromophore. This is the case for compounds of formula (I) in which m1 is 1 which can be described accordingly in formula (I″).

The invention is therefore additionally directed to an ophthalmic device or a precursor article for manufacturing an ophthalmic device comprising at least one polymerized compound of formula (I″),

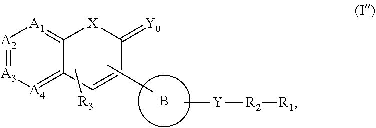

wherein R₁, —R₂—, Y, R₃, X, Y₀, R′, R″ and

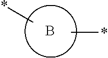

have a meaning as described before or preferably described before or below and

A₁, A₂, A₃, A₄ are each independently of each other N or CR″ provided that only one of A₁, A₂, A₃ and A₄ is N and the others are CR″.

The invention is therefore additionally directed to compounds of formula (I) wherein m1 is 1 which can preferably be described according to formula (I″),

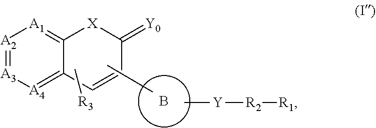

wherein R₁, —R₂—, Y, R₃, X, Y₀, R′, R″ and

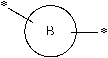

have a meaning as described before or preferably described before or below and

A₁, A₂, A₃, A₄ are each independently of each other N or CR″ provided that only one of A₁, A₂ and A₃ is N and the others and Ar₄ are CR″ and provided that in case of A₂ is CR″ and R″ is a linear or branched, non-halogenated, partially or completely halogenated alkoxy group having 1 to 20 C atoms and a linear or branched, non-halogenated and partially or completely halogenated thioalkyl group having 1 to 20 C atoms,

the divalent group

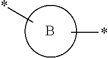

is in position 3 and is selected from formula (B-3), Y₂ within formula (B-3) is CR′ and R′ is H, Y₄ within formula (B-3) is N, and Y is a bond, O or S, c is 1;

provided that in case of Y is a bond, c is 0, R₃ is Cl and

the divalent group

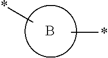

is in position 3, o is 5 to 20; provided that in case of X is O and Y₀ is O, o is 5 to 20; provided that in case of c is 0, Y is a bond or O and

the divalent group

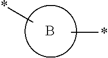

is in position 3, o is 11 to 20 and provided that in case of A₁ or A₃ is N, Y is a bond, c is 0,

the divalent group

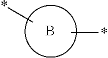

is in position 3, o is 7 to 20.

As described before within the ophthalmic device, precursor article for manufacturing an ophthalmic device, compounds of formulae (I), (I′), (I′-a), (I′-b), (I′-c), (I′-d), (I′-e), (I′-f), (I′-g), (I′-h), (I′-i) or (I″) and any oligomers, polymers or copolymers derived therefrom according to the invention, the substituent R″ is at each occurrence independently selected from the group consisting of H, F, Cl, Br, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated and partially or completely halogenated thioalkyl group having 1 to 20 C atoms.

R″ is at each occurrence independently preferably H, F, Cl, Br, CN or a linear or branched, non-halogenated, partially or completely halogenated alkyl group having 1 to 20 C atoms. R″ is particularly preferably H.

As described before within the ophthalmic device, precursor article for manufacturing an ophthalmic device, compounds of formulae (I), (I′), (I′-a), (I′-b), (I′-c), (I′-d), (I′-e), (I′-f), (I′-g) and (I′-h), (I′-i) or (I″) and any oligomers, polymers or copolymers derived therefrom according to the invention, the divalent group

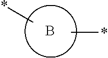

is selected from the group of formulae (B-1), (B-2), (B-3), (B-4) or (B-5)

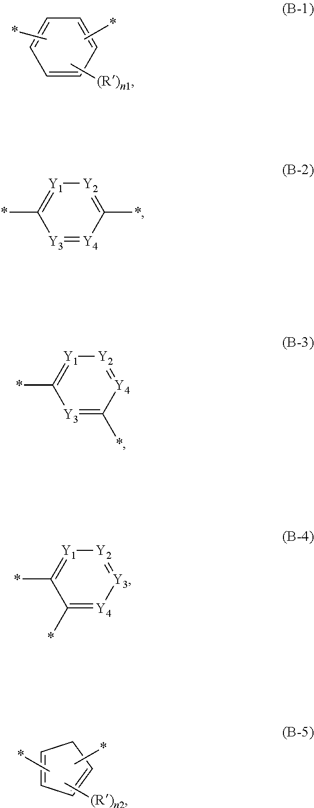

the asterisk * indicates the linkage to the remainder of formulae (I), (I′), (I′-a), (I′-b), (I′-c), (I′-d), (I′-e), (I′-f), (I′-g), (I′-h), (I′-i) or (I″):

Y₁, Y₂, Y₃, Y₄ are each independently of each other CR′ or N, provided that only one of Y₁, Y₂, Y₃ and Y₄ is N and the others are CR′;

Y₅ is O, S or NR_{B};

R_{B} is at each occurrence independently selected from a linear or branched alkyl group having 1 to 10 C atoms or a linear or branched partially or fully fluorinated alkyl group having 1 to 10 C atoms. R_{B} is preferably a linear or branched alkyl group having 1 to 4 C atoms or a linear or branched partially or fully fluorinated alkyl group having 1 to 4 C atoms.

Within said group of formula (B-5), Y₅ is preferably O or S.

In one embodiment of the invention, the divalent group

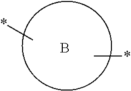

is preferably a group of formulae (B-1) to (B-4).

In one embodiment of the invention, the divalent group

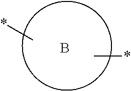

is preferably a group of formula (B-1).

As described before within the ophthalmic device, precursor article for manufacturing an ophthalmic device, compounds of formulae (I), (I′), (I′-a), (I′-b), (I′-c), (I′-d), (I′-e), (I′-f), (I′-g), (I′-h), (I′-i) or (I″) and any oligomers, polymers or copolymers derived therefrom according to the invention or in groups (B-1) to (B-5), the substituent R′ is at each occurrence independently selected from the group consisting H, F, SF₅, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated, partially or completely halogenated thioalkyl group having 1 to 20 C atoms.

R′ is at each occurrence independently preferably H, F, SF₅, CN, SO₂CF₃, a linear or branched, non-halogenated, partially or completely halogenated alkyl group having 1 to 10 C atoms, a non-halogenated, partially or completely halogenated cycloalkyl group having 3 to 6 C atoms, a linear or branched, non-halogenated, partially or completely halogenated alkoxy group having 1 to 10 C atoms and a linear or branched, non-halogenated, partially or completely halogenated thioalkyl group having 1 to 10 C atoms.

In one embodiment of the invention, preferably all R′ are H.

In one embodiment of the invention, preferably one R′ is different from H and the other substituents R′ are selected from the list as described before.

In one embodiment of the invention, preferably two R′ are different from H and the other substituents R′ are selected from the list as described before.

R′ is independently of each other particularly preferably selected from the group consisting of F, CN, SO₂CF₃, SF₅, methyl, ethyl, n-propyl, n-butyl, n-pentyl, n-hexyl, n-heptyl, n-octyl, trifluoromethyl, pentafluoroethyl, heptafluoropropyl, methoxy, ethoxy, propoxy, trifluoromethoxy, pentafluoroethoxy, thiomethyl and thioethyl.

R′ is independently of each other particularly preferably selected from the group consisting of F, ethyl, n-pentyl, trifluoromethyl, methoxy and trifluoromethoxy.

With regards to the compounds according to the invention, the described disclaimers have to be considered for the definition of R′.

As described before within the ophthalmic device, precursor article for manufacturing an ophthalmic device, compounds of formulae (I), (I′), (I′-a), (I′-b), (I′-c), (I′-d), (I′-e), (I′-f), (I′-g), (I′-h), (I′-i) or (I″) and any oligomers, polymers or copolymers derived therefrom according to the invention R₃ is H, F, Cl, Br, CN or a linear or branched, non-halogenated, partially or completely halogenated alkyl group having 1 to 20 C atoms. Preferably, R₃ is H, F or a linear or branched, non-halogenated, partially or completely halogenated alkyl group having 1 to 10 C atoms. Particularly preferably, R₃ is H.

As described before within the ophthalmic device, precursor article for manufacturing an ophthalmic device, compounds of formulae (I′), (I′-a), (I′-b), (I′-c), (I′-d), (I′-e), (I′-f), (I′-g), (I′-h) or (I′-i) and any oligomers, polymers or copolymers derived therefrom according to the invention R₄ is R′ and R′ has a meaning as described before or preferably described before.

As described before within the ophthalmic device, precursor article for manufacturing an ophthalmic device, compounds of formulae (I), (I′), (I′-a), (I′-b), (I′-c), (I′-d), (I′-e), (I′-f), (I′-g), (I′-h), (I′-i) or (I″) and any oligomers, polymers or copolymers derived therefrom according to the invention X is O or S, preferably O.

As described before within the ophthalmic device, precursor article for manufacturing an ophthalmic device, compounds of formulae (I), (I′), (I′-a), (I′-b), (I′-c), (I′-d), (I′-e), (I′-f), (I′-g), (I′-h), (I′-i) or (I″) and any oligomers, polymers or copolymers derived therefrom according to the invention Y₀ is O or S, preferably O.

The invention furthermore relates to an ophthalmic device or a precursor article for manufacturing an ophthalmic device comprising at least one polymerized compound of formulae (I), (I′), (I′-a), (I′-b), (I′-c), (I′-d), (I′-e), (I′-f), (I′-g), (I′-h), (I′-i) or (I″) as described before or preferably described before wherein X is O and Y₀ is O.

In these embodiments of the invention when m1 is 0 and R₄ is R′, the substitution pattern of group (B-1) is preferably selected from (S-1) to (S-12)

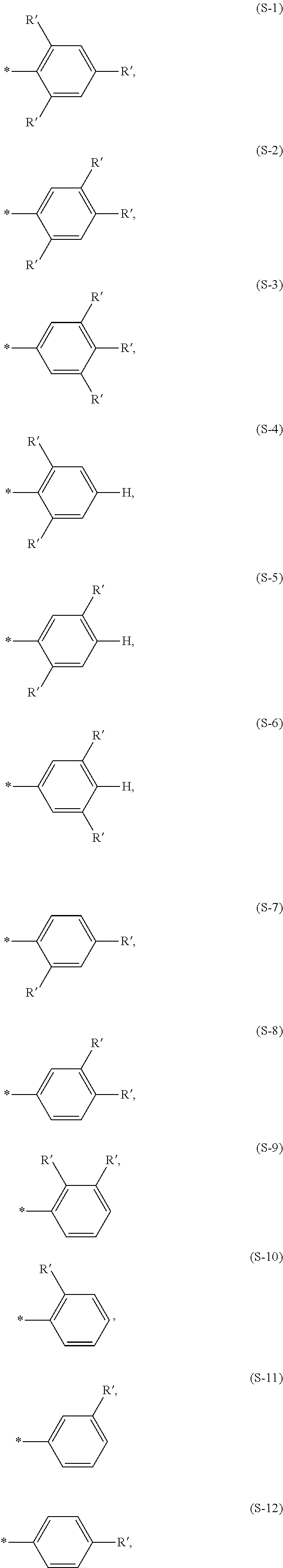

where R′ has independently of each other a meaning as described before or preferably described below. Preferred substitution patterns are (S-1), (S-7), (S-10), (S-11) and (S-12). Particularly preferred substitution patterns are (S-7) and/or (S-10) and/or (S-12). A very particularly preferred substitution pattern is (S-7). A very particularly preferred substitution pattern is (S-10). A very particularly preferred substitution pattern is (S-12).

As described before, the substituent R₄ corresponds to R₁ in case m1 is 1 in formula (I) or in formula (I″) wherein R′ in the divalent groups of formulae (B-1) to (B-5) have a meaning as described before or a preferred or particularly preferred meaning as described before.

In this embodiment where R₄ is R₁ and R₁ is linked via —R₂—Y— to the divalent group of formulae (B-1) to (B-4), such R₁—R₂—Y-group is preferably in ortho, meta or para position to the bond of said divalent group linked to the rest of formulae (I) or (I″). In this embodiment where R₄ is R₁ and R₁ is linked via —R₂—Y— to the divalent group, such R₁—R₂—Y-group is particularly preferably in ortho or para position to the bond of said divalent group of formula (B-1), (B-2) or (B-4) linked to the rest of formulae (I) or (I″). In this embodiment where R₄ is R₁ and R₁ is linked via —R₂—Y— to the divalent group, such R₁—R₂—Y-group is very particularly preferably in para position to the bond of said divalent group of formula (B-1) or (B-2) linked to the rest of formulae (I) or (I″).

Therefore, the invention is furthermore directed to an ophthalmic device or a precursor article for manufacturing an ophthalmic device comprising polymerized compounds of formulae (I) or (I″) wherein R₄ is R₁ and R′ has a meaning as described before or preferably described below and R₁ is linked via —R₂—Y— to the divalent group and such R₁—R₂—Y-group is in ortho or para position to the bond of said divalent group of formulae (B-1), (B-2) and (B-4) linked to the rest of said formulae (I) or (I″).

The preferred position of the divalent group

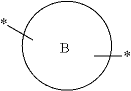

as described before or preferably described before is in position 3 of the photoactive chromophore.

The following formula summarizes the preferred positions of R₃ and the divalent group

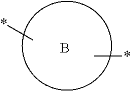

for compounds of formula (I) reading formula (I #),

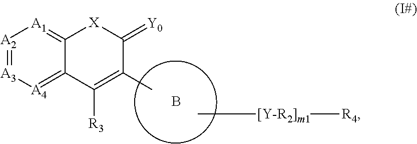

where R₁, —R₂—, X, Y₀, Y, R₃, R₄, m1,

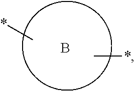

A₁, A₂, A₃ and A₄ have a meaning as described before or preferably described before or below. Such compounds according to formula (I #) act preferably as monomers of formulae (I) for the preparation of the ophthalmic device or the precursor article for manufacturing an ophthalmic device as described before or for the preparation of an oligomer, polymer or copolymer according to the invention.

According to the invention, compounds of formulae (I), (I #), (I′), (I′-a), (I′-b), (I′-c), (I′-d), (I′-e), (I′-f), (I′-g), (I′-h), (I′-i) and (I″) with substituents as described before or preferably described before have a polymerizable group as described before or preferably described before or below and have at least one linking element Y—R₂ or —R₂—.

According to the invention, Y is independently at each occurrence O, S, O═S═O or a bond.

According to the invention, the linking element —R₂— is selected from the group consisting of —(C(R)₂)ₒ—, or —(C(R)₂)ₚ—X₈—(C(R)₂)_{q}—(X₉)ₛ—(C(R)₂)ᵣ—(X₁₀)ᵣ(C(R)₂)ᵤ—, R is at each occurrence independently selected from the group consisting of H, F, a linear or branched alkyl group having 1 to 4 C atoms or a linear or branched partially or fully fluorinated alkyl group having 1 to 4 C atoms and o is selected from the group consisting of 1 to 20, X₈, X₉ and X₁₀ are at each occurrence O, S, SO₂, or NR₀, s and t are at each occurrence independently 0 or 1, p and q are at each occurrence independently selected from the group consisting of 1 to 10, r and u are at each occurrence independently selected from the group consisting of 0 to 10, wherein the overall number of atoms for —(C(R)₂)ₚ—X₈—(C(R)₂)_{q}—(X₉)ₛ—(C(R)₂)ᵣ—(X₁₀)ₜ—(C(R)₂)ᵤ—, is up to 20 C atoms. R₀ in NR₀ is at each occurrence independently selected from the group consisting of a linear or branched alkyl group having 1 to 4 C atoms and a linear or branched partially or fully fluorinated alkyl group having 1 to 4 C atoms. R₀ is independently at each occurrence and preferably Methyl, Ethyl or Trifluoromethyl. R₀ is independently at each occurrence particularly preferably Methyl.

According to the invention, R is at each occurrence independently selected from the group consisting of H, F, a linear or branched alkyl group having 1 to 8 C atoms or a linear or branched partially or fully fluorinated alkyl group having 1 to 4 C atoms.

R is particularly preferably at each occurrence independently H, F, methyl or ethyl. R is very particularly preferably H.

In another preferred embodiment of the invention, o is preferably selected from the group consisting of 7, 8, 9, 10, 11, 12, 13 and 14 within the compounds of formulae (I), (I #), (I′), (I′-a), (I′-b), (I′-c), (I′-d), (I′-e), (I′-f), (I′-g), (I′-h), (I′-i) and (I″) acting as monomers for the preparation of the ophthalmic device or the precursor article for manufacturing an ophthalmic device as described before or for the preparation of an oligomer, polymer or copolymer according to the invention or within the compounds according to the invention. Preferably, o is selected from the group consisting of 5, 6, 7, 8, 9, 10, 11, 12 and 13. Particularly preferably, o is selected from the group consisting of 8, 9, 10, 11, and 12. With regards to the compounds according to the invention, the described disclaimers have to be considered for the definition of o.

In another preferred embodiment of the invention, s, t, X₈, X₉, X₁₀, p, q, r and u within the compounds of formulae (I), (I #), (I′), (I′-a), (I′-b), (I′-c), (I′-d), (I′-e), (I′-f), (I′-g), (I′-h), (I′-i) and (I″) acting as monomers for the preparation of the ophthalmic device or the precursor article for manufacturing an ophthalmic device as described before or for the preparation of an oligomer, polymer or copolymer according to the invention or within the compounds according to the invention have the following preferred meaning:

Preferably, s is 1. Preferably, s is 0.

Preferably t is 0 or 1.

Preferably, s and t are 0.

Preferably, X₈, X₉ and X₁₀ are O, S or SO₂. Particularly preferably, X₈, X₉ and X₁₀ are O. Particularly preferably, X₈, X₉ and X₁₀ are S. Particularly preferably, X₈, X₉ and X₁₀ are SO₂.

Preferably, p and q are each independently 1, 3, 3, 4, 5 or 6, particularly preferably 1 or 2, very particularly preferably 2.

Preferably, r and u are each independently 0, 1, 2 or 3, particularly preferably 0, 1 or 2, very particularly preferably 0.

In case o is 0, —R₂— is a bond.

According to the invention, suitable examples for —R₂— are —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—, —(CH₂)₁₆—, —(CH₂)₁₇—, —(CH₂)₁₈—, —(CH₂)₁₉—, —(CH₂)₂₀—, —(CHCH₃)—, —(CHCH₃)₂—, —(CHCH₃)₃—, —(CHCH₃)₄—, —(CHCH₃)₅—, —(CHCH₃)₆—, —(CHCH₃)₇—, —(CHCH₃)₈—, —(CHCH₃)₉—, —(CHCH₃)₁₀—, —(CHCH₃)₁₁—, —(CHCH₃)₁₂—, —(CHCH₃)₁₃—, —(CHCH₃)₁₄—, —(CHCH₃)₁₅—, —(CHCH₃)₁₆—, —(CHCH₃)₁₇—, —(CHCH₃)₁₈—, —(CHCH₃)₁₉—, —(CHCH₃)₂₀—, —(C(CH₃)₂)—, —(C(CH₃)₂)₂—, —(C(CH₃)₂)₃—, —(C(CH₃)₂)₄—, —(C(CH₃)₂)₅—, —(C(CH₃)₂)₆—, —(C(CH₃)₂)₇—, —(C(CH₃)₂)₈—, —(C(CH₃)₂)₉—, —(C(CH₃)₂)₁₀—, —(C(CH₃)₂)₁₁—, —(C(CH₃)₂)₁₂—, —(C(CH₃)₂)₁₃—, —(C(CH₃)₂)₁₄—, —(C(CH₃)₂)₁₅—, —(C(CH₃)₂)₁₆—, —(C(CH₃)₂)₁₇—, —(C(CH₃)₂)₁₈—, —(C(CH₃)₂)₁₉—, —(C(CH₃)₂)₂₀—, —(CHC₂H₅)—, —(CHC₂H₅)₂—, —(CHC₂H₅)₃—, —(CHC₂H₅)₄—, —(CHC₂H₅)₅—, —(CHC₂H₅)₆—, —(CHC₂H₅)₇—, —(CHC₂H₅)₈—, —(CHC₂H₅)₉—, —(CHC₂H₅)₁₀—, —(CHC₂H₅)₁₁—, —(CHC₂H₅)₁₂—, —(CHC₂H₅)₁₃—, —(CHC₂H₅)₁₄—, —(CHC₂H₅)₁₅—, —(CHC₂H₅)₁₆—, —(CHC₂H₅)₁₇—, —(CHC₂H₅)₁₈—, —(CHC₂H₅)₁₉—, —(CHC₂H₅)₂₀—, —(CH₂)—(CHCH₃)—(CH₂)—, —(CH₂)—(CHCH₃)—(CH₂)₂—, —(CH₂)—(CHCH₃)—(CH₂)₃—, —(CH₂)—(CHCH₃)—(CH₂)₁₁—, —(CH₂)₂—(CHCH₃)—(CH₂)—, —(CH₂)₃—(CHCH₃)—(CH₂)—, —(CH₂)₁₁—(CHCH₃)—(CH₂)—, —(CH₂)₂—O—(CH₂)₂—, —(CH₂)₃—O—(CH₂)₃—, —(CH₂)₂—O—(CH₂)₂—O—(CH₂)₂—, —(CH₂)₃—O—(CH₂)₃—O—(CH₂)₃—, —(CH₂)₂—O—(CH₂)₂—O—(CH₂)₆—, —(CH₂)₆—O—(CH₂)₂—O—(CH₂)₂—, —(CH₂)₂—O—(CH₂)₂—O—(CH₂)₈—, —(CH₂)₈—O—(CH₂)₂—O—(CH₂)₂—, —(CH₂)₂—S—(CH₂)₂—, —(CH₂)₃—S—(CH₂)₃—, —(CH₂)₂—S—(CH₂)₂—S—(CH₂)₂—, —(CH₂)₃—S—(CH₂)₃—S—(CH₂)₃—, —(CH₂)₂—S—(CH₂)₂—S—(CH₂)₆—, —(CH₂)₆—S—(CH₂)₂—S—(CH₂)₂—, —(CH₂)₂—S—(CH₂)₂—S—(CH₂)₈—, —(CH₂)₈—S—(CH₂)₂—S—(CH₂)₂—, —(CH₂)₂—SO₂—(CH₂)₂—, —(CH₂)₃—SO₂—(CH₂)₃—, —(CH₂)₂—SO₂—(CH₂)₂—SO₂—(CH₂)₂—, —(CH₂)₃—SO₂—(CH₂)₃—SO₂—(CH₂)₃—, —(CH₂)₂—SO₂—(CH₂)₂—SO₂—(CH₂)₆—, —(CH₂)₆—SO₂—(CH₂)₂—SO₂—(CH₂)₂—, —(CH₂)₂—SO₂—(CH₂)₂—SO₂—(CH₂)₈—, —(CH₂)₈—SO₂—(CH₂)₂—SO₂—(CH₂)₂—, —(CH₂)—S—(CH₂)₂—O—(CH₂)—, —(CH₂)—SO₂—(CH₂)₂—O—(CH₂)—, —(CH₂)—SO₂—(CH₂)₂—S—(CH₂)—, —(CH₂)—O—(CH₂)₂—S—(CH₂)₂—O—(CH₂)—, —(CH₂)—S—(CH₂)₂—O—(CH₂)₂—S—(CH₂)—, —(CH₂)—SO₂—(CH₂)₂—O—(CH₂)₂—SO₂—(CH₂)—, —(CH₂)—S—(CH₂)₂—S—(CH₂)₂—S—(CH₂)—, —(CH₂)—SO₂—(CH₂)₂—SO₂—(CH₂)₂—SO₂—(CH₂)—, —(CH₂)—O—(CH₂)₂—SO₂—(CH₂)₂—O—(CH₂)—, —(CH₂)₂—(NCH₃)—(CH₂)₂—, —(CH₂)₃—(NCH₃)—(CH₂)₃—, —(CH₂)₂—(NCH₃)—(CH₂)₂—(NCH₃)—(CH₂)₂—, —(CH₂)₃—(NCH₃)—(CH₂)₃—(NCH₃)—(CH₂)₃—, —(CH₂)₂—(NCH₃)—(CH₂)₂—(NCH₃)—(CH₂)₆—, —(CH₂)₆—(NCH₃)—(CH₂)₂—(NCH₃)—(CH₂)₂—, —(CH₂)₂—(NCH₃)—(CH₂)₂—(NCH₃)—(CH₂)₈— and —(CH₂)₈—(NCH₃)—(CH₂)₂—(NCH₃)—(CH₂)₂—; —(CF₂)—(CH₂)—, —(CH₂)—(CF₂)—, —(CH₂)—(CF₂)—(CH₂)—, —(CH₂)—(CF₂)—(CH₂)₂—, —(CH₂)—(CF₂)—(CH₂)₃—, —(CH₂)—(CF₂)—(CH₂)₄—, —(CH₂)—(CF₂)—(CH₂)₅—, —(CH₂)—(CF₂)—(CH₂)₆—, —(CH₂)—(CF₂)—(CH₂)₇—, —(CH₂)—(CF₂)—(CH₂)₈—, —(CH₂)—(CF₂)—(CH₂)₉—, —(CH₂)—(CF₂)—(CH₂)₁₀—, —(CH₂)₂—(CF₂)—(CH₂)—, —(CH₂)₃—(CF₂)—(CH₂)—, —(CH₂)₄—(CF₂)—(CH₂)—, —(CH₂)₅—(CF₂)—(CH₂)—, —(CH₂)₆—(CF₂)—(CH₂)—, —(CH₂)₇—(CF₂)—(CH₂)—, —(CH₂)₈—(CF₂)—(CH₂)—, —(CH₂)₉—(CF₂)—(CH₂)—, —(CH₂)₁₀—(CF₂)—(CH₂)—, —(CH₂)₂—(CF₂)—(CH₂)₂—, —(CH₂)₃—(CF₂)—(CH₂)₃—, —(CH₂)₄—(CF₂)—(CH₂)₄—, —(CH₂)₅—(CF₂)—(CH₂)₅—, —(CH₂)₂—(CF₂)—(CH₂)—, —(CH₂)₂—(CF₂)—(CH₂)₃—, —(CH₂)₂—(CF₂)—(CH₂)₄—, —(CH₂)₂—(CF₂)—(CH₂)₅—, —(CH₂)₂—(CF₂)—(CH₂)₆—, —(CH₂)₂—(CF₂)—(CH₂)₇—, —(CH₂)₂—(CF₂)—(CH₂)₈—, —(CH₂)₂—(CF₂)—(CH₂)₉—, —(CH₂)₃—(CF₂)—(CH₂)—, —(CH₂)₃—(CF₂)—(CH₂)₂—, —(CH₂)₃—(CF₂)—(CH₂)₄—, —(CH₂)₃—(CF₂)—(CH₂)₅—, —(CH₂)₃—(CF₂)—(CH₂)₆—, —(CH₂)₃—(CF₂)—(CH₂)₇—, —(CH₂)₃—(CF₂)—(CH₂)₈—, —(CH₂)₄—(CF₂)—(CH₂)—, —(CH₂)₄—(CF₂)—(CH₂)₂—, —(CH₂)₄—(CF₂)—(CH₂)₃—, —(CH₂)₄—(CF₂)—(CH₂)₅—, —(CH₂)₄—(CF₂)—(CH₂)₆—, —(CH₂)₄—(CF₂)—(CH₂)₇—, —(CH₂)₅—(CF₂)—(CH₂)—, —(CH₂)₅—(CF₂)—(CH₂)₂—, —(CH₂)₅—(CF₂)—(CH₂)₃—, —(CH₂)₅—(CF₂)—(CH₂)₄—, —(CH₂)₅—(CF₂)—(CH₂)₆—, —(CH₂)₆—(CF₂)—(CH₂)—, —(CH₂)₆—(CF₂)—(CH₂)₂—, —(CH₂)₆—(CF₂)—(CH₂)₃—, —(CH₂)₆—(CF₂)—(CH₂)₄—, —(CH₂)₆—(CF₂)—(CH₂)₅—,

—(CFH)—(CH₂)—, —(CH₂)—(CFH)—, —(CH₂)—(CFH)—(CH₂)—, —(CH₂)—(CFH)—(CH₂)₂—, —(CH₂)—(CFH)—(CH₂)₃—, —(CH₂)—(CFH)—(CH₂)₄—, —(CH₂)—(CFH)—(CH₂)₅—, —(CH₂)—(CFH)—(CH₂)₆—, —(CH₂)—(CFH)—(CH₂)₇—, —(CH₂)—(CFH)—(CH₂)₈—, —(CH₂)—(CFH)—(CH₂)₉—, —(CH₂)—(CFH)—(CH₂)₁₀—, —(CH₂)₂—(CFH)—(CH₂)—, —(CH₂)₃—(CFH)—(CH₂)—, —(CH₂)₄—(CFH)—(CH₂)—, —(CH₂)₅—(CFH)—(CH₂)—, —(CH₂)₆—(CFH)—(CH₂)—, —(CH₂)₇—(CFH)—(CH₂)—, —(CH₂)₈—(CFH)—(CH₂)—, —(CH₂)₉—(CFH)—(CH₂)—, —(CH₂)₁₀—(CFH)—(CH₂)—, —(CH₂)₂—(CFH)—(CH₂)₂—, —(CH₂)₃—(CFH)—(CH₂)₃—, —(CH₂)₄—(CFH)—(CH₂)₄—, —(CH₂)₅—(CFH)—(CH₂)₅—, —(CH₂)₂—(CFH)—(CH₂)—, —(CH₂)₂—(CFH)—(CH₂)₃—, —(CH₂)₂—(CFH)—(CH₂)₄—, —(CH₂)₂—(CFH)—(CH₂)₅—, —(CH₂)₂—(CFH)—(CH₂)₆—, —(CH₂)₂—(CFH)—(CH₂)₇—, —(CH₂)₂—(CFH)—(CH₂)₈—, —(CH₂)₂—(CFH)—(CH₂)₉—, —(CH₂)₃—(CFH)—(CH₂)—, —(CH₂)₃—(CFH)—(CH₂)₂—, —(CH₂)₃—(CFH)—(CH₂)₄—, —(CH₂)₃—(CFH)—(CH₂)₅—, —(CH₂)₃—(CFH)—(CH₂)₆—, —(CH₂)₃—(CFH)—(CH₂)₇—, —(CH₂)₃—(CFH)—(CH₂)₈—, —(CH₂)₄—(CFH)—(CH₂)—, —(CH₂)₄—(CFH)—(CH₂)₂—, —(CH₂)₄—(CFH)—(CH₂)₃—, —(CH₂)₄—(CFH)—(CH₂)₅—, —(CH₂)₄—(CFH)—(CH₂)₆—, —(CH₂)₄—(CFH)—(CH₂)₇—, —(CH₂)₅—(CFH)—(CH₂)—, —(CH₂)₅—(CFH)—(CH₂)₂—, —(CH₂)₅—(CFH)—(CH₂)₃—, —(CH₂)₅—(CFH)—(CH₂)₄—, —(CH₂)₅—(CFH)—(CH₂)₆—, —(CH₂)₆—(CFH)—(CH₂)—, —(CH₂)₆—(CFH)—(CH₂)₂—, —(CH₂)₆—(CFH)—(CH₂)₃—, —(CH₂)₆—(CFH)—(CH₂)₄—, —(CH₂)₆—(CFH)—(CH₂)₅—,

—(CF₂)₂—(CH₂)—, —(CH₂)—(CF₂)₂—, —(CH₂)—(CF₂)₂—(CH₂)—, —(CH₂)—(CF₂)₂—(CH₂)₂—, —(CH₂)—(CF₂)₂—(CH₂)₃—, —(CH₂)—(CF₂)₂—(CH₂)₄—, —(CH₂)—(CF₂)₂—(CH₂)₅—, —(CH₂)—(CF₂)₂—(CH₂)₆—, —(CH₂)—(CF₂)₂—(CH₂)₇—, —(CH₂)—(CF₂)₂—(CH₂)₈—, —(CH₂)—(CF₂)₂—(CH₂)₉—, —(CH₂)₂—(CF₂)₂—(CH₂)—, —(CH₂)₃—(CF₂)₂—(CH₂)—, —(CH₂)₄—(CF₂)₂—(CH₂)—, —(CH₂)₅—(CF₂)₂—(CH₂)—, —(CH₂)₆—(CF₂)₂—(CH₂)—, —(CH₂)₇—(CF₂)₂—(CH₂)—, —(CH₂)₈—(CF₂)₂—(CH₂)—, —(CH₂)₉—(CF₂)₂—(CH₂)—, —(CH₂)₂—(CF₂)₂—(CH₂)₂—, —(CH₂)₃—(CF₂)₂—(CH₂)₃—, —(CH₂)₄—(CF₂)₂—(CH₂)₄—, —(CH₂)₅—(CF₂)₂—(CH₂)₅—, —(CH₂)₂—(CF₂)₂—(CH₂)—, —(CH₂)₂—(CF₂)₂—(CH₂)₃—, —(CH₂)₂—(CF₂)₂—(CH₂)₄—, —(CH₂)₂—(CF₂)₂—(CH₂)₅—, —(CH₂)₂—(CF₂)₂—(CH₂)₆—, —(CH₂)₂—(CF₂)₂—(CH₂)₇—, —(CH₂)₂—(CF₂)₂—(CH₂)₈—, —(CH₂)₃—(CF₂)₂—(CH₂)—, —(CH₂)₃—(CF₂)₂—(CH₂)₂—, —(CH₂)₃—(CF₂)₂—(CH₂)₄—, —(CH₂)₃—(CF₂)₂—(CH₂)₅—, —(CH₂)₃—(CF₂)₂—(CH₂)₆—, —(CH₂)₃—(CF₂)₂—(CH₂)₇—, —(CH₂)₄—(CF₂)₂—(CH₂)—, —(CH₂)₄—(CF₂)₂—(CH₂)₂—, —(CH₂)₄—(CF₂)₂—(CH₂)₃—, —(CH₂)₄—(CF₂)₂—(CH₂)₅—, —(CH₂)₄—(CF₂)₂—(CH₂)₆—, —(CH₂)₅—(CF₂)₂—(CH₂)—, —(CH₂)₅—(CF₂)₂—(CH₂)₂—, —(CH₂)₅—(CF₂)₂—(CH₂)₃—, —(CH₂)₅—(CF₂)₂—(CH₂)₄—, —(CH₂)₆—(CF₂)₂—(CH₂)—, —(CH₂)₆—(CF₂)₂—(CH₂)₂—, —(CH₂)₆—(CF₂)₂—(CH₂)₃—, —(CH₂)₆—(CF₂)₂—(CH₂)₄—,

—(CFH)₂—(CH₂)—, —(CH₂)—(CFH)₂—, —(CH₂)—(CFH)₂—(CH₂)—, —(CH₂)—(CFH)₂—(CH₂)₂—, —(CH₂)—(CFH)₂—(CH₂)₃—, —(CH₂)—(CFH)₂—(CH₂)₄—, —(CH₂)—(CFH)₂—(CH₂)₅—, —(CH₂)—(CFH)₂—(CH₂)₆—, —(CH₂)—(CFH)₂—(CH₂)₇—, —(CH₂)—(CFH)₂—(CH₂)₈—, —(CH₂)—(CFH)₂—(CH₂)₉—, —(CH₂)₂—(CFH)₂—(CH₂)—, —(CH₂)₃—(CFH)₂—(CH₂)—, —(CH₂)₄—(CFH)₂—(CH₂)—, —(CH₂)₅—(CFH)₂—(CH₂)—, —(CH₂)₆—(CFH)₂—(CH₂)—, —(CH₂)₇—(CFH)₂—(CH₂)—, —(CH₂)₈—(CFH)₂—(CH₂)—, —(CH₂)₉—(CFH)₂—(CH₂)—, —(CH₂)₂—(CFH)₂—(CH₂)₂—, —(CH₂)₃—(CFH)₂—(CH₂)₃—, —(CH₂)₄—(CFH)₂—(CH₂)₄—, —(CH₂)₅—(CFH)₂—(CH₂)₅—, —(CH₂)₂—(CFH)₂—(CH₂)—, —(CH₂)₂—(CFH)₂—(CH₂)₃—, —(CH₂)₂—(CFH)₂—(CH₂)₄—, —(CH₂)₂—(CFH)₂—(CH₂)₅—, —(CH₂)₂—(CFH)₂—(CH₂)₆—, —(CH₂)₂—(CFH)₂—(CH₂)₇—, —(CH₂)₂—(CFH)₂—(CH₂)₈—, —(CH₂)₃—(CFH)₂—(CH₂)—, —(CH₂)₃—(CFH)₂—(CH₂)₂—, —(CH₂)₃—(CFH)₂—(CH₂)₄—, —(CH₂)₃—(CFH)₂—(CH₂)₅—, —(CH₂)₃—(CFH)₂—(CH₂)₆—, —(CH₂)₃—(CFH)₂—(CH₂)₇—, —(CH₂)₄—(CFH)₂—(CH₂)—, —(CH₂)₄—(CFH)₂—(CH₂)₂—, —(CH₂)₄—(CFH)₂—(CH₂)₃—, —(CH₂)₄—(CFH)₂—(CH₂)₅—, —(CH₂)₄—(CFH)₂—(CH₂)₆—, —(CH₂)₅—(CFH)₂—(CH₂)—, —(CH₂)₅—(CFH)₂—(CH₂)₂—, —(CH₂)₅—(CFH)₂—(CH₂)₃—, —(CH₂)₅—(CFH)₂—(CH₂)₄—, —(CH₂)₆—(CFH)₂—(CH₂)—, —(CH₂)₆—(CFH)₂—(CH₂)₂—, —(CH₂)₆—(CFH)₂—(CH₂)₃—, —(CH₂)₆—(CFH)₂—(CH₂)₄—,

—(CF₂)₃—(CH₂)—, —(CH₂)—(CF₂)₃—, —(CH₂)—(CF₂)₃—(CH₂)—, —(CH₂)—(CF₂)₃—(CH₂)₂—, —(CH₂)—(CF₂)₃—(CH₂)₃—, —(CH₂)—(CF₂)₃—(CH₂)₄—, —(CH₂)—(CF₂)₃—(CH₂)₅—, —(CH₂)—(CF₂)₃—(CH₂)₆—, —(CH₂)—(CF₂)₃—(CH₂)₇—, —(CH₂)—(CF₂)₃—(CH₂)₈—, —(CH₂)₂—(CF₂)₃—(CH₂)—, —(CH₂)₃—(CF₂)₃—(CH₂)—, —(CH₂)₄—(CF₂)₃—(CH₂)—, —(CH₂)₅—(CF₂)₃—(CH₂)—, —(CH₂)₆—(CF₂)₃—(CH₂)—, —(CH₂)₇—(CF₂)₃—(CH₂)—, —(CH₂)₈—(CF₂)₃—(CH₂)—, —(CH₂)₂—(CF₂)₃—(CH₂)₂—, —(CH₂)₃—(CF₂)₃—(CH₂)₃—, —(CH₂)₄—(CF₂)₃—(CH₂)₄—, —(CH₂)₂—(CF₂)₃—(CH₂)—, —(CH₂)₂—(CF₂)₃—(CH₂)₃—, —(CH₂)₂—(CF₂)₃—(CH₂)₄—, —(CH₂)₂—(CF₂)₃—(CH₂)₅—, —(CH₂)₂—(CF₂)₃—(CH₂)₆—, —(CH₂)₂—(CF₂)₃—(CH₂)₇—, —(CH₂)₃—(CF₂)₃—(CH₂)—, —(CH₂)₃—(CF₂)₃—(CH₂)₂—, —(CH₂)₃—(CF₂)₃—(CH₂)₄—, —(CH₂)₃—(CF₂)₃—(CH₂)₅—, —(CH₂)₃—(CF₂)₃—(CH₂)₆—, —(CH₂)₄—(CF₂)₃—(CH₂)—, —(CH₂)₄—(CF₂)₃—(CH₂)₂—, —(CH₂)₄—(CF₂)₃—(CH₂)₃—, —(CH₂)₄—(CF₂)₃—(CH₂)₅—, —(CH₂)₅—(CF₂)₃—(CH₂)—, —(CH₂)₅—(CF₂)₃—(CH₂)₂—, —(CH₂)₅—(CF₂)₃—(CH₂)₃—, —(CH₂)₅—(CF₂)₃—(CH₂)₄—, (CH₂)₆—(CF₂)₃—(CH₂)—, —(CH₂)₆—(CF₂)₃—(CH₂)₂—, —(CH₂)₆—(CF₂)₃—(CH₂)₃—,

—(CF₂)₄—(CH₂)—, —(CH₂)—(CF₂)₄—, —(CH₂)—(CF₂)₄—(CH₂)—, —(CH₂)—(CF₂)₄—(CH₂)₂—, —(CH₂)—(CF₂)₄—(CH₂)₃—, —(CH₂)—(CF₂)₄—(CH₂)₄—, —(CH₂)—(CF₂)₄—(CH₂)₅—, —(CH₂)—(CF₂)₄—(CH₂)₆—, —(CH₂)—(CF₂)₄—(CH₂)₇—, —(CH₂)—(CF₂)₄—(CH₂)₈—, —(CH₂)—(CF₂)₄—(CH₂)₉—, —(CH₂)—(CF₂)₄—(CH₂)₁₀—, —(CH₂)₂—(CF₂)₄—(CH₂)—, —(CH₂)₃—(CF₂)₄—(CH₂)—, —(CH₂)₄—(CF₂)₄—(CH₂)—, —(CH₂)₅—(CF₂)₄—(CH₂)—, —(CH₂)₆—(CF₂)₄—(CH₂)—, —(CH₂)₇—(CF₂)₄—(CH₂)—, —(CH₂)₂—(CF₂)₄—(CH₂)₂—, —(CH₂)₃—(CF₂)₄—(CH₂)₃—, —(CH₂)₄—(CF₂)₄—(CH₂)₄—, —(CH₂)₅—(CF₂)₄—(CH₂)₅—, —(CH₂)₂—(CF₂)₄—(CH₂)₃—, —(CH₂)₂—(CF₂)₄—(CH₂)₄—, —(CH₂)₂—(CF₂)₄—(CH₂)₅—, —(CH₂)₂—(CF₂)₄—(CH₂)₆—, —(CH₂)₃—(CF₂)₄—(CH₂)₂—, —(CH₂)₃—(CF₂)₄—(CH₂)₄—, —(CH₂)₄—(CF₂)₄—(CH₂)₂—, —(CH₂)₄—(CF₂)₄—(CH₂)₃—, —(CH₂)₅—(CF₂)₄—(CH₂)₂—, —(CH₂)₅—(CF₂)₄—(CH₂)₃—, —(CH₂)₆—(CF₂)₄—(CH₂)₂—,

—(CF₂)₅—(CH₂)—, —(CH₂)—(CF₂)₅—, —(CH₂)—(CF₂)₅—(CH₂)—, —(CH₂)—(CF₂)₅—(CH₂)₂—, —(CH₂)—(CF₂)₅—(CH₂)₃—, —(CH₂)—(CF₂)₅—(CH₂)₄—, —(CH₂)—(CF₂)₅—(CH₂)₅—, —(CH₂)—(CF₂)₅—(CH₂)₆—, —(CH₂)₂—(CF₂)₅—(CH₂)—, —(CH₂)₃—(CF₂)₅—(CH₂)—, —(CH₂)₄—(CF₂)₅—(CH₂)—, —(CH₂)₅—(CF₂)₅—(CH₂)—, —(CH₂)₆—(CF₂)₅—(CH₂)—, —(CH₂)₂—(CF₂)₅—(CH₂)₂—, —(CH₂)₃—(CF₂)₅—(CH₂)₃—, —(CH₂)₄—(CF₂)₅—(CH₂)₄—, —(CH₂)₂—(CF₂)₅—(CH₂)₃—, —(CH₂)₂—(CF₂)₅—(CH₂)₄—, —(CH₂)₂—(CF₂)₅—(CH₂)₅—, —(CH₂)₂—(CF₂)₅—(CH₂)₆—, —(CH₂)₃—(CF₂)₅—(CH₂)₂—, —(CH₂)₃—(CF₂)₅—(CH₂)₄—, —(CH₂)₄—(CF₂)₅—(CH₂)₂—, —(CH₂)₄—(CF₂)₅—(CH₂)₃—, —(CH₂)(CF₂)₅—(CH₂)₂—,

—(CHCF₃)—(CH₂)—, —(CH₂)—(CHCF₃)—, —(CH₂)—(CHCF₃)—(CH₂)—, —(CH₂)—(CHCF₃)—(CH₂)₂—, —(CH₂)—(CHCF₃)—(CH₂)₃—, —(CH₂)—(CHCF₃)—(CH₂)₄—, —(CH₂)—(CHCF₃)—(CH₂)₅—, —(CH₂)—(CHCF₃)—(CH₂)₆—, —(CH₂)—(CHCF₃)—(CH₂)₇—, —(CH₂)—(CHCF₃)—(CH₂)₈—, —(CH₂)—(CHCF₃)—(CH₂)₉—, —(CH₂)—(CHCF₃)—(CH₂)₁₀—, —(CH₂)₂—(CHCF₃)—(CH₂)—, —(CH₂)₃—(CHCF₃)—(CH₂)—, —(CH₂)₄—(CHCF₃)—(CH₂)—, —(CH₂)₅—(CHCF₃)—(CH₂)—, —(CH₂)₆—(CHCF₃—(CH₂)—, —(CH₂)₇—(CHCF₃)—(CH₂)—, —(CH₂)₈—(CHCF₃)—(CH₂)—, —(CH₂)₉—(CHCF₃)—(CH₂)—, —(CH₂)₁₀—(CHCF₃)—(CH₂)—, —(CH₂)₂—(CHCF₃)—(CH₂)₂—, —(CH₂)₃—(CHCF₃)—(CH₂)₃—, —(CH₂)₄—(CHCF₃)—(CH₂)₄—, —(CH₂)₅—(CHCF₃)—(CH₂)₅—, —(CH₂)₂—(CHCF₃)—(CH₂)₃—, —(CH₂)₂—(CHCF₃)—(CH₂)₄—, —(CH₂)₂—(CHCF₃)—(CH₂)₅—, —(CH₂)₂—(CHCF₃)—(CH₂)₆—, —(CH₂)₂—(CHCF₃)—(CH₂)₇—, —(CH₂)₂—(CHCF₃)—(CH₂)₈—, —(CH₂)₂—(CHCF₃)—(CH₂)₉—, —(CH₂)₃—(CHCF₃)—(CH₂)₂—, —(CH₂)₃—(CHCF₃)—(CH₂)₄—, —(CH₂)₃—(CHCF₃)—(CH₂)₅—, —(CH₂)₃—(CHCF₃)—(CH₂)₆—, —(CH₂)₃—(CHCF₃)—(CH₂)₇—, —(CH₂)₃—(CHCF₃)—(CH₂)₈—, —(CH₂)₄—(CHCF₃)—(CH₂)₂—, —(CH₂)₄—(CHCF₃)—(CH₂)₃—, —(CH₂)₄—(CHCF₃)—(CH₂)₅—, —(CH₂)₄—(CHCF₃)—(CH₂)₆—, —(CH₂)₄—(CHCF₃)—(CH₂)₇—, —(CH₂)₅—(CHCF₃)—(CH₂)₂—, —(CH₂)₅—(CHCF₃)—(CH₂)₃—, —(CH₂)₅—(CHCF₃)—(CH₂)₄—, —(CH₂)₅—(CHCF₃)—(CH₂)₆—, —(CH₂)₆—(CHCF₃)—(CH₂)₂—, —(CH₂)₆—(CHCF₃)—(CH₂)₃—, —(CH₂)₆—(CHCF₃)—(CH₂)₄—, —(CH₂)₆—(CHCF₃)—(CH₂)₅—,

—(CHCF₃)₂—(CH₂)—, —(CH₂)—(CHCF₃)₂—, —(CH₂)—(CHCF₃)₂—(CH₂)—, —(CH₂)—(CHCF₃)₂—(CH₂)₂—, —(CH₂)—(CHCF₃)₂—(CH₂)₃—, —(CH₂)—(CHCF₃)₂—(CH₂)₄—, —(CH₂)—(CHCF₃)₂—(CH₂)₅—, —(CH₂)—(CHCF₃)₂—(CH₂)₆—, —(CH₂)—(CHCF₃)₂—(CH₂)₇—, —(CH₂)—(CHCF₃)₂—(CH₂)₈—, —(CH₂)—(CHCF₃)₂—(CH₂)₉—, —(CH₂)₂—(CHCF₃)₂—(CH₂)—, —(CH₂)₃—(CHCF₃)₂—(CH₂)—, —(CH₂)₄—(CHCF₃)₂—(CH₂)—, —(CH₂)₅—(CHCF₃)₂—(CH₂)—, —(CH₂)₆—(CHCF₃)₂—(CH₂)—, —(CH₂)₇—(CHCF₃)₂—(CH₂)—, —(CH₂)₈—(CHCF₃)₂—(CH₂)—, —(CH₂)₉—(CHCF₃)₂—(CH₂)—, —(CH₂)₂—(CHCF₃)₂—(CH₂)₂—, —(CH₂)₃—(CHCF₃)₂—(CH₂)₃—, —(CH₂)₄—(CHCF₃)₂—(CH₂)₄—, —(CH₂)₅—(CHCF₃)₂—(CH₂)₅—, —(CH₂)₂—(CHCF₃)₂—(CH₂)₃—, —(CH₂)₂—(CHCF₃)₂—(CH₂)₄—, —(CH₂)₂—(CHCF₃)₂—(CH₂)₅—, —(CH₂)₂—(CHCF₃)₂—(CH₂)₆—, —(CH₂)₂—(CHCF₃)₂—(CH₂)₇—, —(CH₂)₂—(CHCF₃)₂—(CH₂)₈—, —(CH₂)₃—(CHCF₃)₂—(CH₂)₂—, —(CH₂)₃—(CHCF₃)₂—(CH₂)₄—, —(CH₂)₃—(CHCF₃)₂—(CH₂)₅—, —(CH₂)₃—(CHCF₃)₂—(CH₂)₆—, —(CH₂)₃—(CHCF₃)₂—(CH₂)₇—, —(CH₂)₄—(CHCF₃)₂—(CH₂)₂—, —(CH₂)₄—(CHCF₃)₂—(CH₂)₃—, —(CH₂)₄—(CHCF₃)₂—(CH₂)₅—, —(CH₂)₄—(CHCF₃)₂—(CH₂)₆—, —(CH₂)₅—(CHCF₃)₂—(CH₂)₂—, —(CH₂)₅—(CHCF₃)₂—(CH₂)₃—, —(CH₂)₅—(CHCF₃)₂—(CH₂)₄—, —(CH₂)₆—(CHCF₃)₂—(CH₂)₂—, —(CH₂)₆—(CHCF₃)₂—(CH₂)₃—, —(CH₂)₆—(CHCF₃)₂—(CH₂)₄—,

—(CHCF₃)₃—(CH₂)—, —(CH₂)—(CHCF₃)₃—, —(CH₂)—(CHCF₃)₃—(CH₂)—, —(CH₂)—(CHCF₃)₃—(CH₂)₂—, —(CH₂)—(CHCF₃)₃—(CH₂)₃—, —(CH₂)—(CHCF₃)₃—(CH₂)₄—, —(CH₂)—(CHCF₃)₃—(CH₂)₅—, —(CH₂)—(CHCF₃)₃—(CH₂)₆—, —(CH₂)—(CHCF₃)₃—(CH₂)₇—, —(CH₂)—(CHCF₃)₃—(CH₂)₈—, —(CH₂)₂—(CHCF₃)₃—(CH₂)—, —(CH₂)₃—(CHCF₃)₃—(CH₂)—, —(CH₂)₄—(CHCF₃)₃—(CH₂)—, —(CH₂)₅—(CHCF₃)₃—(CH₂)—, —(CH₂)₆—(CHCF₃)₃—(CH₂)—, —(CH₂)₇—(CHCF₃)₃—(CH₂)—, —(CH₂)₈—(CHCF₃)₃—(CH₂)—, —(CH₂)₂—(CHCF₃)₃—(CH₂)₂—, —(CH₂)₃—(CHCF₃)₃—(CH₂)₃—, —(CH₂)₄—(CHCF₃)₃—(CH₂)₄—, —(CH₂)₂—(CHCF₃)₃—(CH₂)₃—, —(CH₂)₂—(CHCF₃)₃—(CH₂)₄—, —(CH₂)₂—(CHCF₃)₃—(CH₂)₅—, —(CH₂)₂—(CHCF₃)₃—(CH₂)₆—, —(CH₂)₂—(CHCF₃)₃—(CH₂)₇—, —(CH₂)₃—(CHCF₃)₃—(CH₂)₂—, —(CH₂)₃—(CHCF₃)₃—(CH₂)₄—, —(CH₂)₃—(CHCF₃)₃—(CH₂)₅—, —(CH₂)₃—(CHCF₃)₃—(CH₂)₆—, —(CH₂)₄—(CHCF₃)₃—(CH₂)₂—, —(CH₂)₄—(CHCF₃)₃—(CH₂)₃—, —(CH₂)₄—(CHCF₃)₃—(CH₂)₅—, —(CH₂)₅—(CHCF₃)₃—(CH₂)₂—, —(CH₂)₅—(CHCF₃)₃—(CH₂)₃—, —(CH₂)₅—(CHCF₃)₃—(CH₂)₄—, —(CH₂)₆—(CHCF₃)₃—(CH₂)₂—, —(CH₂)₆—(CHCF₃)₃—(CH₂)₃—,

—(CHCF₃)₄—(CH₂)—, —(CH₂)—(CHCF₃)₄—, —(CH₂)—(CHCF₃)₄—(CH₂)—, —(CH₂)—(CHCF₃)₄—(CH₂)₂—, —(CH₂)—(CHCF₃)₄—(CH₂)₃—, —(CH₂)—(CHCF₃)₄—(CH₂)₄—, —(CH₂)—(CHCF₃)₄—(CH₂)₅—, —(CH₂)—(CHCF₃)₄—(CH₂)₆—, —(CH₂)—(CHCF₃)₄—(CH₂)₇—, —(CH₂)—(CHCF₃)₄—(CH₂)₈—, —(CH₂)—(CHCF₃)₄—(CH₂)₉—, —(CH₂)—(CHCF₃)₄—(CH₂)₁₀—, —(CH₂)₂—(CHCF₃)₄—(CH₂)—, —(CH₂)₃—(CHCF₃)₄—(CH₂)—, —(CH₂)₄—(CHCF₃)₄—(CH₂)—, —(CH₂)₅—(CHCF₃)₄—(CH₂)—, —(CH₂)₆—(CHCF₃)₄—(CH₂)—, —(CH₂)₇—(CHCF₃)₄—(CH₂)—, —(CH₂)₂—(CHCF₃)₄—(CH₂)₂—, —(CH₂)₃—(CHCF₃)₄—(CH₂)₃—, —(CH₂)₄—(CHCF₃)₄—, (CH₂)₄—, —(CH₂)₅—(CHCF₃)₄—(CH₂)₅—, —(CH₂)₂—(CHCF₃)₄—(CH₂)₃—, —(CH₂)₂—(CHCF₃)₄—(CH₂)₄—, —(CH₂)₂—(CHCF₃)₄—(CH₂)₅—, —(CH₂)₂—(CHCF₃)₄—(CH₂)₆—, —(CH₂)₃—(CHCF₃)₄—(CH₂)₂—, —(CH₂)₃—(CHCF₃)₄—(CH₂)₄—, —(CH₂)₄—(CHCF₃)₄—(CH₂)₂—, —(CH₂)₄—(CHCF₃)₄—(CH₂)₃—, —(CH₂)₅—(CHCF₃)₄—(CH₂)₂—, —(CH₂)₅—(CHCF₃)₄—(CH₂)₃—, —(CH₂)₆—(CHCF₃)₄—(CH₂)₂—, —(CHCF₃)₅—(CH₂)—, —(CH₂)—(CHCF₃)₅—, —(CH₂)—(CHCF₃)₅—(CH₂)—, —(CH₂)—(CHCF₃)₅—(CH₂)₂—, —(CH₂)—(CHCF₃)₅—(CH₂)₃—, —(CH₂)—(CHCF₃)₅—(CH₂)₄—, —(CH₂)—(CHCF₃)₅—(CH₂)₅—, —(CH₂)—(CHCF₃)₅—(CH₂)₆—, —(CH₂)₂—(CHCF₃)₅—(CH₂)—, —(CH₂)₃—(CHCF₃)₅—(CH₂)—, —(CH₂)₄—(CHCF₃)₅—(CH₂)—, —(CH₂)₅—(CHCF₃)₅—(CH₂)—, —(CH₂)₆—(CHCF₃)₅—(CH₂)—, —(CH₂)₂—(CHCF₃)₅—(CH₂)₂—, —(CH₂)₃—(CHCF₃)₅—(CH₂)₃—, —(CH₂)₄—(CHCF₃)₅—(CH₂)₄—, —(CH₂)₂—(CHCF₃)₅—(CH₂)₃—, —(CH₂)₂—(CHCF₃)₅—(CH₂)₄—, —(CH₂)₂—(CHCF₃)₅—(CH₂)₅—, —(CH₂)₂—(CHCF₃)₅—(CH₂)₆—, —(CH₂)₃—(CHCF₃)₅—(CH₂)₂—, —(CH₂)₃—(CHCF₃)₅—(CH₂)₄—, —(CH₂)₄—(CHCF₃)₅—(CH₂)₂—, —(CH₂)₄—(CHCF₃)₅—(CH₂)₃—, —(CH₂)₅—(CHCF₃)₅—(CH₂)₂—,

—[C(CH₃)CF₃]—(CH₂)—, —(CH₂)—[C(CH₃)CF₃]—, —(CH₂)—[C(CH₃)CF₃]—(CH₂)—, —(CH₂)—[C(CH₃)CF₃]—(CH₂)₂—, —(CH₂)—[C(CH₃)CF₃]—(CH₂)₃—, —(CH₂)—[C(CH₃)CF₃]—(CH₂)₄—, —(CH₂)—[C(CH₃)CF₃]—(CH₂)₅—, —(CH₂)—[C(CH₃)CF₃]—(CH₂)₆—, —(CH₂)—[C(CH₃)CF₃]—(CH₂)₇—, —(CH₂)—[C(CH₃)CF₃]—(CH₂)₈—, —(CH₂)—[C(CH₃)CF₃]—(CH₂)₉—, —(CH₂)—[C(CH₃)CF₃]—(CH₂)₁₀—, —(CH₂)₂—[C(CH₃)CF₃]—(CH₂)—, —(CH₂)₃—[C(CH₃)CF₃]—(CH₂)—, —(CH₂)₄—[C(CH₃)CF₃]—(CH₂)—, —(CH₂)₅—[C(CH₃)CF₃]—(CH₂)—, —(CH₂)₆—[C(CH₃)CF₃]—(CH₂)—, —(CH₂)₇—[C(CH₃)CF₃]—(CH₂)—, —(CH₂)₈—[C(CH₃)CF₃]—(CH₂)—, —(CH₂)₉—[C(CH₃)CF₃]—(CH₂)—, —(CH₂)₁₀—[C(CH₃)CF₃]—(CH₂)—, —(CH₂)₂—[C(CH₃)CF₃]—(CH₂)₂—, —(CH₂)₃—[C(CH₃)CF₃]—(CH₂)₃—, —(CH₂)₄—[C(CH₃)CF₃]—(CH₂)₄—, —(CH₂)₅—[C(CH₃)CF₃]—(CH₂)₅—, —(CH₂)₂—[C(CH₃)CF₃]—(CH₂)₃—, —(CH₂)₂—[C(CH₃)CF₃]—(CH₂)₄—, —(CH₂)₂—[C(CH₃)CF₃]—(CH₂)₅—, —(CH₂)₂—[C(CH₃)CF₃]—(CH₂)₆—, —(CH₂)₂—[C(CH₃)CF₃]—(CH₂)₇—, —(CH₂)₂—[C(CH₃)CF₃]—(CH₂)₈—, —(CH₂)₂—[C(CH₃)CF₃]—(CH₂)₉—, —(CH₂)₃—[C(CH₃)CF₃]—(CH₂)₂—, —(CH₂)₃—[C(CH₃)CF₃]—(CH₂)₄—, —(CH₂)₃—[C(CH₃)CF₃]—(CH₂)₅—, —(CH₂)₃—[C(CH₃)CF₃]—(CH₂)₆—, —(CH₂)₃—[C(CH₃)CF₃]—(CH₂)₇—, —(CH₂)₃—[C(CH₃)CF₃]—(CH₂)₈—, —(CH₂)₄—[C(CH₃)CF₃]—(CH₂)₂—, —(CH₂)₄—[C(CH₃)CF₃]—(CH₂)₃—, —(CH₂)₄—[C(CH₃)CF₃]—(CH₂)₅—, —(CH₂)₄—[C(CH₃)CF₃]—(CH₂)₆—, —(CH₂)₄—[C(CH₃)CF₃]—(CH₂)₇—, —(CH₂)₅—[C(CH₃)CF₃]—(CH₂)₂—, —(CH₂)₅—[C(CH₃)CF₃]—(CH₂)₃—, —(CH₂)₅—[C(CH₃)CF₃]—(CH₂)₄—, —(CH₂)₅—[C(CH₃)CF₃]—(CH₂)₆—, —(CH₂)₆—[C(CH₃)CF₃]—(CH₂)₂—, —(CH₂)₆—[C(CH₃)CF₃]—(CH₂)₃—, —(CH₂)₆—[C(CH₃)CF₃]—(CH₂)₄—, —(CH₂)₆—[C(CH₃)CF₃]—(CH₂)₅—,

—[C(CH₃)CF₃]₂—(CH₂)—, —(CH₂)—[C(CH₃)CF₃]₂—, —(CH₂)—[C(CH₃)CF₃]₂—(CH₂)—, —(CH₂)—[C(CH₃)CF₃]₂—(CH₂)₂—, —(CH₂)—[C(CH₃)CF₃]₂—(CH₂)₃—, —(CH₂)—[C(CH₃)CF₃]₂—(CH₂)₄—, —(CH₂)—[C(CH₃)CF₃]₂—(CH₂)₅—, —(CH₂)—[C(CH₃)CF₃]₂—(CH₂)₆—, —(CH₂)—[C(CH₃)CF₃]₂—(CH₂)₇—, —(CH₂)—[C(CH₃)CF₃]₂—(CH₂)₈—, —(CH₂)—[C(CH₃)CF₃]₂—(CH₂)₉—, —(CH₂)₂—[C(CH₃)CF₃]₂—(CH₂)—, —(CH₂)₃—[C(CH₃)CF₃]₂—(CH₂)—, —(CH₂)₄—[C(CH₃)CF₃]₂—(CH₂)—, —(CH₂)₅—[C(CH₃)CF₃]₂—(CH₂)—, —(CH₂)₆—[C(CH₃)CF₃]₂—(CH₂)—, —(CH₂)₇—[C(CH₃)CF₃]₂—(CH₂)—, —(CH₂)₈—[C(CH₃)CF₃]₂—(CH₂)—, —(CH₂)₉—[C(CH₃)CF₃]₂—(CH₂)—, —(CH₂)₂—[C(CH₃)CF₃]₂—(CH₂)₂—, —(CH₂)₃—[C(CH₃)CF₃]₂—(CH₂)₃—, —(CH₂)₄—[C(CH₃)CF₃]₂—(CH₂)₄—, —(CH₂)₅—[C(CH₃)CF₃]₂—(CH₂)₅—, —(CH₂)₂—[C(CH₃)CF₃]₂—(CH₂)₃—, —(CH₂)₂—[C(CH₃)CF₃]₂—(CH₂)₄—, —(CH₂)₂—[C(CH₃)CF₃]₂—(CH₂)₅—, —(CH₂)₂—[C(CH₃)CF₃]₂—(CH₂)₆—, —(CH₂)₂—[C(CH₃)CF₃]₂—(CH₂)₇—, —(CH₂)₂—[C(CH₃)CF₃]₂—(CH₂)₈—, —(CH₂)₃—[C(CH₃)CF₃]₂—(CH₂)₂—, —(CH₂)₃—[C(CH₃)CF₃]₂—(CH₂)₄—, —(CH₂)₃—[C(CH₃)CF₃]₂—(CH₂)₅—, —(CH₂)₃—[C(CH₃)CF₃]₂—(CH₂)₆—, —(CH₂)₃—[C(CH₃)CF₃]₂—(CH₂)₇—, —(CH₂)₄—[C(CH₃)CF₃]₂—(CH₂)₂—, —(CH₂)₄—[C(CH₃)CF₃]₂—(CH₂)₃—, —(CH₂)₄—[C(CH₃)CF₃]₂—(CH₂)₅—, —(CH₂)₄—[C(CH₃)CF₃]₂—(CH₂)₆—, —(CH₂)₅—[C(CH₃)CF₃]₂—(CH₂)₂—, —(CH₂)₅—[C(CH₃)CF₃]₂—(CH₂)₃—, —(CH₂)₅—[C(CH₃)CF₃]₂—(CH₂)₄—, —(CH₂)₆—[C(CH₃)CF₃]₂—(CH₂)₂—, —(CH₂)₆—[C(CH₃)CF₃]₂—(CH₂)₃—, —(CH₂)₆—[C(CH₃)CF₃]₂—(CH₂)₄—,

—[C(CH₃)CF₃]₃—(CH₂)—, —(CH₂)—[C(CH₃)CF₃]₃—, —(CH₂)—[C(CH₃)CF₃]₃—(CH₂)—, —(CH₂)—[C(CH₃)CF₃]₃—(CH₂)₂—, —(CH₂)—[C(CH₃)CF₃]₃—(CH₂)₃—, —(CH₂)—[C(CH₃)CF₃]₃—(CH₂)₄—, —(CH₂)—[C(CH₃)CF₃]₃—(CH₂)₅—, —(CH₂)—[C(CH₃)CF₃]₃—(CH₂)₆—, —(CH₂)—[C(CH₃)CF₃]₃—(CH₂)₇—, —(CH₂)—[C(CH₃)CF₃]₃—(CH₂)₈—, —(CH₂)₂—[C(CH₃)CF₃]₃—(CH₂)—, —(CH₂)₃—[C(CH₃)CF₃]₃—(CH₂)—, —(CH₂)₄—[C(CH₃)CF₃]₃—(CH₂)—, —(CH₂)₅—[C(CH₃)CF₃]₃—(CH₂)—, —(CH₂)₆—[C(CH₃)CF₃]₃—(CH₂)—, —(CH₂)₇—[C(CH₃)CF₃]₃—(CH₂)—, —(CH₂)₈—[C(CH₃)CF₃]₃—(CH₂)—, —(CH₂)₂—[C(CH₃)CF₃]₃—(CH₂)₂—, —(CH₂)₃—[C(CH₃)CF₃]₃—(CH₂)₃—, —(CH₂)₄—[C(CH₃)CF₃]₃—(CH₂)₄—, —(CH₂)₂—[C(CH₃)CF₃]₃—(CH₂)₃—, —(CH₂)₂—[C(CH₃)CF₃]₃—(CH₂)₄—, —(CH₂)₂—[C(CH₃)CF₃]₃—(CH₂)₅—, —(CH₂)₂—[C(CH₃)CF₃]₃—(CH₂)₆—, —(CH₂)₂—[C(CH₃)CF₃]₃—(CH₂)₇—, —(CH₂)₃—[C(CH₃)CF₃]₃—(CH₂)₂—, —(CH₂)₃—[C(CH₃)CF₃]₃—(CH₂)₄—, —(CH₂)₃—[C(CH₃)CF₃]₃—(CH₂)₅—, —(CH₂)₃—[C(CH₃)CF₃]₃—(CH₂)₆—, —(CH₂)₄—[C(CH₃)CF₃]₃—(CH₂)₂—, —(CH₂)₄—[C(CH₃)CF₃]₃—(CH₂)₃—, —(CH₂)₄—[C(CH₃)CF₃]₃—(CH₂)₅—, —(CH₂)₅—[C(CH₃)CF₃]₃—(CH₂)₂—, —(CH₂)₅—[C(CH₃)CF₃]₃—(CH₂)₃—, —(CH₂)₅—[C(CH₃)CF₃]₃—(CH₂)₄—, —(CH₂)₆—[C(CH₃)CF₃]₃—(CH₂)₂—, —(CH₂)₆—[C(CH₃)CF₃]₃—(CH₂)₃—,

—[C(CH₃)CF₃]₄—(CH₂)—, —(CH₂)—[C(CH₃)CF₃]₄—, —(CH₂)—[C(CH₃)CF₃]₄—(CH₂)—, —(CH₂)—[C(CH₃)CF₃]₄—(CH₂)₂—, —(CH₂)—[C(CH₃)CF₃]₄—(CH₂)₃—, —(CH₂)—[C(CH₃)CF₃]₄—(CH₂)₄—, —(CH₂)—[C(CH₃)CF₃]₄—(CH₂)₅—, —(CH₂)—[C(CH₃)CF₃]₄—(CH₂)₆—, —(CH₂)—[C(CH₃)CF₃]₄—(CH₂)₇—, —(CH₂)—[C(CH₃)CF₃]₄—(CH₂)₈—, —(CH₂)—[C(CH₃)CF₃]₄—(CH₂)₉—, —(CH₂)—[C(CH₃)CF₃]₄—(CH₂)₁₀—, —(CH₂)₂—[C(CH₃)CF₃]₄—(CH₂)—, —(CH₂)₃—[C(CH₃)CF₃]₄—(CH₂)—, —(CH₂)₄—[C(CH₃)CF₃]₄—(CH₂)—, —(CH₂)₅—[C(CH₃)CF₃]₄—(CH₂)—, —(CH₂)₆—[C(CH₃)CF₃]₄—(CH₂)—, —(CH₂)₇—[C(CH₃)CF₃]₄—(CH₂)—, —(CH₂)₂—[C(CH₃)CF₃]₄—(CH₂)₂—, —(CH₂)₃—[C(CH₃)CF₃]₄—(CH₂)₃—, —(CH₂)₄—[C(CH₃)CF₃]₄—(CH₂)₄—, —(CH₂)₅—[C(CH₃)CF₃]₄—(CH₂)₅—, —(CH₂)₂—[C(CH₃)CF₃]₄—(CH₂)₃—, —(CH₂)₂—[C(CH₃)CF₃]₄—(CH₂)₄—, —(CH₂)₂—[C(CH₃)CF₃]₄—(CH₂)₅—, —(CH₂)₂—[C(CH₃)CF₃]₄—(CH₂)₆—, —(CH₂)₃—[C(CH₃)CF₃]₄—(CH₂)₂—, —(CH₂)₃—[C(CH₃)CF₃]₄—(CH₂)₄—, —(CH₂)₄—[C(CH₃)CF₃]₄—(CH₂)₂—, —(CH₂)₄—[C(CH₃)CF₃]₄—(CH₂)₃—, —(CH₂)₅—[C(CH₃)CF₃]₄—(CH₂)₂—, —(CH₂)₅—[C(CH₃)CF₃]₄—(CH₂)₃—, —(CH₂)₆—[C(CH₃)CF₃]₄—(CH₂)₂—,

—[C(CH₃)CF₃]₅—(CH₂)—, —(CH₂)—[C(CH₃)CF₃]₅—, —(CH₂)—[C(CH₃)CF₃]₅—(CH₂)—, —(CH₂)—[C(CH₃)CF₃]₅—(CH₂)₂—, —(CH₂)—[C(CH₃)CF₃]₅—(CH₂)₃—, —(CH₂)—[C(CH₃)CF₃]₅—(CH₂)₄—, —(CH₂)—[C(CH₃)CF₃]₅—(CH₂)₅—, —(CH₂)—[C(CH₃)CF₃]₅—(CH₂)₆—, —(CH₂)₂—[C(CH₃)CF₃]₅—(CH₂)—, —(CH₂)₃—[C(CH₃)CF₃]₅—(CH₂)—, —(CH₂)₄—[C(CH₃)CF₃]₅—(CH₂)—, —(CH₂)₅—[C(CH₃)CF₃]₅—(CH₂)—, —(CH₂)₆—[C(CH₃)CF₃]₅—(CH₂)—, —(CH₂)₂—[C(CH₃)CF₃]₅—(CH₂)₂—, —(CH₂)₃—[C(CH₃)CF₃]₅—(CH₂)₃—, —(CH₂)₄—[C(CH₃)CF₃]₅—(CH₂)₄—, —(CH₂)₂—[C(CH₃)CF₃]₅—(CH₂)₃—, —(CH₂)₂—[C(CH₃)CF₃]₅—(CH₂)₄—, —(CH₂)₂—[C(CH₃)CF₃]₅—(CH₂)₅—, —(CH₂)₂—[C(CH₃)CF₃]₅—(CH₂)₆—, —(CH₂)₃—[C(CH₃)CF₃]₅—(CH₂)₂—, —(CH₂)₃—[C(CH₃)CF₃]₅—(CH₂)₄—, —(CH₂)₄—[C(CH₃)CF₃]₅—(CH₂)₂—, —(CH₂)₄—[C(CH₃)CF₃]₅—(CH₂)₃—, —(CH₂)₅—[C(CH₃)CF₃]₅—(CH₂)₂—,

—[CH(CH₂CF₃)]—(CH₂)—, —(CH₂)—[CH(CH₂CF₃)]—, —(CH₂)—[CH(CH₂CF₃)]—(CH₂)—, —(CH₂)—[CH(CH₂CF₃)]—(CH₂)₂—, —(CH₂)—[CH(CH₂CF₃)]—(CH₂)₃—, —(CH₂)—[CH(CH₂CF₃)]—(CH₂)₄—, —(CH₂)—[CH(CH₂CF₃)]—(CH₂)₅—, —(CH₂)—[CH(CH₂CF₃)]—(CH₂)₆—, —(CH₂)—[CH(CH₂CF₃)]—(CH₂)₇—, —(CH₂)—[CH(CH₂CF₃)]—(CH₂)₈—, —(CH₂)—[CH(CH₂CF₃)]—(CH₂)₉—, —(CH₂)—[CH(CH₂CF₃)]—(CH₂)₁₀—, —(CH₂)₂—[CH(CH₂CF₃)]—(CH₂)—, —(CH₂)₃—[CH(CH₂CF₃)]—(CH₂)—, —(CH₂)₄—[CH(CH₂CF₃)]—(CH₂)—, —(CH₂)₅—[CH(CH₂CF₃)]—(CH₂)—, —(CH₂)₆—[CH(CH₂CF₃)]—(CH₂)—, —(CH₂)₇—[CH(CH₂CF₃)]—(CH₂)—, —(CH₂)₈—[CH(CH₂CF₃)]—(CH₂)—, —(CH₂)₉—[CH(CH₂CF₃)]—(CH₂)—, —(CH₂)₁₀—[CH(CH₂CF₃)]—(CH₂)—, —(CH₂)₂—[CH(CH₂CF₃)]—(CH₂)₂—, —(CH₂)₃—[CH(CH₂CF₃)]—(CH₂)₃—, —(CH₂)₄—[CH(CH₂CF₃)]—(CH₂)₄—, —(CH₂)₅—[CH(CH₂CF₃)]—(CH₂)₅—, —(CH₂)₂—[CH(CH₂CF₃)]—(CH₂)₃—, —(CH₂)₂—[CH(CH₂CF₃)]—(CH₂)₄—, —(CH₂)₂—[CH(CH₂CF₃)]—(CH₂)₅—, —(CH₂)₂—[CH(CH₂CF₃)]—(CH₂)₆—, —(CH₂)₂—[CH(CH₂CF₃)]—(CH₂)₇—, —(CH₂)₂—[CH(CH₂CF₃)]—(CH₂)₈—, —(CH₂)₂—[CH(CH₂CF₃)]—(CH₂)₉—, —(CH₂)₃—[CH(CH₂CF₃)]—(CH₂)₂—, —(CH₂)₃—[CH(CH₂CF₃)]—(CH₂)₄—, —(CH₂)₃—[CH(CH₂CF₃)]—(CH₂)₅—, —(CH₂)₃—[CH(CH₂CF₃)]—(CH₂)₆—, —(CH₂)₃—[CH(CH₂CF₃)]—(CH₂)₇—, —(CH₂)₃—[CH(CH₂CF₃)]—(CH₂)₈—, —(CH₂)₄—[CH(CH₂CF₃)]—(CH₂)₂—, —(CH₂)₄—[CH(CH₂CF₃)]—(CH₂)₃—, —(CH₂)₄—[CH(CH₂CF₃)]—(CH₂)₅—, —(CH₂)₄—[CH(CH₂CF₃)]—(CH₂)₆—, —(CH₂)₄—[CH(CH₂CF₃)]—(CH₂)₇—, —(CH₂)₅—[CH(CH₂CF₃)]—(CH₂)₂—, —(CH₂)₅—[CH(CH₂CF₃)]—(CH₂)₃—, —(CH₂)₅—[CH(CH₂CF₃)]—(CH₂)₄—, —(CH₂)₅—[CH(CH₂CF₃)]—(CH₂)₆—, —(CH₂)₆—[CH(CH₂CF₃)]—(CH₂)₂—, —(CH₂)₆—[CH(CH₂CF₃)]—(CH₂)₃—, —(CH₂)₆—[CH(CH₂CF₃)]—(CH₂)₄—, —(CH₂)₆—[CH(CH₂CF₃)]—(CH₂)₅—,

—[CH(CH₂CF₃)]₂—(CH₂)—, —(CH₂)—[CH(CH₂CF₃)]₂—, —(CH₂)—[CH(CH₂CF₃)]₂—(CH₂)—, —(CH₂)—[CH(CH₂CF₃)]₂—(CH₂)₂—, —(CH₂)—[CH(CH₂CF₃)]₂—(CH₂)₃—, —(CH₂)—[CH(CH₂CF₃)]₂—(CH₂)₄—, —(CH₂)—[CH(CH₂CF₃)]₂—(CH₂)₅—, —(CH₂)—[CH(CH₂CF₃)]₂—(CH₂)₆—, —(CH₂)—[CH(CH₂CF₃)]₂—(CH₂)₇—, —(CH₂)—[CH(CH₂CF₃)]₂—(CH₂)₈—, —(CH₂)—[CH(CH₂CF₃)]₂—(CH₂)₉—, —(CH₂)₂—[CH(CH₂CF₃)]₂—(CH₂)—, —(CH₂)₃—[CH(CH₂CF₃)]₂—(CH₂)—, —(CH₂)₄—[CH(CH₂CF₃)]₂—(CH₂)—, —(CH₂)₅—[CH(CH₂CF₃)]₂—(CH₂)—, —(CH₂)₆—[CH(CH₂CF₃)]₂—(CH₂)—, —(CH₂)₇—[CH(CH₂CF₃)]₂—(CH₂)—, —(CH₂)₈—[CH(CH₂CF₃)]₂—(CH₂)—, —(CH₂)₉—[CH(CH₂CF₃)]₂—(CH₂)—, —(CH₂)₂—[CH(CH₂CF₃)]₂—(CH₂)₂—, —(CH₂)₃—[CH(CH₂CF₃)]₂—(CH₂)₃—, —(CH₂)₄—[CH(CH₂CF₃)]₂—(CH₂)₄—, —(CH₂)₅—[CH(CH₂CF₃)]₂—(CH₂)₅—, —(CH₂)₂—[CH(CH₂CF₃)]₂—(CH₂)₃—, —(CH₂)₂—[CH(CH₂CF₃)]₂—(CH₂)₄—, —(CH₂)₂—[CH(CH₂CF₃)]₂—(CH₂)₅—, —(CH₂)₂—[CH(CH₂CF₃)]₂—(CH₂)₆—, —(CH₂)₂—[CH(CH₂CF₃)]₂—(CH₂)₇—, —(CH₂)₂—[CH(CH₂CF₃)]₂—(CH₂)₈—, —(CH₂)₃—[CH(CH₂CF₃)]₂—(CH₂)₂—, —(CH₂)₃—[CH(CH₂CF₃)]₂—(CH₂)₄—, —(CH₂)₃—[CH(CH₂CF₃)]₂—(CH₂)₅—, —(CH₂)₃—[CH(CH₂CF₃)]₂—(CH₂)₆—, —(CH₂)₃—[CH(CH₂CF₃)]₂—(CH₂)₇—, —(CH₂)₄—[CH(CH₂CF₃)]₂—(CH₂)₂—, —(CH₂)₄—[CH(CH₂CF₃)]₂—(CH₂)₃—, —(CH₂)₄—[CH(CH₂CF₃)]₂—(CH₂)₅—, —(CH₂)₄—[CH(CH₂CF₃)]₂—(CH₂)₆—, —(CH₂)₅—[CH(CH₂CF₃)]₂—(CH₂)₂—, —(CH₂)₅—[CH(CH₂CF₃)]₂—(CH₂)₃—, —(CH₂)₅—[CH(CH₂CF₃)]₂—(CH₂)₄—, —(CH₂)₆—[CH(CH₂CF₃)]₂—(CH₂)₂—, —(CH₂)₆—[CH(CH₂CF₃)]₂—(CH₂)₃—, —(CH₂)₆—[CH(CH₂CF₃)]₂—(CH₂)₄—,

—[CH(CH₂CF₃)]₃—(CH₂)—, —(CH₂)—[CH(CH₂CF₃)]₃—, —(CH₂)—[CH(CH₂CF₃)]₃—(CH₂)—, —(CH₂)—[CH(CH₂CF₃)]₃—(CH₂)₂—, —(CH₂)—[CH(CH₂CF₃)]₃—(CH₂)₃—, —(CH₂)—[CH(CH₂CF₃)]₃—(CH₂)₄—, —(CH₂)—[CH(CH₂CF₃)]₃—(CH₂)₅—, —(CH₂)—[CH(CH₂CF₃)]₃—(CH₂)₆—, —(CH₂)—[CH(CH₂CF₃)]₃—(CH₂)₇—, —(CH₂)—[CH(CH₂CF₃)]₃—(CH₂)₈—, —(CH₂)₂—[CH(CH₂CF₃)]₃—(CH₂)—, —(CH₂)₃—[CH(CH₂CF₃)]₃—(CH₂)—, —(CH₂)₄—[CH(CH₂CF₃)]₃—(CH₂)—, —(CH₂)₅—[CH(CH₂CF₃)]₃—(CH₂)—, —(CH₂)₆—[CH(CH₂CF₃)]₃—(CH₂)—, —(CH₂)₇—[CH(CH₂CF₃)]₃—(CH₂)—, —(CH₂)₈—[CH(CH₂CF₃)]₃—(CH₂)—, —(CH₂)₂—[CH(CH₂CF₃)]₃—(CH₂)₂—, —(CH₂)₃—[CH(CH₂CF₃)]₃—(CH₂)₃—, —(CH₂)₄—[CH(CH₂CF₃)]₃—(CH₂)₄—, —(CH₂)₂—[CH(CH₂CF₃)]₃—(CH₂)₃—, —(CH₂)₂—[CH(CH₂CF₃)]₃—(CH₂)₄—, —(CH₂)₂—[CH(CH₂CF₃)]₃—(CH₂)₅—, —(CH₂)₂—[CH(CH₂CF₃)]₃—(CH₂)₆—, —(CH₂)₂—[CH(CH₂CF₃)]₃—(CH₂)₇—, —(CH₂)₃—[CH(CH₂CF₃)]₃—(CH₂)₂—, —(CH₂)₃—[CH(CH₂CF₃)]₃—(CH₂)₄—, —(CH₂)₃—[CH(CH₂CF₃)]₃—(CH₂)₅—, —(CH₂)₃—[CH(CH₂CF₃)]₃—(CH₂)₆—, —(CH₂)₄—[CH(CH₂CF₃)]₃—(CH₂)₂—, —(CH₂)₄—[CH(CH₂CF₃)]₃—(CH₂)₃—, —(CH₂)₄—[CH(CH₂CF₃)]₃—(CH₂)₅—, —(CH₂)₅—[CH(CH₂CF₃)]₃—(CH₂)₂—, —(CH₂)₅—[CH(CH₂CF₃)]₃—(CH₂)₃—, —(CH₂)₅—[CH(CH₂CF₃)]₃—(CH₂)₄—, —(CH₂)₆—[CH(CH₂CF₃)]₃—(CH₂)₂—, —(CH₂)₆—[CH(CH₂CF₃)]₃—(CH₂)₃—,

—[CH(CH₂CF₃)]₄—(CH₂)—, —(CH₂)—[CH(CH₂CF₃)]₄—, —(CH₂)—[CH(CH₂CF₃)]₄—(CH₂)—, —(CH₂)—[CH(CH₂CF₃)]₄—(CH₂)₂—, —(CH₂)—[CH(CH₂CF₃)]₄—(CH₂)₃—, —(CH₂)—[CH(CH₂CF₃)]₄—(CH₂)₄—, —(CH₂)—[CH(CH₂CF₃)]₄—(CH₂)₅—, —(CH₂)—[CH(CH₂CF₃)]₄—(CH₂)₆—, —(CH₂)—[CH(CH₂CF₃)]₄—(CH₂)₇—, —(CH₂)—[CH(CH₂CF₃)]₄—(CH₂)₈—, —(CH₂)—[CH(CH₂CF₃)]₄—(CH₂)₉—, —(CH₂)—[CH(CH₂CF₃)]₄—(CH₂)₁₀—, —(CH₂)₂—[CH(CH₂CF₃)]₄—(CH₂)—, —(CH₂)₃—[CH(CH₂CF₃)]₄—(CH₂)—, —(CH₂)₄—[CH(CH₂CF₃)]₄—(CH₂)—, —(CH₂)₅—[CH(CH₂CF₃)]₄—(CH₂)—, —(CH₂)₆—[CH(CH₂CF₃)]₄—(CH₂)—, —(CH₂)₇—[CH(CH₂CF₃)]₄—(CH₂)—, —(CH₂)₂—[CH(CH₂CF₃)]₄—(CH₂)₂—, —(CH₂)₃—[CH(CH₂CF₃)]₄—(CH₂)₃—, —(CH₂)₄—[CH(CH₂CF₃)]₄—(CH₂)₄—, —(CH₂)₅—[CH(CH₂CF₃)]₄—(CH₂)₅—, —(CH₂)₂—[CH(CH₂CF₃)]₄—(CH₂)₃—, —(CH₂)₂—[CH(CH₂CF₃)]₄—(CH₂)₄—, —(CH₂)₂—[CH(CH₂CF₃)]₄—(CH₂)₅—, —(CH₂)₂—[CH(CH₂CF₃)]₄—(CH₂)₆—, —(CH₂)₃—[CH(CH₂CF₃)]₄—(CH₂)₂—, —(CH₂)₃—[CH(CH₂CF₃)]₄—(CH₂)₄—, —(CH₂)₄—[CH(CH₂CF₃)]₄—(CH₂)₂—, —(CH₂)₄—[CH(CH₂CF₃)]₄—(CH₂)₃—, —(CH₂)₅—[CH(CH₂CF₃)]₄—(CH₂)₂—, —(CH₂)₅—[CH(CH₂CF₃)]₄—(CH₂)₃—, —(CH₂)₆—[CH(CH₂CF₃)]₄—(CH₂)₂—,

—[CH(CH₂CF₃)]₅—(CH₂)—, —(CH₂)—[CH(CH₂CF₃)₅—, —(CH₂)—[CH(CH₂CF₃)]₅—(CH₂)—, —(CH₂)—[CH(CH₂CF₃)]₅—(CH₂)₂—, —(CH₂)—[CH(CH₂CF₃)]₅—(CH₂)₃—, —(CH₂)—[CH(CH₂CF₃)]₅—(CH₂)₄—, —(CH₂)—[CH(CH₂CF₃)]₅—(CH₂)₅—, —(CH₂)—[CH(CH₂CF₃)]₅—(CH₂)₆—, —(CH₂)₂—[CH(CH₂CF₃)]₅—(CH₂)—, —(CH₂)₃—[CH(CH₂CF₃)]₅—(CH₂)—, —(CH₂)₄—[CH(CH₂CF₃)]₅—(CH₂)—, —(CH₂)₅—[CH(CH₂CF₃)]₅—(CH₂)—, —(CH₂)₆—[CH(CH₂CF₃)]₅—(CH₂)—, —(CH₂)₂—[CH(CH₂CF₃)]₅—(CH₂)₂—, —(CH₂)₃—[CH(CH₂CF₃)]₅—(CH₂)₃—, —(CH₂)₄—[CH(CH₂CF₃)]₅—(CH₂)₄—, —(CH₂)₂—[CH(CH₂CF₃)]₅—(CH₂)₃—, —(CH₂)₂—[CH(CH₂CF₃)]₅—(CH₂)₄—, —(CH₂)₂—[CH(CH₂CF₃)]₅—(CH₂)₅—, —(CH₂)₂—[CH(CH₂CF₃)]₅—(CH₂)₆—, —(CH₂)₃—[CH(CH₂CF₃)]₅—(CH₂)₂—, —(CH₂)₃—[CH(CH₂CF₃)]₅—(CH₂)₄—, —(CH₂)₄—[CH(CH₂CF₃)]₅—(CH₂)₂—, —(CH₂)₄—[CH(CH₂CF₃)]₅—(CH₂)₃—, —(CH₂)₅—[CH(CH₂CF₃)]₅—(CH₂)₂—,

—[C(CH₃)(CH₂CF₃)]—(CH₂)—, —(CH₂)—[C(CH₃)(CH₂CF₃)]—, —(CH₂)—[C(CH₃)(CH₂CF₃)]—(CH₂)—, —(CH₂)—[C(CH₃)(CH₂CF₃)]—(CH₂)₂—, —(CH₂)—[C(CH₃)(CH₂CF₃)]—(CH₂)₃—, —(CH₂)—[C(CH₃)(CH₂CF₃)]—(CH₂)₄—, —(CH₂)—[C(CH₃)(CH₂CF₃)]—(CH₂)₅—, —(CH₂)—[C(CH₃)(CH₂CF₃)]—(CH₂)₆—, —(CH₂)—[C(CH₃)(CH₂CF₃)]—(CH₂)₇—, —(CH₂)—[C(CH₃)(CH₂CF₃)]—(CH₂)₈—, —(CH₂)—[C(CH₃)(CH₂CF₃)]—(CH₂)₉—, —(CH₂)—[C(CH₃)(CH₂CF₃)]—(CH₂)₁₀—, —(CH₂)₂—[C(CH₃)(CH₂CF₃)]—(CH₂)—, —(CH₂)₃—[C(CH₃)(CH₂CF₃)]—(CH₂)—, —(CH₂)₄—[C(CH₃)(CH₂CF₃)]—(CH₂)—, —(CH₂)₅—[C(CH₃)(CH₂CF₃)]—(CH₂)—, —(CH₂)₆—[C(CH₃)(CH₂CF₃)]—(CH₂)—, —(CH₂)₇—[C(CH₃)(CH₂CF₃)]—(CH₂)—, —(CH₂)₈—[C(CH₃)(CH₂CF₃)]—(CH₂)—, —(CH₂)₉—[C(CH₃)(CH₂CF₃)]—(CH₂)—, —(CH₂)₁₀—[C(CH₃)(CH₂CF₃)]—(CH₂)—, —(CH₂)₂—[C(CH₃)(CH₂CF₃)]—(CH₂)₂—, —(CH₂)₃—[C(CH₃)(CH₂CF₃)]—(CH₂)₃—, —(CH₂)₄—[C(CH₃)(CH₂CF₃)]—(CH₂)₄—, —(CH₂)₅—[C(CH₃)(CH₂CF₃)]—(CH₂)₅—, —(CH₂)₂—[C(CH₃)(CH₂CF₃)]—(CH₂)₃—, —(CH₂)₂—[C(CH₃)(CH₂CF₃)]—(CH₂)₄—, —(CH₂)₂—[C(CH₃)(CH₂CF₃)]—(CH₂)₅—, —(CH₂)₂—[C(CH₃)(CH₂CF₃)]—(CH₂)₆—, —(CH₂)₂—[C(CH₃)(CH₂CF₃)]—(CH₂)₇—, —(CH₂)₂—[C(CH₃)(CH₂CF₃)]—(CH₂)₈—, —(CH₂)₂—[C(CH₃)(CH₂CF₃)]—(CH₂)₉—, —(CH₂)₃—[C(CH₃)(CH₂CF₃)]—(CH₂)₂—, —(CH₂)₃—[C(CH₃)(CH₂CF₃)]—(CH₂)₄—, —(CH₂)₃—[C(CH₃)(CH₂CF₃)]—(CH₂)₅—, —(CH₂)₃—[C(CH₃)(CH₂CF₃)]—(CH₂)₆—, —(CH₂)₃—[C(CH₃)(CH₂CF₃)]—(CH₂)₇—, —(CH₂)₃—[C(CH₃)(CH₂CF₃)]—(CH₂)₈—, —(CH₂)₄—[C(CH₃)(CH₂CF₃)]—(CH₂)₂—, —(CH₂)₄—[C(CH₃)(CH₂CF₃)]—(CH₂)₃—, —(CH₂)₄—[C(CH₃)(CH₂CF₃)]—(CH₂)₅—, —(CH₂)₄—[C(CH₃)(CH₂CF₃)]—(CH₂)₆—, —(CH₂)₄—[C(CH₃)(CH₂CF₃)]—(CH₂)₇—, —(CH₂)₅—[C(CH₃)(CH₂CF₃)]—(CH₂)₂—, —(CH₂)₅—[C(CH₃)(CH₂CF₃)]—(CH₂)₃—, —(CH₂)₅—[C(CH₃)(CH₂CF₃)]—(CH₂)₄—, —(CH₂)₅—[C(CH₃)(CH₂CF₃)]—(CH₂)₆—, —(CH₂)₆—[C(CH₃)(CH₂CF₃)]—(CH₂)₂—, —(CH₂)₆—[C(CH₃)(CH₂CF₃)]—(CH₂)₃—, —(CH₂)₆—[C(CH₃)(CH₂CF₃)]—(CH₂)₄—, —(CH₂)₆—[C(CH₃)(CH₂CF₃)]—(CH₂)₅—,

—[C(CH₃)(CH₂CF₃)]₂—(CH₂)—, —(CH₂)—[C(CH₃)(CH₂CF₃)]₂—, —(CH₂)—[C(CH₃)(CH₂CF₃)]₂—(CH₂)—, —(CH₂)—[C(CH₃)(CH₂CF₃)]₂—(CH₂)₂—, —(CH₂)—[C(CH₃)(CH₂CF₃)]₂—(CH₂)₃—, —(CH₂)—[C(CH₃)(CH₂CF₃)]₂—(CH₂)₄—, —(CH₂)—[C(CH₃)(CH₂CF₃)]₂—(CH₂)₅—, —(CH₂)—[C(CH₃)(CH₂CF₃)]₂—(CH₂)₆—, —(CH₂)—[C(CH₃)(CH₂CF₃)]₂—(CH₂)₇—, —(CH₂)—[C(CH₃)(CH₂CF₃)]₂—(CH₂)₈—, —(CH₂)—[C(CH₃)(CH₂CF₃)]₂—(CH₂)₉—, —(CH₂)₂—[C(CH₃)(CH₂CF₃)]₂—(CH₂)—, —(CH₂)₃—[C(CH₃)(CH₂CF₃)]₂—(CH₂)—, —(CH₂)₄—[C(CH₃)(CH₂CF₃)]₂—(CH₂)—, —(CH₂)₅—[C(CH₃)(CH₂CF₃)]₂—(CH₂)—, —(CH₂)₆—[C(CH₃)(CH₂CF₃)]₂—(CH₂)—, —(CH₂)₇—[C(CH₃)(CH₂CF₃)]₂—(CH₂)—, —(CH₂)₈—[C(CH₃)(CH₂CF₃)]₂—(CH₂)—, —(CH₂)₉—[C(CH₃)(CH₂CF₃)]₂—(CH₂)—, —(CH₂)₂—[C(CH₃)(CH₂CF₃)]₂—(CH₂)₂—, —(CH₂)₃—[C(CH₃)(CH₂CF₃)]₂—(CH₂)₃—, —(CH₂)₄—[C(CH₃)(CH₂CF₃)]₂—(CH₂)₄—, —(CH₂)₅—[C(CH₃)(CH₂CF₃)]₂—(CH₂)₅—, —(CH₂)₂—[C(CH₃)(CH₂CF₃)]₂—(CH₂)₃—, —(CH₂)₂—[C(CH₃)(CH₂CF₃)]₂—(CH₂)₄—, —(CH₂)₂—[C(CH₃)(CH₂CF₃)]₂—(CH₂)₅—, —(CH₂)₂—[C(CH₃)(CH₂CF₃)]₂—(CH₂)₆—, —(CH₂)₂—[C(CH₃)(CH₂CF₃)]₂—(CH₂)₇—, —(CH₂)₂—[C(CH₃)(CH₂CF₃)]₂—(CH₂)₈—, —(CH₂)₃—[C(CH₃)(CH₂CF₃)]₂—(CH₂)₂—, —(CH₂)₃—[C(CH₃)(CH₂CF₃)]₂—(CH₂)₄—, —(CH₂)₃—[C(CH₃)(CH₂CF₃)]₂—(CH₂)₅—, —(CH₂)₃—[C(CH₃)(CH₂CF₃)]₂—(CH₂)₆—, —(CH₂)₃—[C(CH₃)(CH₂CF₃)]₂—(CH₂)₇—, —(CH₂)₄—[C(CH₃)(CH₂CF₃)]₂—(CH₂)₂—, —(CH₂)₄—[C(CH₃)(CH₂CF₃)]₂—(CH₂)₃—, —(CH₂)₄—[C(CH₃)(CH₂CF₃)]₂—(CH₂)₅—, —(CH₂)₄—[C(CH₃)(CH₂CF₃)]₂—(CH₂)₆—, —(CH₂)₅—[C(CH₃)(CH₂CF₃)]₂—(CH₂)₂—, —(CH₂)₅—[C(CH₃)(CH₂CF₃)]₂—(CH₂)₃—, —(CH₂)₅—[C(CH₃)(CH₂CF₃)]₂—(CH₂)₄—, —(CH₂)₆—[C(CH₃)(CH₂CF₃)]₂—(CH₂)₂—, —(CH₂)₆—[C(CH₃)(CH₂CF₃)]₂—(CH₂)₃—, —(CH₂)₆—[C(CH₃)(CH₂CF₃)]₂—(CH₂)₄—,

—[C(CH₃)(CH₂CF₃)]₃—(CH₂)—, —(CH₂)—[C(CH₃)(CH₂CF₃)]₃—, —(CH₂)—[C(CH₃)(CH₂CF₃)]₃—(CH₂)—, —(CH₂)—[C(CH₃)(CH₂CF₃)]₃—(CH₂)₂—, —(CH₂)—[C(CH₃)(CH₂CF₃)]₃—(CH₂)₃—, —(CH₂)—[C(CH₃)(CH₂CF₃)]₃—(CH₂)₄—, —(CH₂)—[C(CH₃)(CH₂CF₃)]₃—(CH₂)₅—, —(CH₂)—[C(CH₃)(CH₂CF₃)]₃—(CH₂)₆—, —(CH₂)—[C(CH₃)(CH₂CF₃)]₃—(CH₂)₇—, —(CH₂)—[C(CH₃)(CH₂CF₃)]₃—(CH₂)₈—, —(CH₂)₂—[C(CH₃)(CH₂CF₃)]₃—(CH₂)—, —(CH₂)₃—[C(CH₃)(CH₂CF₃)]₃—(CH₂)—, —(CH₂)₄—[C(CH₃)(CH₂CF₃)]₃—(CH₂)—, —(CH₂)₅—[C(CH₃)(CH₂CF₃)]₃—(CH₂)—, —(CH₂)₆—[C(CH₃)(CH₂CF₃)]₃—(CH₂)—, —(CH₂)₇—[C(CH₃)(CH₂CF₃)]₃—(CH₂)—, —(CH₂)₈—[C(CH₃)(CH₂CF₃)]₃—(CH₂)—, —(CH₂)₂—[C(CH₃)(CH₂CF₃)]₃—(CH₂)₂—, —(CH₂)₃—[C(CH₃)(CH₂CF₃)]₃—(CH₂)₃—, —(CH₂)₄—[C(CH₃)(CH₂CF₃)]₃—(CH₂)₄—, —(CH₂)₂—[C(CH₃)(CH₂CF₃)]₃—(CH₂)₃—, —(CH₂)₂—[C(CH₃)(CH₂CF₃)]₃—(CH₂)₄—, —(CH₂)₂—[C(CH₃)(CH₂CF₃)]₃—(CH₂)₅—, —(CH₂)₂—[C(CH₃)(CH₂CF₃)]₃—(CH₂)₆—, —(CH₂)₂—[C(CH₃)(CH₂CF₃)]₃—(CH₂)₇—, —(CH₂)₃—[C(CH₃)(CH₂CF₃)]₃—(CH₂)₂—, —(CH₂)₃—[C(CH₃)(CH₂CF₃)]₃—(CH₂)₄—, —(CH₂)₃—[C(CH₃)(CH₂CF₃)]₃—(CH₂)₅—, —(CH₂)₃—[C(CH₃)(CH₂CF₃)]₃—(CH₂)₆—, —(CH₂)₄—[C(CH₃)(CH₂CF₃)]₃—(CH₂)₂—, —(CH₂)₄—[C(CH₃)(CH₂CF₃)]₃—(CH₂)₃—, —(CH₂)₄—[C(CH₃)(CH₂CF₃)]₃—(CH₂)₅—, —(CH₂)₅—[C(CH₃)(CH₂CF₃)]₃—(CH₂)₂—, —(CH₂)₅—[C(CH₃)(CH₂CF₃)]₃—(CH₂)₃—, —(CH₂)₅—[C(CH₃)(CH₂CF₃)]₃—(CH₂)₄—, —(CH₂)₆—[C(CH₃)(CH₂CF₃)]₃—(CH₂)₂—, —(CH₂)₆—[C(CH₃)(CH₂CF₃)]₃—(CH₂)₃—,

—[C(CH₃)(CH₂CF₃)]₄—(CH₂)—, —(CH₂)—[C(CH₃)(CH₂CF₃)]₄—, —(CH₂)—[C(CH₃)(CH₂CF₃)]₄—(CH₂)—, —(CH₂)—[C(CH₃)(CH₂CF₃)]₄—(CH₂)₂—, —(CH₂)—[C(CH₃)(CH₂CF₃)]₄—(CH₂)₃—, —(CH₂)—[C(CH₃)(CH₂CF₃)]₄—(CH₂)₄—, —(CH₂)—[C(CH₃)(CH₂CF₃)]₄—(CH₂)₅—, —(CH₂)—[C(CH₃)(CH₂CF₃)]₄—(CH₂)₆—, —(CH₂)—[C(CH₃)(CH₂CF₃)]₄—(CH₂)₇—, —(CH₂)—[C(CH₃)(CH₂CF₃)]₄—(CH₂)₈—, —(CH₂)—[C(CH₃)(CH₂CF₃)]₄—(CH₂)₉—, —(CH₂)—[C(CH₃)(CH₂CF₃)]₄—(CH₂)₁₀—, —(CH₂)₂—[C(CH₃)(CH₂CF₃)]₄—(CH₂)—, —(CH₂)₃—[C(CH₃)(CH₂CF₃)]₄—(CH₂)—, —(CH₂)₄—[C(CH₃)(CH₂CF₃)]₄—(CH₂)—, —(CH₂)₅—[C(CH₃)(CH₂CF₃)]₄—(CH₂)—, —(CH₂)₆—[C(CH₃)(CH₂CF₃)]₄—(CH₂)—, —(CH₂)₇—[C(CH₃)(CH₂CF₃)]₄—(CH₂)—, —(CH₂)₂—[C(CH₃)(CH₂CF₃)]₄—(CH₂)₂—, —(CH₂)₃—[C(CH₃)(CH₂CF₃)]₄—(CH₂)₃—, —(CH₂)₄—[C(CH₃)(CH₂CF₃)]₄—(CH₂)₄—, —(CH₂)₅—[C(CH₃)(CH₂CF₃)]₄—(CH₂)₅—, —(CH₂)₂—[C(CH₃)(CH₂CF₃)]₄—(CH₂)₃—, —(CH₂)₂—[C(CH₃)(CH₂CF₃)]₄—(CH₂)₄—, —(CH₂)₂—[C(CH₃)(CH₂CF₃)]₄—(CH₂)₅—, —(CH₂)₂—[C(CH₃)(CH₂CF₃)]₄—(CH₂)₆—, —(CH₂)₃—[C(CH₃)(CH₂CF₃)]₄—(CH₂)₂—, —(CH₂)₃—[C(CH₃)(CH₂CF₃)]₄—(CH₂)₄—, —(CH₂)₄—[C(CH₃)(CH₂CF₃)]₄—(CH₂)₂—, —(CH₂)₄—[C(CH₃)(CH₂CF₃)]₄—(CH₂)₃—, —(CH₂)₅—[C(CH₃)(CH₂CF₃)]₄—(CH₂)₂—, —(CH₂)₅—[C(CH₃)(CH₂CF₃)]₄—(CH₂)₃—, —(CH₂)₆—[C(CH₃)(CH₂CF₃)]₄—(CH₂)₂—,

—[C(CH₃)(CH₂CF₃)]₅—(CH₂)—, —(CH₂)—[C(CH₃)(CH₂CF₃)]₅—, —(CH₂)—[C(CH₃)(CH₂CF₃)]₅—(CH₂)—, —(CH₂)—[C(CH₃)(CH₂CF₃)]₅—(CH₂)₂—, —(CH₂)—[C(CH₃)(CH₂CF₃)]₅—(CH₂)₃—, —(CH₂)—[C(CH₃)(CH₂CF₃)]₅—(CH₂)₄—, —(CH₂)—[C(CH₃)(CH₂CF₃)]₅—(CH₂)₅—, —(CH₂)—[C(CH₃)(CH₂CF₃)]₅—(CH₂)₆—, —(CH₂)₂—[C(CH₃)(CH₂CF₃)]₅—(CH₂)—, —(CH₂)₃—[C(CH₃)(CH₂CF₃)]₅—(CH₂)—, —(CH₂)₄—[C(CH₃)(CH₂CF₃)]₅—(CH₂)—, —(CH₂)₅—[C(CH₃)(CH₂CF₃)]₅—(CH₂)—, —(CH₂)₆—[C(CH₃)(CH₂CF₃)]₅—(CH₂)—, —(CH₂)₂—[C(CH₃)(CH₂CF₃)]₅—(CH₂)₂—, —(CH₂)₃—[C(CH₃)(CH₂CF₃)]₅—(CH₂)₃—, —(CH₂)₄—[C(CH₃)(CH₂CF₃)]₅—(CH₂)₄—, —(CH₂)₂—[C(CH₃)(CH₂CF₃)]₅—(CH₂)₃—, —(CH₂)₂—[C(CH₃)(CH₂CF₃)]₅—(CH₂)₄—, —(CH₂)₂—[C(CH₃)(CH₂CF₃)]₅—(CH₂)₅—, —(CH₂)₂—[C(CH₃)(CH₂CF₃)]₅—(CH₂)₆—, —(CH₂)₃—[C(CH₃)(CH₂CF₃)]₅—(CH₂)₂—, —(CH₂)₃—[C(CH₃)(CH₂CF₃)]₅—(CH₂)₄—, —(CH₂)₄—[C(CH₃)(CH₂CF₃)]₅—(CH₂)₂—, —(CH₂)₄—[C(CH₃)(CH₂CF₃)]₅—(CH₂)₃—, —(CH₂)₅—[C(CH₃)(CH₂CF₃)]₅—(CH₂)₂—,

—(CH₂)₂—(CF₂)—O—(CF₂)—(CH₂)₂—, —(CH₂)₂—(CF₂)—O—(CH₂)—O—(CF₂)—(CH₂)₂—, —(CH₂)₂—(CF₂)—O—(CH₂)₂—O—(CF₂)—(CH₂)₂,

—(CH₂)—(CF₂)—O—(CF₂)—O—(CF₂)—(CH₂)—, —(CH₂)₂—(CF₂)—O—(CF₂)—O—(CF₂)—(CH₂)₂—, —(CH₂)₃—(CF₂)—O—(CF₂)—O—(CF₂)—(CH₂)₃—, —(CH₂)₄—(CF₂)—O—(CF₂)—O—(CF₂)—(CH₂)₄—, —(CH₂)—(CF₂)—O—(CF₂)—O—(CF₂)—(CH₂)₂—, —(CH₂)—(CF₂)—O—(CF₂)—O—(CF₂)—(CH₂)₃—, —(CH₂)—(CF₂)—O—(CF₂)—O—(CF₂)—(CH₂)₄—, —(CH₂)—(CF₂)—O—(CF₂)—O—(CF₂)—(CH₂)₅—, —(CH₂)—(CF₂)—O—(CF₂)—O—(CF₂)—(CH₂)₆—, —(CH₂)—(CF₂)—O—(CF₂)—O—(CF₂)—(CH₂)₇—, —(CH₂)₂—(CF₂)—O—(CF₂)—O—(CF₂)—(CH₂)—, —(CH₂)₃—(CF₂)—O—(CF₂)—O—(CF₂)—(CH₂)—, —(CH₂)₄—(CF₂)—O—(CF₂)—O—(CF₂)—(CH₂)—, —(CH₂)₅—(CF₂)—O—(CF₂)—O—(CF₂)—(CH₂)—, —(CH₂)₆—(CF₂)—O—(CF₂)—O—(CF₂)—(CH₂)—, —(CH₂)₇—(CF₂)—O—(CF₂)—O—(CF₂)—(CH₂)—, —(CH₂)₂—(CF₂)—O—(CF₂)—O—(CF₂)—(CH₂)₃—, —(CH₂)₂—(CF₂)—O—(CF₂)—O—(CF₂)—(CH₂)₄—, —(CH₂)₂—(CF₂)—O—(CF₂)—O—(CF₂)—(CH₂)₅—, —(CH₂)₂—(CF₂)—O—(CF₂)—O—(CF₂)—(CH₂)₆—, —(CH₂)₃—(CF₂)—O—(CF₂)—O—(CF₂)—(CH₂)₂—, —(CH₂)₄—(CF₂)—O—(CF₂)—O—(CF₂)—(CH₂)₂—, —(CH₂)₅—(CF₂)—O—(CF₂)—O—(CF₂)—(CH₂)₂—, —(CH₂)₆—(CF₂)—O—(CF₂)—O—(CF₂)—(CH₂)₂—, —(CH₂)₃—(CF₂)—O—(CF₂)—O—(CF₂)—(CH₂)₄—, —(CH₂)₄—(CF₂)—O—(CF₂)—O—(CF₂)—(CH₂)₃—, —(CH₂)₃—(CF₂)—O—(CF₂)—O—(CF₂)—(CH₂)₅—, —(CH₂)₅—(CF₂)—O—(CF₂)—O—(CF₂)—(CH₂)₃—,

—(CH₂)—(CF₂)—O—(CF₂)₂—O—(CF₂)—(CH₂)—, —(CH₂)₂—(CF₂)—O—(CF₂)₂—O—(CF₂)—(CH₂)₂—, —(CH₂)₃—(CF₂)—O—(CF₂)₂—O—(CF₂)—(CH₂)₃—, —(CH₂)₄—(CF₂)—O—(CF₂)₂—O—(CF₂)—(CH₂)₄—, —(CH₂)—(CF₂)—O—(CF₂)₂—O—(CF₂)—(CH₂)₂—, —(CH₂)—(CF₂)—O—(CF₂)₂—O—(CF₂)—(CH₂)₃—, —(CH₂)—(CF₂)—O—(CF₂)₂—O—(CF₂)—(CH₂)₄—, —(CH₂)—(CF₂)—O—(CF₂)₂—O—(CF₂)—(CH₂)₅—, —(CH₂)—(CF₂)—O—(CF₂)₂—O—(CF₂)—(CH₂)₆—, —(CH₂)—(CF₂)—O—(CF₂)₂—O—(CF₂)—(CH₂)₇—, —(CH₂)₂—(CF₂)—O—(CF₂)₂—O—(CF₂)—(CH₂)—, —(CH₂)₃—(CF₂)—O—(CF₂)₂—O—(CF₂)—(CH₂)—, —(CH₂)₄—(CF₂)—O—(CF₂)₂—O—(CF₂)—(CH₂)—, —(CH₂)₅—(CF₂)—O—(CF₂)₂—O—(CF₂)—(CH₂)—, —(CH₂)₆—(CF₂)—O—(CF₂)₂—O—(CF₂)—(CH₂)—, —(CH₂)₇—(CF₂)—O—(CF₂)₂—O—(CF₂)—(CH₂)—, —(CH₂)₂—(CF₂)—O—(CF₂)₂—O—(CF₂)—(CH₂)₃—, —(CH₂)₂—(CF₂)—O—(CF₂)₂—O—(CF₂)—(CH₂)₄—, —(CH₂)₂—(CF₂)—O—(CF₂)₂—O—(CF₂)—(CH₂)₅—, —(CH₂)₂—(CF₂)—O—(CF₂)₂—O—(CF₂)—(CH₂)₆—, —(CH₂)₃—(CF₂)—O—(CF₂)₂—O—(CF₂)—(CH₂)₂—, —(CH₂)₄—(CF₂)—O—(CF₂)₂—O—(CF₂)—(CH₂)₂—, —(CH₂)₅—(CF₂)—O—(CF₂)₂—O—(CF₂)—(CH₂)₂—, —(CH₂)₆—(CF₂)—O—(CF₂)₂—O—(CF₂)—(CH₂)₂—, —(CH₂)₃—(CF₂)—O—(CF₂)₂—O—(CF₂)—(CH₂)₄—, —(CH₂)₄—(CF₂)—O—(CF₂)₂—O—(CF₂)—(CH₂)₃—, —(CH₂)₃—(CF₂)—O—(CF₂)₂—O—(CF₂)—(CH₂)₅—, —(CH₂)₅—(CF₂)—O—(CF₂)₂—O—(CF₂)—(CH₂)₃—,

—(CH₂)—(CF₂)₂—O—(CF₂)₂—O—(CF₂)₂—(CH₂)—, —(CH₂)₂—(CF₂)₂—O—(CF₂)₂—O—(CF₂)₂—(CH₂)₂—, —(CH₂)₃—(CF₂)₂—O—(CF₂)₂—O—(CF₂)₂—(CH₂)₃—, —(CH₂)₄—(CF₂)₂—O—(CF₂)₂—O—(CF₂)₂—(CH₂)₄—, —(CH₂)—(CF₂)₂—O—(CF₂)₂—O—(CF₂)₂—(CH₂)₂—, —(CH₂)—(CF₂)₂—O—(CF₂)₂—O—(CF₂)₂—(CH₂)₃—, —(CH₂)—(CF₂)₂—O—(CF₂)₂—O—(CF₂)₂—(CH₂)₃—, —(CH₂)—(CF₂)₂—O—(CF₂)₂—O—(CF₂)₂—(CH₂)₄—, —(CH₂)—(CF₂)₂—O—(CF₂)₂—O—(CF₂)₂—(CH₂)₅—, —(CH₂)—(CF₂)₂—O—(CF₂)₂—O—(CF₂)₂—(CH₂)₆—, —(CH₂)—(CF₂)₂—O—(CF₂)₂—O—(CF₂)₂—(CH₂)₇—, —(CH₂)₂—(CF₂)₂—O—(CF₂)₂—O—(CF₂)₂—(CH₂)—, —(CH₂)₃—(CF₂)₂—O—(CF₂)₂—O—(CF₂)₂—(CH₂)—, —(CH₂)₄—(CF₂)₂—O—(CF₂)₂—O—(CF₂)₂—(CH₂)—, —(CH₂)₅—(CF₂)₂—O—(CF₂)₂—O—(CF₂)₂—(CH₂)—, —(CH₂)₆—(CF₂)₂—O—(CF₂)₂—O—(CF₂)₂—(CH₂)—, —(CH₂)₇—(CF₂)₂—O—(CF₂)₂—O—(CF₂)₂—(CH₂)—, —(CH₂)₂—(CF₂)₂—O—(CF₂)₂—O—(CF₂)₂—(CH₂)₃—, —(CH₂)₂—(CF₂)₂—O—(CF₂)₂—O—(CF₂)₂—(CH₂)₄—, —(CH₂)₂—(CF₂)₂—O—(CF₂)₂—O—(CF₂)₂—(CH₂)₅—, —(CH₂)₂—(CF₂)₂—O—(CF₂)₂—O—(CF₂)₂—(CH₂)₆—, —(CH₂)₃—(CF₂)₂—O—(CF₂)₂—O—(CF₂)₂—(CH₂)₂—, —(CH₂)₄—(CF₂)₂—O—(CF₂)₂—O—(CF₂)₂—(CH₂)₂—, —(CH₂)₅—(CF₂)₂—O—(CF₂)₂—O—(CF₂)₂—(CH₂)₂—, —(CH₂)₆—(CF₂)₂—O—(CF₂)₂—O—(CF₂)₂—(CH₂)₂—, —(CH₂)₃—(CF₂)₂—O—(CF₂)₂—O—(CF₂)₂—(CH₂)₄—, —(CH₂)₄—(CF₂)₂—O—(CF₂)₂—O—(CF₂)₂—(CH₂)₃—, —(CH₂)₃—(CF₂)₂—O—(CF₂)₂—O—(CF₂)₂—(CH₂)₅—, —(CH₂)₅—(CF₂)₂—O—(CF₂)₂—O—(CF₂)₂—(CH₂)₃—,

—(CH₂)—(CF₂)—O—(CF₂)—O—(CF₂)—O—(CF₂)—(CH₂)—, —(CH₂)₂—(CF₂)—O—(CF₂)—O—(CF₂)—O—(CF₂)—(CH₂)₂—, —(CH₂)₃—(CF₂)—O—(CF₂)—O—(CF₂)—O—(CF₂)—(CH₂)₃—, —(CH₂)₄—(CF₂)—O—(CF₂)—O—(CF₂)—O—(CF₂)—(CH₂)₄—, —(CH₂)—(CF₂)—O—(CF₂)—O—(CF₂)—O—(CF₂)—(CH₂)₂—, —(CH₂)—(CF₂)—O—(CF₂)—O—(CF₂)—O—(CF₂)—(CH₂)₃—, —(CH₂)—(CF₂)—O—(CF₂)—O—(CF₂)—O—(CF₂)—(CH₂)₄—, —(CH₂)—(CF₂)—O—(CF₂)—O—(CF₂)—O—(CF₂)—(CH₂)₅—, —(CH₂)—(CF₂)—O—(CF₂)—O—(CF₂)—O—(CF₂)—(CH₂)₆—, —(CH₂)—(CF₂)—O—(CF₂)—O—(CF₂)—O—(CF₂)—(CH₂)₇—, —(CH₂)₂—(CF₂)—O—(CF₂)—O—(CF₂)—O—(CF₂)—(CH₂)—, —(CH₂)₃—(CF₂)—O—(CF₂)—O—(CF₂)—O—(CF₂)—(CH₂)—, —(CH₂)₄—(CF₂)—O—(CF₂)—O—(CF₂)—O—(CF₂)—(CH₂)—, —(CH₂)₅—(CF₂)—O—(CF₂)—O—(CF₂)—O—(CF₂)—(CH₂)—, —(CH₂)₆—(CF₂)—O—(CF₂)—O—(CF₂)—O—(CF₂)—(CH₂)—, —(CH₂)₇—(CF₂)—O—(CF₂)—O—(CF₂)—O—(CF₂)—(CH₂)—, —(CH₂)₂—(CF₂)—O—(CF₂)—O—(CF₂)—O—(CF₂)—(CH₂)₃—, —(CH₂)₂—(CF₂)—O—(CF₂)—O—(CF₂)—O—(CF₂)—(CH₂)₄—, —(CH₂)₂—(CF₂)—O—(CF₂)—O—(CF₂)—O—(CF₂)—(CH₂)₅—, —(CH₂)₂—(CF₂)—O—(CF₂)—O—(CF₂)—O—(CF₂)—(CH₂)₆—, —(CH₂)₃—(CF₂)—O—(CF₂)—O—(CF₂)—O—(CF₂)—(CH₂)₂—, —(CH₂)₄—(CF₂)—O—(CF₂)—O—(CF₂)—O—(CF₂)—(CH₂)₂—, —(CH₂)₅—(CF₂)—O—(CF₂)—O—(CF₂)—O—(CF₂)—(CH₂)₂—, —(CH₂)₆—(CF₂)—O—(CF₂)—O—(CF₂)—O—(CF₂)—(CH₂)₂—, —(CH₂)₃—(CF₂)—O—(CF₂)—O—(CF₂)—O—(CF₂)—(CH₂)₄—, —(CH₂)₄—(CF₂)—O—(CF₂)—O—(CF₂)—O—(CF₂)—(CH₂)₃—, —(CH₂)₃—(CF₂)—O—(CF₂)—O—(CF₂)—O—(CF₂)—(CH₂)₅—, —(CH₂)₅—(CF₂)—O—(CF₂)—O—(CF₂)—O—(CF₂)—(CH₂)₃—,

—(CH₂)—(CF₂)₂—O—(CF₂)₂—O—(CF₂)₂—O—(CF₂)₂—(CH₂)—, —(CH₂)₂—(CF₂)₂—O—(CF₂)₂—O—(CF₂)₂—O—(CF₂)₂—(CH₂)₂—, —(CH₂)₃—(CF₂)₂—O—(CF₂)₂—O—(CF₂)₂—O—(CF₂)₂—(CH₂)₃—, —(CH₂)₄—(CF₂)₂—O—(CF₂)₂—O—(CF₂)₂—O—(CF₂)₂—(CH₂)₄—, —(CH₂)—(CF₂)₂—O—(CF₂)₂—O—(CF₂)₂—O—(CF₂)₂—(CH₂)₂—, —(CH₂)—(CF₂)₂—O—(CF₂)₂—O—(CF₂)₂—O—(CF₂)₂—(CH₂)₃—, —(CH₂)—(CF₂)₂—O—(CF₂)₂—O—(CF₂)₂—O—(CF₂)₂—(CH₂)₄—, —(CH₂)—(CF₂)₂—O—(CF₂)₂—O—(CF₂)₂—O—(CF₂)₂—(CH₂)₅—, —(CH₂)—(CF₂)₂—O—(CF₂)₂—O—(CF₂)₂—O—(CF₂)₂—(CH₂)₆—, —(CH₂)—(CF₂)₂—O—(CF₂)₂—O—(CF₂)₂—O—(CF₂)₂—(CH₂)₇—, —(CH₂)₂—(CF₂)₂—O—(CF₂)₂—O—(CF₂)₂—O—(CF₂)₂—(CH₂)—, —(CH₂)₃—(CF₂)₂—O—(CF₂)₂—O—(CF₂)₂—O—(CF₂)₂—(CH₂)—, —(CH₂)₄—(CF₂)₂—O—(CF₂)₂—O—(CF₂)₂—O—(CF₂)₂—(CH₂)—, —(CH₂)₅—(CF₂)₂—O—(CF₂)₂—O—(CF₂)₂—O—(CF₂)₂—(CH₂)—, —(CH₂)₆—(CF₂)₂—O—(CF₂)₂—O—(CF₂)₂—O—(CF₂)₂—(CH₂)—, —(CH₂)₇—(CF₂)₂—O—(CF₂)₂—O—(CF₂)₂—O—(CF₂)₂—(CH₂)—, —(CH₂)₂—(CF₂)₂—O—(CF₂)₂—O—(CF₂)₂—O—(CF₂)₂—(CH₂)₃—, —(CH₂)₂—(CF₂)₂—O—(CF₂)₂—O—(CF₂)₂—O—(CF₂)₂—(CH₂)₄—, —(CH₂)₂—(CF₂)₂—O—(CF₂)₂—O—(CF₂)₂—O—(CF₂)₂—(CH₂)₅—, —(CH₂)₂—(CF₂)₂—O—(CF₂)₂—O—(CF₂)₂—O—(CF₂)₂—(CH₂)₆—, —(CH₂)₃—(CF₂)₂—O—(CF₂)₂—O—(CF₂)₂—O—(CF₂)₂—(CH₂)₂—, —(CH₂)₄—(CF₂)₂—O—(CF₂)₂—O—(CF₂)₂—O—(CF₂)₂—(CH₂)₂—, —(CH₂)₅—(CF₂)₂—O—(CF₂)₂—O—(CF₂)₂—O—(CF₂)₂—(CH₂)₂—, —(CH₂)₆—(CF₂)₂—O—(CF₂)₂—O—(CF₂)₂—O—(CF₂)₂—(CH₂)₂—, —(CH₂)₃—(CF₂)₂—O—(CF₂)₂—O—(CF₂)₂—O—(CF₂)₂—(CH₂)₄—, —(CH₂)₄—(CF₂)₂—O—(CF₂)₂—O—(CF₂)₂—O—(CF₂)₂—(CH₂)₃—, —(CH₂)₃—(CF₂)₂—O—(CF₂)₂—O—(CF₂)₂—O—(CF₂)₂—(CH₂)₅—, —(CH₂)₅—(CF₂)₂—O—(CF₂)₂—O—(CF₂)₂—O—(CF₂)₂—(CH₂)₃—.

Preferred examples for —R₂— are —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₂—S—(CH₂)₂—, —(CH₂)₂—SO₂—(CH₂)₂—, —(CH₂)₂—S—(CH₂)₂—S—(CH₂)₂—, —(CH₂)₂—O—(CH₂)₂—O—(CH₂)₂—, —(CH₂)₂—S—(CH₂)₂—O—(CH₂)₂—, —(CH₂)₂—SO₂—(CH₂)₂—O—(CH₂)₂—, —(CH₂)₂—SO₂—(CH₂)₂—S—(CH₂)₂—, —(CH₂)₂—O—(CH₂)₂—O—(CH₂)₂—O—(CH₂)₂—, —(CH₂)₂—O—(CH₂)₂—S—(CH₂)₂—O—(CH₂)₂—, —(CH₂)₂—S—(CH₂)₂—O—(CH₂)₂—S—(CH₂)₂—, —(CH₂)₂—SO₂—(CH₂)₂—O—(CH₂)₂—SO₂—(CH₂)₂—, —(CH₂)₂—S—(CH₂)₂—S—(CH₂)₂—S—(CH₂)₂—, —(CH₂)₂—SO₂—(CH₂)₂—S—(CH₂)₂—SO₂—(CH₂)₂—, —(CH₂)₂—SO₂—(CH₂)₂—SO₂—(CH₂)₂—SO₂—(CH₂)₂—, —(CH₂)₂—O—(CH₂)₂—SO₂—(CH₂)₂—O—(CH₂)₂—, —(CH₂)₃—(CF₂)—(CH₂)₃—, —(CH₂)—(CF₂)₃—(CH₂)—, —(CH₂)₂—(CF₂)₄—(CH₂)₂—, —(CH₂)—[CH(CF₃)]—(CH₂)—, —(CH₂)—[C(CH₃)CF₃]—(CH₂)—, —(CH₂)—[CH(CH₂CF₃)]—(CH₂)—, —(CH₂)—[C(CH₃)(CH₂CF₃)]—(CH₂)—, —(CH₂)₂—(CF₂)—O—(CF₂)—O—(CF₂)—(CH₂)₂—and —(CH₂)—(CF₂)—O—(CF₂)—O—(CF₂)—O—(CF₂)—(CH₂)— according to the invention.

Particularly preferred examples for —R₂— are —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₂—S—(CH₂)₂—, —(CH₂)₂—SO₂—(CH₂)₂—, —(CH₂)₂—S—(CH₂)₂—S—(CH₂)₂—, —(CH₂)₂—O—(CH₂)₂—O—(CH₂)₂—, —(CH₂)₂—O—(CH₂)₂—O—(CH₂)₂—O—(CH₂)₂—, —(CH₂)₂—O—(CH₂)₂—S—(CH₂)₂—O—(CH₂)₂—, —(CH₂)₂—S—(CH₂)₂—O—(CH₂)₂—S—(CH₂)₂—, —(CH₂)₂—S—(CH₂)₂—S—(CH₂)₂—S—(CH₂)₂— and —(CH₂)₂—SO₂—(CH₂)₂—S—(CH₂)₂—SO₂—(CH₂)₂— according to the invention.

Compounds of formulae (I), (I #), (I′), (I′-a), (I′-b), (I′-c), (I′-d), (I′-e), (I′-f), (I′-g), (I′-h), (I′-i) and (I″) with substituents as described before or preferably described before having a polymerizable group as described before or preferably described before or below are preferred in case the substituent —R₂— within the at least one linking element Y—R₂— or in N—R₂—R₁ corresponds to —(C(R)₂)ₒ—, wherein R and o has a meaning as described or preferably described before.

Accordingly monomers of formulae (I), (I #), (I′), (I′-a), (I′-b), (I′-c), (I′-d), (I′-e), (I′-f), (I′-g), (I′-h), (I′-i) and (I″) for the preparation of an ophthalmic device or a precursor article for manufacturing an ophthalmic device as described before with substituents as described before or preferably described before having a polymerizable group as described before or preferably described before or below are preferred in case the substituent —R₂— within the at least one linking element Y—R₂— or within N—R₂—R₁ corresponds to —(C(R)₂)ₒ—, wherein R and o has a meaning as described or preferably described before. Such ophthalmic devices and precursor articles prepared by using these monomers are especially preferred.

Particularly preferred examples for —R₂— are —(CH₂)₈—, —(CH₂)₉—, —(CH₂)₁₀—, —(CH₂)₁₁—, and —(CH₂)₁₂— according to the invention. Very particularly preferably, —R₂— is —(CH₂)₁₂— according to the invention.

Therefore, the invention is furthermore directed to an ophthalmic device or a precursor article for manufacturing an ophthalmic device comprising polymerized compounds of formulae (I), (I #), (I′), (I′-a), (I′-b), (I′-c), (I′-d), (I′-e), (I′-f), (I′-g), (I′-h), (I′-i) and (I″) as described before or preferably described before wherein —R₂— is at each occurrence independently —(C(R)₂)ₒ—, wherein R and o have a meaning as described or preferably described before.

The invention therefore relates to compounds of formulae (I), (I #), (I′), (I′-a), (I′-b), (I′-c), (I′-d), (I′-e), (I′-f), (I′-g), (I′-h), (I′-i) and (I″) as described before or preferably described before wherein —R₂— is at each occurrence independently —(C(R)₂)ₒ—, wherein R and o have a meaning as described or preferably described before and the disclosed disclaimers are considered.

The substituent Y—R₂— within formulae (I), (I #), (I′), (I′-a), (I′-b), (I′-c), (I′-d), (I′-e), (I′-f), (I′-g), (I′-h), (I′-i) and (I″) is selected from the group consisting of O—R₂—, —R₂— where Y is a bond, SO₂—R₂— and S—R₂—, wherein —R₂— has a meaning as described before or preferably or particularly preferably described before.

The substituent Y—R₂— is preferably selected from the group consisting of O—R₂— and —R₂— where Y is a bond wherein —R₂— has a meaning as described before or preferably or particularly preferably described before.

The substituent Y—R₂—R₁ within formulae (I), (I #), (I′), (I′-a), (I′-b), (I′-c), (I′-d), (I′-e), (I′-f), (I′-g), (I′-h), (I′-i) and (I″) is selected from the group consisting of O—R₂—R₁, —R₂—R₁, SO₂—R₂—R₁ and S—R₂—R₁, or preferably selected from the group consisting of O—R₂—R₁ and —R₂—R₁, wherein —R₂— has a meaning as described before or preferably or particularly preferably described before and wherein R₁ is trimethoxysilyl, triethoxysilyl, dimethoxymethylsilyl, diethoxymethylsilyl or a polymerizable group according to formula (4),

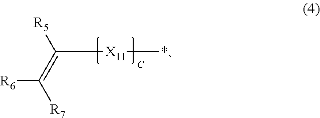

wherein

X₁₁ is selected from the group consisting of O, S, O—SO₂, SO₂—O, C(═O), OC(═O), C(═O)O, S(C═O) and (C═O)S,
R₅, R₆, R₇ are at each occurrence independently of each other selected from the group consisting of H, F, a linear or branched, non-fluorinated, partially or completely fluorinated alkyl group having 1 to 20 C atoms or aryl with 6 to 14 C atoms and

c is 0 or 1.

The substituent N—R₂—R₁ within formulae (I), (I #), (I′), (I′-a), (I′-b), (I′-c), (I′-d), (I′-e), (I′-f), (I′-g), (I′-h), (I′-i) and (I″) is preferred wherein —R₂— has a meaning as described before or preferably before and wherein R₁ is trimethoxysilyl, triethoxysilyl, dimethoxymethylsilyl, diethoxymethylsilyl or a polymerizable group according to formula (4),

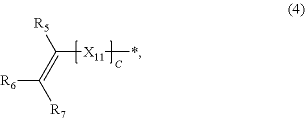

wherein

X₁₁ is selected from the group consisting of O, S, O—SO₂, SO₂—O, C(═O), OC(═O), C(═O)O, S(C═O) and (C═O)S,
R₅, R₆, R₇ are at each occurrence independently of each other selected from the group consisting of H, F, a linear or branched, non-fluorinated, partially or completely fluorinated alkyl group having 1 to 20 C atoms or aryl with 6 to 14 C atoms and

c is 0 or 1.

In another preferred embodiment of the invention, c, X₁₁, R₅, R₆ and R₇ within the compounds of formulae (I), (I #), (I′), (I′-a), (I′-b), (I′-c), (I′-d), (I′-e), (I′-f), (I′-g), (I′-h), (I′-i) and (I″) acting as monomers for the preparation of the ophthalmic device or the precursor article for manufacturing an ophthalmic device as described before or for the preparation of an oligomer, polymer or copolymer according to the invention or within the compounds according to the invention have the following preferred meaning:

Preferably, R₆ and R₇ are H. Preferably, c is 1.

Preferably, R₅ is H, methyl, ethyl or phenyl. Particularly preferably, R₅ is H or methyl.

Preferably, X₁₁ is C(═O), OC(═O) or C(═O)O. Particularly preferably, X₁₁ is C(═O)O.

Preferred alkenyl groups of formula (4) as polymerizable groups R₁ according to the invention are therefore represented by any one selected from the group consisting of formulae (4-1), (4-2), (4-3), (4-4), (4-5), (4-6), (4-7), (4-8), (4-9), (4-10), (4-11) and (4-12):

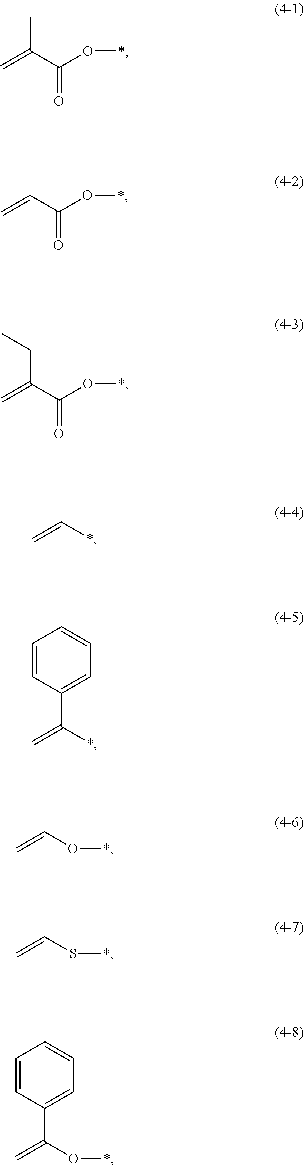

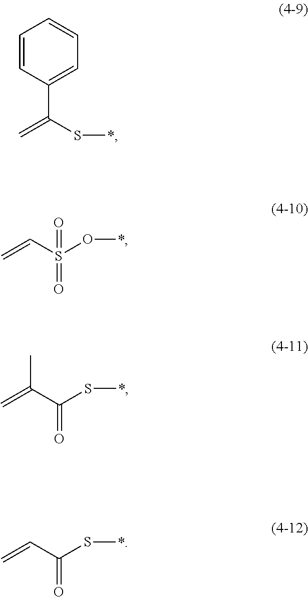

Particularly preferred alkenyl groups of formula (4) as polymerizable groups R₁ according to the invention are represented by any one selected from the group consisting of formulae (4-1), (4-2), (4-3), (4-5), (4-6), (4-11) and (4-12) as described before.

The alkenyl group represented by formula (4-1) is called methacrylate. The alkenyl group represented by formula (4-2) is called acrylate.

The preferred groups R₁ are preferably combined with preferred groups of the linking element —R₂— and/or the linking element Y—R₂—. Combinations are excluded where two O atoms or one O atom and one S atom are directly bonded to each other as known for a skilled artisan in the field of organic chemistry.

The substituent Y—R₂—R₁ within formulae (I), (I #), (I′), (I′-a), (I′-b), (I′-c), (I′-d), (I′-e), (I-′f), (I′-g), (I-′h), (I′-i) and (I″) is therefore particularly preferably selected from the group consisting of

O—(CH₂)₅—R¹, O—(CH₂)₆—R¹, O—(CH₂)₇—R¹, O—(CH₂)₈—R¹, O—(CH₂)₉—R¹, O—(CH₂)₁₀—R¹, O—(CH₂)₁₁—R¹, O—(CH₂)₁₂—R¹, O—(CH₂)₁₃—R¹, O—(CH₂)₂—S—(CH₂)₂—R¹, O—(CH₂)₂—SO₂—(CH₂)₂—R¹, O—(CH₂)₂—S—(CH₂)₂—S—(CH₂)₂—R¹, O—(CH₂)₂—O—(CH₂)₂—O—(CH₂)₂—R¹, O—(CH₂)₂—S—(CH₂)₂—O—(CH₂)₂—R¹, O—(CH₂)₂—SO₂—(CH₂)₂—O—(CH₂)₂—R¹, O—(CH₂)₂—SO₂—(CH₂)₂—S—(CH₂)₂—R¹, O—(CH₂)₂—O—(CH₂)₂—O—(CH₂)₂—O—(CH₂)₂—R¹, O—(CH₂)₂—O—(CH₂)₂—S—(CH₂)₂—O—(CH₂)₂—R¹, O—(CH₂)₂—S—(CH₂)₂—O—(CH₂)₂—S—(CH₂)₂—R¹, O—(CH₂)₂—SO₂—(CH₂)₂—O—(CH₂)₂—SO₂—(CH₂)₂—R¹, O—(CH₂)₂—S—(CH₂)₂—S—(CH₂)₂—S—(CH₂)₂—R¹, O—(CH₂)₂—SO₂—(CH₂)₂—S—(CH₂)₂—SO₂—(CH₂)₂—R¹, O—(CH₂)₂—SO₂—(CH₂)₂—SO₂—(CH₂)₂—SO₂—(CH₂)₂—R¹, O—(CH₂)₂—O—(CH₂)₂—SO₂—(CH₂)₂—O—(CH₂)₂—R¹, O—(CH₂)₃—(CF₂)—(CH₂)₃—R¹, O—(CH₂)—(CF₂)₃—(CH₂)—R¹, O—(CH₂)₂—(CF₂)₄—(CH₂)₂—R¹, O—(CH₂)—[CH(CF₃)]—(CH₂)—R¹, O—(CH₂)—[C(CH₃)CF₃]—(CH₂)—R¹, O—(CH₂)—[CH(CH₂CF₃)]—(CH₂)—R¹, O—(CH₂)—[C(CH₃)(CH₂CF₃)]—(CH₂)—R¹, O—(CH₂)₂—(CF₂)—O—(CF₂)—O—(CF₂)—(CH₂)₂—R¹ and O—(CH₂)—(CF₂)—O—(CF₂)—O—(CF₂)—O—(CF₂)—(CH₂)—R¹ wherein R₁ is selected from the group consisting of an alkenyl of formula (4-1), (4-2), (4-3), (4-4), (4-5), (4-6), (4-7), (4-8), (4-9), (4-10), (4-11), or (4-12);

—(CH₂)₅—R¹, —(CH₂)₆—R¹, —(CH₂)₇—R¹, —(CH₂)₅—R¹, —(CH₂)₉—R¹, —(CH₂)₁₀—R¹, —(CH₂)₁₁—R¹, —(CH₂)₁₂—R¹, —(CH₂)₁₃—R¹, —(CH₂)₂—S—(CH₂)₂—R¹, —(CH₂)₂—SO₂—(CH₂)₂—R¹, —(CH₂)₂—S—(CH₂)₂—S—(CH₂)₂—R¹, —(CH₂)₂—O—(CH₂)₂—O—(CH₂)₂—R¹, —(CH₂)₂—S—(CH₂)₂—O—(CH₂)₂—R¹, —(CH₂)₂—SO₂—(CH₂)₂—O—(CH₂)₂—R¹, —(CH₂)₂—SO₂—(CH₂)₂—S—(CH₂)₂—R¹, —(CH₂)₂—O—(CH₂)₂—O—(CH₂)₂—O—(CH₂)₂—R¹, —(CH₂)₂—O—(CH₂)₂—S—(CH₂)₂—O—(CH₂)₂—R¹, —(CH₂)₂—S—(CH₂)₂—O—(CH₂)₂—S—(CH₂)₂—R¹, —(CH₂)₂—SO₂—(CH₂)₂—O—(CH₂)₂—SO₂—(CH₂)₂—R¹, —(CH₂)₂—S—(CH₂)₂—S—(CH₂)₂—S—(CH₂)₂—R¹, —(CH₂)₂—SO₂—(CH₂)₂—S—(CH₂)₂—SO₂—(CH₂)₂—R¹, —(CH₂)₂—SO₂—(CH₂)₂—SO₂—(CH₂)₂—SO₂—(CH₂)₂—R¹, —(CH₂)₂—O—(CH₂)₂—SO₂—(CH₂)₂—O—(CH₂)₂—R¹, —(CH₂)₃—(CF₂)—(CH₂)₃—R¹, —(CH₂)—(CF₂)₃—(CH₂)—R¹, —(CH₂)₂—(CF₂)₄—(CH₂)₂—R¹, —(CH₂)—[CH(CF₃)]—(CH₂)—R¹, —(CH₂)—[C(CH₃)CF₃]—(CH₂)—R¹, —(CH₂)— [CH(CH₂CF₃)]—(CH₂)—R¹, —(CH₂)—[C(CH₃)(CH₂CF₃)]—(CH₂)—R¹, —(CH₂)₂—(CF₂)—O—(CF₂)—O—(CF₂)—(CH₂)₂—R¹ and —(CH₂)—(CF₂)—O—(CF₂)—O—(CF₂)—O—(CF₂)—(CH₂)—R¹ where Y is a bond and wherein R₁ is selected from the group consisting of an alkenyl of formula (4-1), (4-2), (4-3), (4-4), (4-5), (4-6), (4-7), (4-8), (4-9), (4-10), (4-11), or (4-12);

S—(CH₂)₅—R¹, S—(CH₂)₆—R¹, S—(CH₂)₇—R¹, S—(CH₂)₅—R¹, S—(CH₂)₉—R¹, S—(CH₂)₁₀—R¹, S—(CH₂)₁₁—R¹, S—(CH₂)₁₂—R¹, S—(CH₂)₁₃—R¹, S—(CH₂)₂—S—(CH₂)₂—R¹, S—(CH₂)₂—SO₂—(CH₂)₂—R¹, S—(CH₂)₂—S—(CH₂)₂—S—(CH₂)₂—R¹, S—(CH₂)₂—O—(CH₂)₂—O—(CH₂)₂—R¹, S—(CH₂)₂—S—(CH₂)₂—O—(CH₂)₂—R¹, S—(CH₂)₂—SO₂—(CH₂)₂—O—(CH₂)₂—R¹, S—(CH₂)₂—SO₂—(CH₂)₂—S—(CH₂)₂—R¹, S—(CH₂)₂—O—(CH₂)₂—O—(CH₂)₂—O—(CH₂)₂—R¹, S—(CH₂)₂—O—(CH₂)₂—S—(CH₂)₂—O—(CH₂)₂—R¹, S—(CH₂)₂—S—(CH₂)₂—O—(CH₂)₂—S—(CH₂)₂—R¹, S—(CH₂)₂—SO₂—(CH₂)₂—O—(CH₂)₂—SO₂—(CH₂)₂—R¹, S—(CH₂)₂—S—(CH₂)₂—S—(CH₂)₂—S—(CH₂)₂—R¹, S—(CH₂)₂—SO₂—(CH₂)₂—S—(CH₂)₂—SO₂—(CH₂)₂—R¹, S—(CH₂)₂—SO₂—(CH₂)₂—SO₂—(CH₂)₂—SO₂—(CH₂)₂—R¹, S—(CH₂)₂—O—(CH₂)₂—SO₂—(CH₂)₂—O—(CH₂)₂—R¹, S—(CH₂)₃—(CF₂)—(CH₂)₃—R¹, S—(CH₂)—(CF₂)₃—(CH₂)—R¹, S—(CH₂)₂—(CF₂)₄—(CH₂)₂—R¹, S—(CH₂)—[CH(CF₃)]—(CH₂)—R¹, S—(CH₂)—[C(CH₃)CF₃]—(CH₂)—R¹, S—(CH₂)—[CH(CH₂CF₃)]—(CH₂)—R¹, S—(CH₂)—[C(CH₃)(CH₂CF₃)]—(CH₂)—R¹, S—(CH₂)₂—(CF₂)—O—(CF₂)—O—(CF₂)—(CH₂)₂—R¹ and S—(CH₂)—(CF₂)—O—(CF₂)—O—(CF₂)—O—(CF₂)—(CH₂)—R¹ wherein R₁ is selected from the group consisting of an alkenyl of formula (4-1), (4-2), (4-3), (4-4), (4-5), (4-6), (4-7), (4-8), (4-9), (4-10), (4-11), or (4-12);

SO₂—(CH₂)₅—R¹, SO₂—(CH₂)₆—R¹, SO₂—(CH₂)₇—R¹, SO₂—(CH₂)₅—R¹, SO₂—(CH₂)₉—R¹, SO₂—(CH₂)₁₀—R¹, SO₂—(CH₂)₁₁—R¹, SO₂—(CH₂)₁₂—R¹, SO₂—(CH₂)₁₃—R¹, SO₂—(CH₂)₂—S—(CH₂)₂—R¹, SO₂—(CH₂)₂—SO₂—(CH₂)₂—R¹, SO₂—(CH₂)₂—S—(CH₂)₂—S—(CH₂)₂—R¹, SO₂—(CH₂)₂—O—(CH₂)₂—O—(CH₂)₂—R¹, SO₂—(CH₂)₂—S—(CH₂)₂—O—(CH₂)₂—R¹, SO₂—(CH₂)₂—SO₂—(CH₂)₂—O—(CH₂)₂—R¹, SO₂—(CH₂)₂—SO₂—(CH₂)₂—S—(CH₂)₂—R¹, SO₂—(CH₂)₂—O—(CH₂)₂—O—(CH₂)₂—O—(CH₂)₂—R¹, SO₂—(CH₂)₂—O—(CH₂)₂—S—(CH₂)₂—O—(CH₂)₂—R¹, SO₂—(CH₂)₂—S—(CH₂)₂—O—(CH₂)₂—S—(CH₂)₂—R¹, SO₂—(CH₂)₂—SO₂—(CH₂)₂—O—(CH₂)₂—SO₂—(CH₂)₂—R¹, SO₂—(CH₂)₂—S—(CH₂)₂—S—(CH₂)₂—S—(CH₂)₂—R¹, SO₂—(CH₂)₂—SO₂—(CH₂)₂—S—(CH₂)₂—SO₂—(CH₂)₂—R¹, SO₂—(CH₂)₂—SO₂—(CH₂)₂—SO₂—(CH₂)₂—SO₂—(CH₂)₂—R¹, SO₂—(CH₂)₂—O—(CH₂)₂—SO₂—(CH₂)₂—O—(CH₂)₂—R¹, SO₂—(CH₂)₃—(CF₂)—(CH₂)₃—R¹, SO₂—(CH₂)—(CF₂)₃—(CH₂)—R¹, SO₂—(CH₂)₂—(CF₂)₄—(CH₂)₂—R¹, SO₂—(CH₂)—[CH(CF₃)]—(CH₂)—R¹, SO₂—(CH₂)—[C(CH₃)CF₃]—(CH₂)—R¹, SO₂—(CH₂)—[CH(CH₂CF₃)]—(CH₂)—R¹, SO₂—(CH₂)—[C(CH₃)(CH₂CF₃)]—(CH₂)—R¹, SO₂—(CH₂)₂—(CF₂)—O—(CF₂)—O—(CF₂)—(CH₂)₂—R¹ and SO₂—(CH₂)—(CF₂)—O—(CF₂)—O—(CF₂)—O—(CF₂)—(CH₂)—R¹ wherein R₁ is selected from the group consisting of an alkenyl of formula (4-1), (4-2), (4-3), (4-4), (4-5), (4-6), (4-7), (4-8), (4-9), (4-10), (4-11), or (4-12).

The substituent N—R₂—R₁ within formulae (I), (I #), (I′), (I′-a), (I′-b), (I′-c), (I′-d), (I′-e), (I′-f), (I′-g), (I′-h), (I′-i) and (I″) is therefore particularly preferably selected from the group consisting of

N—(CH₂)₅—R¹, N—(CH₂)₆—R¹, N—(CH₂)₇—R¹, N—(CH₂)₈—R¹, N—(CH₂)₉—R¹, N—(CH₂)₁₀—R¹, N—(CH₂)₁₁—R¹, N—(CH₂)₁₂—R¹, N—(CH₂)₁₃—R¹, N—(CH₂)₂—S—(CH₂)₂—R¹, N—(CH₂)₂—SO₂—(CH₂)₂—R¹, N—(CH₂)₂—S—(CH₂)₂—S—(CH₂)₂—R¹, N—(CH₂)₂—O—(CH₂)₂—O—(CH₂)₂—R¹, N—(CH₂)₂—S—(CH₂)₂—O—(CH₂)₂—R¹, N—(CH₂)₂—SO₂—(CH₂)₂—O—(CH₂)₂—R¹, N—(CH₂)₂—SO₂—(CH₂)₂—S—(CH₂)₂—R¹, N—(CH₂)₂—O—(CH₂)₂—O—(CH₂)₂—O—(CH₂)₂—R¹, N—(CH₂)₂—O—(CH₂)₂—S—(CH₂)₂—O—(CH₂)₂—R¹, N—(CH₂)₂—S—(CH₂)₂—O—(CH₂)₂—S—(CH₂)₂—R¹, N—(CH₂)₂—SO₂—(CH₂)₂—O—(CH₂)₂—SO₂—(CH₂)₂—R¹, N—(CH₂)₂—S—(CH₂)₂—S—(CH₂)₂—S—(CH₂)₂—R¹, N—(CH₂)₂—SO₂—(CH₂)₂—S—(CH₂)₂—SO₂—(CH₂)₂—R¹, N—(CH₂)₂—SO₂—(CH₂)₂—SO₂—(CH₂)₂—SO₂—(CH₂)₂—R¹, N—(CH₂)₂—O—(CH₂)₂—SO₂—(CH₂)₂—O—(CH₂)₂—R¹, N—(CH₂)₃—(CF₂)—(CH₂)₃—R¹, N—(CH₂)—(CF₂)₃—(CH₂)—R¹, N—(CH₂)₂—(CF₂)₄—(CH₂)₂—R¹, N—(CH₂)—[CH(CF₃)]—(CH₂)—R¹, N—(CH₂)—[C(CH₃)CF₃]—(CH₂)—R¹, N—(CH₂)—[CH(CH₂CF₃)]—(CH₂)—R¹, N—(CH₂)—[C(CH₃)(CH₂CF₃)]—(CH₂)—R¹, N—(CH₂)₂—(CF₂)—O—(CF₂)—O—(CF₂)—(CH₂)₂—R¹ and N—(CH₂)—(CF₂)—O—(CF₂)—O—(CF₂)—O—(CF₂)—(CH₂)—R¹ wherein R₁ is selected from the group consisting of an alkenyl of formula (4-1), (4-2), (4-3), (4-4), (4-5), (4-6), (4-7), (4-8), (4-9), (4-10), (4-11), or (4-12).

Particularly preferably, the compounds of formulae (I), (I #), (I′), (I′-a), (I′-b), (I′-c), (I′-d), (I′-e), (I′-f), (I′-g), (I′-h), (I′-i) and (I″) comprise a polymerizable group R₁ which is represented by formulae (4-1), (4-2), (4-5), (4-6), (4-11) and (4-12).

Very particularly preferably, the compounds of formulae (I), (I #), (I′), (I′-a), (I′-b), (I′-c), (I′-d), (I′-e), (I′-f), (I′-g), (I′-h), (I′-i) and (I″) comprise a polymerizable group R₁ which is a methacryl or an acryl group represented by formula (4-1) and (4-2).

The invention therefore relates further to an ophthalmic device or a precursor article for manufacturing an ophthalmic device comprising polymerized compounds of formulae (I), (I #), (I′), (I′-a), (I′-b), (I′-c), (I′-d), (I′-e), (I′-f), (I′-g), (I′-h), (I′-i) and/or (I″) as described before or preferably described before wherein R₁ is at each occurrence independently an acryl or methacryl group.

The invention therefore relates further to compounds of formulae I), (I #), (I′), (I′-a), (I′-b), (I′-c), (I′-d), (I′-e), (I′-f), (I′-g), (I′-h), (I′-i) and/or (I″) as described before or preferably described before wherein R₁ is at each occurrence independently an acryl or methacryl group.

Examples for compounds/monomers of formulae I), (I #), (I′), (I′-a), (I′-b), (I′-c), (I′-d), (I′-e), (I′-f), (I′-g), (I′-h), (I′-i) and/or (I″) are the following compounds (A-001) to (A-162) as shown in table 1.

TABLE 1

| | |
|---|---|
| 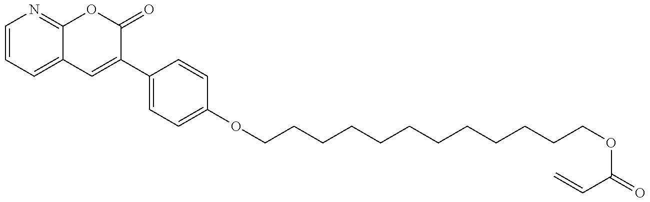 [SMILES: C=CC(=O)OCCCCCCCCCCCCOc1ccc(-c2cc3cccnc3oc2=O)cc1] | A-001 |
| 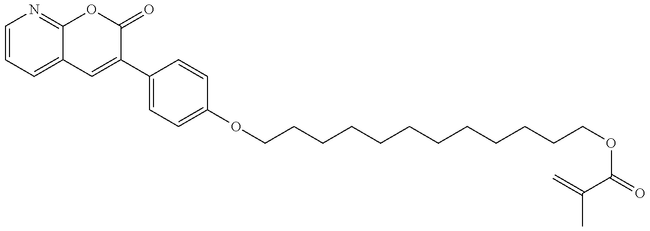 [SMILES: C=C(C)C(=O)OCCCCCCCCCCCCOc1ccc(-c2cc3cccnc3oc2=O)cc1] | A-002 |
| 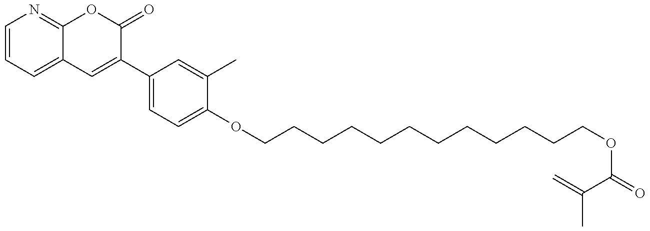 [SMILES: C=C(C)C(=O)OCCCCCCCCCCCCOc1ccc(-c2cc3cccnc3oc2=O)cc1C] | A-003 |
| 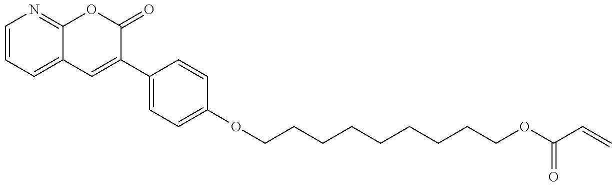 [SMILES: C=CC(=O)OCCCCCCCCCOc1ccc(-c2cc3cccnc3oc2=O)cc1] | A-004 |
| 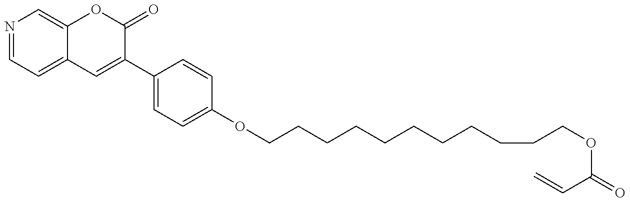 [SMILES: C=CC(=O)OCCCCCCCCCCCOc1ccc(-c2cc3ccncc3oc2=O)cc1] | A-005 |
| 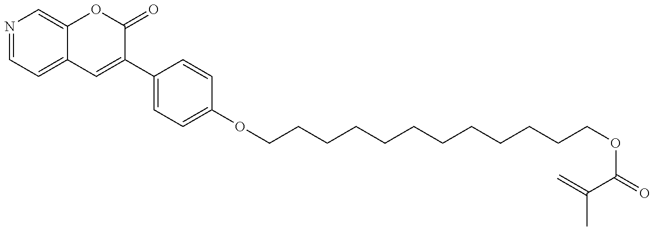 [SMILES: C=C(C)C(=O)OCCCCCCCCCCCCOc1ccc(-c2cc3ccncc3oc2=O)cc1] | A-006 |
| 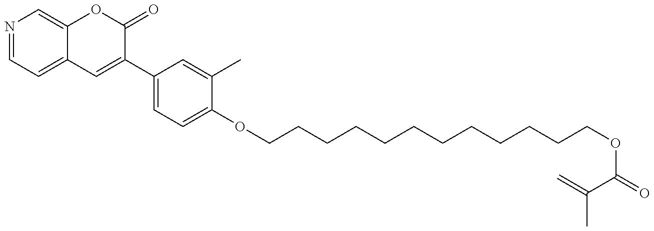 [SMILES: C=C(C)C(=O)OCCCCCCCCCCCCOc1ccc(-c2cc3ccncc3oc2=O)cc1C] | A-007 |
| 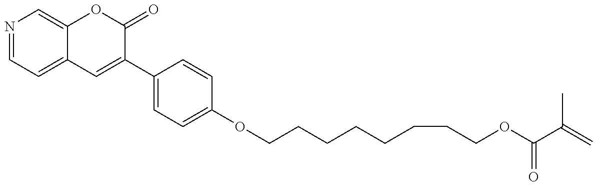 [SMILES: C=C(C)C(=O)OCCCCCCCCOc1ccc(-c2cc3ccncc3oc2=O)cc1] | A-008 |
| 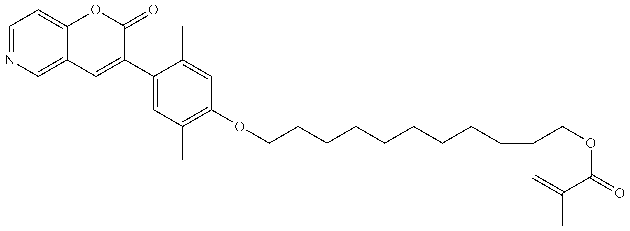 [SMILES: C=C(C)C(=O)OCCCCCCCCCCCOc1cc(C)c(-c2cc3cnccc3oc2=O)cc1C] | A-009 |
| 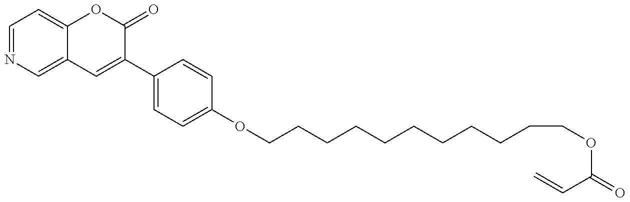 [SMILES: C=CC(=O)OCCCCCCCCCCCOc1ccc(-c2cc3cnccc3oc2=O)cc1] | A-010 |
| 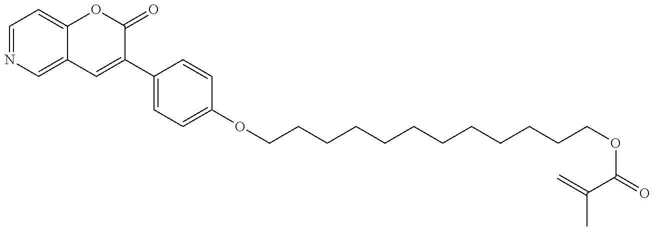 [SMILES: C=C(C)C(=O)OCCCCCCCCCCCCOc1ccc(-c2cc3cnccc3oc2=O)cc1] | A-011 |
| 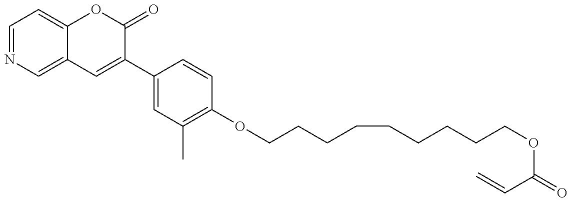 [SMILES: C=CC(=O)OCCCCCCCCCOc1ccc(-c2cc3cnccc3oc2=O)cc1C] | A-012 |
| 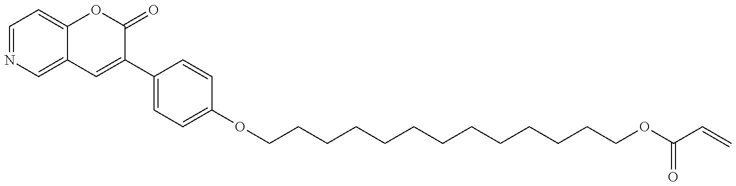 [SMILES: C=CC(=O)OCCCCCCCCCCCCCOc1ccc(-c2cc3cnccc3oc2=O)cc1] | A-013 |
| 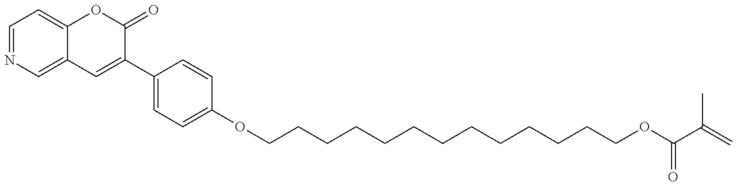 [SMILES: C=C(C)C(=O)OCCCCCCCCCCCCCOc1ccc(-c2cc3cnccc3oc2=O)cc1] | A-014 |
| 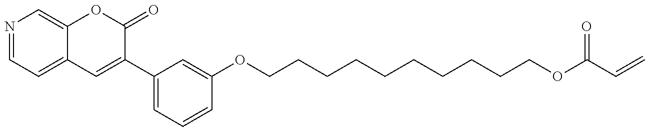 [SMILES: C=CC(=O)OCCCCCCCCCCOc1cccc(-c2cc3ccncc3oc2=O)c1] | A-015 |
| 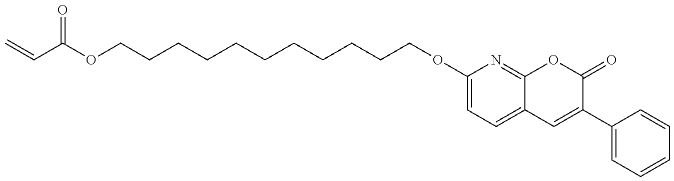 [SMILES: C=CC(=O)OCCCCCCCCCCCOc1ccc2cc(-c3ccccc3)c(=O)oc2n1] | A-016 |
| 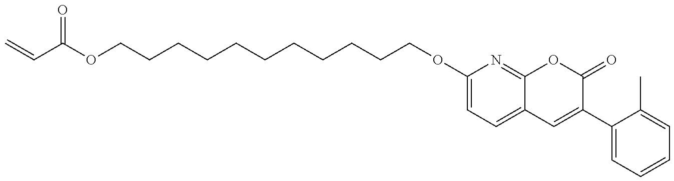 [SMILES: C=CC(=O)OCCCCCCCCCCCOc1ccc2cc(-c3ccccc3C)c(=O)oc2n1] | A-017 |
| 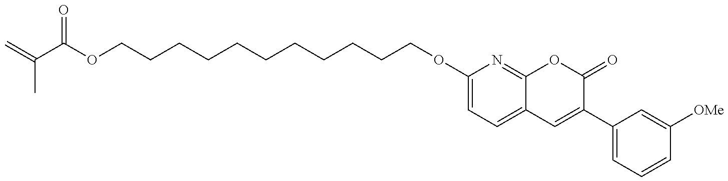 [SMILES: C=C(C)C(=O)OCCCCCCCCCCCOc1ccc2cc(-c3cccc(OC)c3)c(=O)oc2n1] | A-018 |
| 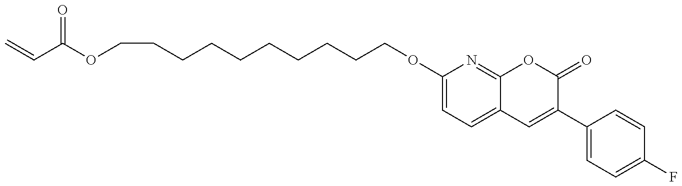 [SMILES: C=CC(=O)OCCCCCCCCCCOc1ccc2cc(-c3ccc(F)cc3)c(=O)oc2n1] | A-019 |
| 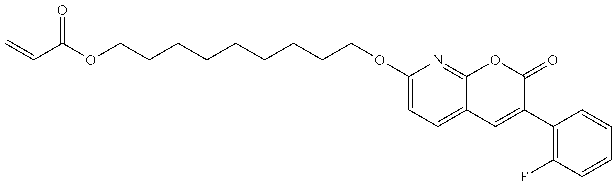 [SMILES: C=CC(=O)OCCCCCCCCCOc1ccc2cc(-c3ccccc3F)c(=O)oc2n1] | A-020 |
| 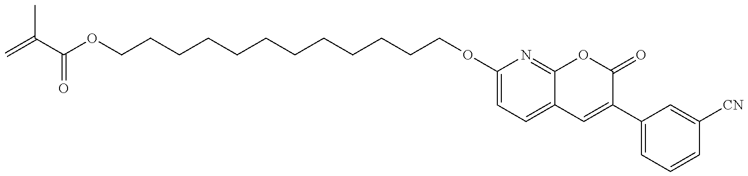 [SMILES: C=C(C)C(=O)OCCCCCCCCCCCCOc1ccc2cc(-c3cccc(C#N)c3)c(=O)oc2n1] | A-021 |
| 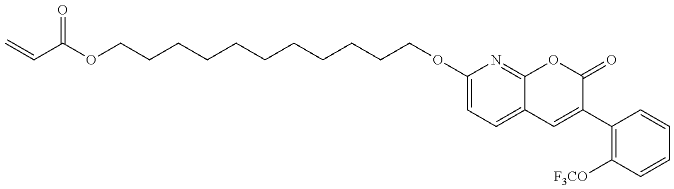 [SMILES: C=CC(=O)OCCCCCCCCCCCOc1ccc2cc(-c3ccccc3OC(F)(F)F)c(=O)oc2n1] | A-022 |
| 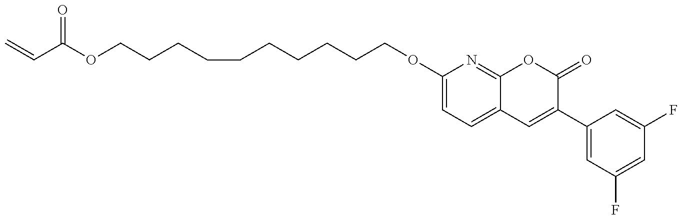 [SMILES: C=CC(=O)OCCCCCCCCCCOc1ccc2cc(-c3cc(F)cc(F)c3)c(=O)oc2n1] | A-023 |
| 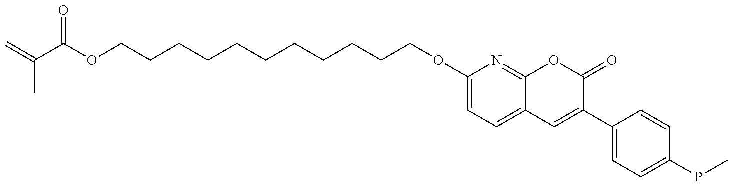 | A-024 |
| 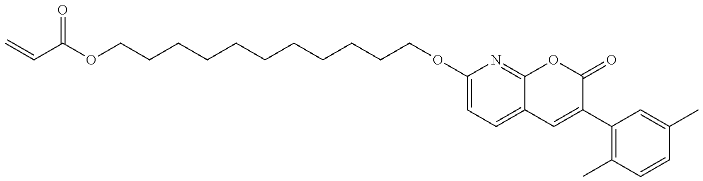 [SMILES: C=CC(=O)OCCCCCCCCCCCOc1ccc2cc(-c3cc(C)ccc3C)c(=O)oc2n1] | A-025 |
| 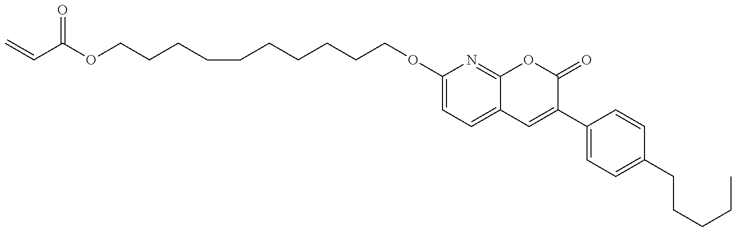 [SMILES: C=CC(=O)OCCCCCCCCCCOc1ccc2cc(-c3ccc(CCCCC)cc3)c(=O)oc2n1] | A-026 |
| 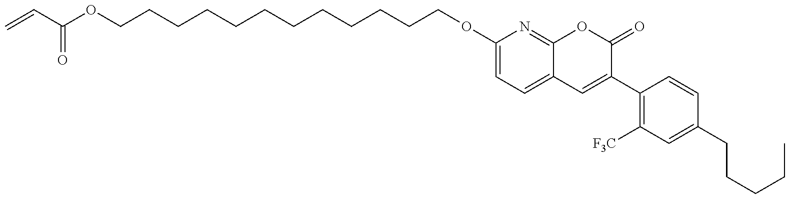 [SMILES: C=CC(=O)OCCCCCCCCCCCCOc1ccc2cc(-c3ccc(CCCCC)cc3C(F)(F)F)c(=O)oc2n1] | A-027 |
| 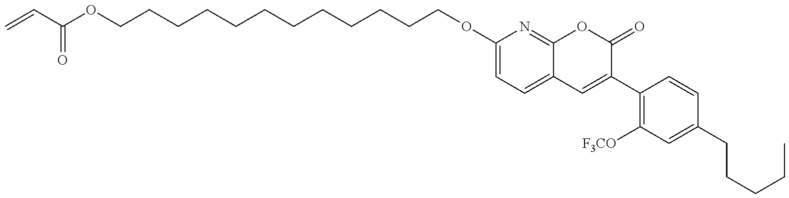 [SMILES: C=CC(=O)OCCCCCCCCCCCCOc1ccc2cc(-c3ccc(CCCCC)cc3OC(F)(F)F)c(=O)oc2n1] | A-028 |
| 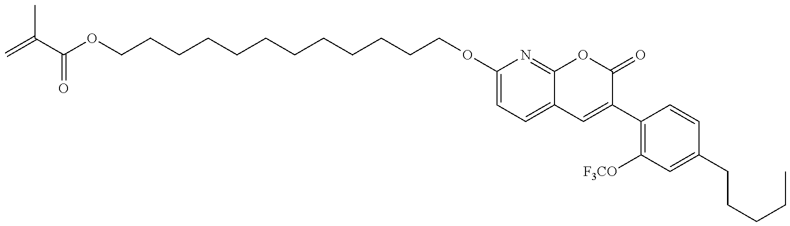 [SMILES: C=C(C)C(=O)OCCCCCCCCCCCCOc1ccc2cc(-c3ccc(CCCCC)cc3OC(F)(F)F)c(=O)oc2n1] | A-029 |
| 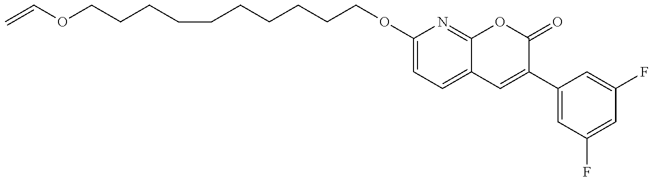 [SMILES: C=COCCCCCCCCCCOc1ccc2cc(-c3cc(F)cc(F)c3)c(=O)oc2n1] | A-030 |
| 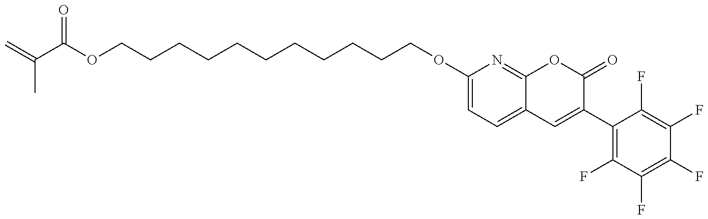 [SMILES: C=C(C)C(=O)OCCCCCCCCCCCOc1ccc2cc(-c3c(F)c(F)c(F)c(F)c3F)c(=O)oc2n1] | A-031 |
| 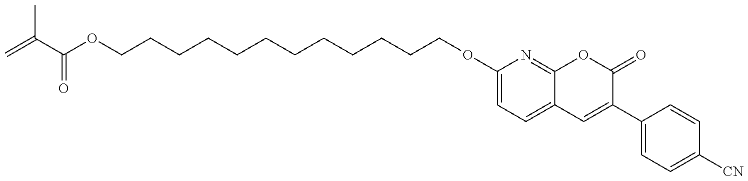 [SMILES: C=C(C)C(=O)OCCCCCCCCCCCCOc1ccc2cc(-c3ccc(C#N)cc3)c(=O)oc2n1] | A-032 |
| 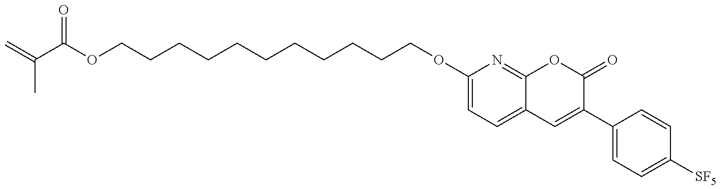 | A-033 |
| 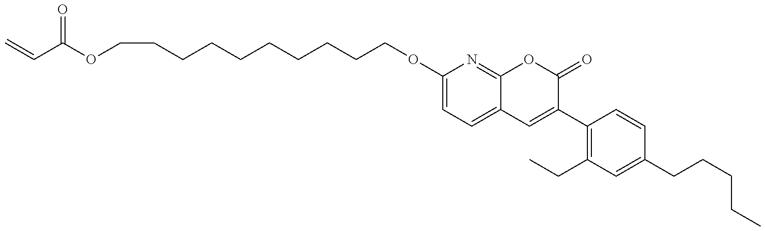 [SMILES: C=CC(=O)OCCCCCCCCCCOc1ccc2cc(-c3ccc(CCCCC)cc3CC)c(=O)oc2n1] | A-034 |
| 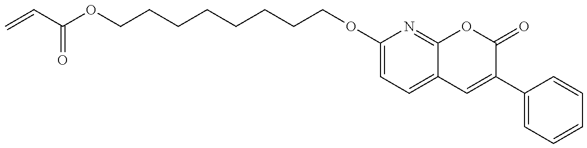 [SMILES: C=CC(=O)OCCCCCCCCOc1ccc2cc(-c3ccccc3)c(=O)oc2n1] | A-035 |
| 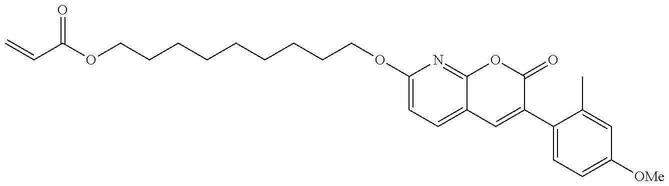 [SMILES: C=CC(=O)OCCCCCCCCCOc1ccc2cc(-c3ccc(OC)cc3C)c(=O)oc2n1] | A-036 |
| 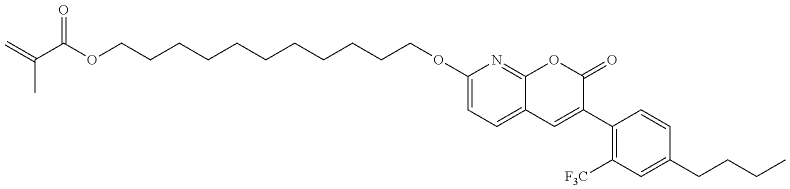 [SMILES: C=C(C)C(=O)OCCCCCCCCCCCOc1ccc2cc(-c3ccc(CCCC)cc3C(F)(F)F)c(=O)oc2n1] | A-037 |
| 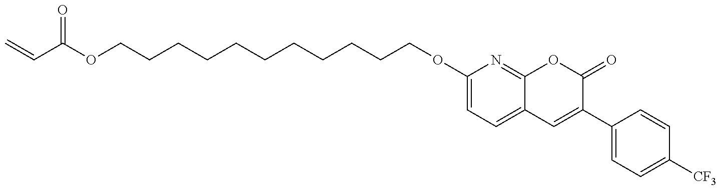 [SMILES: C=CC(=O)OCCCCCCCCCCCOc1ccc2cc(-c3ccc(C(F)(F)F)cc3)c(=O)oc2n1] | A-038 |
| 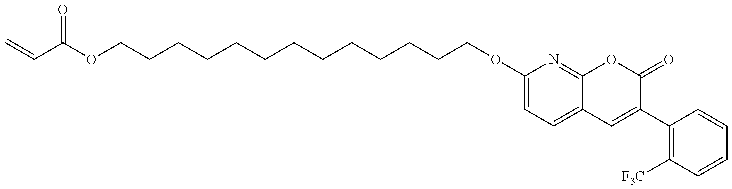 [SMILES: C=CC(=O)OCCCCCCCCCCCCCOc1ccc2cc(-c3ccccc3C(F)(F)F)c(=O)oc2n1] | A-039 |
| 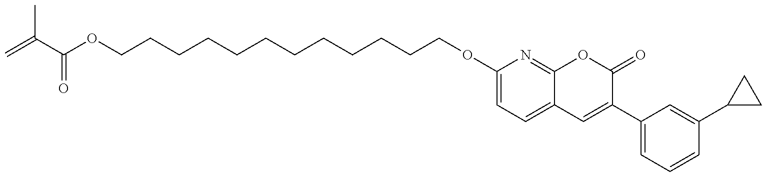 [SMILES: C=C(C)C(=O)OCCCCCCCCCCCCOc1ccc2cc(-c3cccc(C4CC4)c3)c(=O)oc2n1] | A-040 |
| 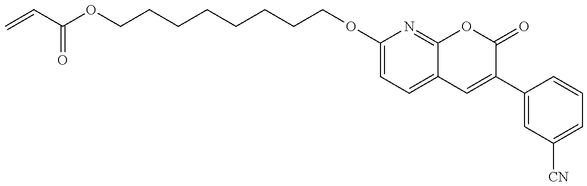 [SMILES: C=CC(=O)OCCCCCCCCOc1ccc2cc(-c3cccc(C#N)c3)c(=O)oc2n1] | A-041 |
| 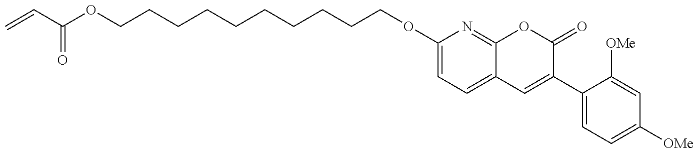 [SMILES: C=CC(=O)OCCCCCCCCCCOc1ccc2cc(-c3ccc(OC)cc3OC)c(=O)oc2n1] | A-042 |
| 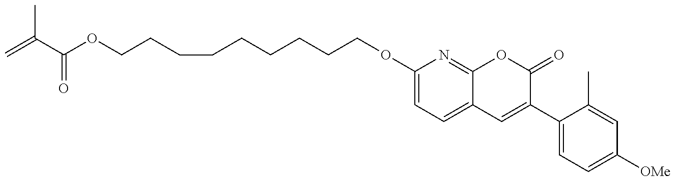 [SMILES: C=C(C)C(=O)OCCCCCCCCCOc1ccc2cc(-c3ccc(OC)cc3C)c(=O)oc2n1] | A-043 |
| 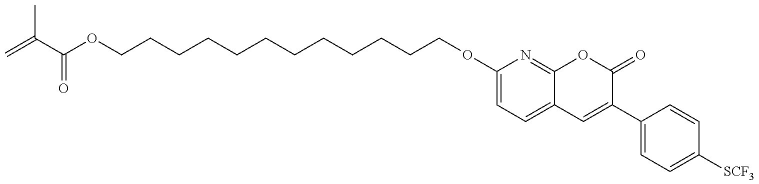 | A-044 |
| 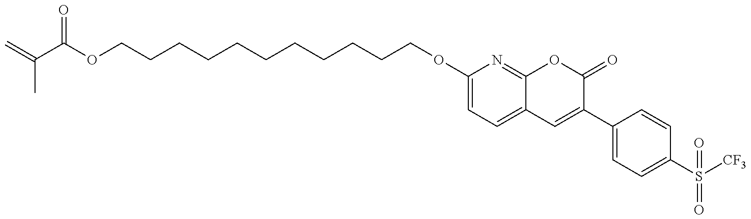 [SMILES: C=C(C)C(=O)OCCCCCCCCCCCOc1ccc2cc(-c3ccc(S(=O)(=O)C(F)(F)F)cc3)c(=O)oc2n1] | A-045 |
| 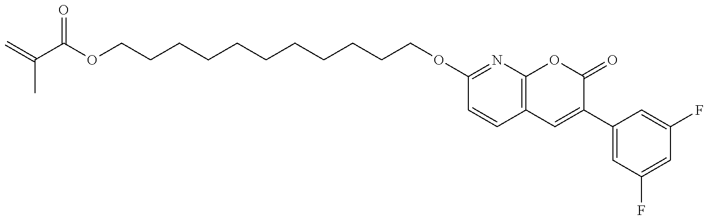 [SMILES: C=C(C)C(=O)OCCCCCCCCCCCOc1ccc2cc(-c3cc(F)cc(F)c3)c(=O)oc2n1] | A-046 |
| 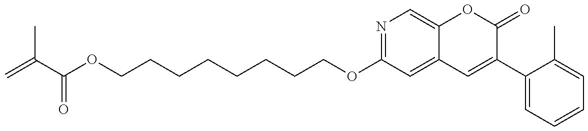 [SMILES: C=C(C)C(=O)OCCCCCCCCOc1cc2cc(-c3ccccc3C)c(=O)oc2cn1] | A-047 |
| 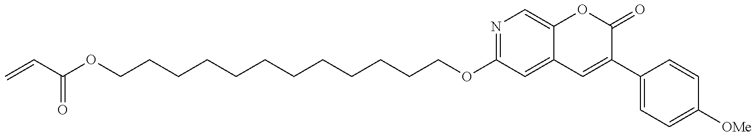 [SMILES: C=CC(=O)OCCCCCCCCCCCCOc1cc2cc(-c3ccc(OC)cc3)c(=O)oc2cn1] | A-048 |
| 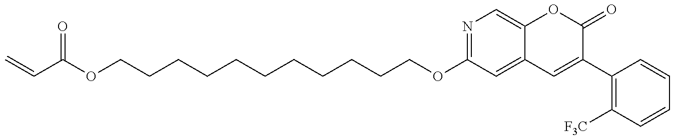 [SMILES: C=CC(=O)OCCCCCCCCCCCOc1cc2cc(-c3ccccc3C(F)(F)F)c(=O)oc2cn1] | A-049 |
| 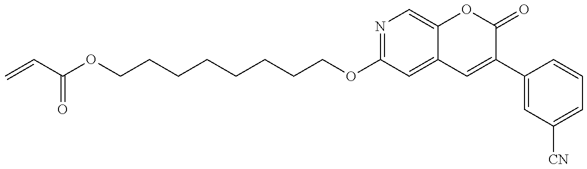 [SMILES: C=CC(=O)OCCCCCCCCOc1cc2cc(-c3cccc(C#N)c3)c(=O)oc2cn1] | A-050 |
| 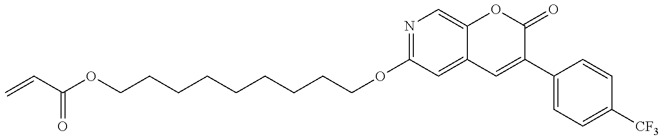 [SMILES: C=CC(=O)OCCCCCCCCCOc1cc2cc(-c3ccc(C(F)(F)F)cc3)c(=O)oc2cn1] | A-051 |
| 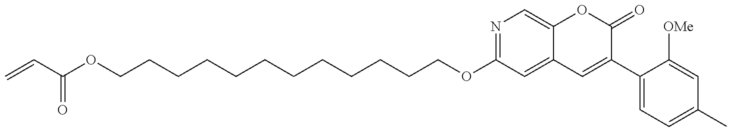 [SMILES: C=CC(=O)OCCCCCCCCCCCCOc1cc2cc(-c3ccc(C)cc3OC)c(=O)oc2cn1] | A-052 |
| 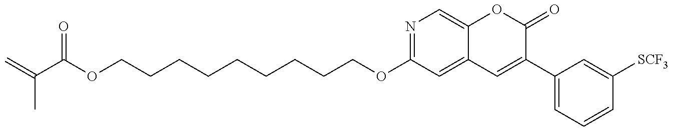 | A-053 |
| 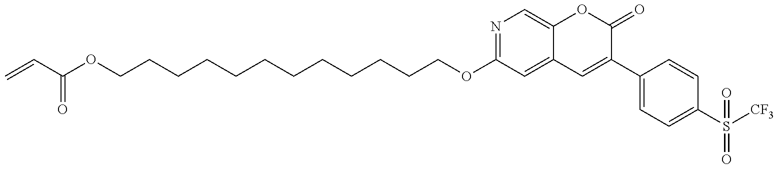 [SMILES: C=CC(=O)OCCCCCCCCCCCCOc1cc2cc(-c3ccc(S(=O)(=O)C(F)(F)F)cc3)c(=O)oc2cn1] | A-054 |
| 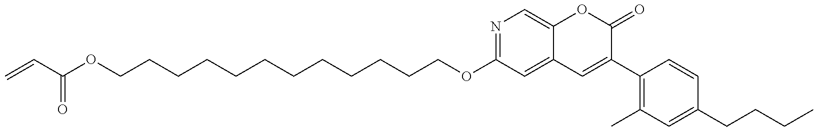 [SMILES: C=CC(=O)OCCCCCCCCCCCCOc1cc2cc(-c3ccc(CCCC)cc3C)c(=O)oc2cn1] | A-055 |
| 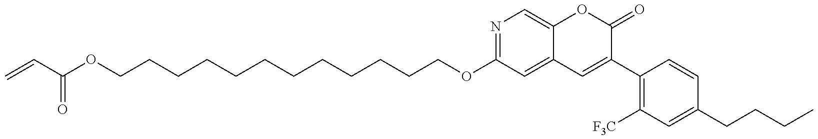 [SMILES: C=CC(=O)OCCCCCCCCCCCCOc1cc2cc(-c3ccc(CCCC)cc3C(F)(F)F)c(=O)oc2cn1] | A-056 |
| 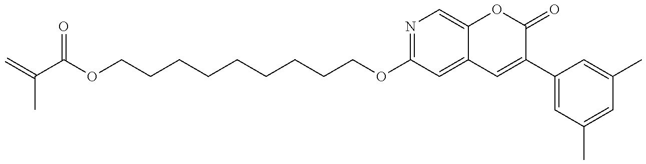 [SMILES: C=C(C)C(=O)OCCCCCCCCCOc1cc2cc(-c3cc(C)cc(C)c3)c(=O)oc2cn1] | A-057 |
| 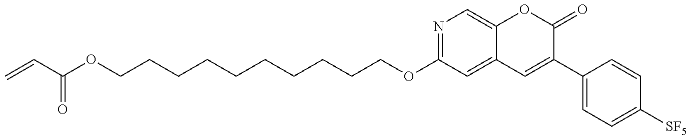 | A-058 |
| 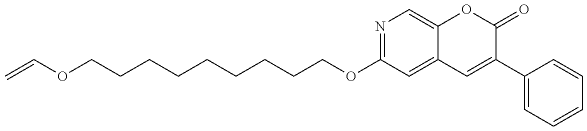 [SMILES: C=COCCCCCCCCCOc1cc2cc(-c3ccccc3)c(=O)oc2cn1] | A-059 |
| 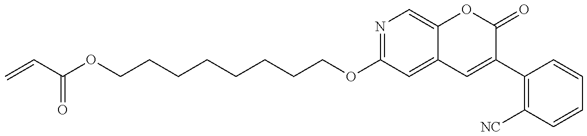 [SMILES: C=CC(=O)OCCCCCCCCOc1cc2cc(-c3ccccc3C#N)c(=O)oc2cn1] | A-060 |
| 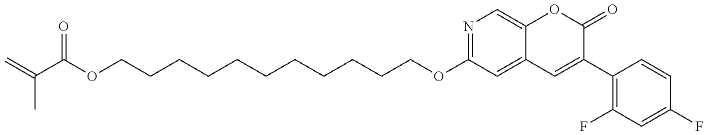 [SMILES: C=C(C)C(=O)OCCCCCCCCCCCOc1cc2cc(-c3ccc(F)cc3F)c(=O)oc2cn1] | A-061 |
| 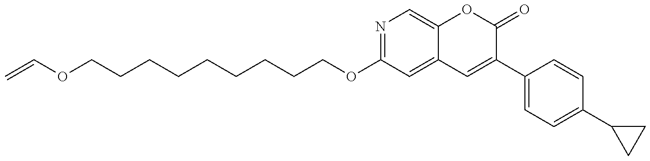 [SMILES: C=COCCCCCCCCCOc1cc2cc(-c3ccc(C4CC4)cc3)c(=O)oc2cn1] | A-062 |
| 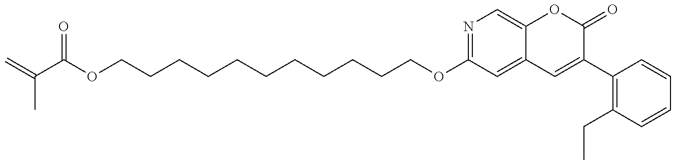 [SMILES: C=C(C)C(=O)OCCCCCCCCCCCOc1cc2cc(-c3ccccc3CC)c(=O)oc2cn1] | A-063 |
| 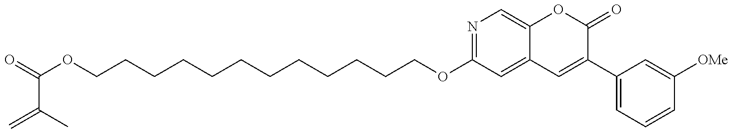 [SMILES: C=C(C)C(=O)OCCCCCCCCCCCCOc1cc2cc(-c3cccc(OC)c3)c(=O)oc2cn1] | A-064 |
| 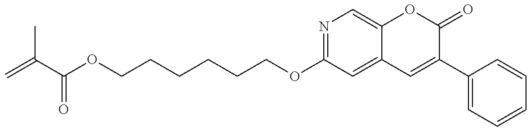 [SMILES: C=C(C)C(=O)OCCCCCCOc1cc2cc(-c3ccccc3)c(=O)oc2cn1] | A-065 |
| 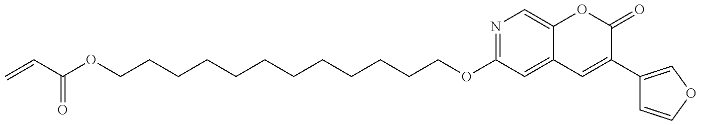 [SMILES: C=CC(=O)OCCCCCCCCCCCCOc1cc2cc(-c3ccoc3)c(=O)oc2cn1] | A-066 |
| 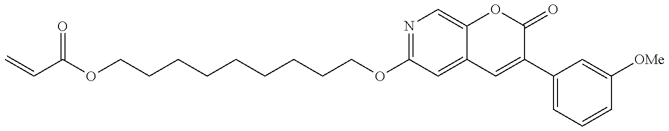 [SMILES: C=CC(=O)OCCCCCCCCCOc1cc2cc(-c3cccc(OC)c3)c(=O)oc2cn1] | A-067 |
| 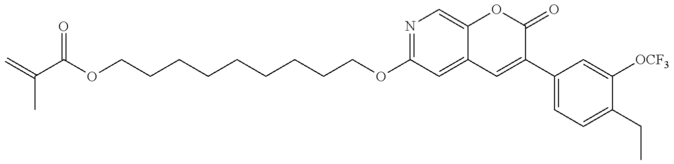 [SMILES: C=C(C)C(=O)OCCCCCCCCCOc1cc2cc(-c3ccc(CC)c(OC(F)(F)F)c3)c(=O)oc2cn1] | A-068 |
| 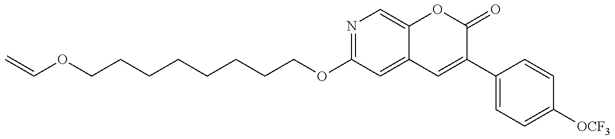 [SMILES: C=COCCCCCCCCOc1cc2cc(-c3ccc(OC(F)(F)F)cc3)c(=O)oc2cn1] | A-069 |
| 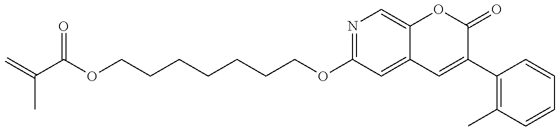 [SMILES: C=C(C)C(=O)OCCCCCCCOc1cc2cc(-c3ccccc3C)c(=O)oc2cn1] | A-070 |
| 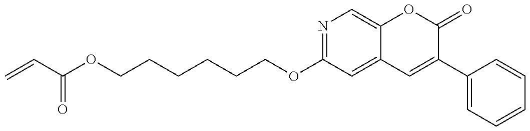 [SMILES: C=CC(=O)OCCCCCCOc1cc2cc(-c3ccccc3)c(=O)oc2cn1] | A-071 |
| 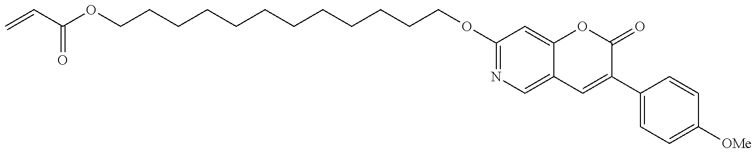 [SMILES: C=CC(=O)OCCCCCCCCCCCCOc1cc2oc(=O)c(-c3ccc(OC)cc3)cc2cn1] | A-072 |
| 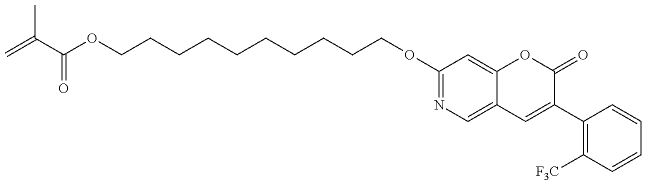 [SMILES: C=C(C)C(=O)OCCCCCCCCCCOc1cc2oc(=O)c(-c3ccccc3C(F)(F)F)cc2cn1] | A-073 |
| 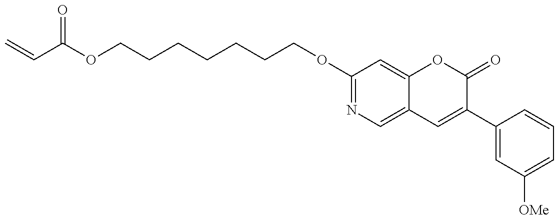 [SMILES: C=CC(=O)OCCCCCCCOc1cc2oc(=O)c(-c3cccc(OC)c3)cc2cn1] | A-074 |
| 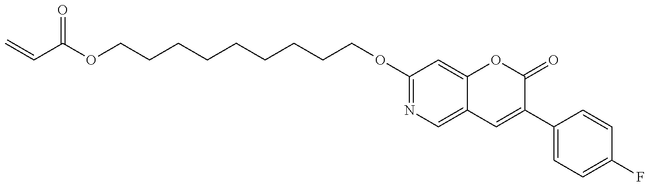 [SMILES: C=CC(=O)OCCCCCCCCCOc1cc2oc(=O)c(-c3ccc(F)cc3)cc2cn1] | A-075 |
| 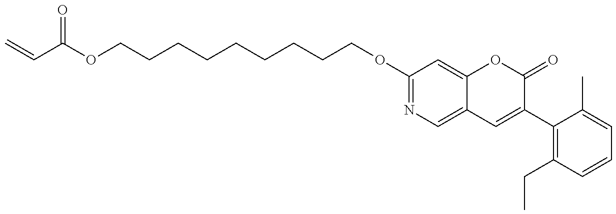 [SMILES: C=CC(=O)OCCCCCCCCCOc1cc2oc(=O)c(-c3c(C)cccc3CC)cc2cn1] | A-076 |
| 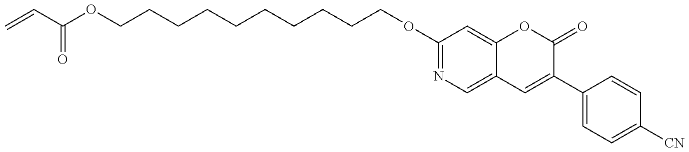 [SMILES: C=CC(=O)OCCCCCCCCCCOc1cc2oc(=O)c(-c3ccc(C#N)cc3)cc2cn1] | A-077 |
| 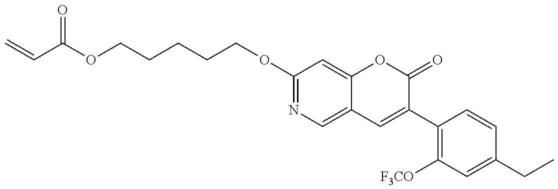 [SMILES: C=CC(=O)OCCCCCOc1cc2oc(=O)c(-c3ccc(CC)cc3OC(F)(F)F)cc2cn1] | A-078 |
| 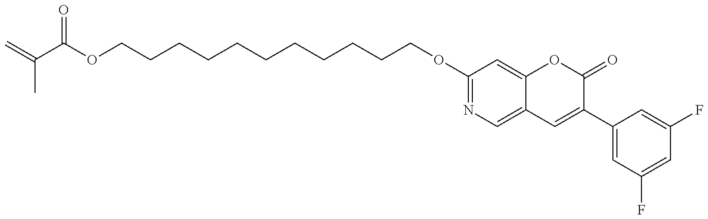 [SMILES: C=C(C)C(=O)OCCCCCCCCCCCOc1cc2oc(=O)c(-c3cc(F)cc(F)c3)cc2cn1] | A-079 |
| 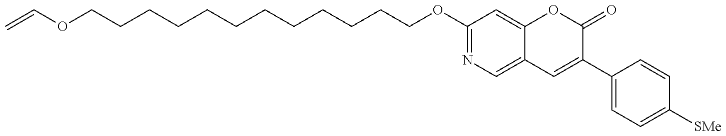 [SMILES: C=COCCCCCCCCCCCCOc1cc2oc(=O)c(-c3ccc(SC)cc3)cc2cn1] | A-080 |
| 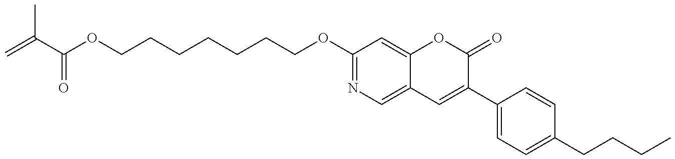 [SMILES: C=C(C)C(=O)OCCCCCCCOc1cc2oc(=O)c(-c3ccc(CCCC)cc3)cc2cn1] | A-081 |
| 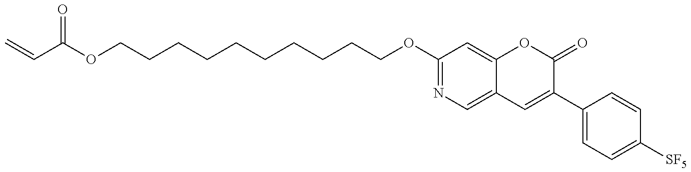 | A-082 |
| 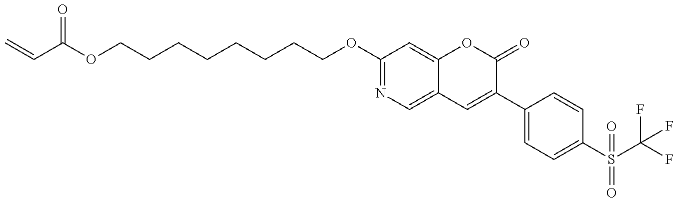 [SMILES: C=CC(=O)OCCCCCCCCOc1cc2oc(=O)c(-c3ccc(S(=O)(=O)C(F)(F)F)cc3)cc2cn1] | A-083 |
| 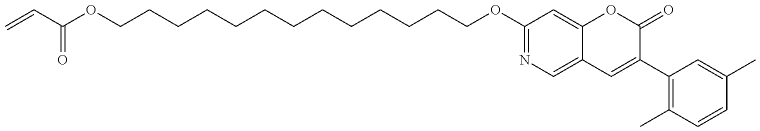 [SMILES: C=CC(=O)OCCCCCCCCCCCCCOc1cc2oc(=O)c(-c3cc(C)ccc3C)cc2cn1] | A-084 |
| 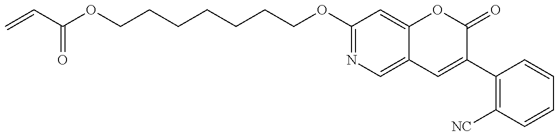 [SMILES: C=CC(=O)OCCCCCCCOc1cc2oc(=O)c(-c3ccccc3C#N)cc2cn1] | A-085 |
| 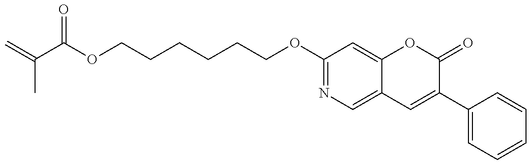 [SMILES: C=C(C)C(=O)OCCCCCCOc1cc2oc(=O)c(-c3ccccc3)cc2cn1] | A-086 |
| 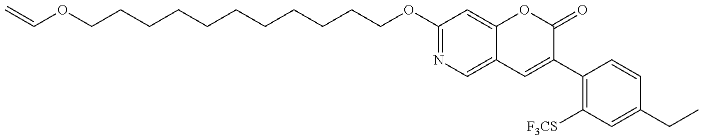 | A-087 |
| 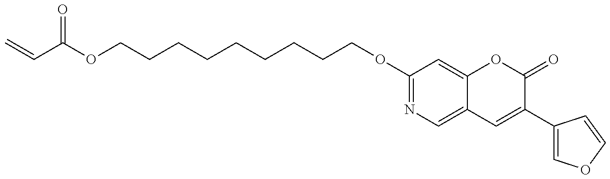 [SMILES: C=CC(=O)OCCCCCCCCCOc1cc2oc(=O)c(-c3ccoc3)cc2cn1] | A-088 |
| 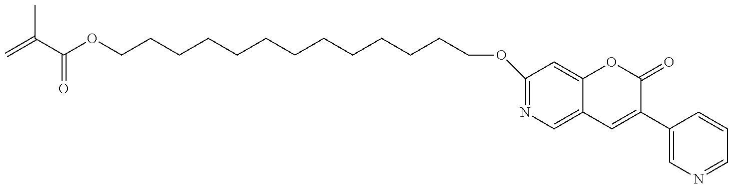 [SMILES: C=C(C)C(=O)OCCCCCCCCCCCCCOc1cc2oc(=O)c(-c3cccnc3)cc2cn1] | A-089 |
| 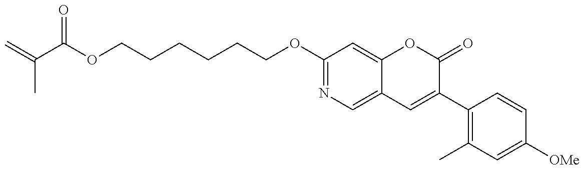 [SMILES: C=C(C)C(=O)OCCCCCCOc1cc2oc(=O)c(-c3ccc(OC)cc3C)cc2cn1] | A-090 |
| 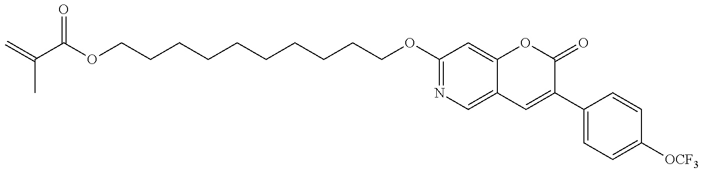 [SMILES: C=C(C)C(=O)OCCCCCCCCCCOc1cc2oc(=O)c(-c3ccc(OC(F)(F)F)cc3)cc2cn1] | A-091 |
| 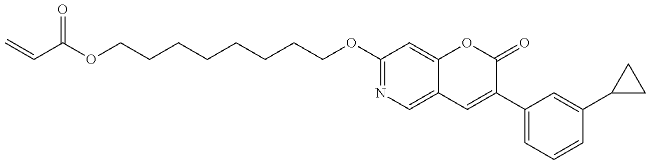 [SMILES: C=CC(=O)OCCCCCCCCOc1cc2oc(=O)c(-c3cccc(C4CC4)c3)cc2cn1] | A-092 |
| 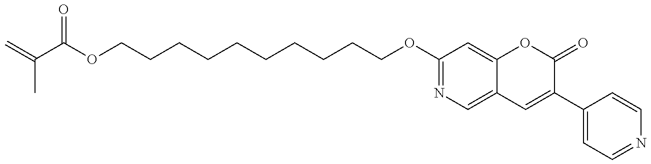 [SMILES: C=C(C)C(=O)OCCCCCCCCCCOc1cc2oc(=O)c(-c3ccncc3)cc2cn1] | A-093 |
| 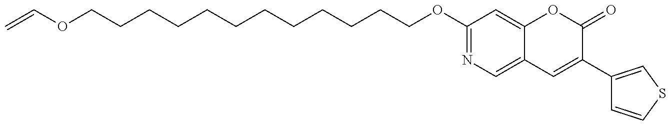 [SMILES: C=COCCCCCCCCCCCCOc1cc2oc(=O)c(-c3ccsc3)cc2cn1] | A-094 |
| 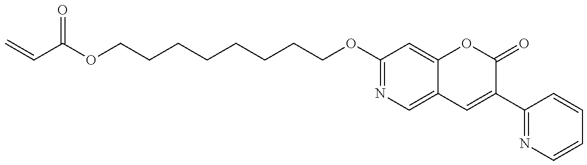 [SMILES: C=CC(=O)OCCCCCCCCOc1cc2oc(=O)c(-c3ccccn3)cc2cn1] | A-095 |
| 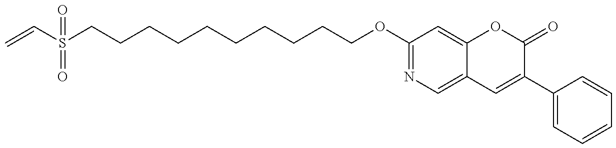 [SMILES: C=CS(=O)(=O)CCCCCCCCCCOc1cc2oc(=O)c(-c3ccccc3)cc2cn1] | A-096 |
| 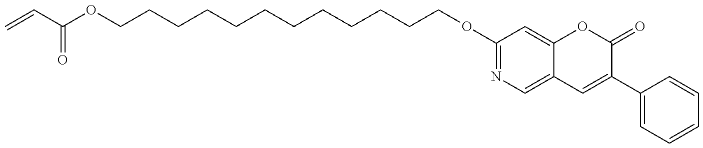 [SMILES: C=CC(=O)OCCCCCCCCCCCCOc1cc2oc(=O)c(-c3ccccc3)cc2cn1] | A-097 |
| 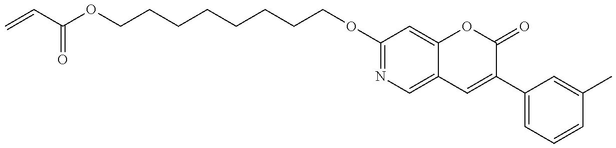 [SMILES: C=CC(=O)OCCCCCCCCOc1cc2oc(=O)c(-c3cccc(C)c3)cc2cn1] | A-098 |
| 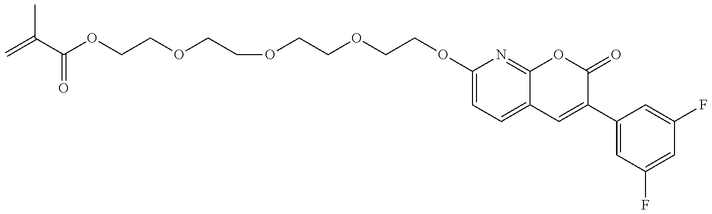 [SMILES: C=C(C)C(=O)OCCOCCOCCOCCOc1ccc2cc(-c3cc(F)cc(F)c3)c(=O)oc2n1] | A-099 |
| 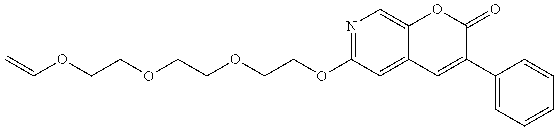 [SMILES: C=COCCOCCOCCOc1cc2cc(-c3ccccc3)c(=O)oc2cn1] | A-100 |
| 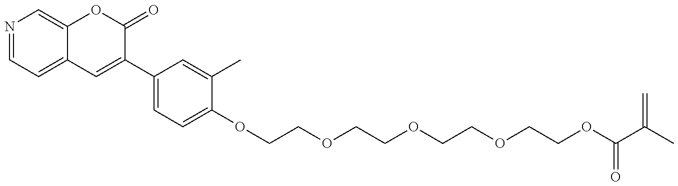 [SMILES: C=C(C)C(=O)OCCOCCOCCOCCOc1ccc(-c2cc3ccncc3oc2=O)cc1C] | A-101 |
| 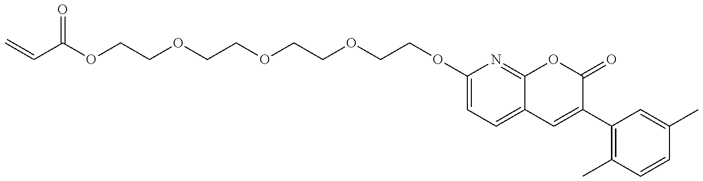 [SMILES: C=CC(=O)OCCOCCOCCOCCOc1ccc2cc(-c3cc(C)ccc3C)c(=O)oc2n1] | A-102 |
| 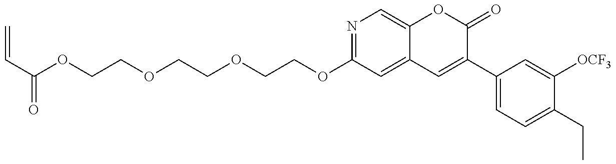 [SMILES: C=CC(=O)OCCOCCOCCOc1cc2cc(-c3ccc(CC)c(OC(F)(F)F)c3)c(=O)oc2cn1] | A-103 |
| 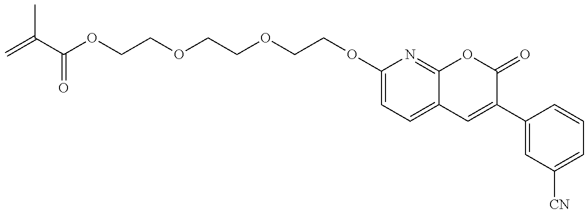 [SMILES: C=C(C)C(=O)OCCOCCOCCOc1ccc2cc(-c3cccc(C#N)c3)c(=O)oc2n1] | A-104 |
| 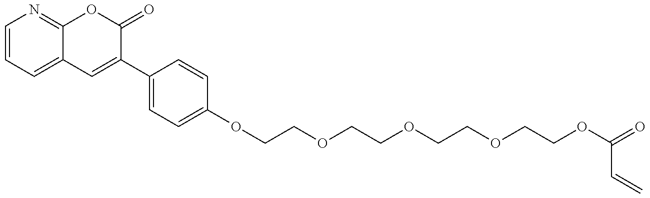 [SMILES: C=CC(=O)OCCOCCOCCOCCOc1ccc(-c2cc3cccnc3oc2=O)cc1] | A-105 |
| 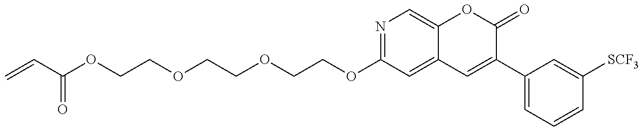 | A-106 |
| 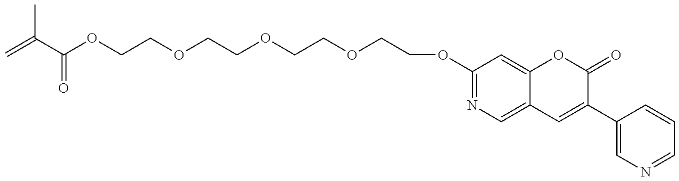 [SMILES: C=C(C)C(=O)OCCOCCOCCOCCOc1cc2oc(=O)c(-c3cccnc3)cc2cn1] | A-107 |
| 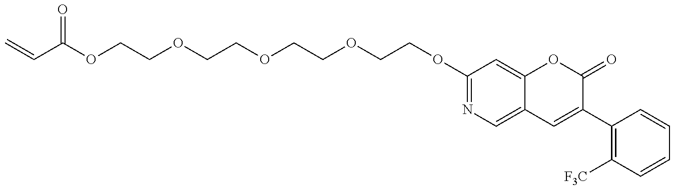 [SMILES: C=CC(=O)OCCOCCOCCOCCOc1cc2oc(=O)c(-c3ccccc3C(F)(F)F)cc2cn1] | A-108 |
| 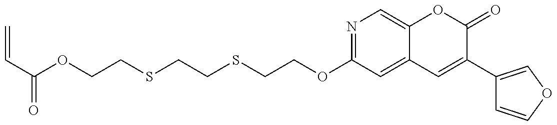 [SMILES: C=CC(=O)OCCSCCSCCOc1cc2cc(-c3ccoc3)c(=O)oc2cn1] | A-109 |
| 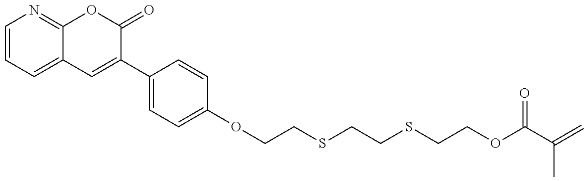 [SMILES: C=C(C)C(=O)OCCSCCSCCOc1ccc(-c2cc3cccnc3oc2=O)cc1] | A-110 |
| 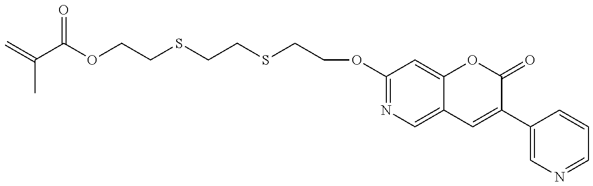 [SMILES: C=C(C)C(=O)OCCSCCSCCOc1cc2oc(=O)c(-c3cccnc3)cc2cn1] | A-111 |
| 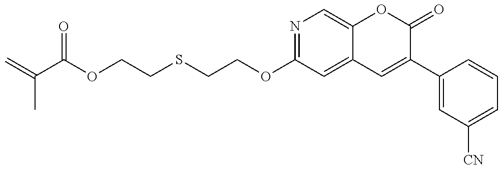 [SMILES: C=C(C)C(=O)OCCSCCOc1cc2cc(-c3cccc(C#N)c3)c(=O)oc2cn1] | A -112 |
| 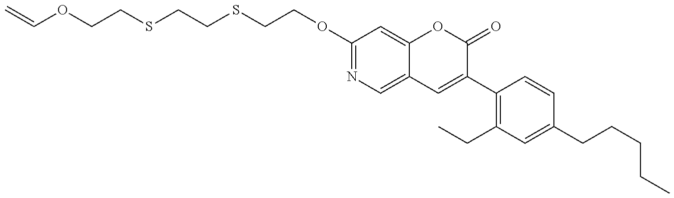 [SMILES: C=COCCSCCSCCOc1cc2oc(=O)c(-c3ccc(CCCCC)cc3CC)cc2cn1] | A-113 |
| 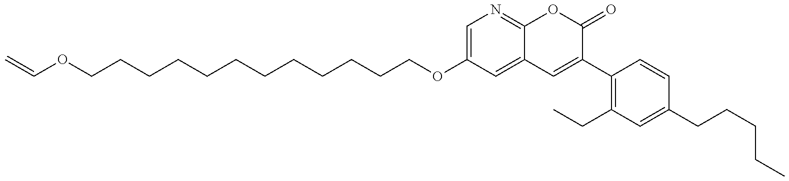 [SMILES: C=COCCCCCCCCCCCCOc1cnc2oc(=O)c(-c3ccc(CCCCC)cc3CC)cc2c1] | A-114 |
| 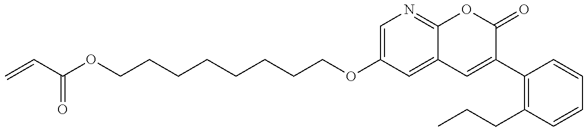 [SMILES: C=CC(=O)OCCCCCCCCOc1cnc2oc(=O)c(-c3ccccc3CCC)cc2c1] | A-115 |
| 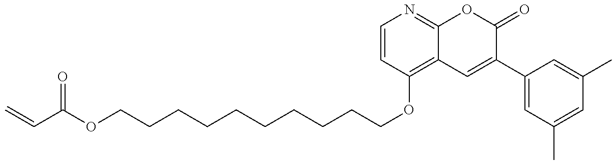 [SMILES: C=CC(=O)OCCCCCCCCCCOc1ccnc2oc(=O)c(-c3cc(C)cc(C)c3)cc12] | A-116 |
| 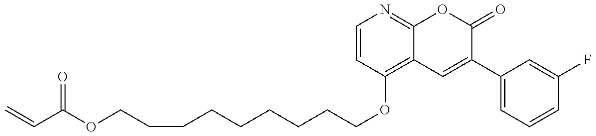 [SMILES: C=CC(=O)OCCCCCCCCCOc1ccnc2oc(=O)c(-c3cccc(F)c3)cc12] | A-117 |
| 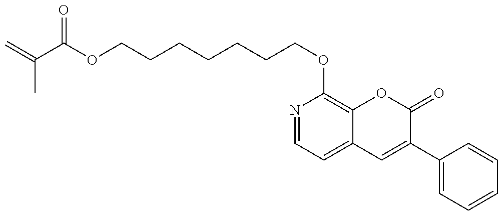 [SMILES: C=C(C)C(=O)OCCCCCCCOc1nccc2cc(-c3ccccc3)c(=O)oc12] | A-118 |
| 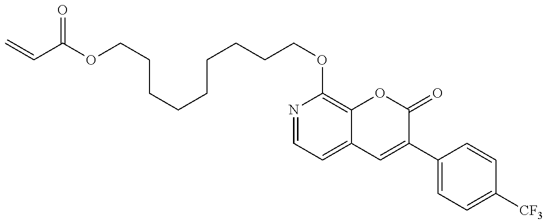 [SMILES: C=CC(=O)OCCCCCCCCCOc1nccc2cc(-c3ccc(C(F)(F)F)cc3)c(=O)oc12] | A-119 |
| 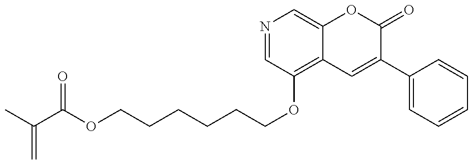 [SMILES: C=C(C)C(=O)OCCCCCCOc1cncc2oc(=O)c(-c3ccccc3)cc12] | A-120 |
| 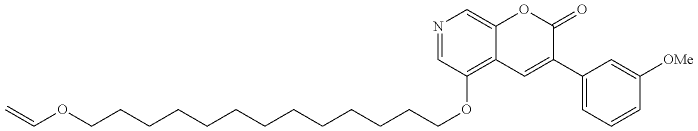 [SMILES: C=COCCCCCCCCCCCCCOc1cncc2oc(=O)c(-c3cccc(OC)c3)cc12] | A-121 |
| 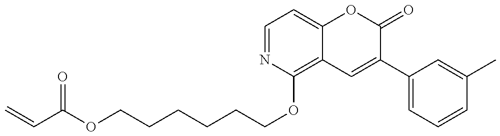 [SMILES: C=CC(=O)OCCCCCCOc1nccc2oc(=O)c(-c3cccc(C)c3)cc12] | A-122 |
| 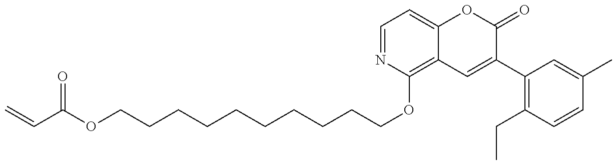 [SMILES: C=CC(=O)OCCCCCCCCCCOc1nccc2oc(=O)c(-c3cc(C)ccc3CC)cc12] | A-123 |
| 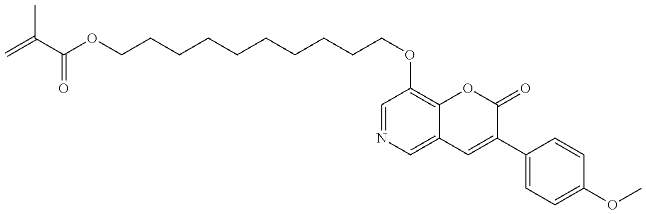 [SMILES: C=C(C)C(=O)OCCCCCCCCCCOc1cncc2cc(-c3ccc(OC)cc3)c(=O)oc12] | A-124 |
| 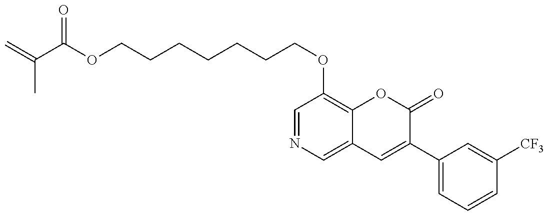 [SMILES: C=C(C)C(=O)OCCCCCCCOc1cncc2cc(-c3cccc(C(F)(F)F)c3)c(=O)oc12] | A-125 |
| 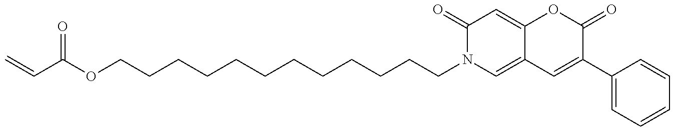 [SMILES: C=CC(=O)OCCCCCCCCCCCCn1cc2cc(-c3ccccc3)c(=O)oc2cc1=O] | A-126 |
| 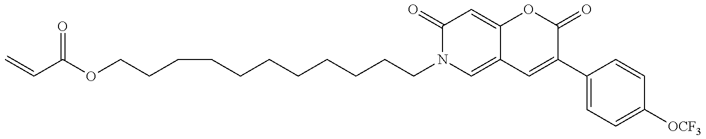 [SMILES: C=CC(=O)OCCCCCCCCCCCn1cc2cc(-c3ccc(OC(F)(F)F)cc3)c(=O)oc2cc1=O] | A-127 |
| 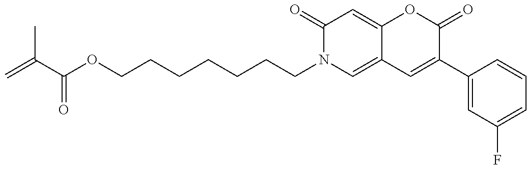 [SMILES: C=C(C)C(=O)OCCCCCCCn1cc2cc(-c3cccc(F)c3)c(=O)oc2cc1=O] | A-128 |
| 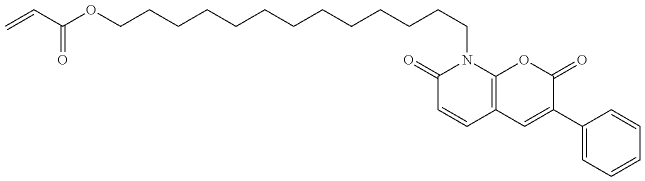 [SMILES: C=CC(=O)OCCCCCCCCCCCCCn1c(=O)ccc2cc(-c3ccccc3)c(=O)oc21] | A-129 |
| 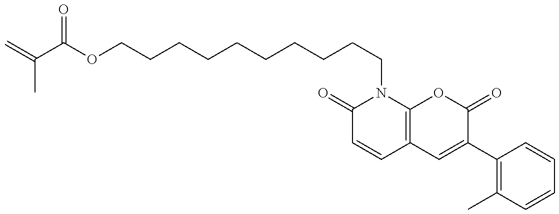 [SMILES: C=C(C)C(=O)OCCCCCCCCCCn1c(=O)ccc2cc(-c3ccccc3C)c(=O)oc21] | A-130 |
| 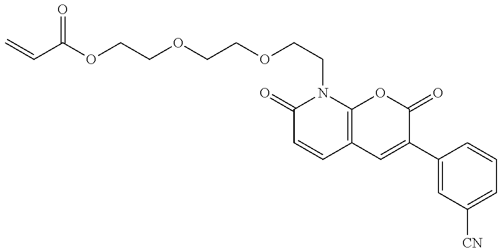 [SMILES: C=CC(=O)OCCOCCOCCn1c(=O)ccc2cc(-c3cccc(C#N)c3)c(=O)oc21] | A-131 |
| 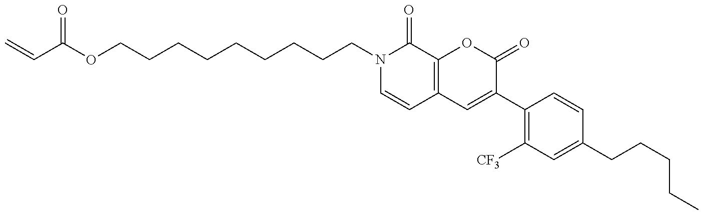 [SMILES: C=CC(=O)OCCCCCCCCCn1ccc2cc(-c3ccc(CCCCC)cc3C(F)(F)F)c(=O)oc2c1=O] | A-132 |
| 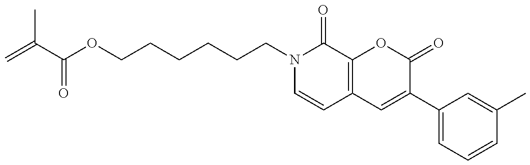 [SMILES: C=C(C)C(=O)OCCCCCCn1ccc2cc(-c3cccc(C)c3)c(=O)oc2c1=O] | A-133 |
| 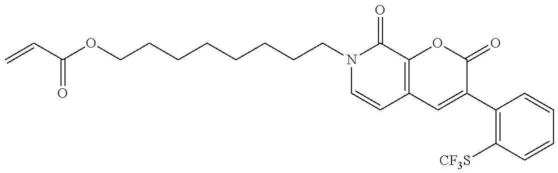 | A-134 |
| 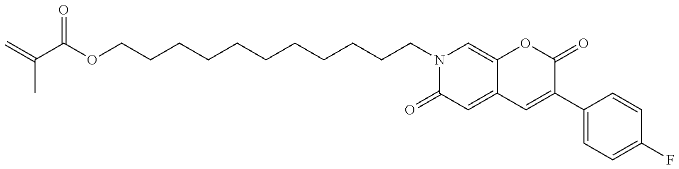 [SMILES: C=C(C)C(=O)OCCCCCCCCCCCn1cc2oc(=O)c(-c3ccc(F)cc3)cc2cc1=O] | A-135 |
| 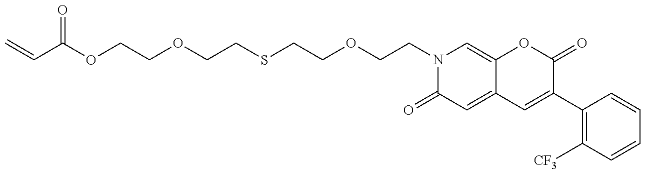 [SMILES: C=CC(=O)OCCOCCSCCOCCn1cc2oc(=O)c(-c3ccccc3C(F)(F)F)cc2cc1=O] | A-136 |
| 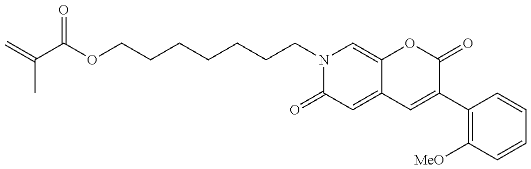 [SMILES: C=C(C)C(=O)OCCCCCCCn1cc2oc(=O)c(-c3ccccc3OC)cc2cc1=O] | A-137 |
| 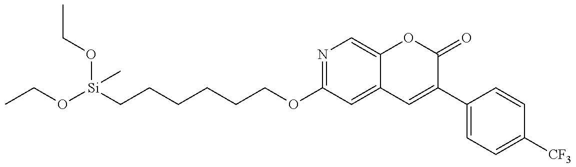 [SMILES: CCO[Si](C)(CCCCCCOc1cc2cc(-c3ccc(C(F)(F)F)cc3)c(=O)oc2cn1)OCC] | A-138 |
| 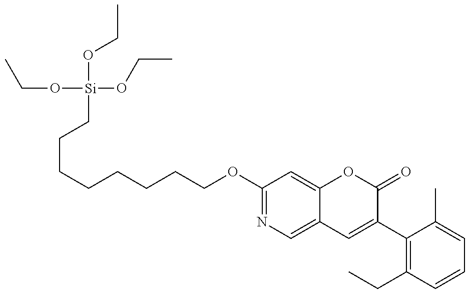 [SMILES: CCO[Si](CCCCCCCCOc1cc2oc(=O)c(-c3c(C)cccc3CC)cc2cn1)(OCC)OCC] | A-139 |
| 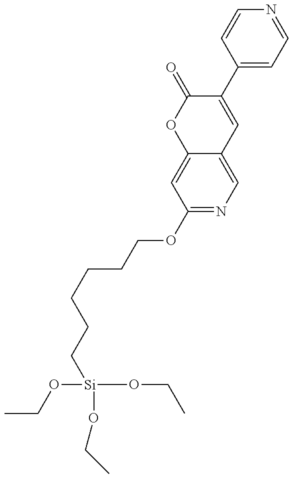 [SMILES: CCO[Si](CCCCCCOc1cc2oc(=O)c(-c3ccncc3)cc2cn1)(OCC)OCC] | A-140 |
| 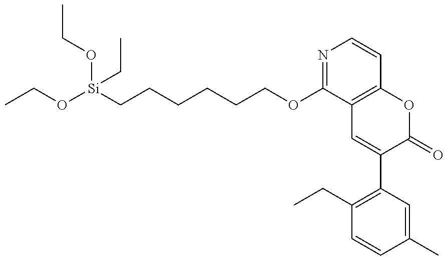 [SMILES: CCO[Si](CC)(CCCCCCOc1nccc2oc(=O)c(-c3cc(C)ccc3CC)cc12)OCC] | A-141 |
| 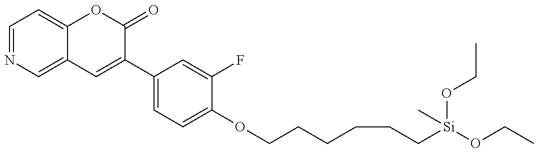 [SMILES: CCO[Si](C)(CCCCCCOc1ccc(-c2cc3cnccc3oc2=O)cc1F)OCC] | A-142 |
| 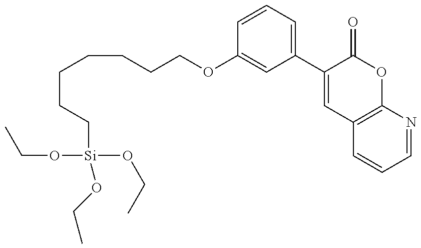 [SMILES: CCO[Si](CCCCCCCOc1cccc(-c2cc3cccnc3oc2=O)c1)(OCC)OCC] | A-143 |
| 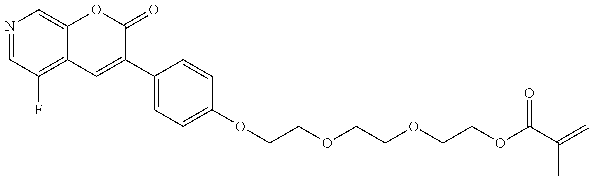 [SMILES: C=C(C)C(=O)OCCOCCOCCOc1ccc(-c2cc3c(F)cncc3oc2=O)cc1] | A-144 |
| 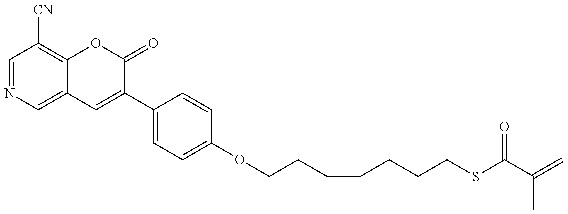 [SMILES: C=C(C)C(=O)SCCCCCCCOc1ccc(-c2cc3cncc(C#N)c3oc2=O)cc1] | A-145 |
| 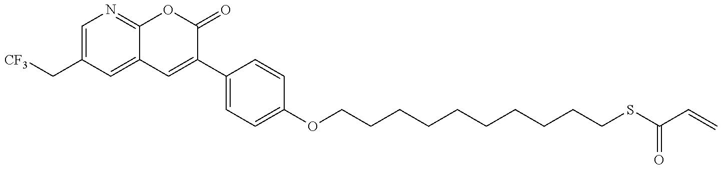 [SMILES: C=CC(=O)SCCCCCCCCCCOc1ccc(-c2cc3cc(CC(F)(F)F)cnc3oc2=O)cc1] | A-146 |
| 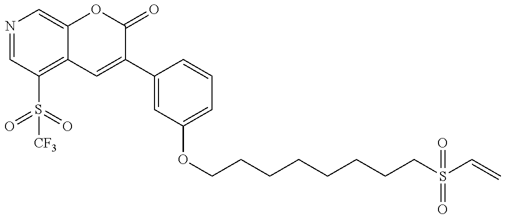 [SMILES: C=CS(=O)(=O)CCCCCCCCOc1cccc(-c2cc3c(S(=O)(=O)C(F)(F)F)cncc3oc2=O)c1] | A-147 |
| 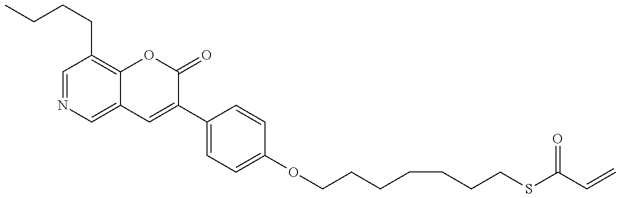 [SMILES: C=CC(=O)SCCCCCCCOc1ccc(-c2cc3cncc(CCCC)c3oc2=O)cc1] | A-148 |
| 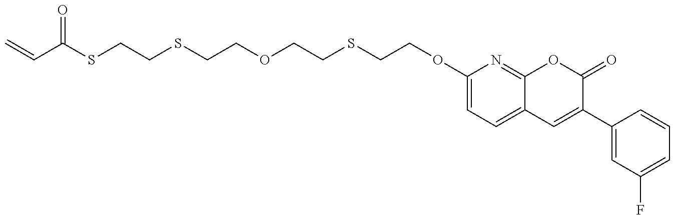 [SMILES: C=CC(=O)SCCSCCOCCSCCOc1ccc2cc(-c3cccc(F)c3)c(=O)oc2n1] | A-149 |
| 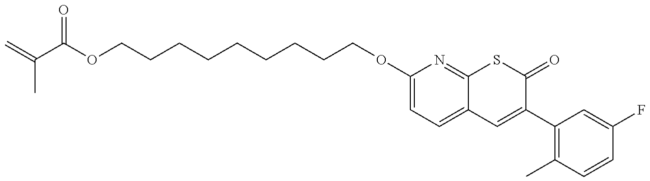 [SMILES: C=C(C)C(=O)OCCCCCCCCCOc1ccc2cc(-c3cc(F)ccc3C)c(=O)sc2n1] | A-150 |
| 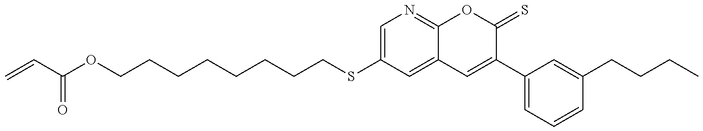 [SMILES: C=CC(=O)OCCCCCCCCSc1cnc2oc(=S)c(-c3cccc(CCCC)c3)cc2c1] | A-151 |
| 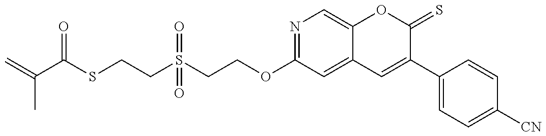 [SMILES: C=C(C)C(=O)SCCS(=O)(=O)CCOc1cc2cc(-c3ccc(C#N)cc3)c(=S)oc2cn1] | A-152 |
| 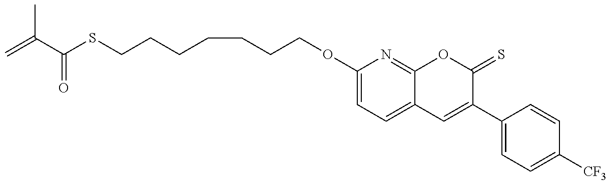 [SMILES: C=C(C)C(=O)SCCCCCCCOc1ccc2cc(-c3ccc(C(F)(F)F)cc3)c(=S)oc2n1] | A-153 |
| 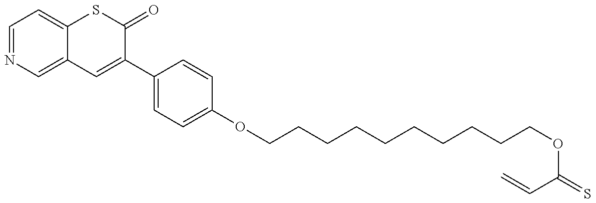 [SMILES: C=CC(=S)OCCCCCCCCCCOc1ccc(-c2cc3cnccc3sc2=O)cc1] | A-154 |
| 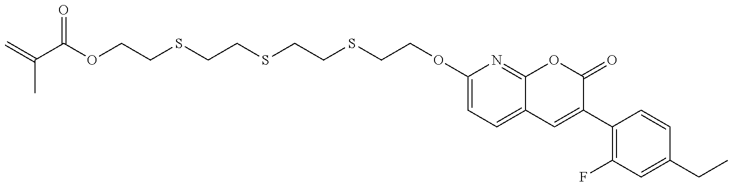 [SMILES: C=C(C)C(=O)OCCSCCSCCSCCOc1ccc2cc(-c3ccc(CC)cc3F)c(=O)oc2n1] | A-155 |
| 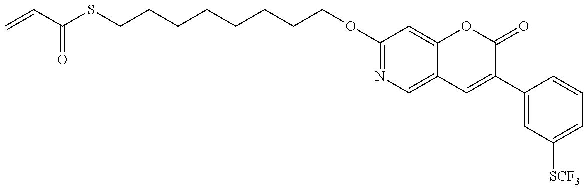 | A-156 |
| 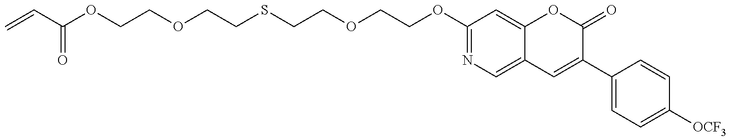 [SMILES: C=CC(=O)OCCOCCSCCOCCOc1cc2oc(=O)c(-c3ccc(OC(F)(F)F)cc3)cc2cn1] | A-157 |
| 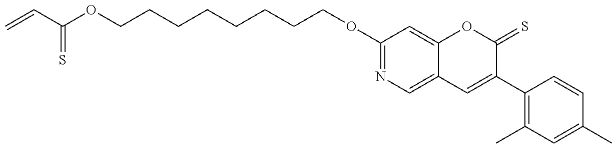 [SMILES: C=CC(=S)OCCCCCCCCOc1cc2oc(=S)c(-c3ccc(C)cc3C)cc2cn1] | A-158 |
| 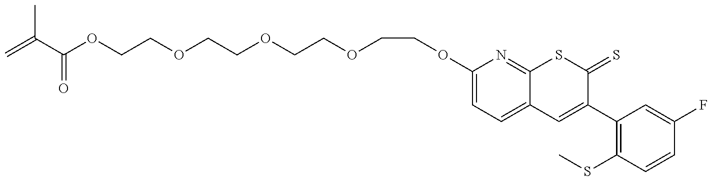 [SMILES: C=C(C)C(=O)OCCOCCOCCOCCOc1ccc2cc(-c3cc(F)ccc3SC)c(=S)sc2n1] | A-159 |
| 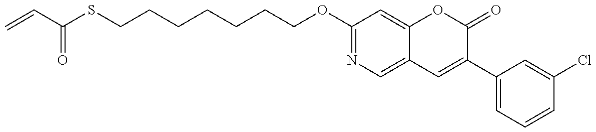 [SMILES: C=CC(=O)SCCCCCCCOc1cc2oc(=O)c(-c3cccc(Cl)c3)cc2cn1] | A-160 |
| 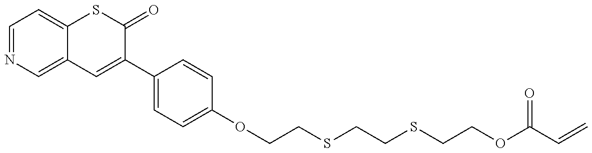 [SMILES: C=CC(=O)OCCSCCSCCOc1ccc(-c2cc3cnccc3sc2=O)cc1] | A-161 |
| 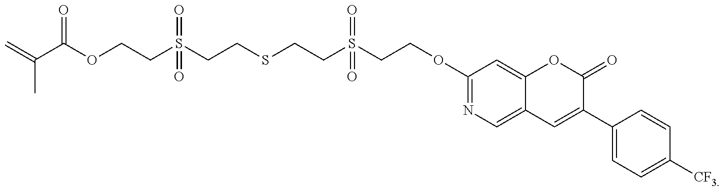 [SMILES: C=C(C)C(=O)OCCS(=O)(=O)CCSCCS(=O)(=O)CCOc1cc2oc(=O)c(-c3ccc(C(F)(F)F)cc3)cc2cn1] | A-162 |

Parts of the compounds of the present application may be synthesized by methods well known to the skilled person. However, for specific precursor materials of the present application, a novel method of preparation is described herein. Preferably, all syntheses are carried out under an inert atmosphere using dried solvents.

An exemplary reaction sequence is shown in Scheme 1 for the compounds of formula (I #) where X is O, Y₀ is O, m1 is 1, A₁ is N and A₂, A₃ and A₄ are CR″ and all further symbols and indices have a meaning as described before and the polymerizable group R₁ is as shown in scheme 1.

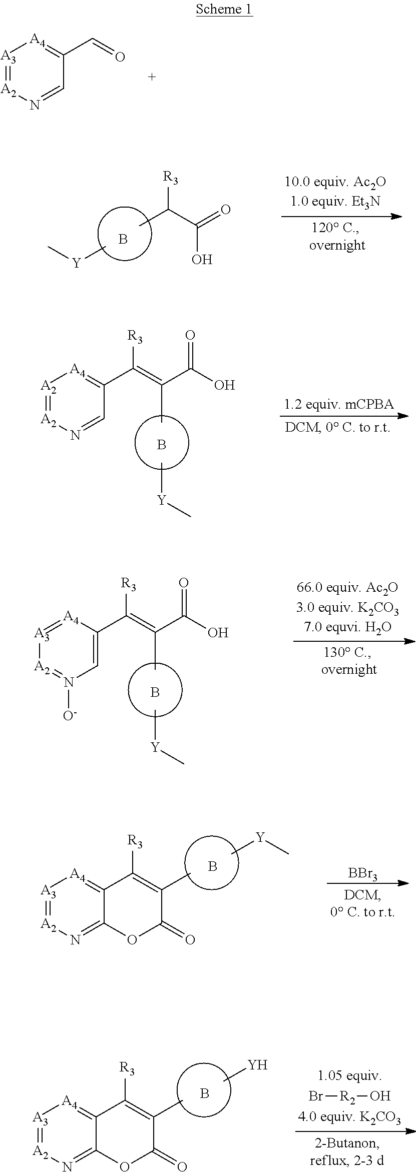

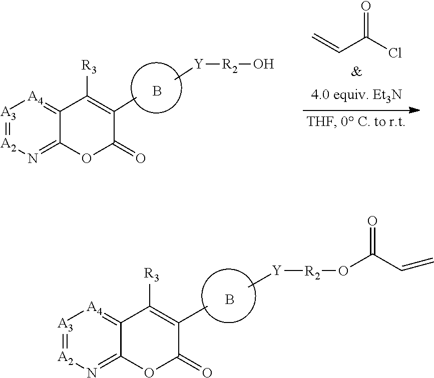

The first type of reaction is the formation of a cinnamic acid derivative.

The second type of reaction is the oxidative formation of pyridine 1-oxide.

The third type of reaction is a base-induced ring-closure reaction.

The fourth type of reaction is a methoxy-deprotection reaction.

The fifth type of reaction is a Williamson ether synthesis reaction.

The sixth type of reaction is an esterification reaction.

A representative synthesis for compound A-001 is described in Scheme 1-1. Parts of the synthesis sequence are adapted to the desired chemical structure of A-001 by the previously described method of D. Wang et al., Org. Lett. 2017, 19, 984-987.

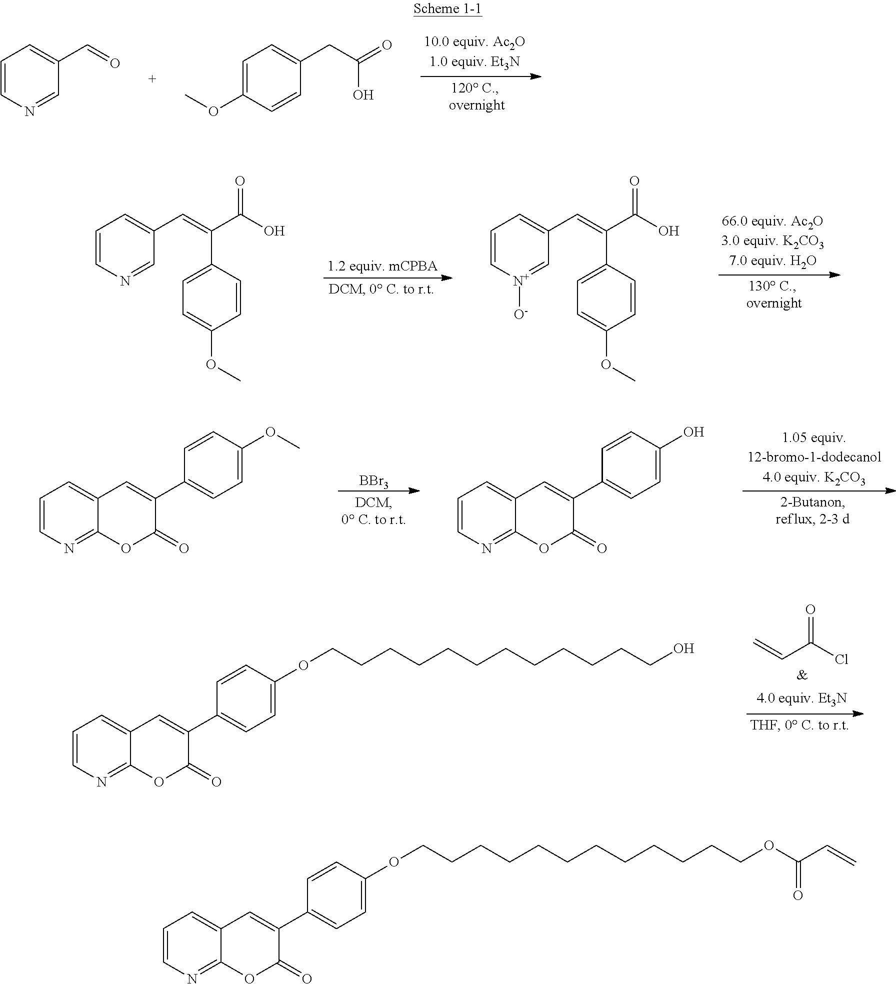

An alternative reaction sequence is shown in Scheme 2-1 for the compounds of formula (I #) where X is O, Y₀ is O, m1 is 0, A₃ is N and A₁ and A₄ are CR″ and all further symbols and indices have a meaning as described before and the polymerizable group R₁ is as shown in scheme 2-1.

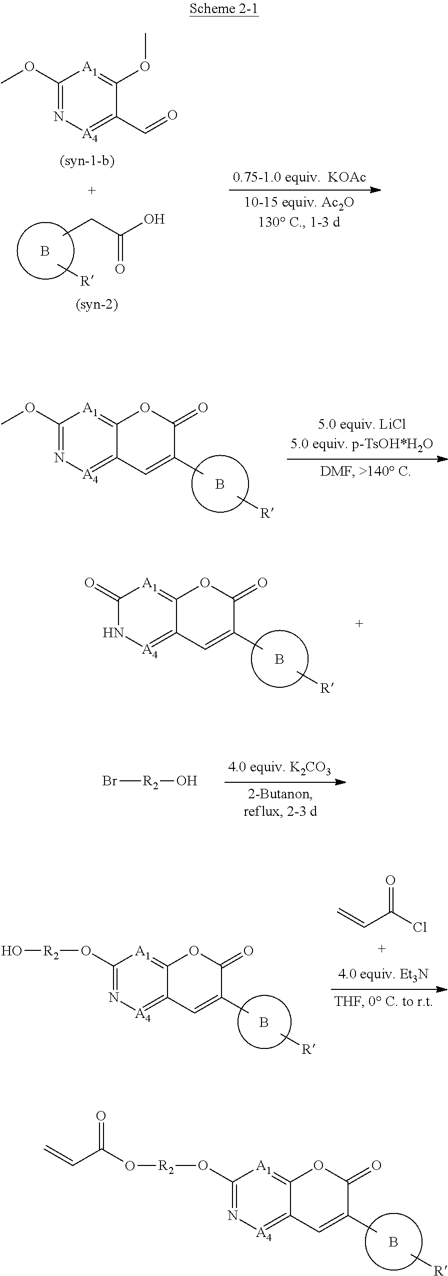

The first step of the described synthesis in Scheme 2-1 is not known in the art and therefore another embodiment of the invention.

The second type of reaction is a methoxy-deprotection reaction.

The third type of reaction is a Williamson ether synthesis reaction.

The fourth type of reaction is an esterification reaction.

An alternative reaction sequence is further shown in Scheme 2-2 for the compounds of formula (I #) where X is O, Y₀ is O, m1 is 1, A₂ is N and A₁, A₃ and A₄ are CR″ and all further symbols and indices have a meaning as described before and the polymerizable group R₁ is as shown in scheme 2-2.

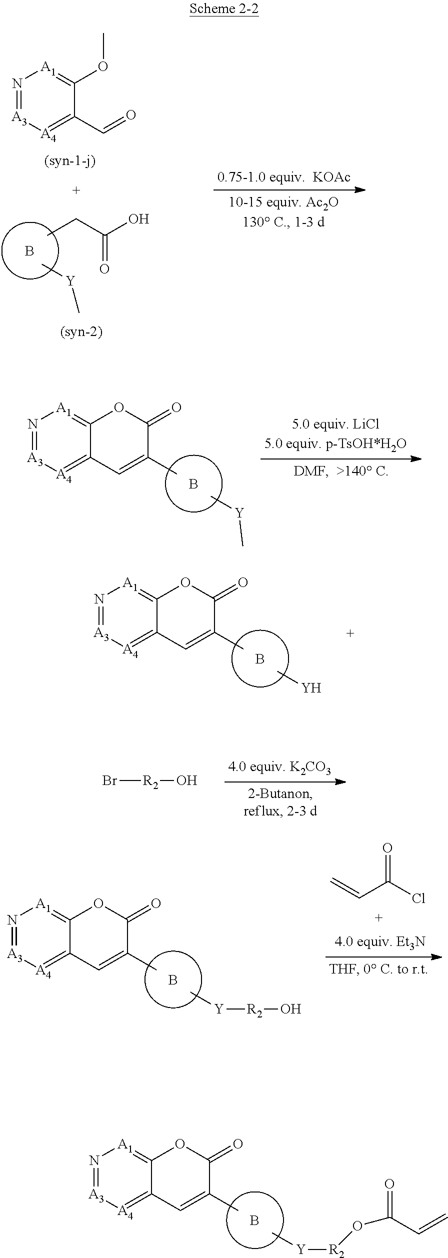

Again, the first type of reaction is a reaction according to the inventive process as described further in detail below.

The second type of reaction is a methoxy-deprotection reaction.

The third type of reaction is a Williamson ether synthesis reaction.

The fourth type of reaction is an esterification reaction.

An alternative reaction sequence is further shown in Scheme 2-3 for the compounds of formula (I #) where X is O, Y₀ is O, m1 is 0, A₃ is N and A₁ and A₄ are CR″ and all further symbols and indices have a meaning as described before.

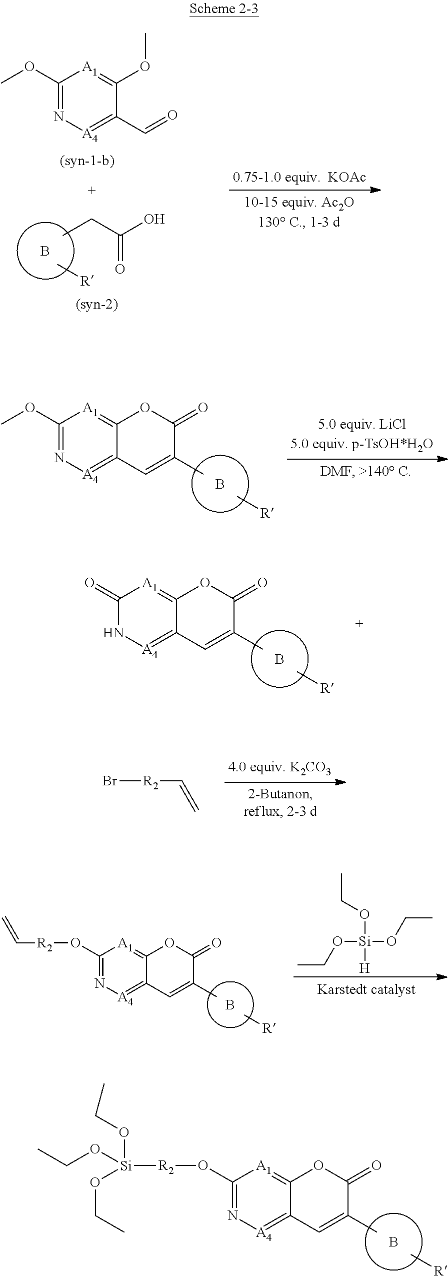

The first type of reaction is a reaction according to the inventive process as described further below.

The second type of reaction is a methoxy-deprotection reaction.

The third type of reaction is a Williamson ether synthesis reaction.

The fourth type of reaction is a hydrosilylation reaction.

The invention therefore relates further to a process for the preparation of compounds of formulae (syn-I-a), (syn-I-b), (syn-I-c), (syn-I-d), (syn-I-e), (syn-I-f), (syn-I-g), (syn-I-h), (syn-I-i), (syn-I-j), (syn-I-k), (syn-I-L), (syn-I-m), (syn-I-n), (syn-I-o), (syn-I-p), (syn-I-q), (syn-I-r) or (syn-I-s),

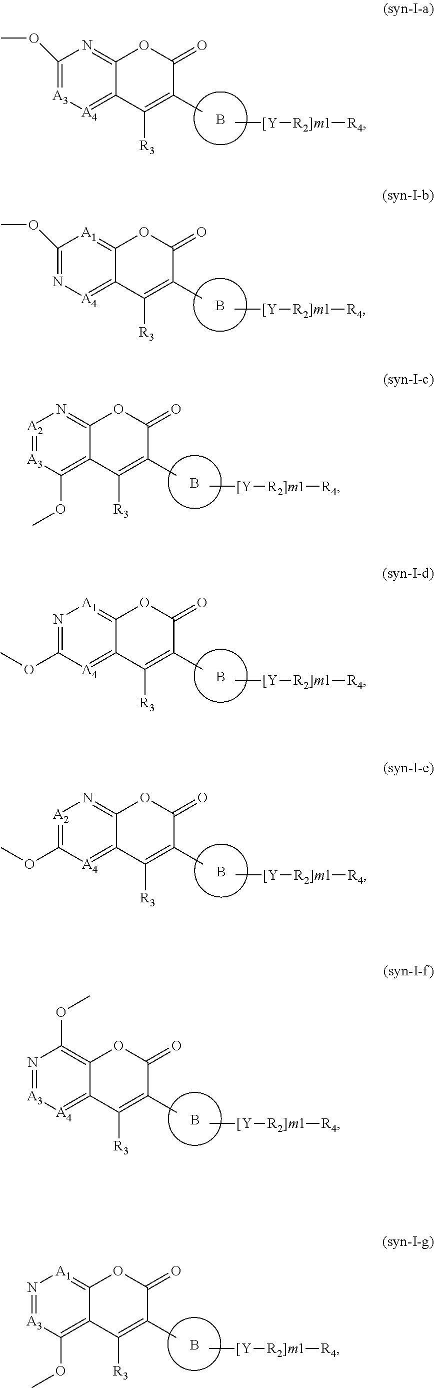

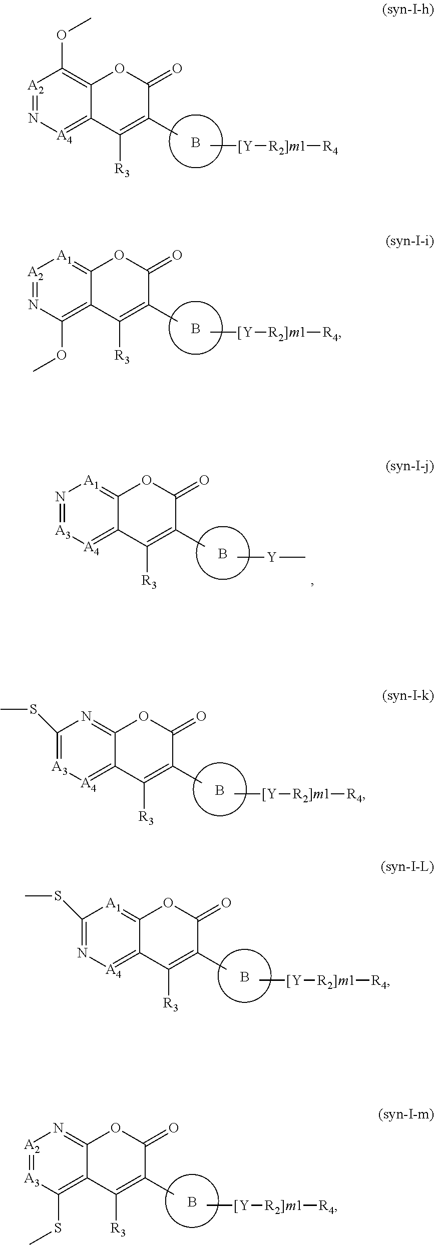

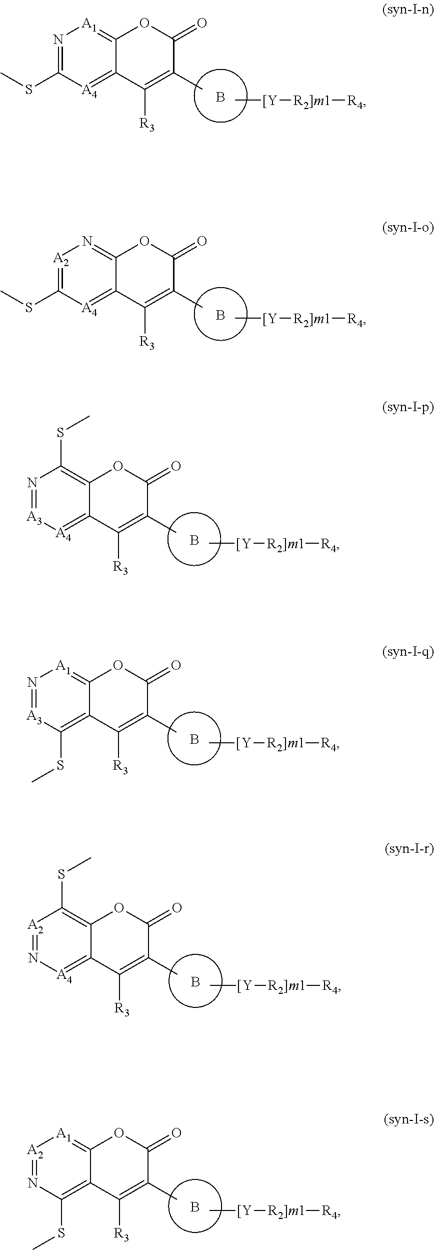

where A₁, A₂, A₃ and A₄ are each independently CR″, R₃ is H and m1, Y, R₂, R₄ and

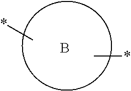

and R″ have a meaning as described before or preferably described before,

by reaction of a compound of (syn-1-a), (syn-1-b), (syn-1-c), (syn-1-d), (syn-1-e), (syn-1-f), (syn-1-g), (syn-1-h), (syn-1-i), (syn-1-j), (syn-1-k), (syn-1-L), (syn-1-m), (syn-1-n), (syn-1-o), (syn-1-p), (syn-1-q), (syn-1-r) or (syn-1-s),

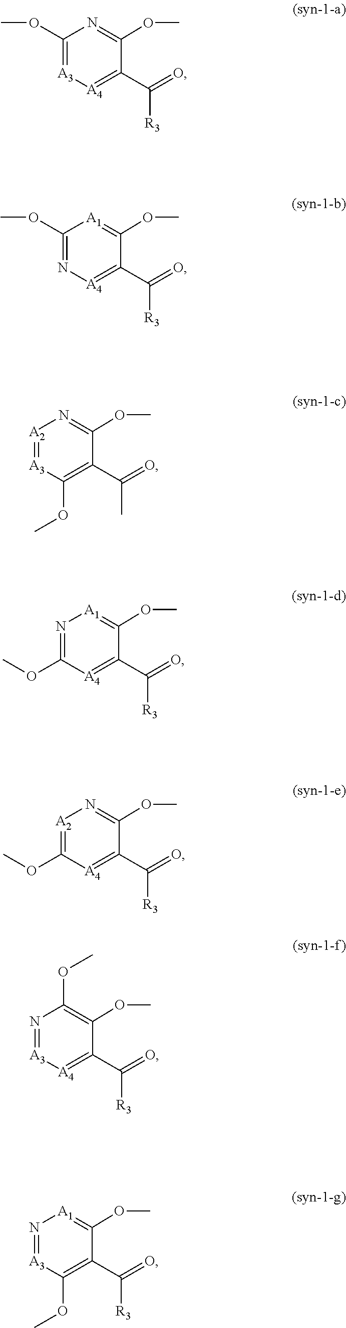

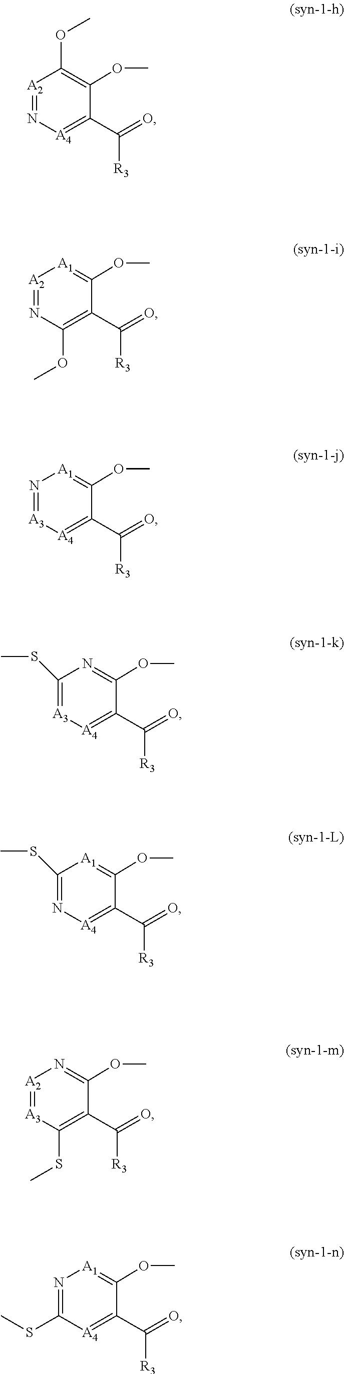

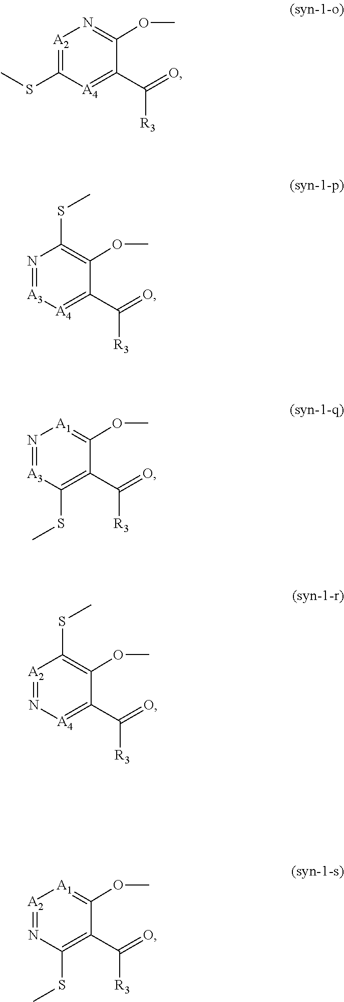

where A₁, A₂, A₃ and A₄ are each independently CR″ and R₃ is H and R″ is defined as described before or preferably described before,

with a compound of formula (syn-2)

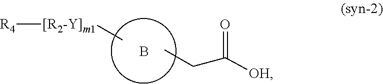

where m1, Y, R₂, R₄ and

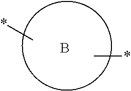

have a meaning as described before ore preferably described before, in the presence of a buffer system.

Suitable buffer systems are lithium methoxide with acetic anhydride, potassium carbonate with acetic anhydride, cesium carbonate with acetic anhydride, potassium tert-butoxide with acetic anhydride, triethylamine with acetic anhydride, pyridine with acetic anhydride, potassium acetate with acetic anhydride.

A preferred buffer system is potassium acetate with acetic anhydride.

The compounds of formulae (syn-1-a), (syn-1-b), (syn-1-c), (syn-1-d), (syn-1-e), (syn-1-f), (syn-1-g), (syn-1-h), (syn-1-i), (syn-1-j), (syn-1-k), (syn-1-L), (syn-1-m), (syn-1-n), (syn-1-o), (syn-1-p), (syn-1-q), (syn-1-r), (syn-1-s) and (syn-2) are commercially available or are accessible by known synthetic processes.

The reaction can be carried out both in an open apparatus and also in a closed apparatus.

It is preferred to mix the starting materials in an inert-gas atmosphere whose oxygen content is a maximum of 1000 ppm. It is particularly preferred if the oxygen content is less than 500 ppm, very particularly preferably a maximum of 100 ppm.

The invention therefore furthermore relates to the process, as described above, where the compounds of formulae (syn-1-a) to (syn-1-s) and (syn-2) are used in an equimolar amount.

The invention therefore furthermore relates to the process, as described above, where parts of the buffer system, namely potassium acetate, is used in an amount of 0.17 to 1.45 equiv. in relation to the starting materials. The process uses preferably and amount of 0.38 to 1.25 equiv. of potassium acetate. The process uses more preferably and amount of 0.45 to 1.00 equiv. of potassium acetate.

The invention therefore furthermore relates to the process, as described above, where parts of the buffer system, namely acetic anhydride, is used in an amount of 5.0 to 50.0 equiv. in relation to the starting materials. The process uses preferably and amount of 10.0 to 25.0 equiv. of acetic anhydride. The process uses more preferably and amount of 12.5 to 20.0 equiv. of acetic anhydride.

In said process according to the invention, it is furthermore preferred if the reaction of the reactants is followed by a purification step in order to separate the end product of the formula I, as described above, off from by-products or reaction products.

Suitable purification steps include the separation of readily volatile components by distillation or condensation, extraction with an organic solvent or a combination of these methods. Each known separation method can be used for this purpose or combined.

The invention therefore furthermore relates to the process, as described above, characterised in that the reaction is followed by a purification step.

The reaction mixture obtained from the reaction is preferably cooled to room temperature, and a protic solvent such as water is added. The solid obtained is preferably further recrystallized from an organic solvent.

Suitable solvents for recrystallisation are alcohols, such as methanol, ethanol, propanol, ethers, such as tetrahydrofuran, diethyl ether, methyl t-butyl ether or dimethoxyethane, or acetone. 2-Propanol is preferably used.

In an embodiment of the process according to the invention, as described above, the reaction takes place without an organic solvent (neat) and merely takes place in the buffer system as described before.

In a preferred embodiment of the process according to the invention, as described above, described in its embodiments, or described as preferred, the reaction of the compounds (syn-1-a), (syn-1-b), (syn-1-c), (syn-1-d), (syn-1-e), (syn-1-f), (syn-1-g), (syn-1-h), (syn-1-i), (syn-1-j), (syn-1-k), (syn-1-L), (syn-1-m), (syn-1-n), (syn-1-o), (syn-1-p), (syn-1-q), (syn-1-r) or (syn-1-s) and (syn-2) takes place at a reaction temperature of 50° C. to 160° C. The reaction preferably takes place at 60° C. to 140° C.

Further representative processes according to the invention are described in the following Scheme 2-4, where the starting material of formula (syn-1-a) may be replaced with any of materials of formulae (syn-1-c) to (syn-1-s) accordingly.

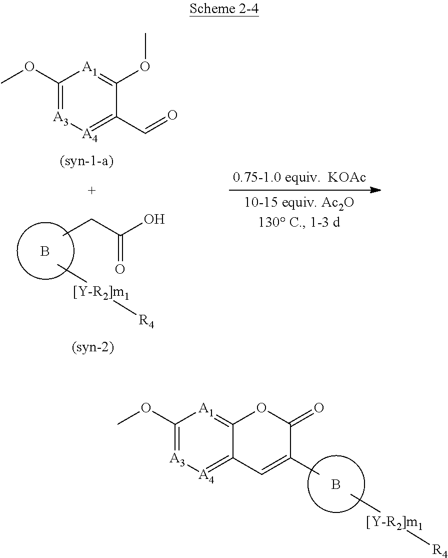

The advantage of the process of preparation according to the invention is that the reaction does not need further organic solvents and that the solubility of the materials of formulae (syn-I-a), (syn-I-b), (syn-I-c), (syn-I-d), (syn-I-e), (syn-I-f), (syn-I-g), (syn-I-h), (syn-I-i), (syn-I-j), (syn-I-k), (syn-I-L), (syn-I-m), (syn-I-n), (syn-I-o), (syn-I-p), (syn-I-q), (syn-I-r) and (syn-I-s) is high in organic solvents, preferably in organic solvents as described before.

The precursor materials of formulae (syn-I-a), (syn-I-b), (syn-I-c), (syn-I-d), (syn-I-e), (syn-I-f), (syn-I-g), (syn-I-h), (syn-I-i), (syn-I-j), (syn-I-k), (syn-I-L), (syn-I-m), (syn-I-n), (syn-I-o), (syn-I-p), (syn-I-q), (syn-I-r) and (syn-I-s) can further be easily isolated from the reaction mixture.

The invention relates further to a process for the synthesis of compounds of formula (I), (I′) or (I″) where X is O and Y₀ is O and further symbols and indices have a meaning as described before or below wherein in step 1, materials of formulae (syn-I-a), (syn-I-b), (syn-I-c), (syn-I-d), (syn-I-e), (syn-I-f), (syn-I-g), (syn-I-h), (syn-I-i), (syn-I-j), (syn-I-k), (syn-I-L), (syn-I-m), (syn-I-n), (syn-I-o), (syn-I-p), (syn-I-q), (syn-I-r) or (syn-I-s) are prepared,

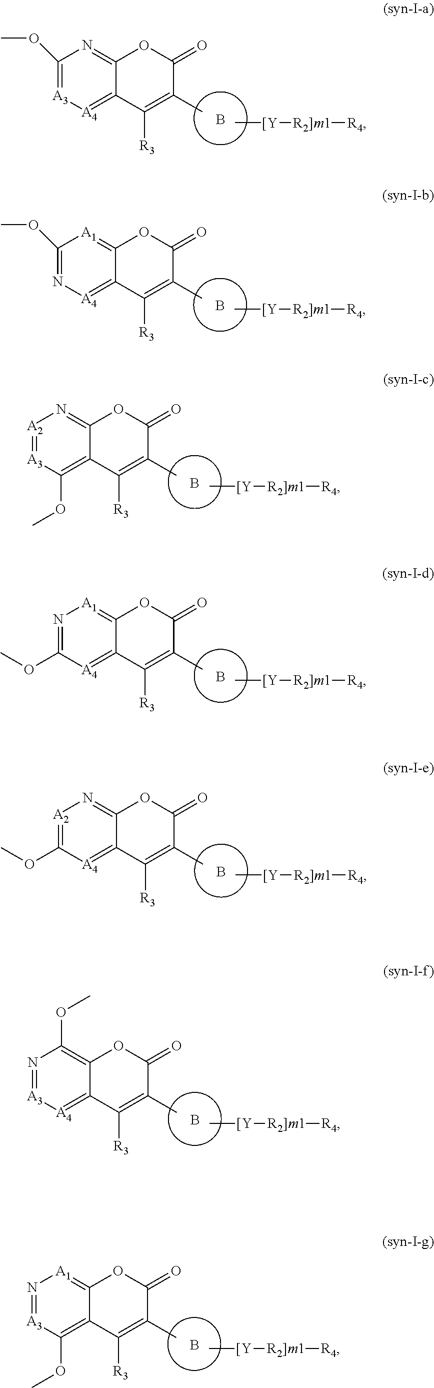

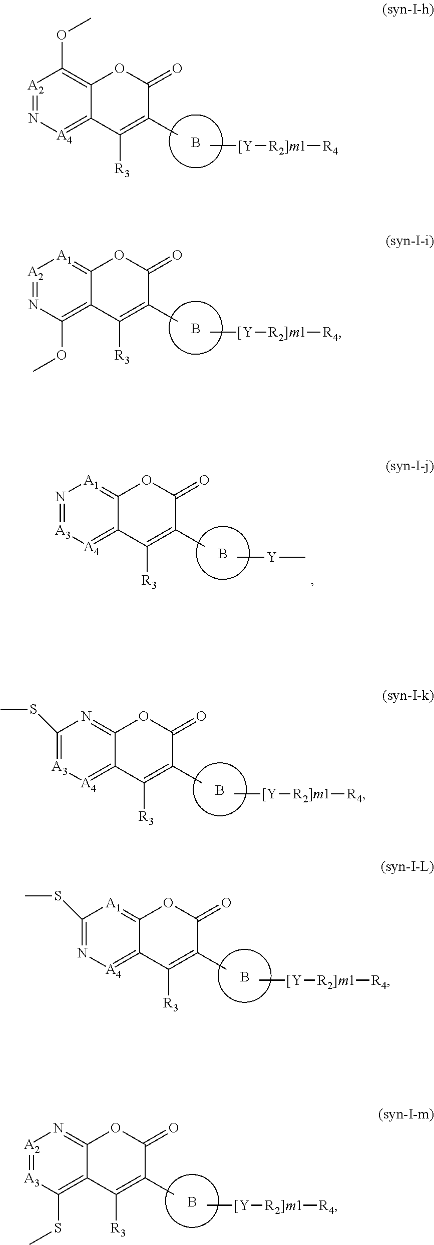

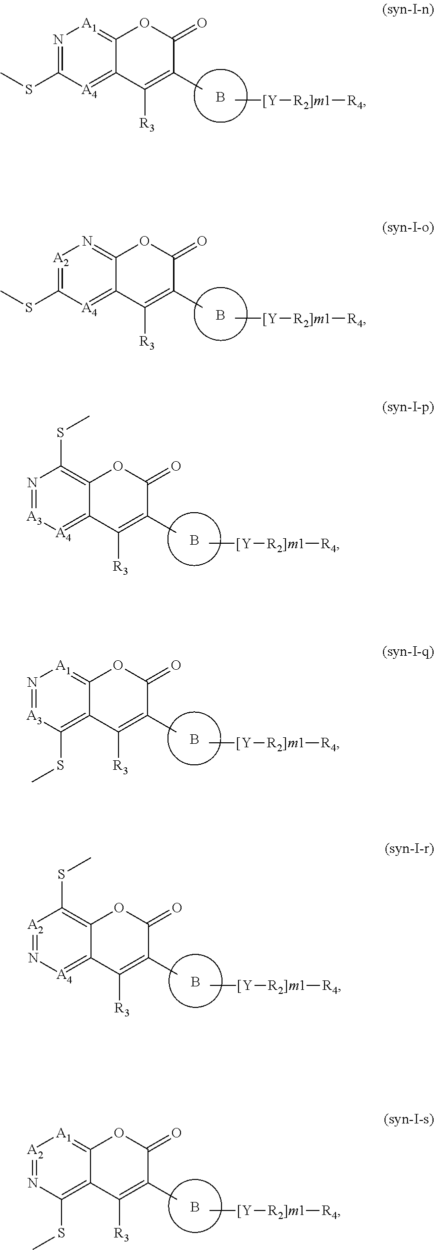

where A₁, A₂, A₃ and A₄ are each independently CR″, R₃ is H and m1, Y, R₂, R₄ and

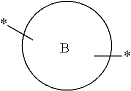

and R″ have a meaning as described before or preferably described before,

by reaction of a compound of (syn-1-a), (syn-1-b), (syn-1-c), (syn-1-d), (syn-1-e), (syn-1-f), (syn-1-g), (syn-1-h), (syn-1-i), (syn-1-j), (syn-1-k), (syn-1-L), (syn-1-m), (syn-1-n), (syn-1-o), (syn-1-p), (syn-1-q), (syn-1-r) or (syn-1-s),

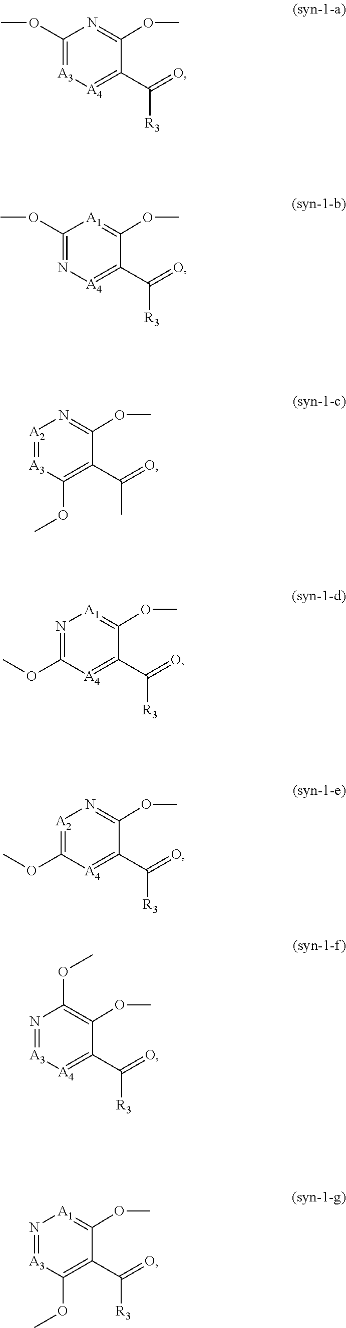

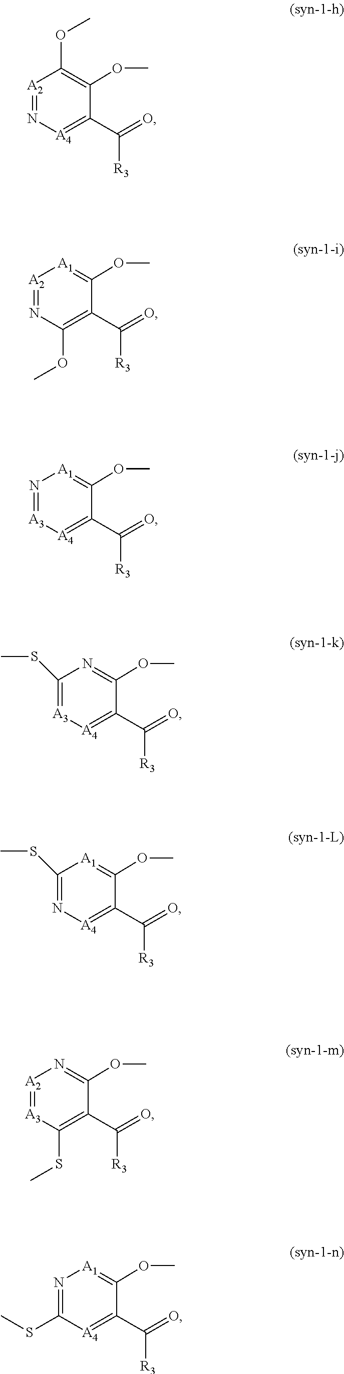

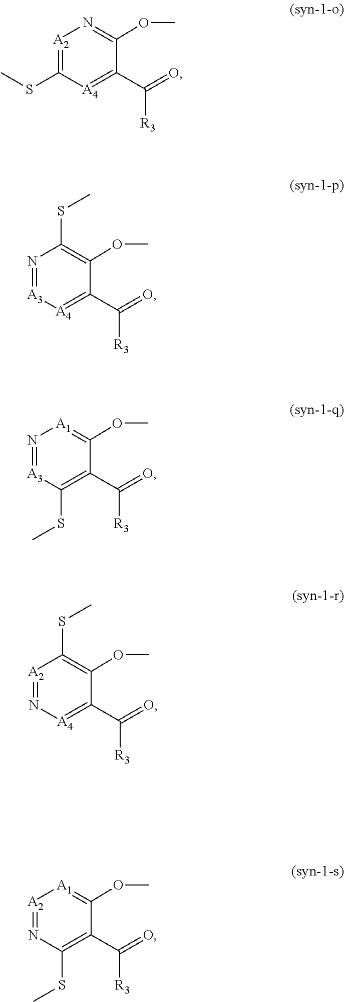

where A₁, A₂, A₃ and A₄ are each independently CR″ and R₃ is H and R″ is defined as described before or preferably described before, with a compound of formula (syn-2)

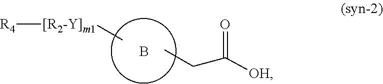

where m1, Y, R₂, R₄ and

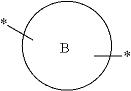

have a meaning as described before ore preferably described before, in the presence of a buffer system as described or preferably described before, followed by a deprotection reaction, a Williamson ether synthesis reaction or thioether synthesis reaction and optionally an esterification reaction or a silylation reaction.

A representative synthesis for compound A-097 is described in Scheme 2-5. As described before, the first step of the described synthesis is an example of the process according to the invention.

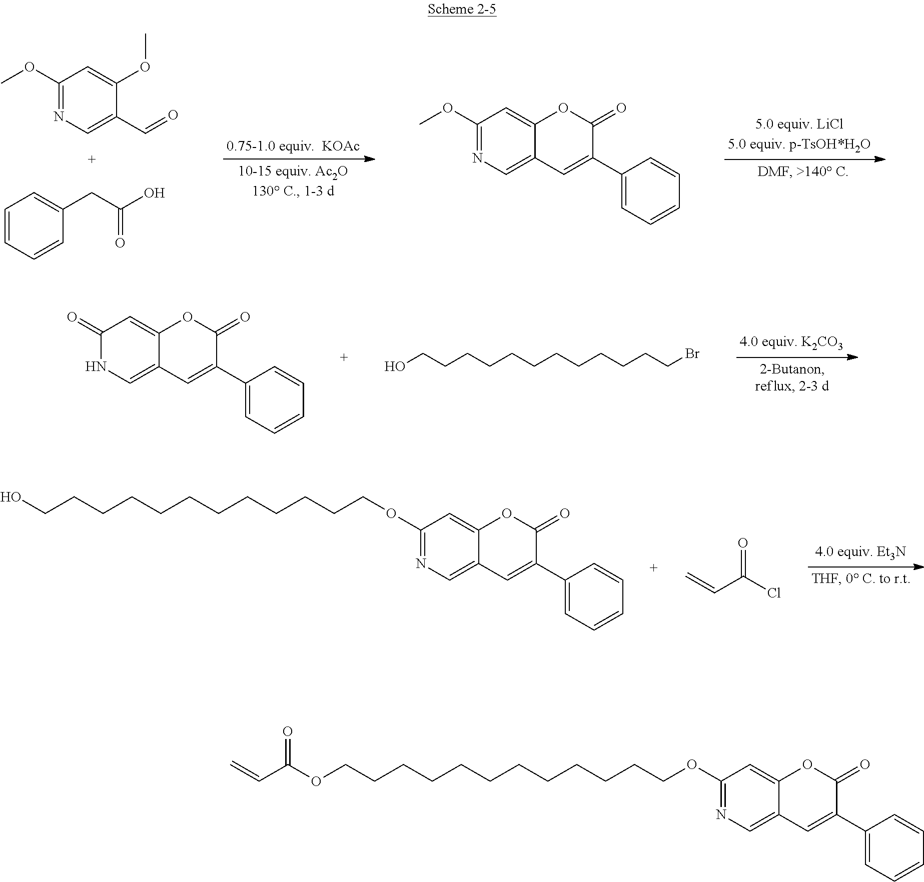

The first type of reaction is the inventive process as described before.

The second type of reaction is a methoxy-deprotection reaction.

The third type of reaction is a Williamson ether synthesis reaction.

The fourth type of reaction is an esterification reaction.

As described before, the compounds/monomers of formulae (I), (I #), (I′), (I′-a), (I′-b), (I′-c), (I′-d), (I′-e), (I′-f), (I′-g), (I′-h), (I′-i) and (I″) as described before or preferably described before contain a polymerizable group and are predestinated as monomers for an oligomerization or a polymerization.

The invention is therefore further directed to an oligomer, polymer or copolymer comprising at least one polymerized compound of formulae (I), (I #), (I′), (I′-a), (I′-b), (I′-c), (I′-d), (I′-e), (I′-f), (I′-g), (I′-h), (I′-i) or (I″) as described before or preferably described before.

The term “polymer” generally means a molecule of high relative molecular mass, the structure of which essentially comprises the multiple repetition of units derived, actually or conceptually, from molecules of low relative molecular mass (PAC, 1996, 68, 2291). The term “polymer” includes homopolymers and copolymers if not mentioned otherwise within the description. The term “oligomer” generally means a molecule of intermediate relative molecular mass, the structure of which essentially comprises a small plurality of units derived, actually or conceptually, from molecules of lower relative molecular mass (PAC, 1996, 68, 2291). In a preferred sense according to the present invention a polymer means a compound having ≥30 repeating units, and an oligomer means a compound with >1 and <30 repeating units.

Above and below, in formulae showing a polymer, an oligomer, a compound of formulae (I), (I #), (I′), (I′-a), (I′-b), (I′-c), (I′-d), (I′-e), (I′-f), (I′-g), (I′-h), (I′-i) or (I″) or a monomeric unit or a polymer formed from a compound of formulae (I), (I #), (I′), (I′-a), (I′-b), (I′-c), (I′-d), (I′-e), (I′-f), (I′-g), (I′-h), (I′-i) or (I″), an asterisk (“*”) denotes a linkage to the adjacent repeating unit in the polymer chain or oligomer chain or to a terminal end group.

Suitable terminal end groups are known to the skilled artisan and depend on the polymerization method used.

The terms “repeating unit” and “monomeric unit” mean the constitutional repeating unit (CRU), which is the smallest constitutional unit the repetition of which constitutes a regular macromolecule, a regular oligomer molecule, a regular block or a regular chain (PAC, 1996, 68, 2291).

Unless stated otherwise, the molecular weight is given as the number average molecular weight Mn or weight average molecular weight Mw, which is determined by gel permeation chromatography (GPC) against polystyrene standards in eluent solvents such as tetrahydrofuran, trichloromethane (TCM, chloroform), chlorobenzene or 1,2,4-trichloro-benzene. Unless stated otherwise, tetrahydrofuran is used as solvent. The degree of polymerization (n) means the number average degree of polymerization given as n=Mn/Mu, wherein Mu is the molecular weight of the single repeating unit as described in J. M. G. Cowie, Polymers: Chemistry & Physics of Modern Materials, Blackie, Glasgow, 1991.

In the polymers including copolymers according to the present invention, the total number of repeating units n is preferably ≥30, very preferably ≥100, most preferably ≥200, and preferably up to 5000, very preferably up to 3000, most preferably up to 2000, including any combination of the aforementioned lower and upper limits of n.

The polymers of the present invention include homopolymers, statistical copolymers, random copolymers, alternating copolymers and block copolymers, and combinations of the aforementioned.

Throughout the description and claims of this specification, the words “comprise” and “contain” and variations of the words, for example “comprising” and “comprises”, mean “including but not limited to”, and are not intended to (and do not) exclude other components

Preferably the polymerizable group R₁ forms the regioregular, alternated, regiorandom, statistical, block or random homopolymer or copolymer backbone or is part of the polymer backbone where R₁ has a meaning as described or preferably described before.

Preferably, such oligomer, polymer or copolymer according to the invention comprises a constitutional unit M⁰ based on formulae (I), (I′) or (I″)

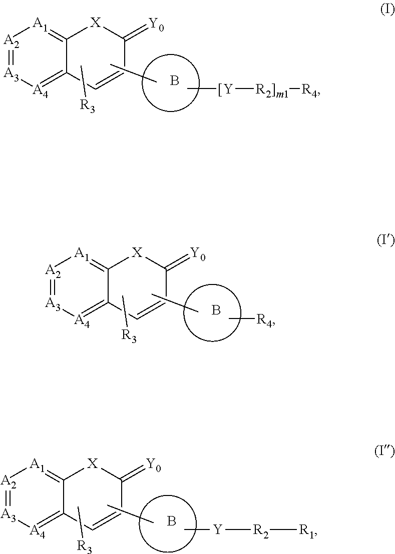

where the polymerizable group R₁ on each occurrence is polymerized and forms the regioregular, alternated, regiorandom, statistical, block or random oligomer or polymer backbone or is part of the copolymer backbone and where all the symbols and indices used within the formulae (I), (I′) and (I″) have a meaning as described before or preferably described before.

The invention is furthermore directed to an ophthalmic device or a precursor article for manufacturing an ophthalmic device as described before or preferably described below comprising an oligomer, polymer or copolymer comprising a constitutional unit M⁰ based on formulae (I), (I′) or (I″) as described before or preferably described before where R₁ on each occurrence is polymerized and forms the regioregular, alternated, regiorandom, statistical, block or random oligomer or polymer backbone or is part of the copolymer backbone.

Preferably, such polymerized groups R₁ are of formulae (1-p), (2-p), (3-p) or (4-p)

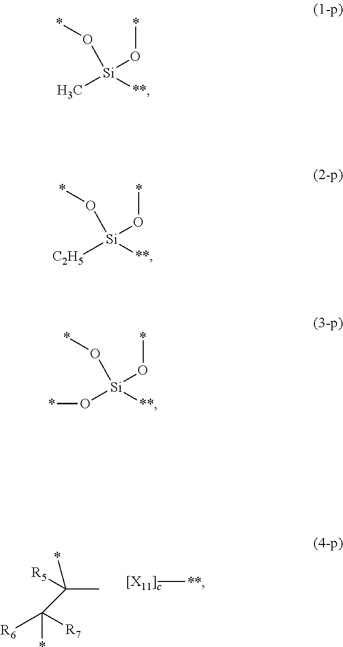

where the asterisk “*” within formulae (1-p) to (4-p) denotes a linkage to the adjacent repeating unit in the polymer chain or oligomer chain or to a terminal end group, the asterisk “**” within formulae (1-p) to (4-p) denotes the linkage to the remainder of formula (I) as described before or preferably described before and R₅, R₆, R₇, X₁₁ and c have a meaning as described before or preferably described before.

The invention is furthermore directed to an ophthalmic device or a precursor article for manufacturing an ophthalmic device as described before or preferably described below where said polymerized group R₁ is of formulae (1-p), (2-p), (3-p) or (4-p) as described before.

The invention is furthermore directed to an oligomer, polymer or copolymer as described before or preferably described below where said polymerized group R₁ is of formulae (1-p), (2-p), (3-p) or (4-p) as described before.

Particularly preferably, such oligomer, polymer or copolymer according to the invention comprises a constitutional unit M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I”).

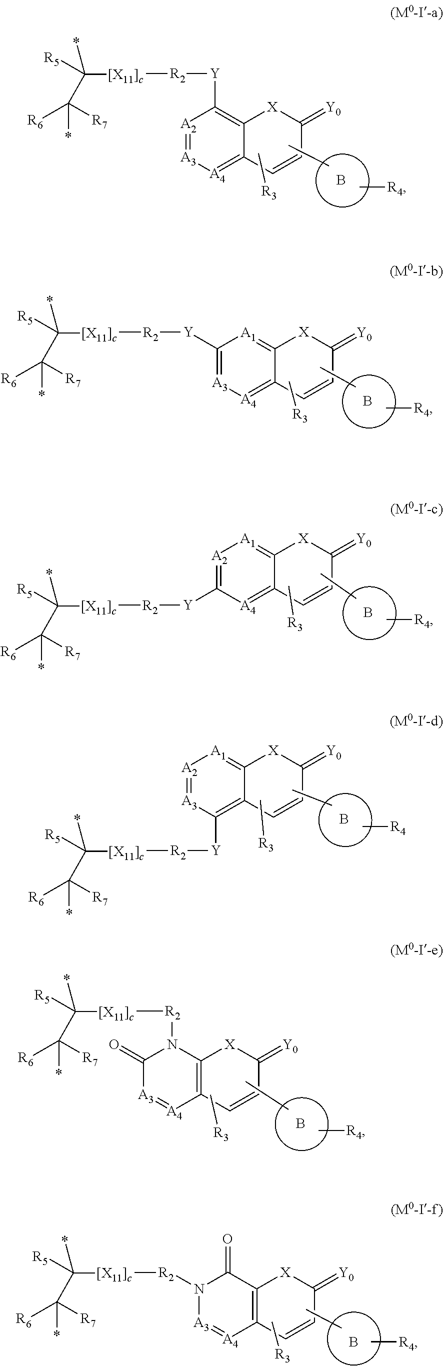

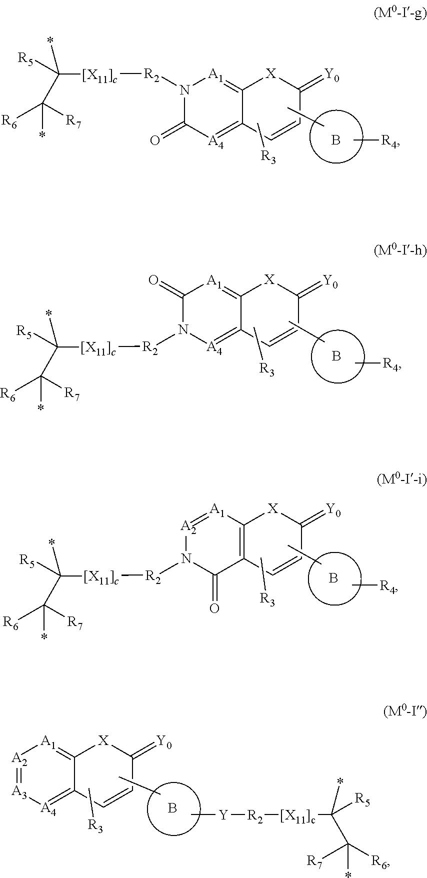

wherein R₁, —R₂—, X, Y₀, Y, A₁, A₂, A₃, A₄, R₃, R₄, R₅, R₆, R₇, X₁₁, c and

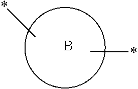

have a meaning as described before or preferably described before or below for the compounds of formulae (I), (I #), (I′), (I′-a), (I′-b), (I′-c), (I′-d), (I′-e), (I′-f), (I′-g), (I′-h), (I′-i) or (I″).

Combinations are excluded where two O atoms or an O atom and a S atom are directly linked to each other as known for a skilled artisan in the field of organic chemistry.

The invention is furthermore directed to an ophthalmic device or a precursor article for manufacturing an ophthalmic device as described before or preferably described below wherein the constitutional unit M⁰ is of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) as described before and where the asterisk “*” denotes at each occurrence a linkage to the adjacent repeating unit in the polymer chain or oligomer chain or to a terminal end group.

Preferably, such oligomer, polymer or copolymer according to the invention comprises a constitutional unit (M⁰-I′-a), (M⁰-I′-b), (M⁰-I′-c), (M⁰-1′-d), (M⁰-I′-e), (M⁰-I′-f), (M⁰-I′-g), (M⁰-I′-h), (M⁰-I′-i) or (M⁰-I″) as described before, wherein

—R₂— is selected from —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₂—S—(CH₂)₂—, —(CH₂)₂—SO₂—(CH₂)₂—, —(CH₂)₂—S—(CH₂)₂—S—(CH₂)₂—, —(CH₂)₂—O—(CH₂)₂—O—(CH₂)₂—, —(CH₂)₂—S—(CH₂)₂—O—(CH₂)₂—, —(CH₂)₂—SO₂—(CH₂)₂—O—(CH₂)₂—, —(CH₂)₂—SO₂—(CH₂)₂—S—(CH₂)₂—, —(CH₂)₂—O—(CH₂)₂—O—(CH₂)₂—O—(CH₂)₂—, —(CH₂)₂—O—(CH₂)₂—S—(CH₂)₂—O—(CH₂)₂—, —(CH₂)₂—S—(CH₂)₂—O—(CH₂)₂—S—(CH₂)₂—, —(CH₂)₂—SO₂—(CH₂)₂—O—(CH₂)₂—SO₂—(CH₂)₂—, —(CH₂)₂—S—(CH₂)₂—S—(CH₂)₂—S—(CH₂)₂—, —(CH₂)₂—SO₂—(CH₂)₂—S—(CH₂)₂—SO₂—(CH₂)₂—, —(CH₂)₂—SO₂—(CH₂)₂—SO₂—(CH₂)₂—SO₂—(CH₂)₂—, —(CH₂)₂—O—(CH₂)₂—SO₂—(CH₂)₂—O—(CH₂)₂—, —(CH₂)₃—(CF₂)—(CH₂)₃—, —(CH₂)—(CF₂)₃—(CH₂)—, —(CH₂)₂—(CF₂)₄—(CH₂)₂—, —(CH₂)—[CH(CF₃)]—(CH₂)—, —(CH₂)—[C(CH₃)CF₃]—(CH₂)—, —(CH₂)—[CH(CH₂CF₃)]—(CH₂)—, —(CH₂)—[C(CH₃)(CH₂CF₃)]—(CH₂)—, —(CH₂)₂—(CF₂)—O—(CF₂)—O—(CF₂)—(CH₂)₂— and —(CH₂)—(CF₂)—O—(CF₂)—O—(CF₂)—O—(CF₂)—(CH₂)— or has a meaning as preferably described before;

Y is O, S, SO₂, or a bond or has a meaning as preferably described before;

R₃ is H, F, a straight-chain alkyl group with 1 to 4 C atoms or a straight-chain alkoxy group with 1 to 4 C atoms or has a meaning as preferably described before;

X₁₁ is selected from the group consisting of O, S, O—SO₂, SO₂—O, C(═O), OC(═O), C(═O)O, S(C═O) and (C═O)S, or has a meaning as preferably described before;

R₆ and R₇ are H;

R₅ is H, methyl, ethyl or phenyl, or has a meaning as preferably described before; and

c is 1 and

X, Y₀, Y, A₁, A₂, A₃, A₄ and

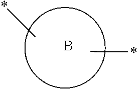

have a meaning as described before or preferably described before or below for the compounds of formulae (I), (I #), (I′), (I′-a), (I′-b), (I′-c), (I′-d), (I′-e), (I′-f), (I′-g), (I′-h), (I′-i) or (I″).

Preferably, such oligomer, polymer or copolymer according is comprised in the ophthalmic device or the precursor article for manufacturing an ophthalmic device according to the invention.

Particularly preferably, such oligomer, polymer or copolymer according to the invention comprises a constitutional unit (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) as described before, wherein

—R₂— is selected from —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₂—S—(CH₂)₂—, —(CH₂)₂—SO₂—(CH₂)₂—, —(CH₂)₂—S—(CH₂)₂—S—(CH₂)₂—, —(CH₂)₂—O—(CH₂)₂—O—(CH₂)₂—, —(CH₂)₂—O—(CH₂)₂—O—(CH₂)₂—O—(CH₂)₂—, —(CH₂)₂—O—(CH₂)₂—S—(CH₂)₂—O—(CH₂)₂—, —(CH₂)₂—S—(CH₂)₂—O—(CH₂)₂—S—(CH₂)₂—, —(CH₂)₂—S—(CH₂)₂—S—(CH₂)₂—S—(CH₂)₂—, —(CH₂)₂—SO₂—(CH₂)₂—S—(CH₂)₂—SO₂—(CH₂)₂— or has a meaning as preferably described before;

Y is O, S, SO₂, or a bond or has a meaning as preferably described before;

R₃ is H, F, a straight-chain alkyl group with 1 to 4 C atoms or a straight-chain alkoxy group with 1 to 4 C atoms or has a meaning as preferably described before;

X₁₁ is selected from the group consisting of O, S, O—SO₂, SO₂—O, C(═O), OC(═O), C(═O)O, S(C═O) and (C═O)S, or has a meaning as preferably described before;

R₆ and R₇ are H;

R₅ is H, methyl, ethyl or phenyl, or has a meaning as preferably described before; and

c is 1,

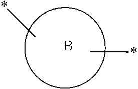

is in position 3 of the photoactive chromophore as described before or in formula (I #), and

X, Y₀, Y, A₁, A₂, A₃, A₄ and

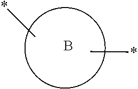

have a meaning as described before or preferably described before or below for the compounds of formulae (I), (I #), (I′), (I′-a), (I′-b), (I′-c), (I′-d), (I′-e), (I′-f), (I′-g), (I′-h), (I′-i) or (I″).

Particularly preferably, such oligomer, polymer or copolymer is comprised in the ophthalmic device or the precursor article for manufacturing an ophthalmic device according to the invention.

The copolymer may be an oligomer or polymer comprising one or more polymerized compounds of formulae (I), (l #), (I′), (I′-a), (I′-b), (I′-c), (I′-d), (I′-e), (I′-f), (I′-g), (I′-h), (I′-i), or (I″) as described before or preferably described before or one or more constitutional units M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) and/or (M⁰-I″) as described before or preferably described before or one or more constitutional units (M⁰-001) to (M⁰-162) as described below, which may be the same or different from one another, and one or more constitutional units M², which may be the same or different from one another. Said one or more constitutional units M² are chemically different from the units M⁰. Preferably, said one or more constitutional units M² are derived by polymerization of one or more monomers selected from the group consisting of styrene, ethoxyethyl methacrylate (EOEMA), methyl methacrylate (MMA), methyl acrylate, n-alkyl acrylates (the n-alkyl group comprising 2-20 C-atoms), n-alkyl methacrylates (the n-alkyl group comprising 2-20 C-atoms), i-alkyl acrylates (the i-alkyl group comprising 3-20 C-atoms), i-alkyl methacrylates (the i-alkyl group comprising 3-20 C-atoms), ethoxyethoxy ethylacrylate (EEEA), 2-hydroxyethyl methacrylate (HEMA), tetrahydrofuryl methacrylate (THFMA), glycidylmethacrylate (GMA), 16-hydroxyhexadecyl acrylate, 16-hydroxyhexadecyl methacrylate, 18-hydroxyoctadecyl acrylate, 18-hydroxyoctadecyl methacrylate, 2-phenoxyethyl acrylate (EGPEA), heptafluorobutyl acrylate, heptafluorobutyl methacrylate, hexafluorobutyl acrylate, hexafluorobutyl methacrylate, hexafluoroisopropyl acrylate, hexafluoroisopropyle methacrylate, octafluoropentyl acrylate, octafluoropentyl methacrylate, petanfluoropropyl acrylate, pentafluoropropyl methacrylate, tetrafluoropropyl methacrylate, trifluoroethyl acrylate, trifluoroethyl methacrylate, Bisphenol A diacrylate-1 EO/Phenol (BPADA), 2-[3′-2′H-benzotriazol-2′-yl)-4′-hydroxyphenyl]ethyl methacrylate (BTPEM) or ehtyleneglycoldimethacrylate.

The invention therefore relates further to an ophthalmic device or a precursor article for manufacturing an ophthalmic device as described or preferably described before comprising beside of the at least one polymerized compound of formulae (I), (I′) or (I″) or the constitutional unit M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) and/or (M⁰-I″) as described before or preferably described before or one or more constitutional units (M⁰-001) to (M⁰-162) as described below at least one further polymerized monomer selected from the group consisting of styrene, ethoxyethyl methacrylate (EOEMA), methyl methacrylate (MMA), methyl acrylate, n-alkyl acrylates (the n-alkyl group comprising 2-20 C-atoms), n-alkyl methacrylates (the n-alkyl group comprising 2-20 C-atoms), i-alkyl acrylates (the i-alkyl group comprising 3-20 C-atoms), i-alkyl methacrylates (the i-alkyl group comprising 3-20 C-atoms), ethoxyethoxy ethylacrylate (EEEA), 2-hydroxyethyl methacrylate (HEMA), tetrahydrofuryl methacrylate (THFMA), glycidylmethacrylate (GMA), 16-hydroxyhexadecyl acrylate, 16-hydroxyhexadecyl methacrylate, 18-hydroxyoctadecyl acrylate, 18-hydroxyoctadecyl methacrylate, 2-phenoxyethyl acrylate (EGPEA), heptafluorobutyl acrylate, heptafluorobutyl methacrylate, hexafluorobutyl acrylate, hexafluorobutyl methacrylate, hexafluoroisopropyl acrylate, hexafluoroisopropyle methacrylate, octafluoropentyl acrylate, octafluoropentyl methacrylate, petanfluoropropyl acrylate, pentafluoropropyl methacrylate, tetrafluoropropyl methacrylate, trifluoroethyl acrylate, trifluoroethyl methacrylate, Bisphenol A diacrylate-1 EO/Phenol (BPADA), 2-[3′-2′H-benzotriazol-2′-yl)-4′-hydroxyphenyl]ethyl methacrylate (BTPEM) or ehtyleneglycoldimethacrylate.

Particularly preferably, the at least one further polymerized monomer is selected from methyl methacrylate, 2-hydroxyethyl methacrylate, 2-phenoxyethyl acrylate, ethoxyethoxy ethylacrylate, 8-methylnonyl methacrylate, n-butyl methacrylate, 2-ethyl hexylmethacrylate or a mixture thereof.

Particularly preferably, such copolymer is comprised in the ophthalmic device or the precursor article for manufacturing an ophthalmic device according to the invention.

Alternatively the oligomer or polymer, preferably the polymer, according to the invention is a homopolymer, i.e. an oligomer or polymer, preferably a polymer, comprising one or more constitutional units M⁰ of formula (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) as described before or preferably described before or (M⁰-001) to (M⁰-162) as described below and wherein all constitutional units M⁰ are the same.

Exemplary homopolymeric compounds based on compounds of formulae are the following compounds (P-001) to (P-162) as shown in table 2.

TABLE 2

| | |
|---|---|
| 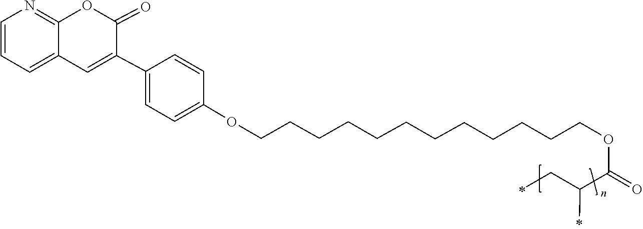 | P-001 |
| 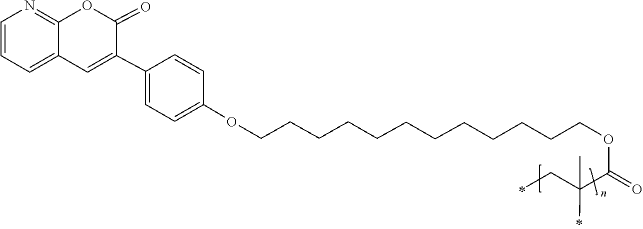 | P-002 |
| 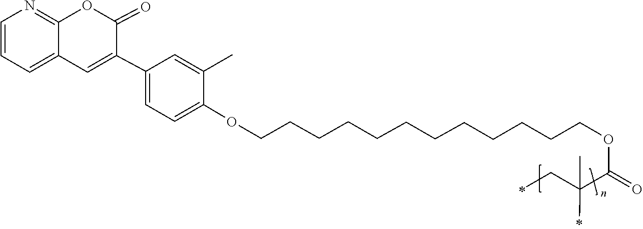 | P-003 |
| 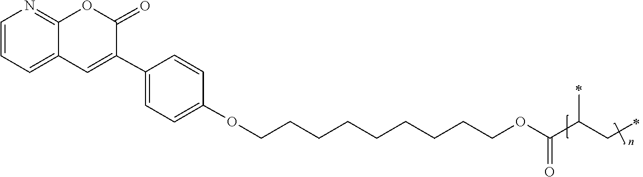 | P-004 |
| 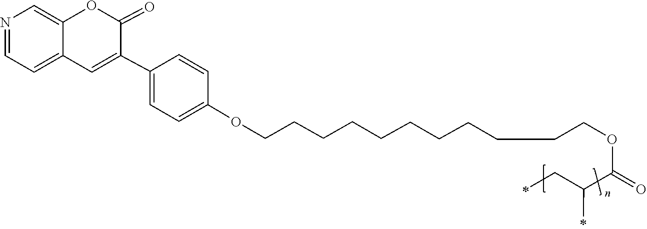 | P-005 |
| 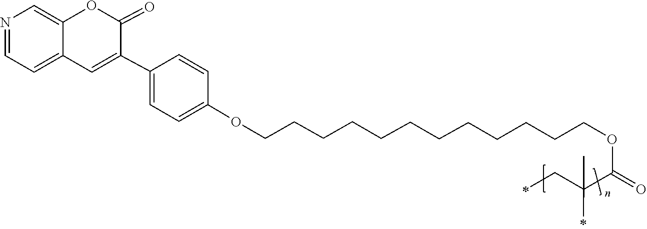 | P-006 |
| 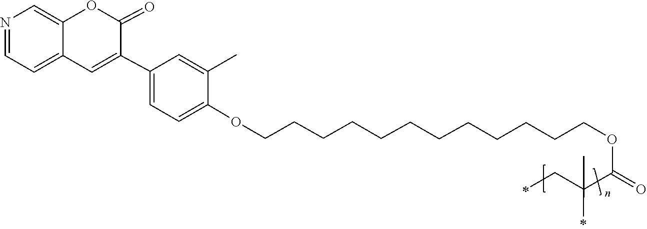 | P-007 |
| 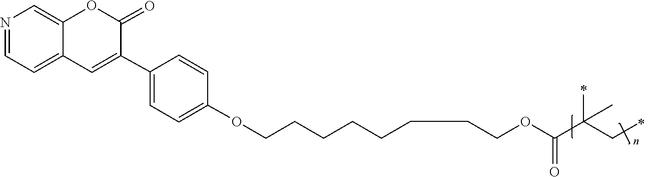 | P-008 |
| 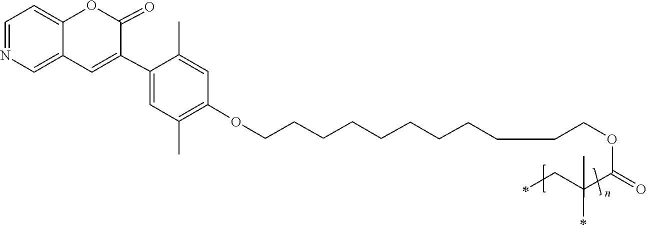 | P-009 |
| 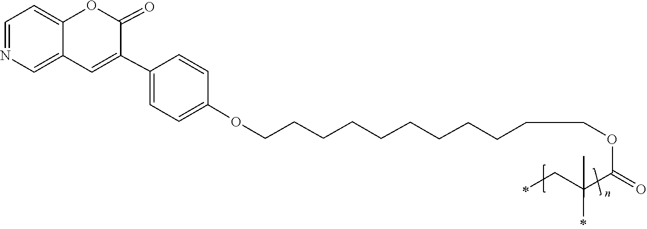 | P-010 |
| 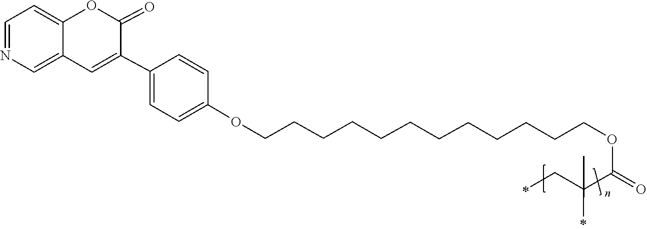 | P-011 |
| 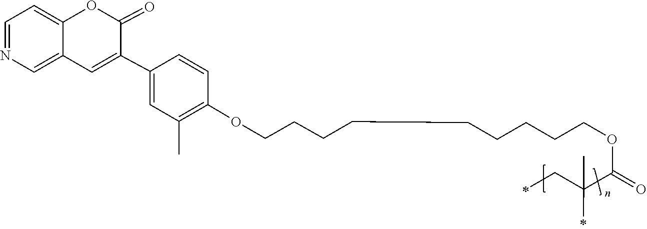 | P-012 |
| 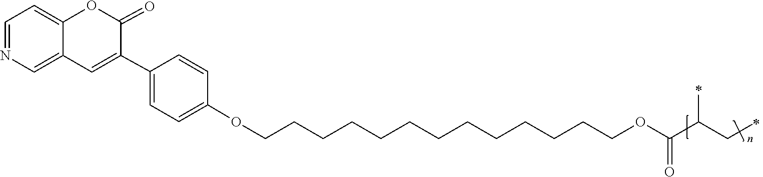 | P-013 |
| 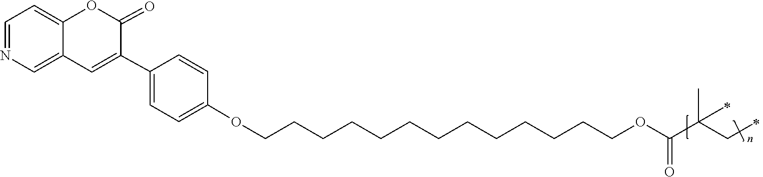 | P-014 |
| 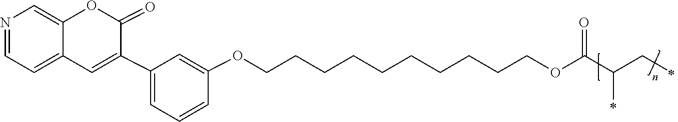 | P-015 |
| 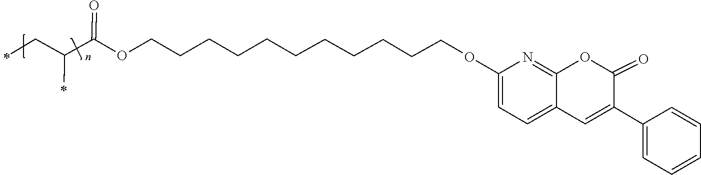 | P-016 |
| 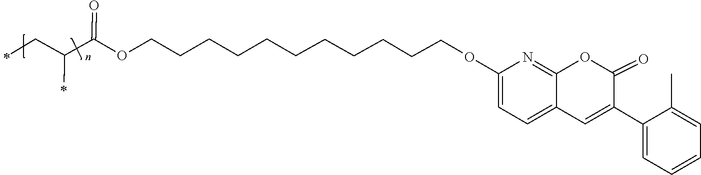 | P-017 |
| 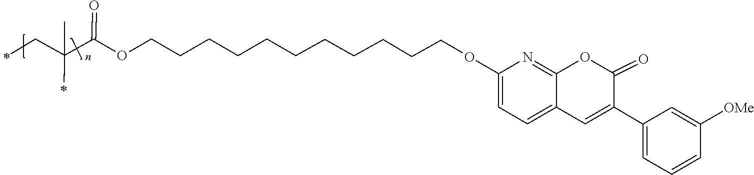 | P-018 |
| 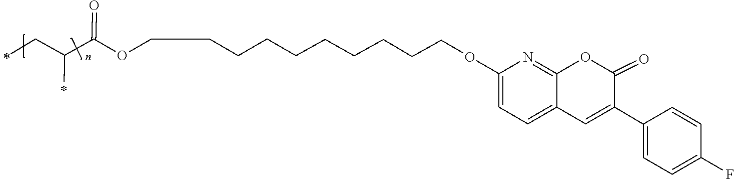 | P-019 |
| 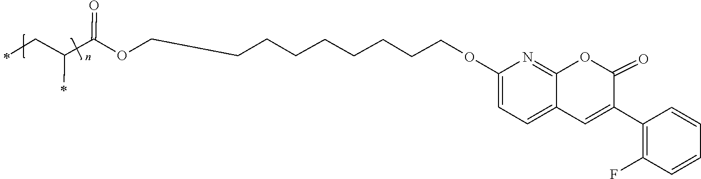 | P-020 |
| 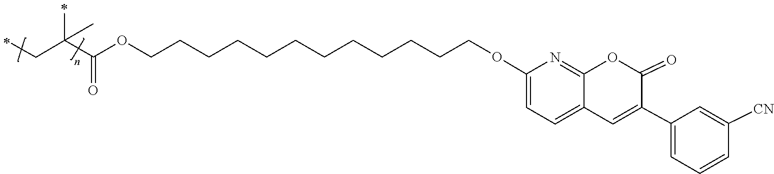 | P-021 |
| 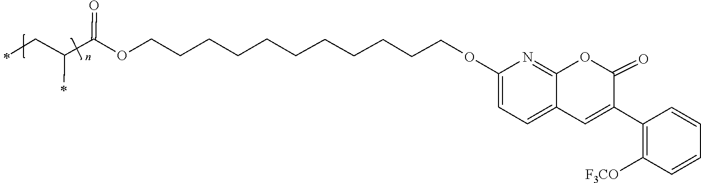 | P-022 |
| 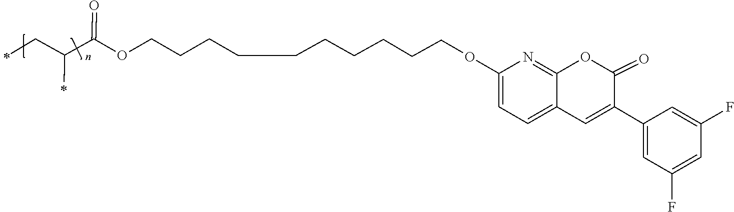 | P-023 |
| 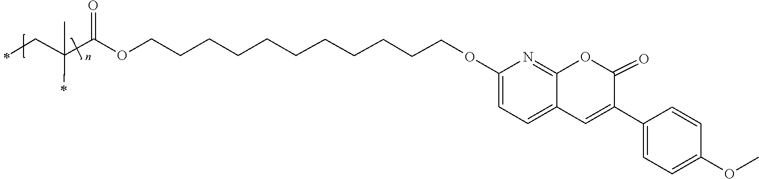 | P-024 |
| 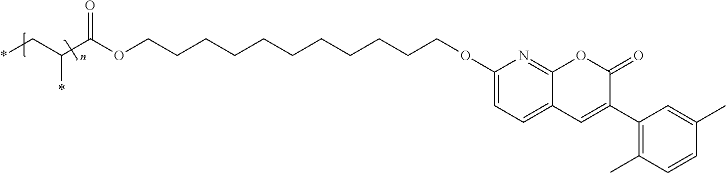 | P-025 |
| 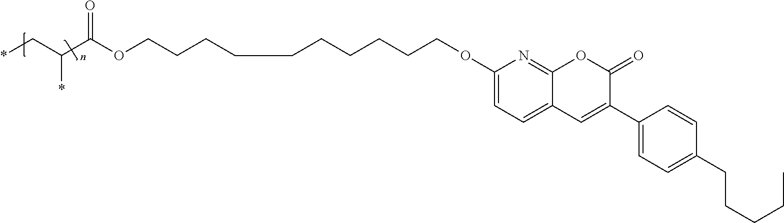 | P-026 |
| 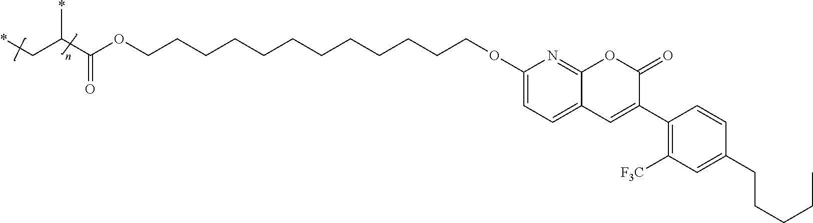 | P-027 |
| 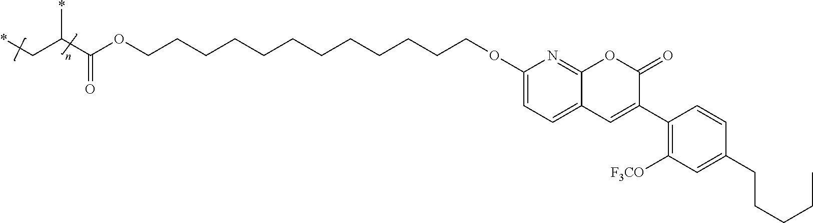 | P-028 |
| 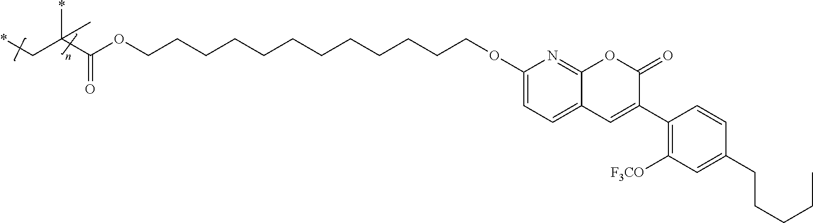 | P-029 |
| 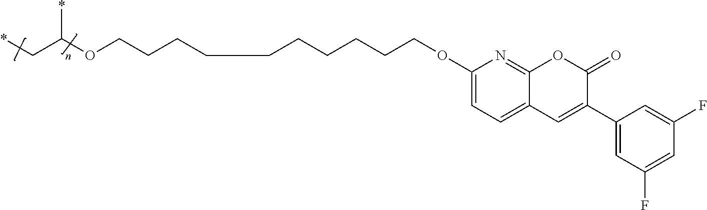 | P-030 |
| 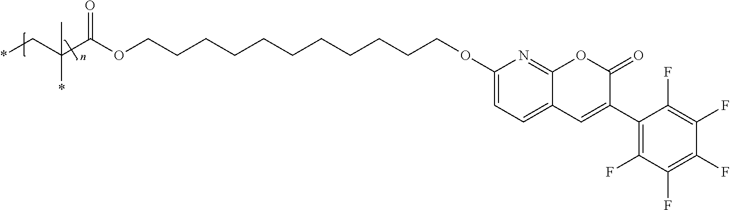 | P-031 |
| 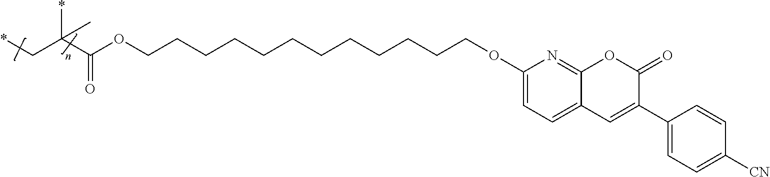 | P-032 |
| 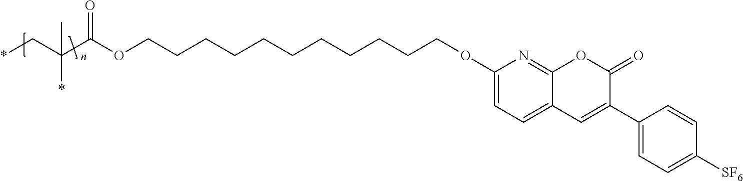 | P-033 |
| 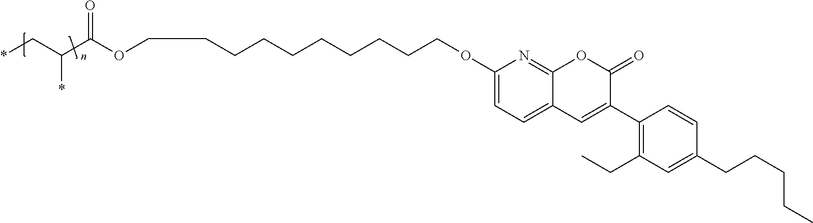 | P-034 |
| 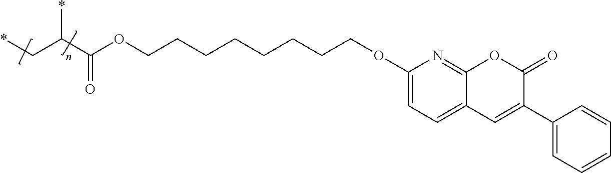 | P-035 |
| 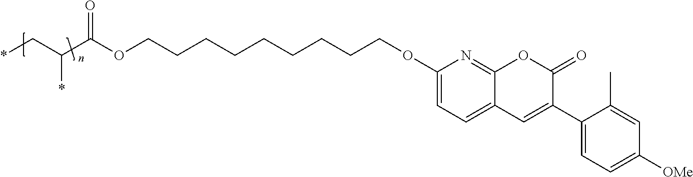 | P-036 |
| 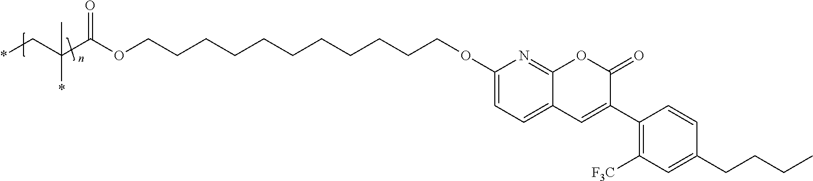 | P-037 |
| 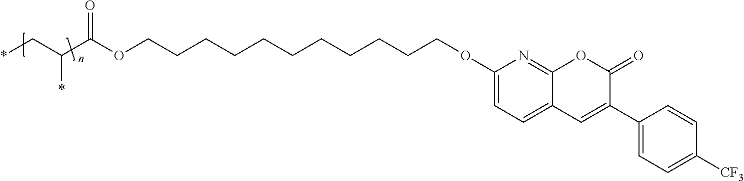 | P-038 |
| 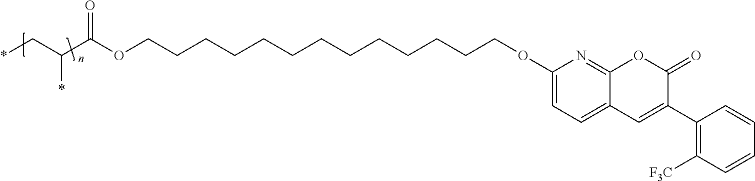 | P-039 |
| 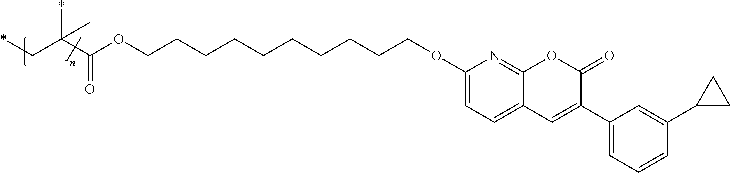 | P-040 |
| 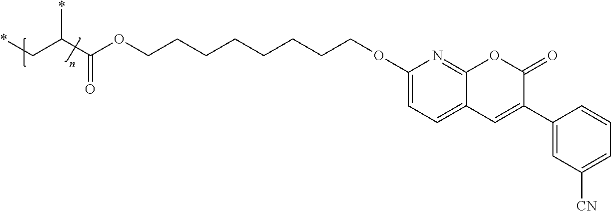 | P-041 |
| 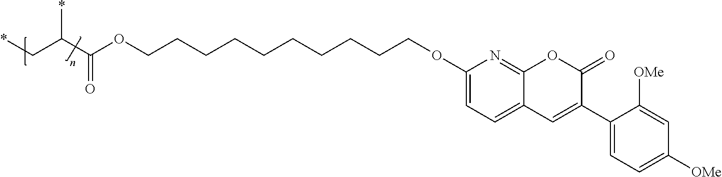 | P-042 |
| 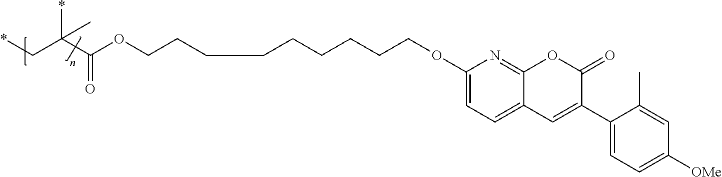 | P-043 |
| 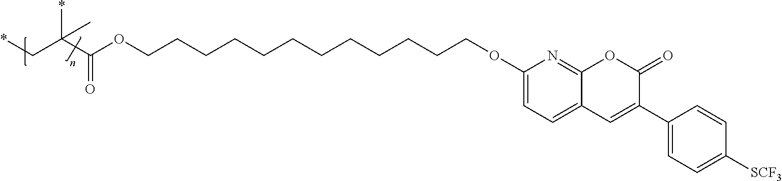 | P-044 |
| 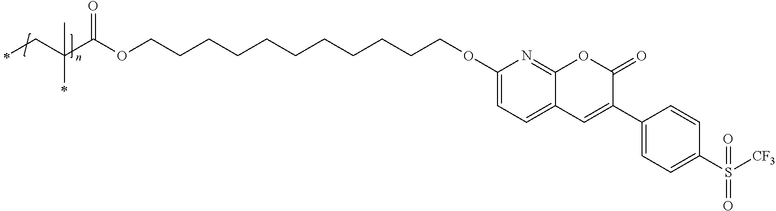 | P-045 |
| 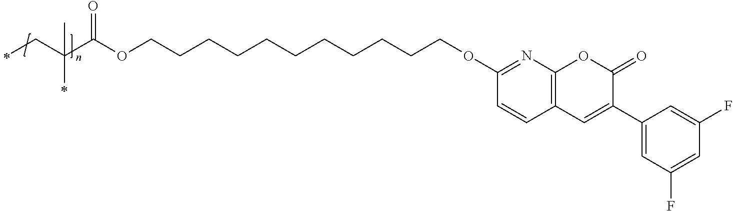 | P-046 |
| 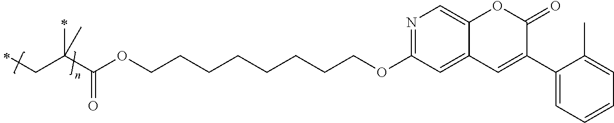 | P-047 |
| 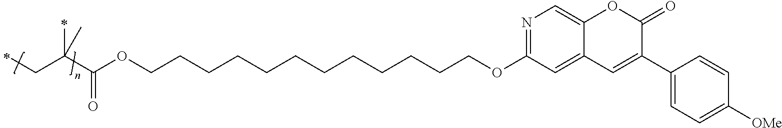 | P-048 |
| 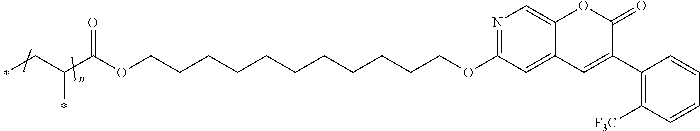 | P-049 |
| 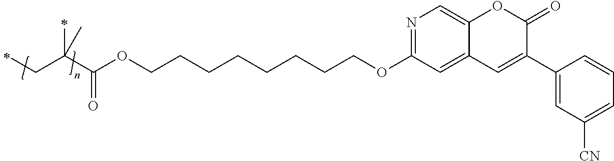 | P-050 |
| 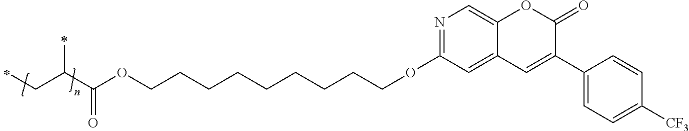 | P-051 |
| 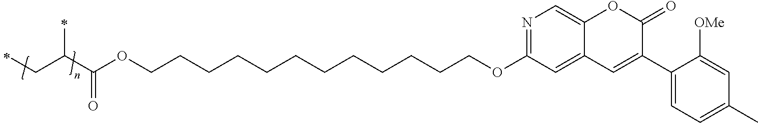 | P-052 |
| 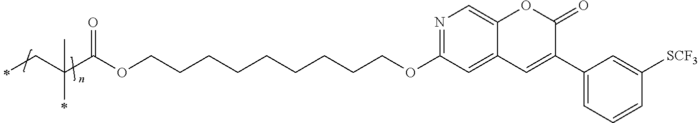 | P-053 |
| 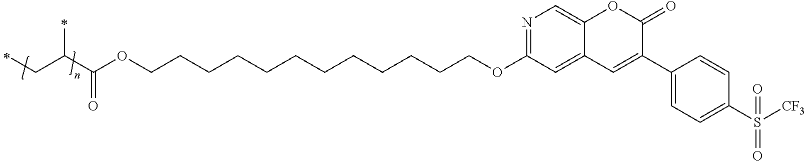 | P-054 |
| 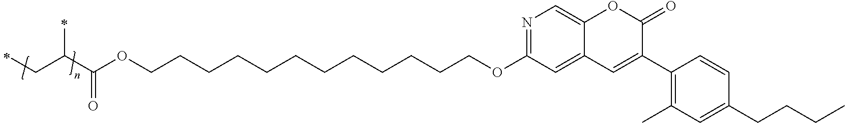 | P-055 |
| 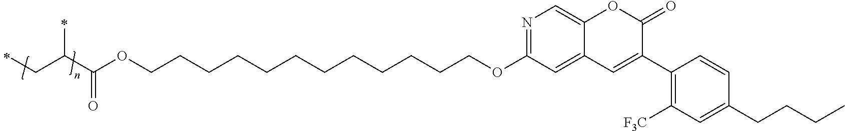 | P-056 |
| 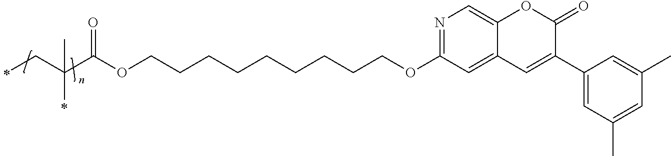 | P-057 |
| 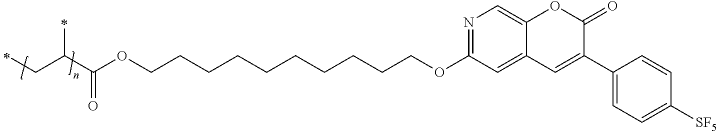 | P-058 |
| 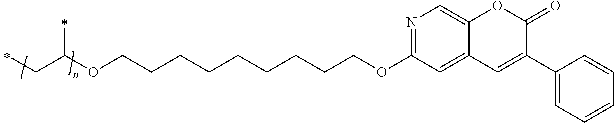 | P-059 |
| 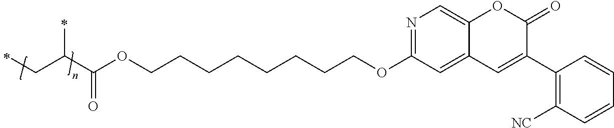 | P-060 |
| 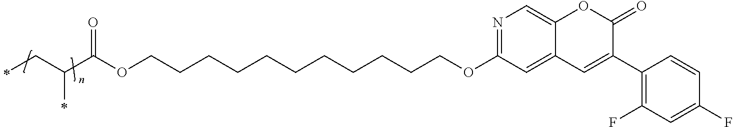 | P-061 |
| 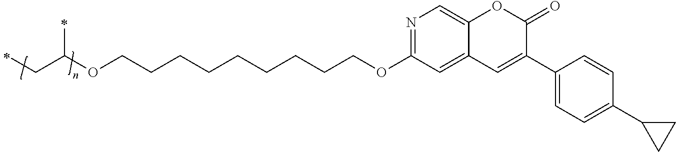 | P-062 |
| 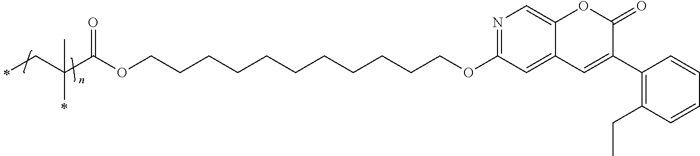 | P-063 |
| 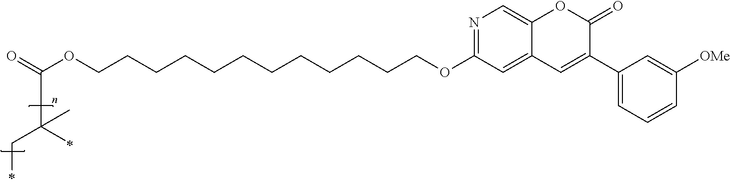 | P-064 |
| 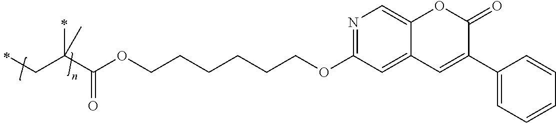 | P-065 |
| 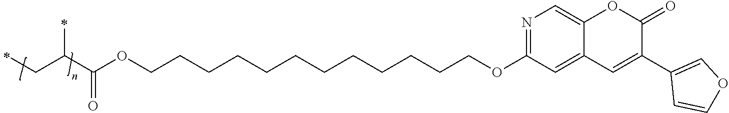 | P-066 |
| 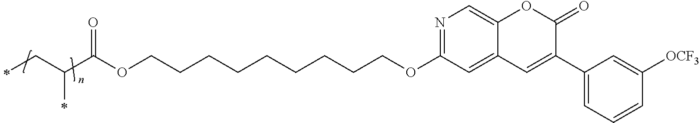 | P-067 |
| 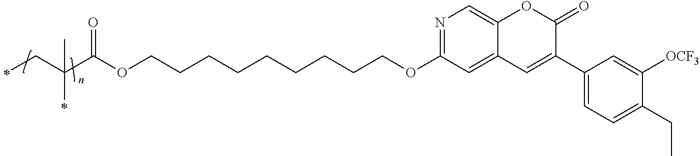 | P-068 |
| 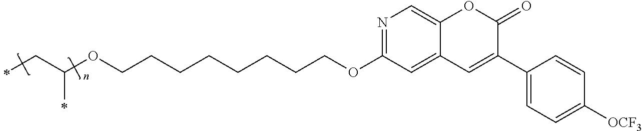 | P-069 |
| 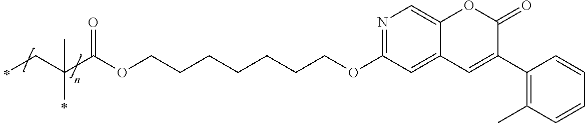 | P-070 |
| 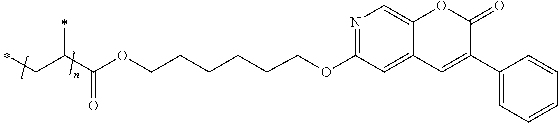 | P-071 |
| 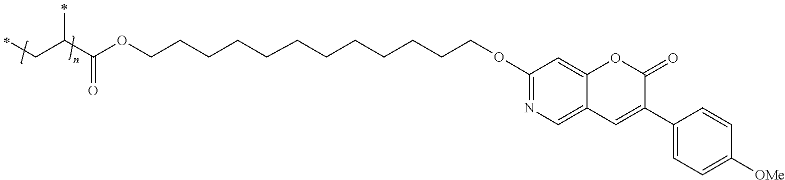 | P-072 |
| 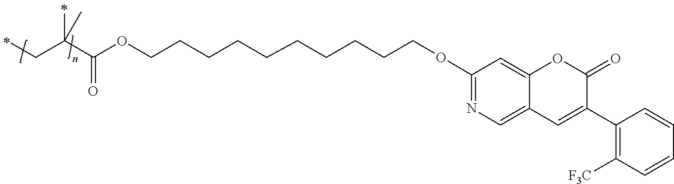 | P-073 |
| 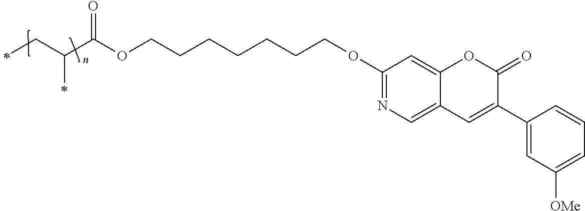 | P-074 |
| 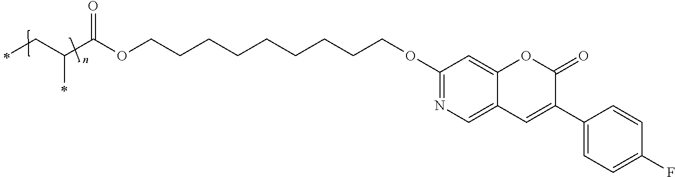 | P-075 |
| 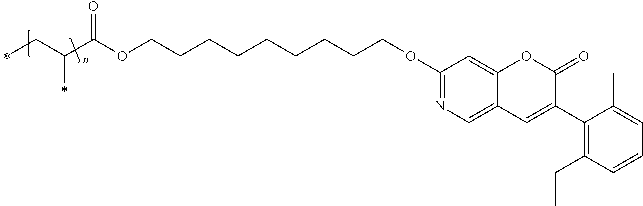 | P-076 |
| 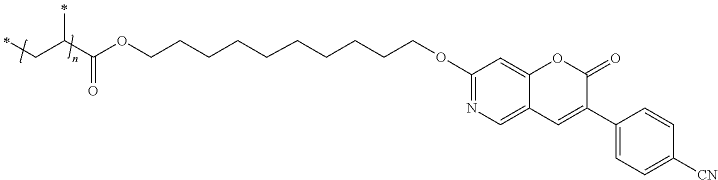 | P-077 |
| 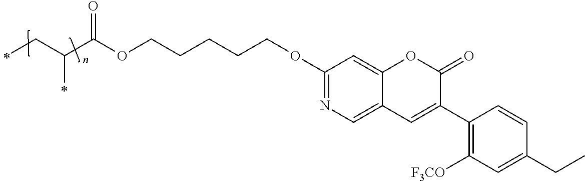 | P-078 |
| 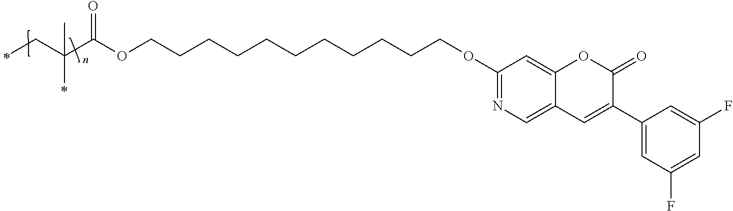 | P-079 |
| 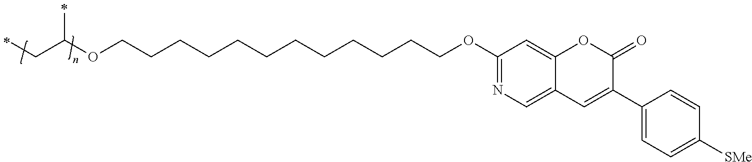 | P-080 |
| 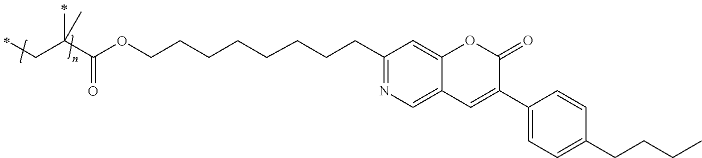 | P-081 |
| 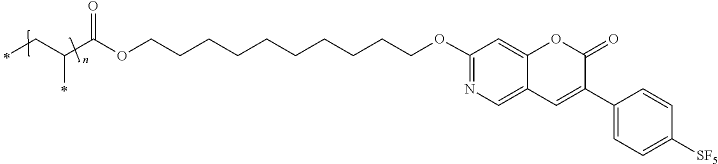 | P-082 |
| 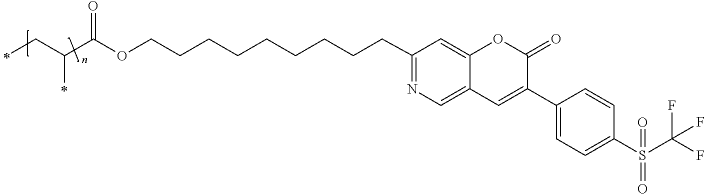 | P-083 |
| 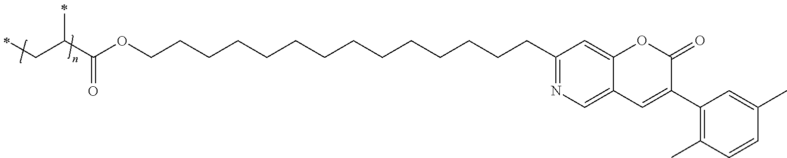 | P-084 |
| 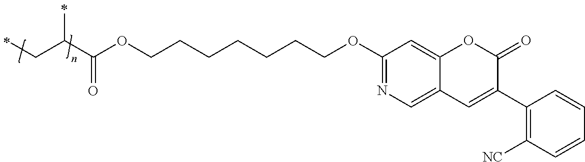 | P-085 |
| 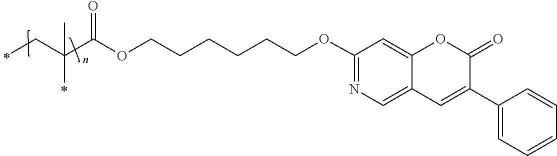 | P-086 |
| 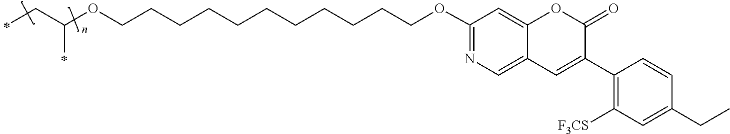 | P-087 |
| 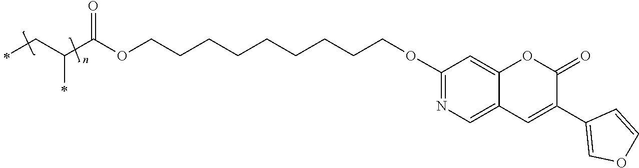 | P-088 |
| 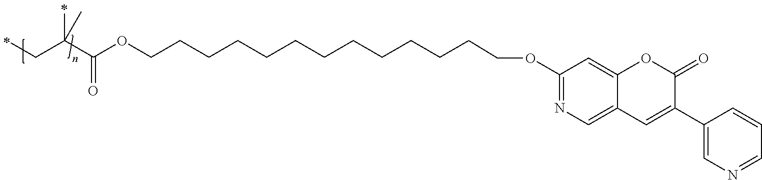 | P-089 |
| 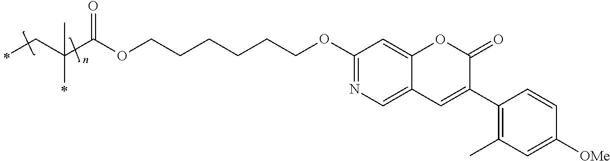 | P-090 |
| 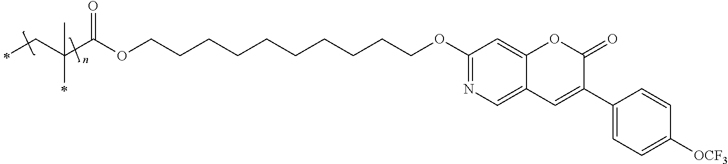 | P-091 |
| 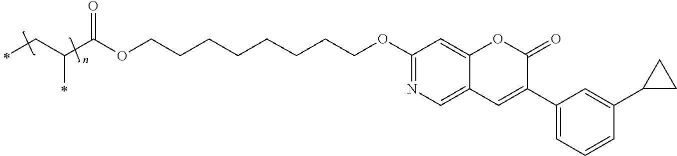 | P-092 |
| 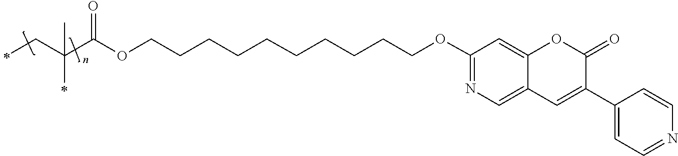 | P-093 |
| 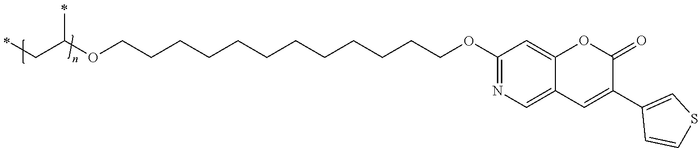 | P-094 |
| 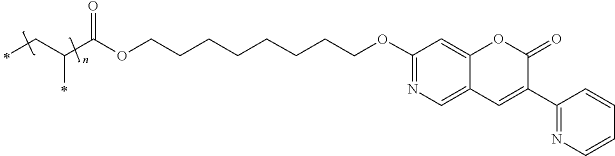 | P-095 |
| 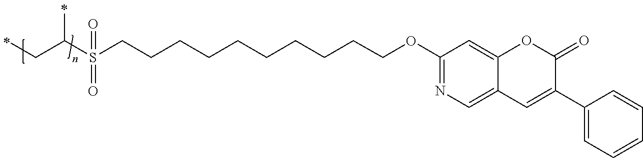 | P-096 |
| 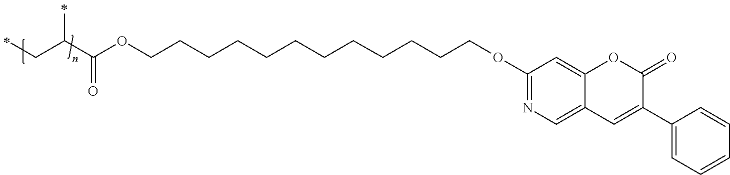 | P-097 |
| 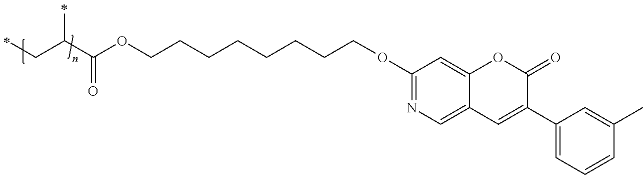 | P-098 |
| 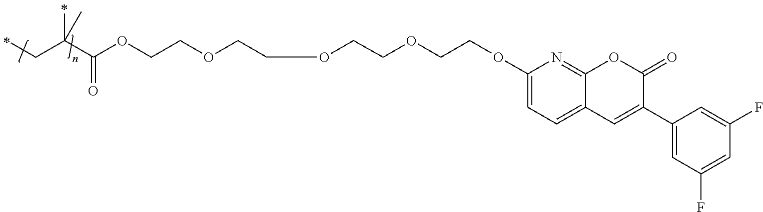 | P-099 |
| 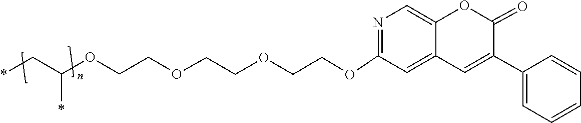 | P-100 |
| 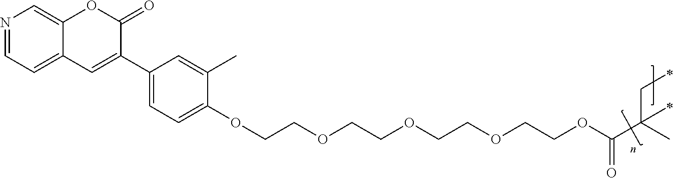 | P-101 |
| 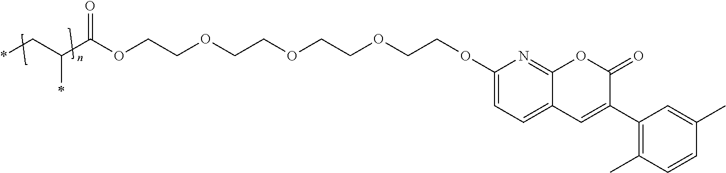 | P-102 |
| 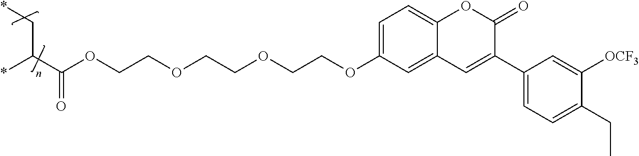 | P-103 |
| 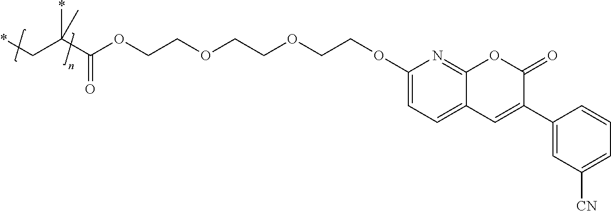 | P-104 |
| 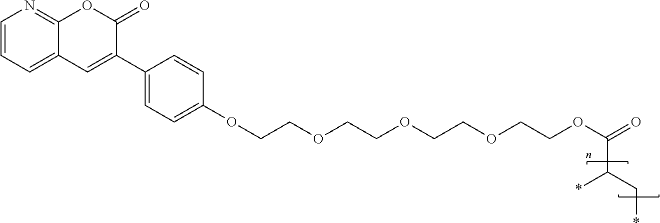 | P-105 |
| 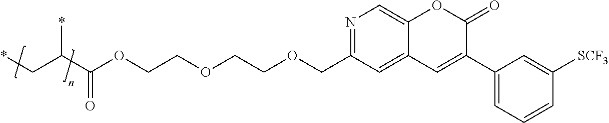 | P-106 |
| 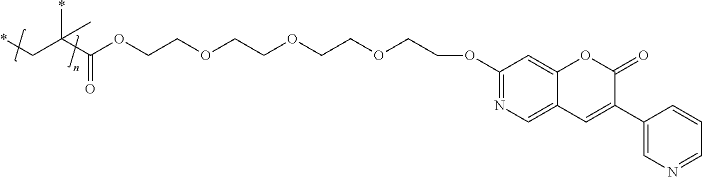 | P-107 |
| 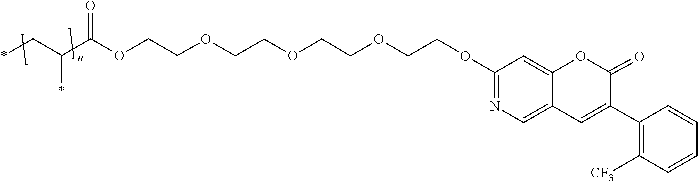 | P-108 |
| 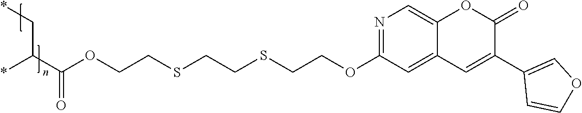 | P-109 |
| 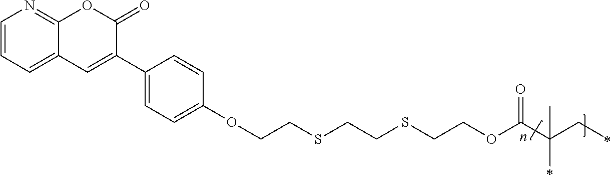 | P-110 |
| 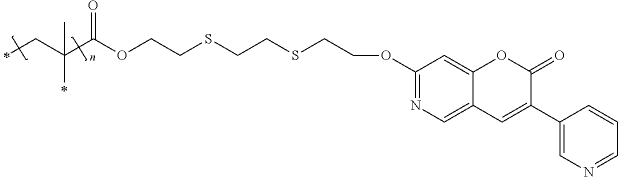 | P-111 |
| 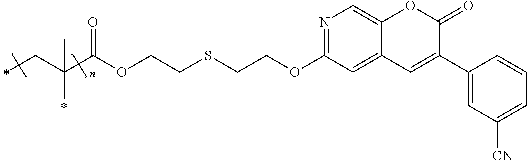 | P-112 |
| 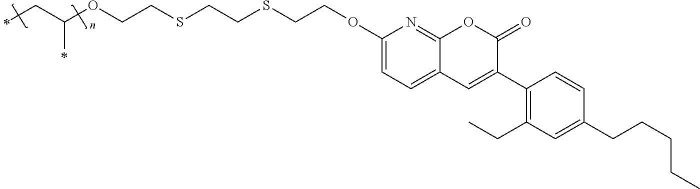 | P-113 |
| 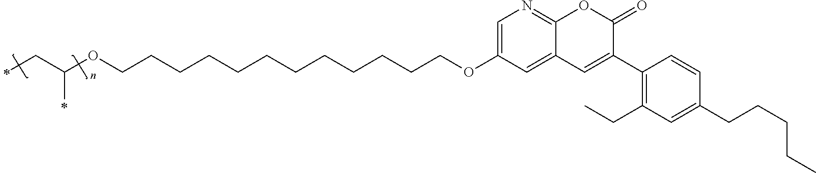 | P-114 |
| 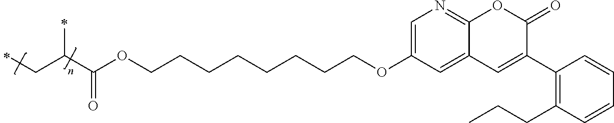 | P-115 |
| 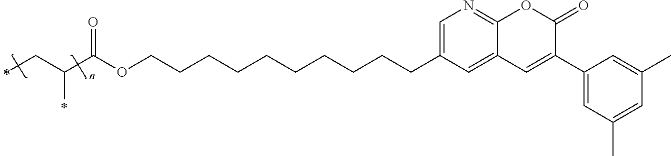 | P-116 |
| 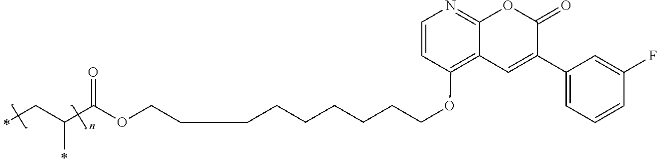 | P-117 |
| 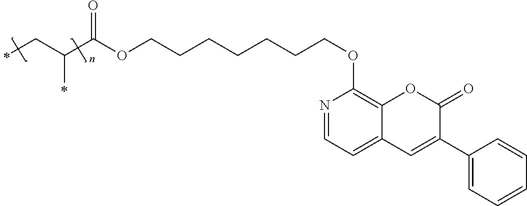 | P-118 |
| 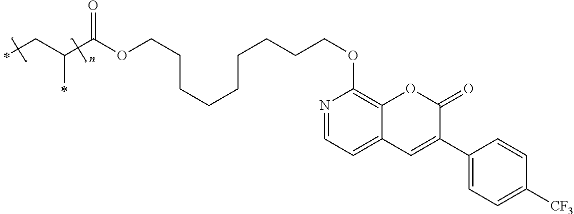 | P-119 |
| 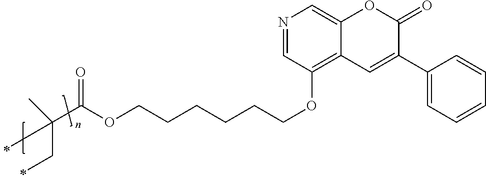 | P-120 |
| 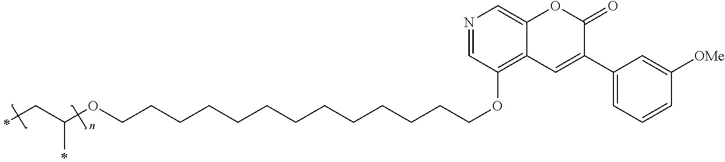 | P-121 |
| 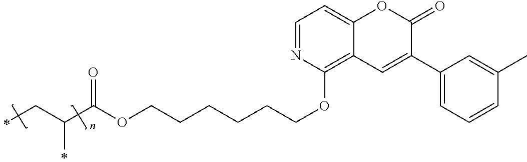 | P-122 |
| 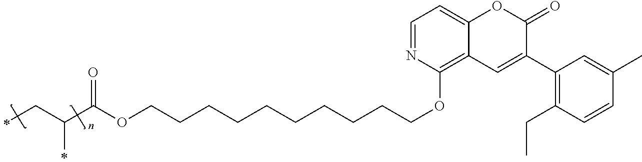 | P-123 |
| 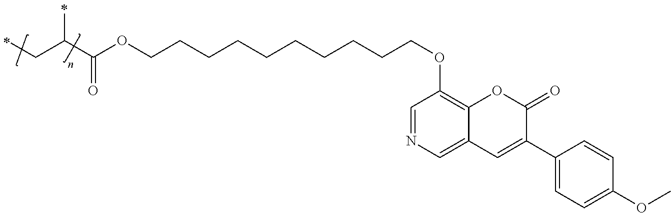 | P-124 |
| 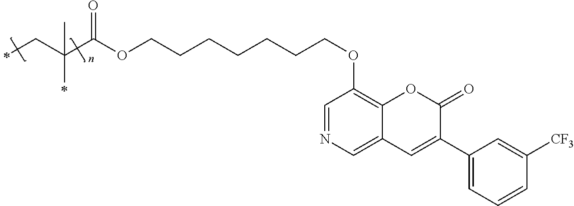 | P-125 |
| 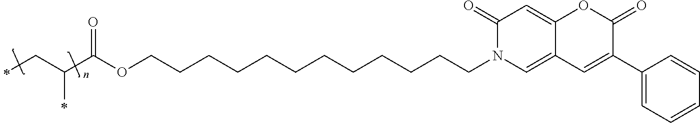 | P-126 |
| 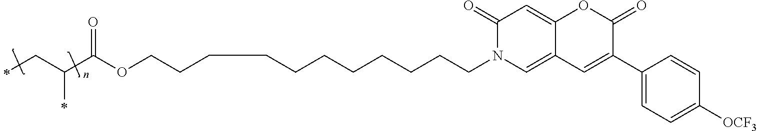 | P-127 |
| 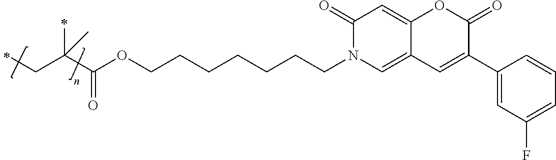 | P-128 |
| 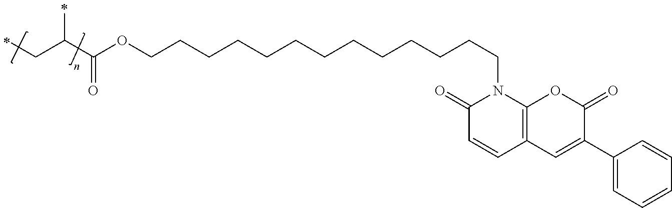 | P-129 |
| 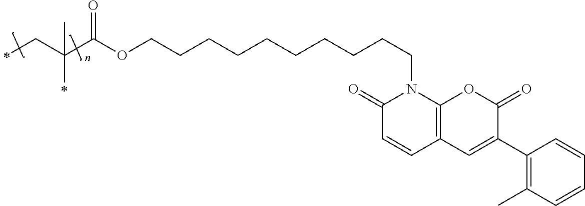 | P-130 |
| 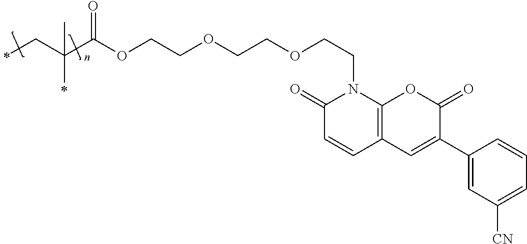 | P-131 |
| 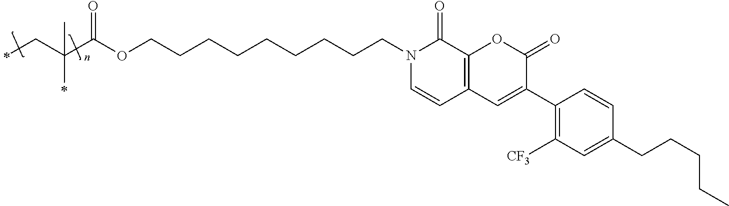 | P-132 |
| 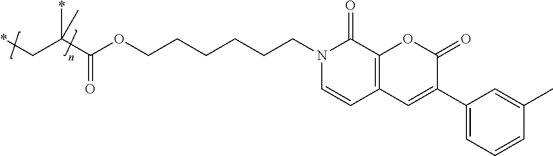 | P-133 |
| 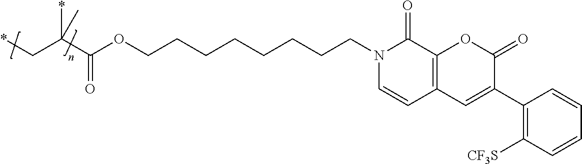 | P-134 |
| 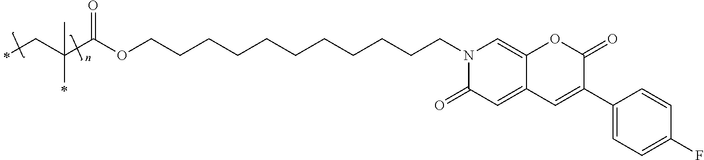 | P-135 |
| 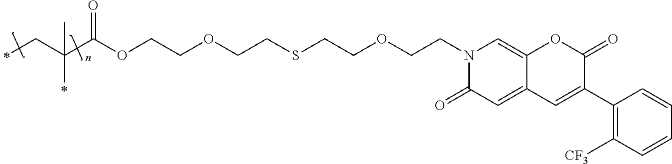 | P-136 |
| 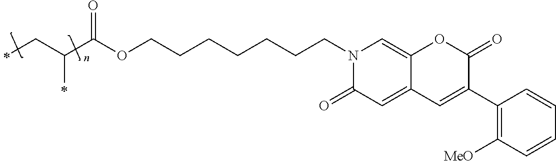 | P-137 |
| 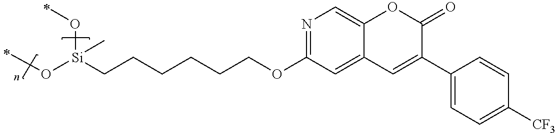 | P-138 |
| 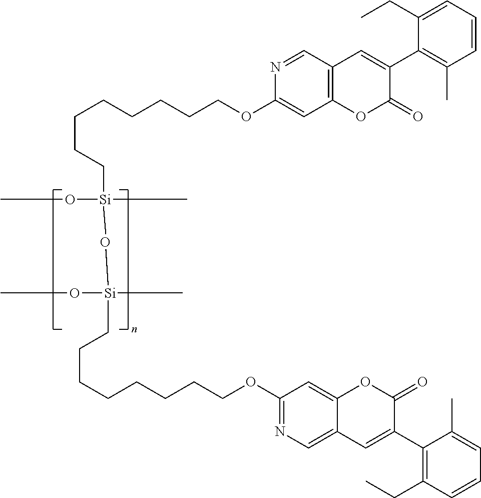 | P-139 |
| 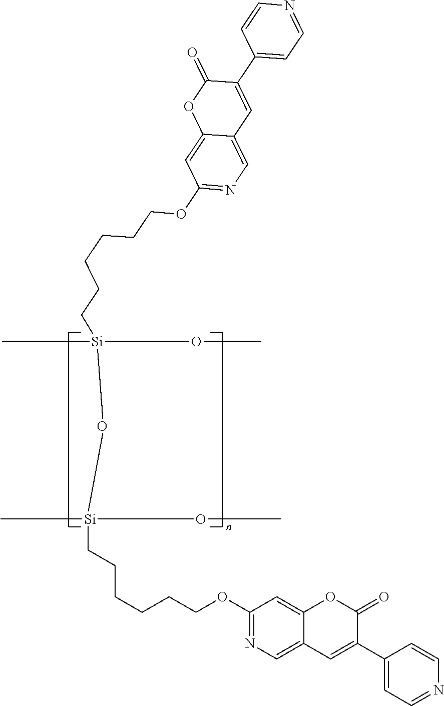 | P-140 |
| 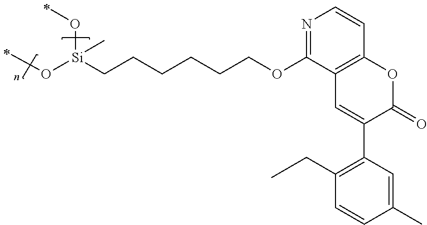 | P-141 |
| 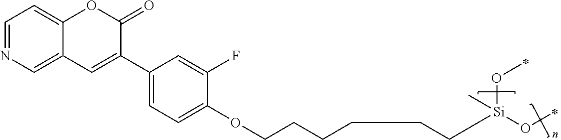 | P-142 |
| 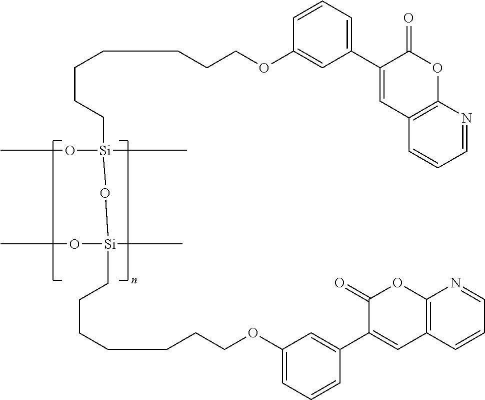 | P-143 |
| 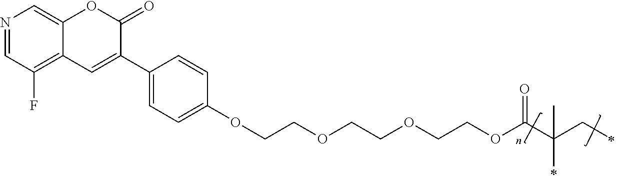 | P-144 |
| 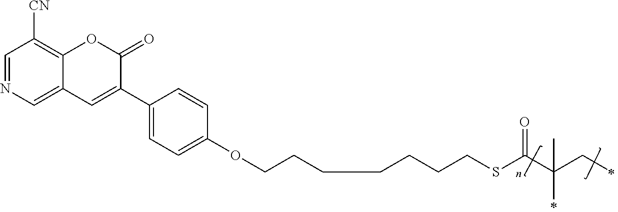 | P-145 |
| 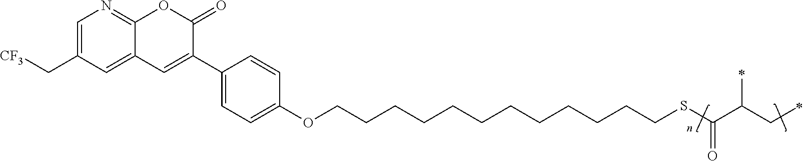 | P-146 |
| 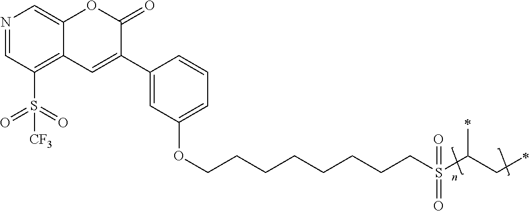 | P-147 |
| 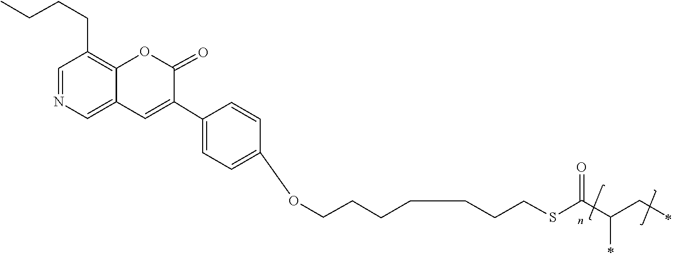 | P-148 |
| 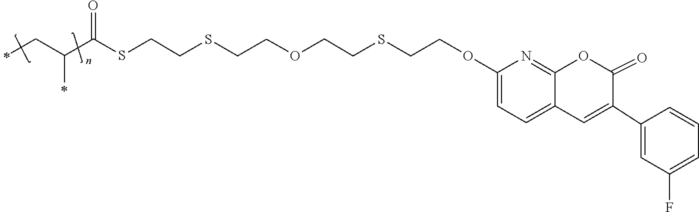 | P-149 |
| 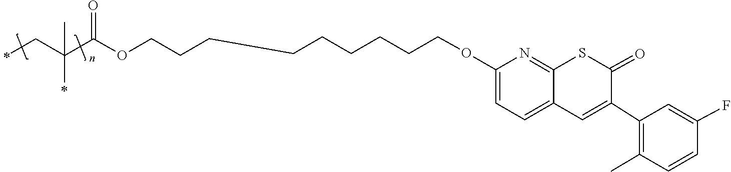 | P-150 |
| 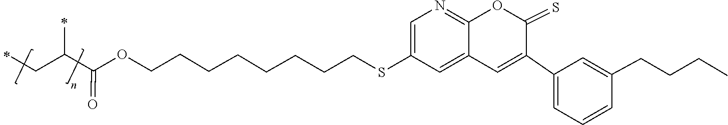 | P-151 |
| 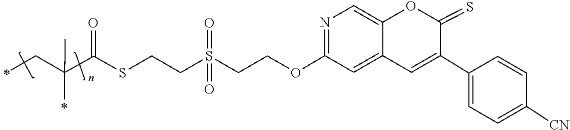 | P-152 |
| 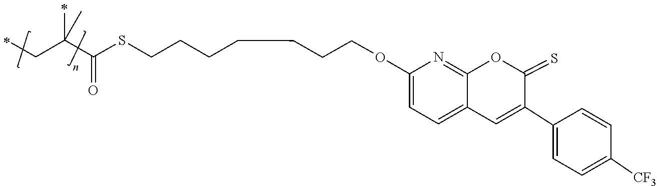 | P-153 |
| 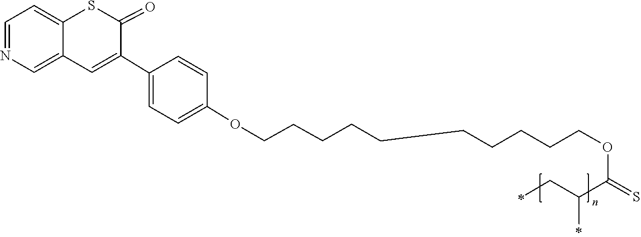 | P-154 |
| 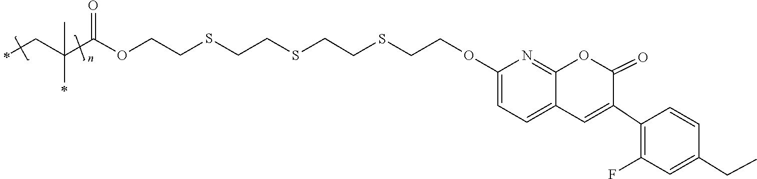 | P-155 |
| 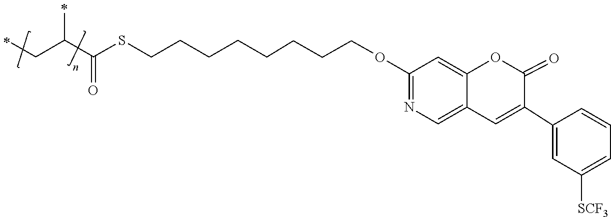 | P-156 |
| 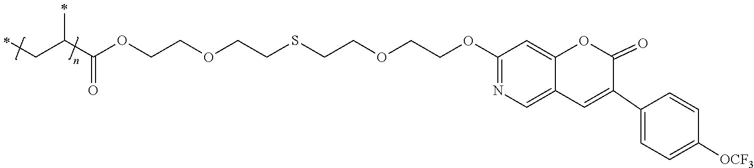 | P-157 |
| 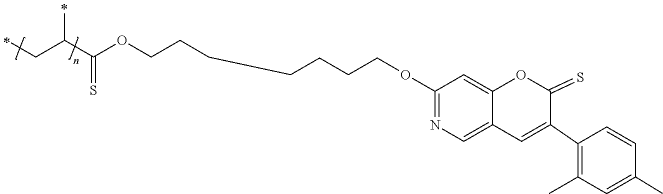 | P-158 |
| 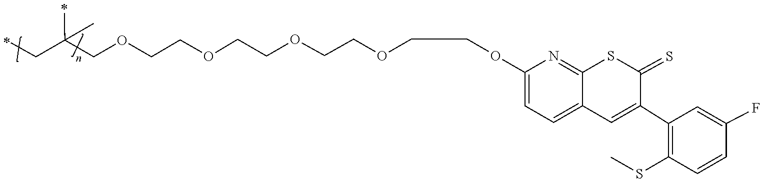 | P-159 |
| 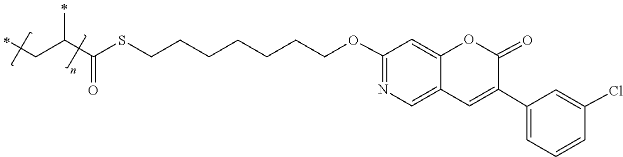 | P-160 |
| 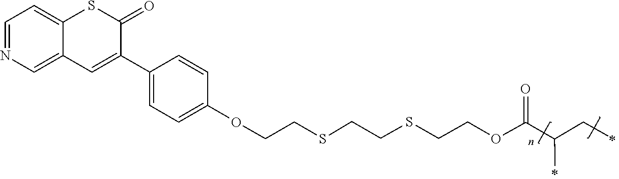 | P-161 |
| 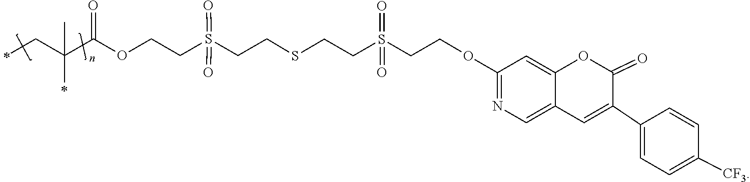 | P-162 |

The letter n gives the degree of polymerization as explained before.

Exemplary constitutional units M⁰ based on compounds of formulae (I), (I #), constitutional units M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) are the following compounds (M⁰-001) to (M⁰-162) as shown in table 2-1.

TABLE 2-1

| | |
|---|---|
| 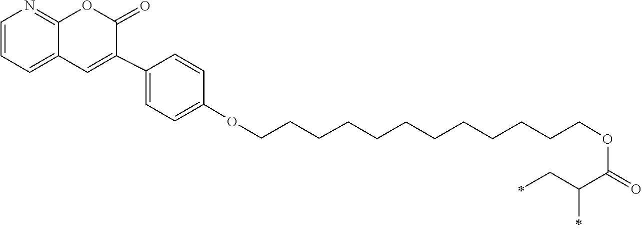 | M⁰-001 |
| 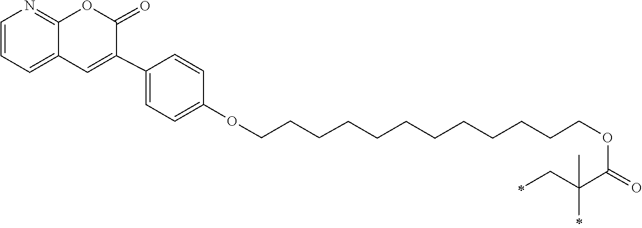 | M⁰-002 |
| 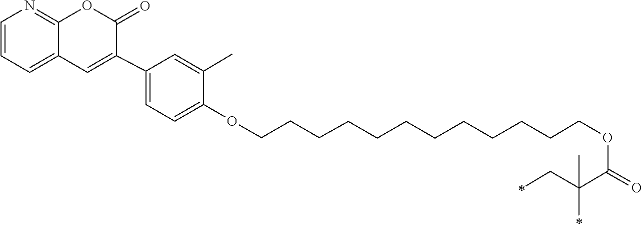 | M⁰-003 |
| 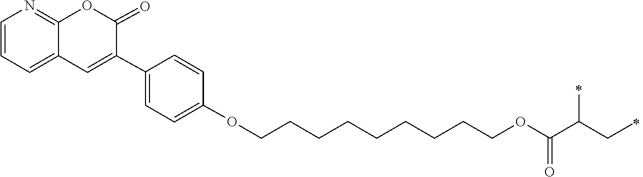 | M⁰-004 |
| 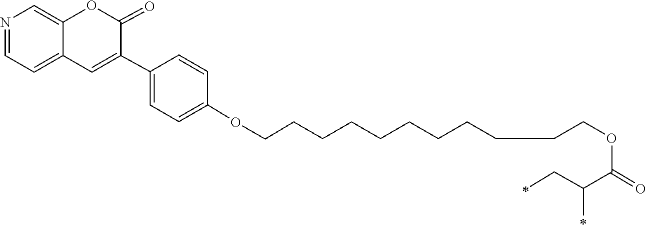 | M⁰-005 |
| 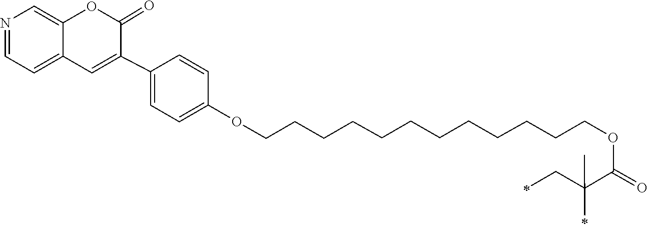 | M⁰-006 |
| 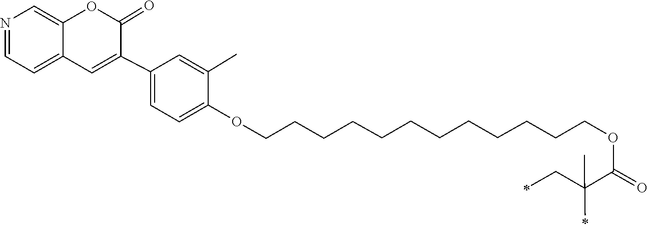 | M⁰-007 |
| 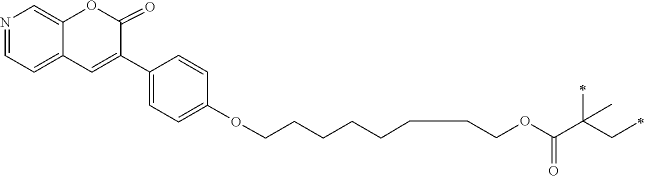 | M⁰-008 |
| 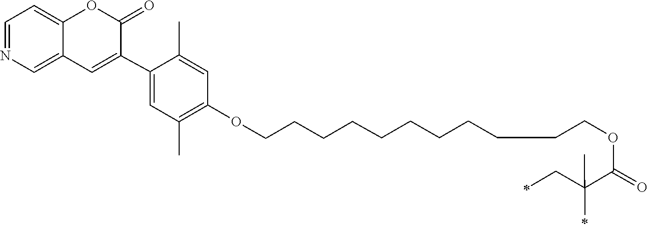 | M⁰-009 |
| 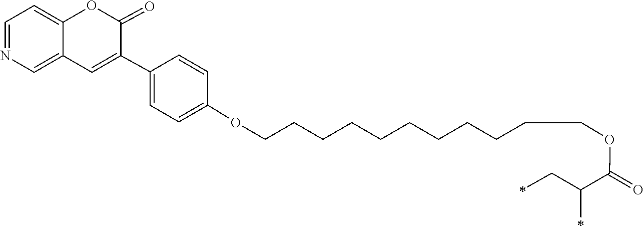 | M⁰-010 |
| 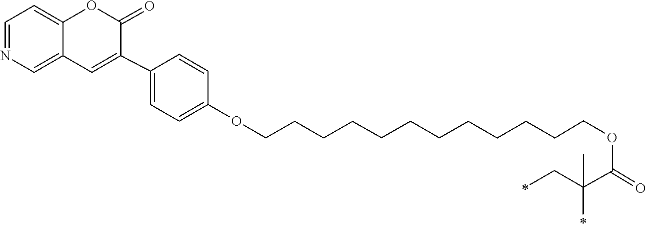 | M⁰-011 |
| 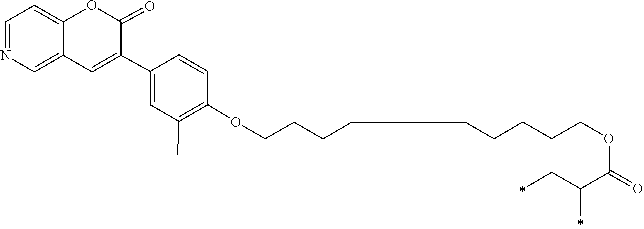 | M⁰-012 |
| 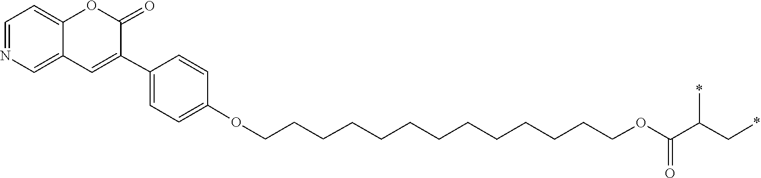 [SMILES: *CC(*)C(=O)OCCCCCCCCCCCCCOc1ccc(-c2cc3cnccc3oc2=O)cc1] | M⁰-013 |
| 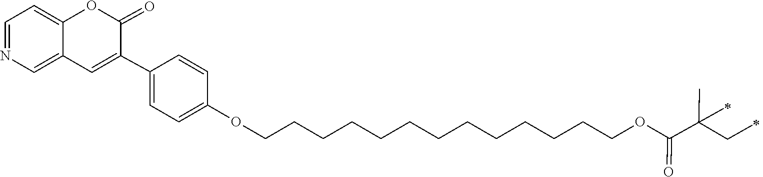 | M⁰-014 |
| 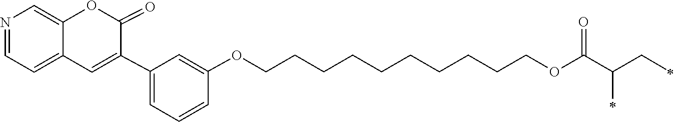 | M⁰-015 |
| 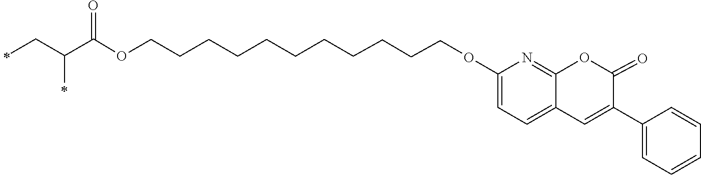 | M⁰-016 |
| 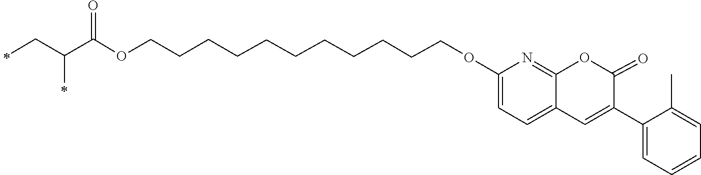 | M⁰-017 |
| 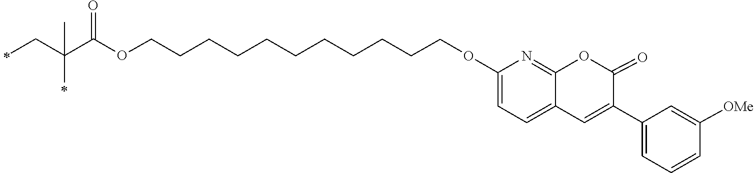 | M⁰-018 |
| 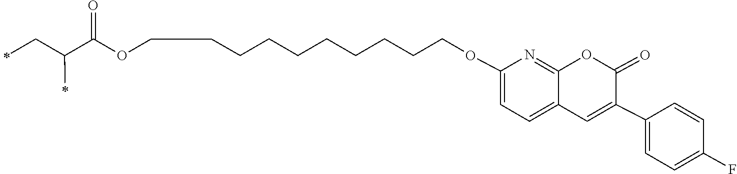 | M⁰-019 |
| 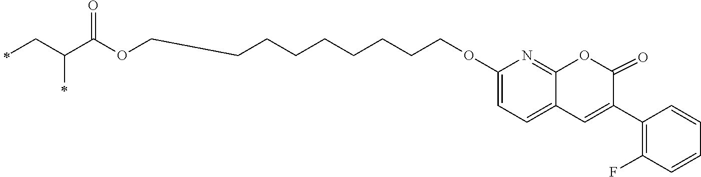 | M⁰-020 |
| 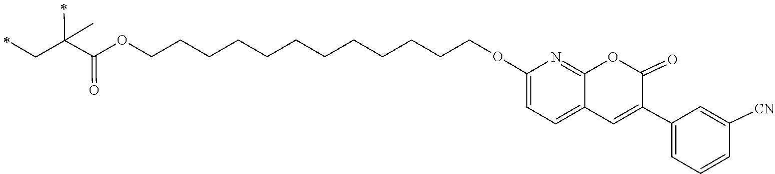 | M⁰-021 |
| 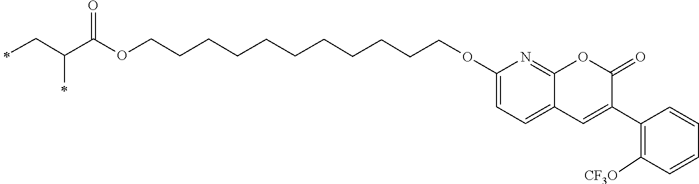 | M⁰-022 |
| 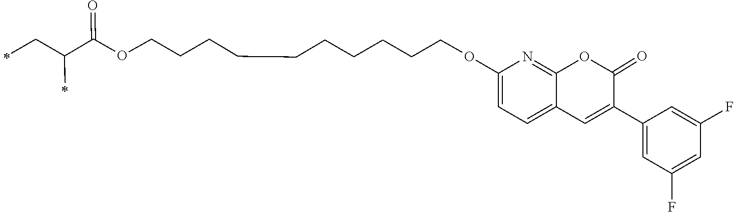 | M⁰-023 |
| 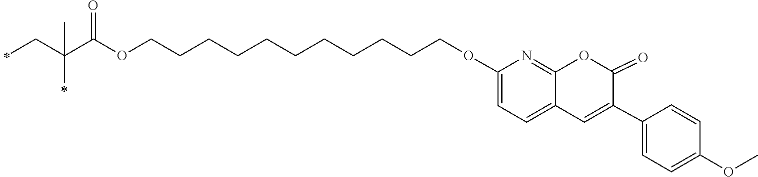 | M⁰-024 |
| 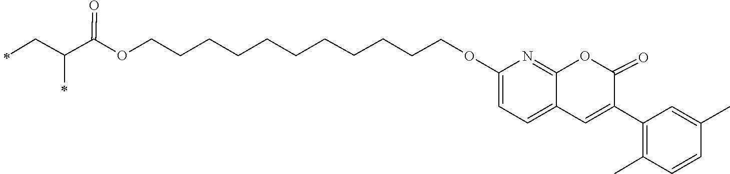 | M⁰-025 |
| 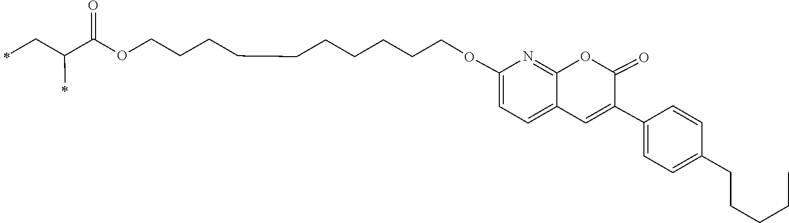 | M⁰-026 |
| 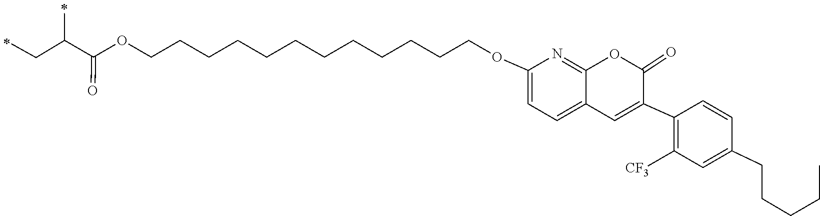 | M⁰-027 |
| 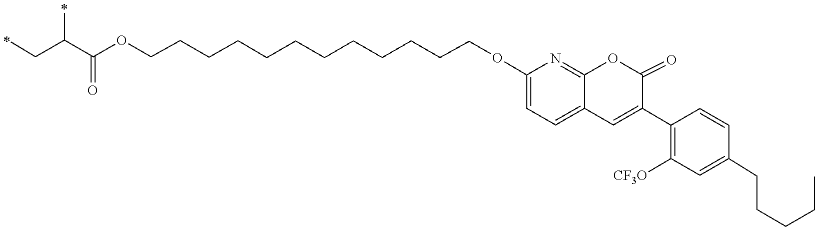 | M⁰-028 |
| 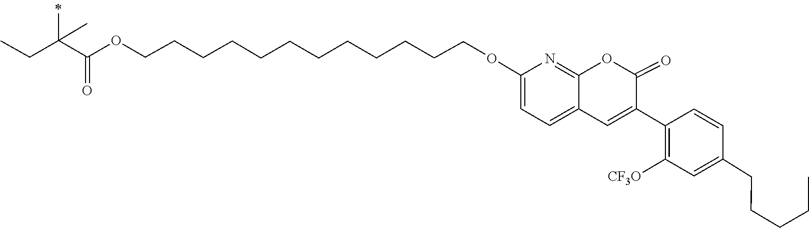 | M⁰-029 |
| 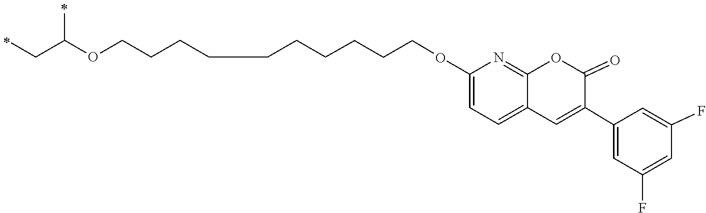 | M⁰-030 |
| 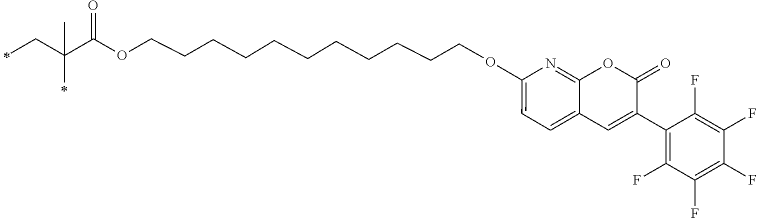 | M⁰-031 |
| 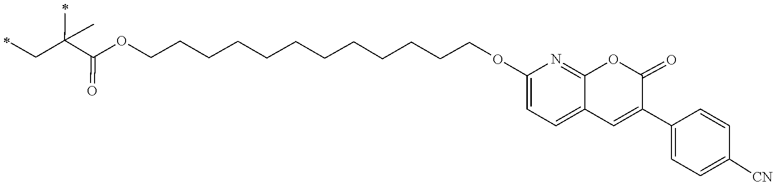 | M⁰-032 |
| 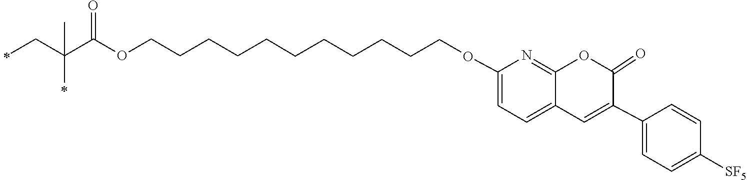 | M⁰-033 |
| 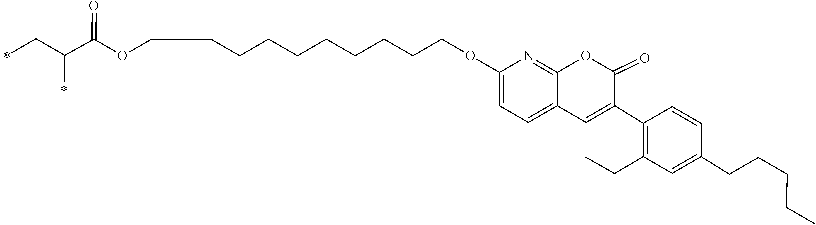 | M⁰-034 |
| 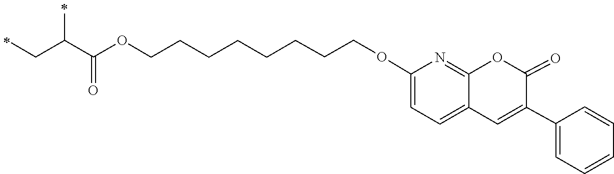 | M⁰-035 |
| 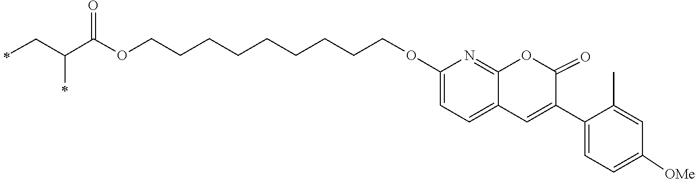 | M⁰-036 |
| 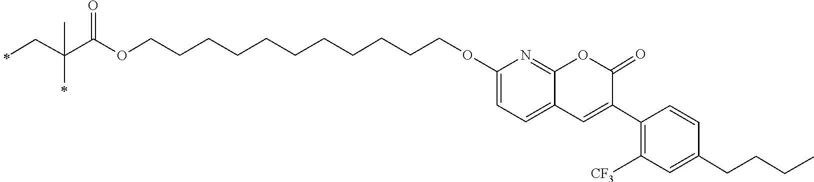 | M⁰-037 |
| 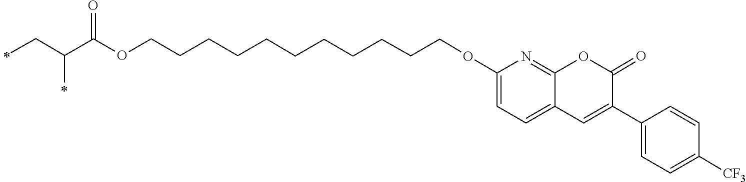 | M⁰-038 |
| 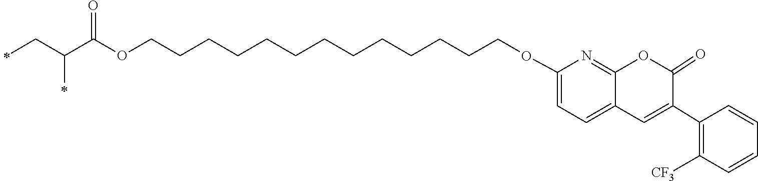 | M⁰-039 |
| 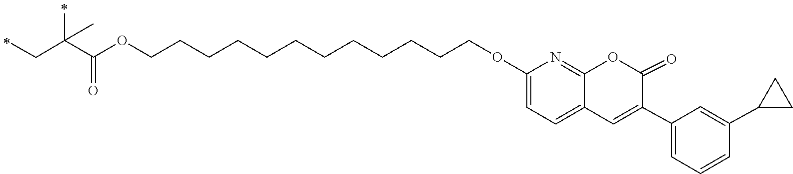 | M⁰-040 |
| 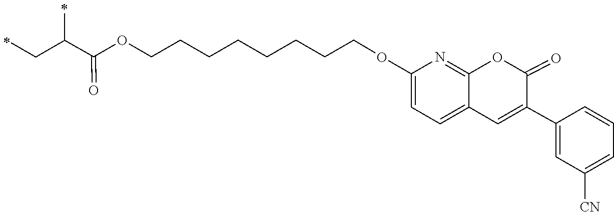 | M⁰-041 |
| 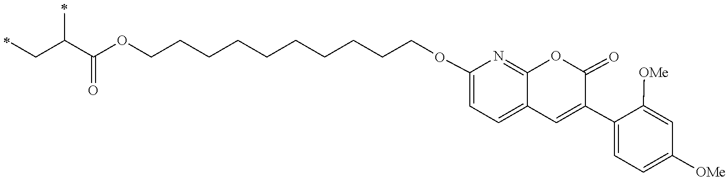 | M⁰-042 |
| 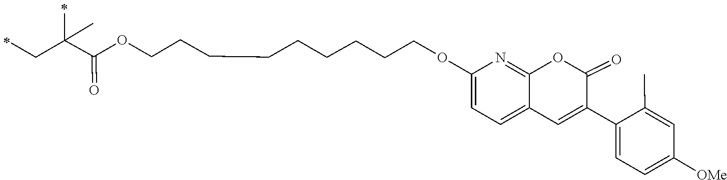 | M⁰-043 |
| 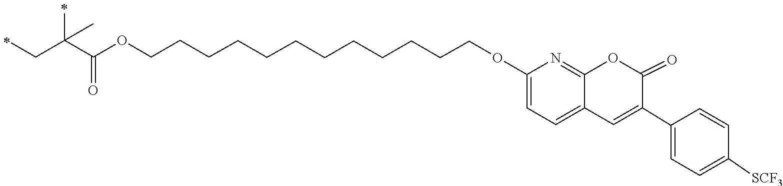 | M⁰-044 |
| 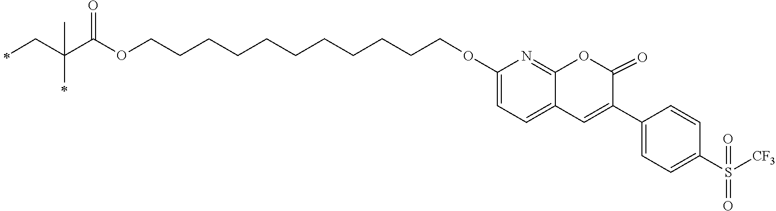 | M⁰-045 |
| 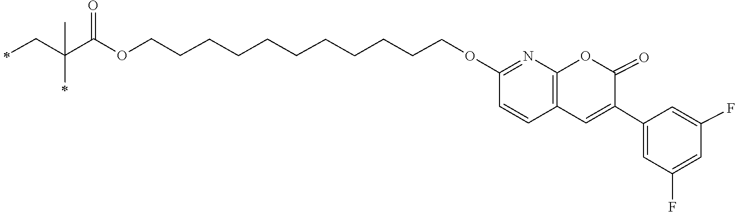 | M⁰-046 |
| 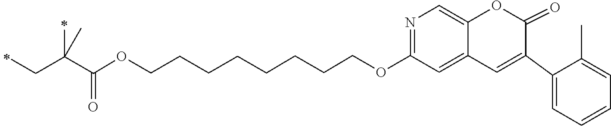 | M⁰-047 |
| 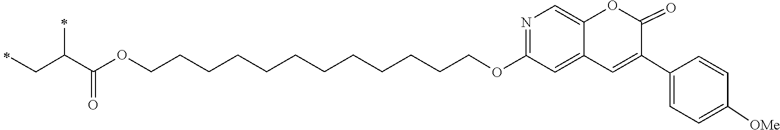 | M⁰-048 |
| 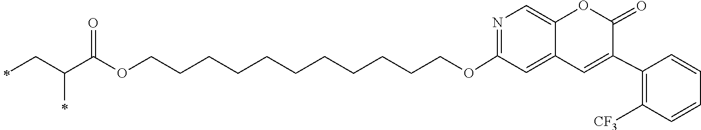 | M⁰-049 |
| 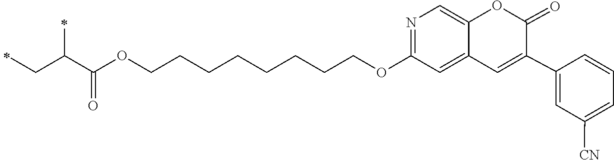 | M⁰-050 |
| 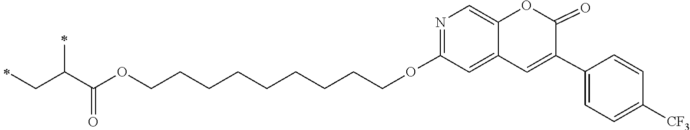 | M⁰-051 |
| 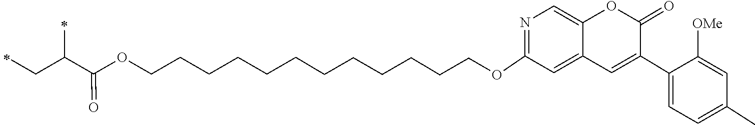 | M⁰-052 |
| 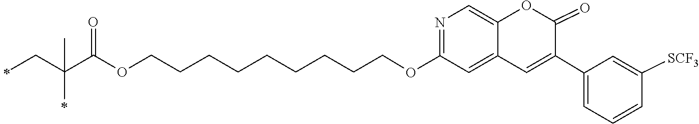 | M⁰-053 |
| 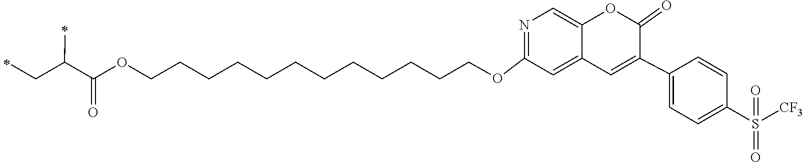 | M⁰-054 |
| 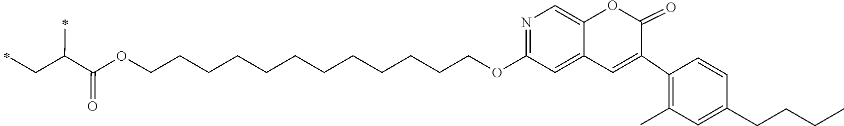 | M⁰-055 |
| 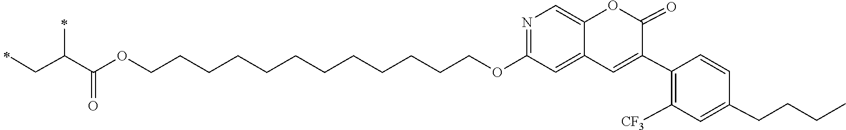 | M⁰-056 |
| 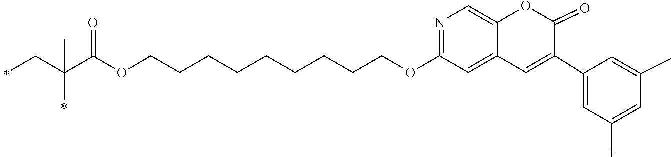 | M⁰-057 |
| 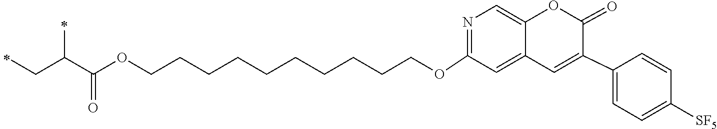 | M⁰-058 |
| 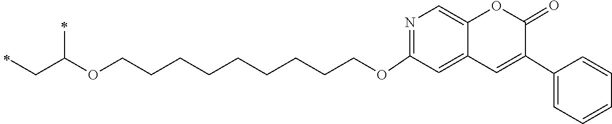 | M⁰-059 |
| 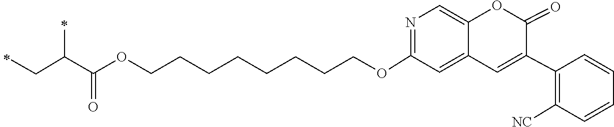 | M⁰-060 |
| 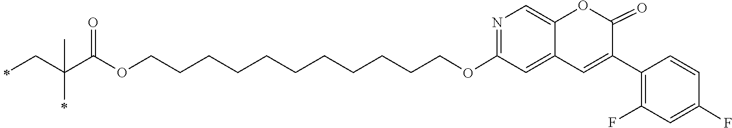 | M⁰-061 |
| 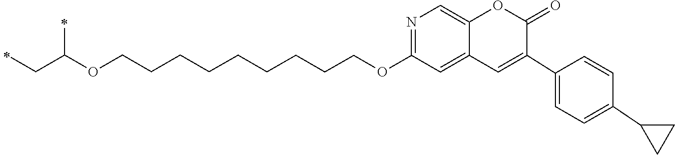 | M⁰-062 |
| 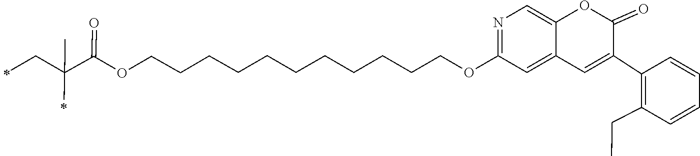 | M⁰-063 |
| 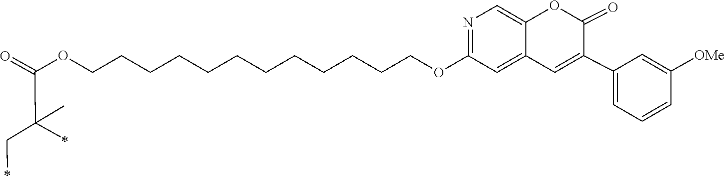 | M⁰-064 |
| 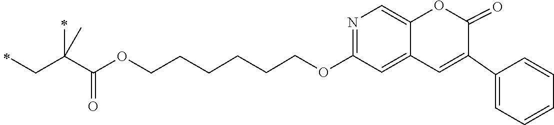 | M⁰-065 |
| 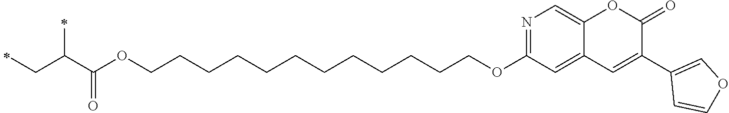 | M⁰-066 |
| 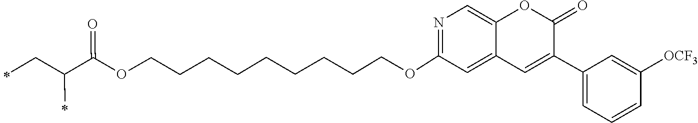 | M⁰-067 |
| 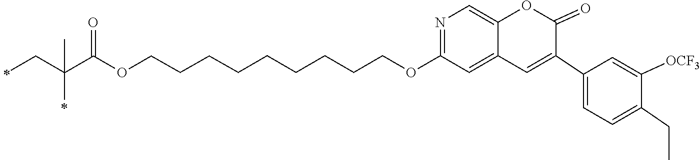 | M⁰-068 |
| 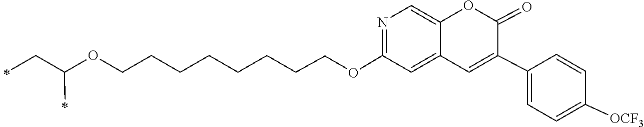 | M⁰-069 |
| 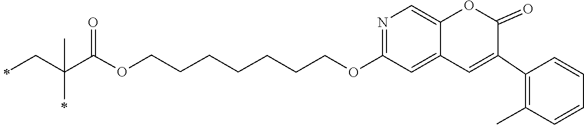 | M⁰-070 |
| 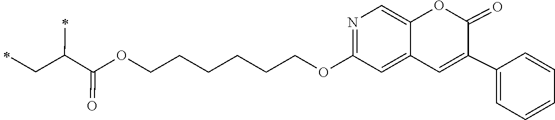 | M⁰-071 |
| 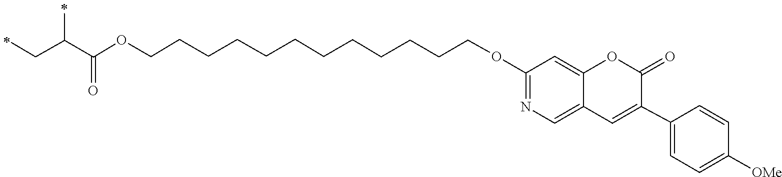 | M⁰-072 |
| 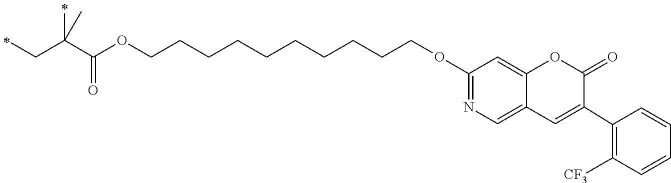 | M⁰-073 |
| 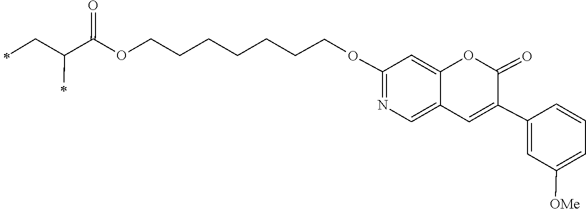 | M⁰-074 |
| 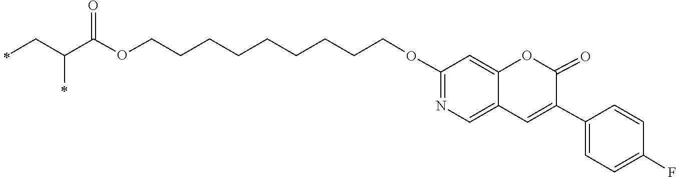 | M⁰-075 |
| 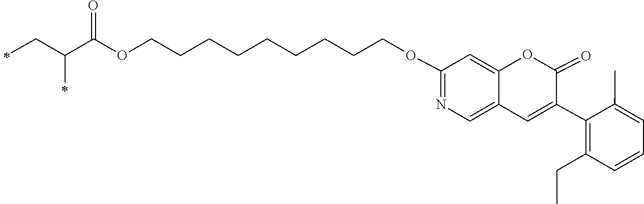 | M⁰-076 |
| 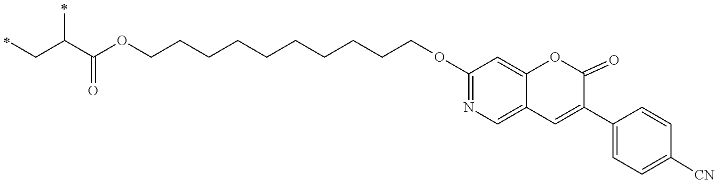 | M⁰-077 |
| 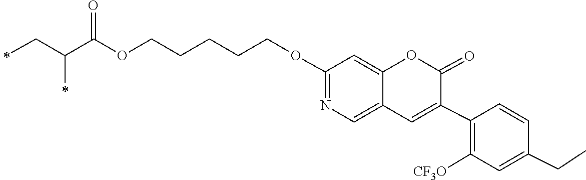 | M⁰-078 |
| 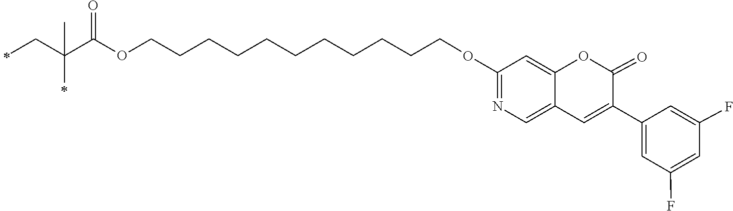 | M⁰-079 |
| 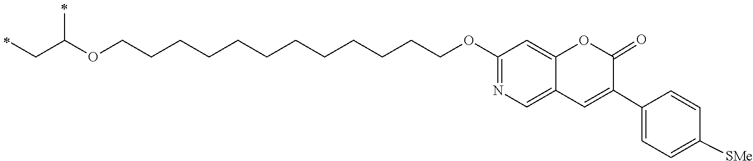 | M⁰-080 |
| 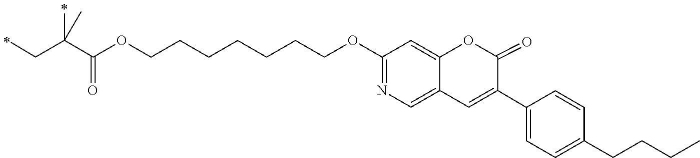 | M⁰-081 |
| 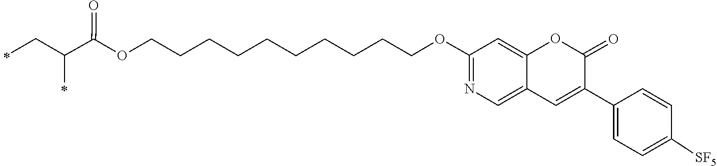 | M⁰-082 |
| 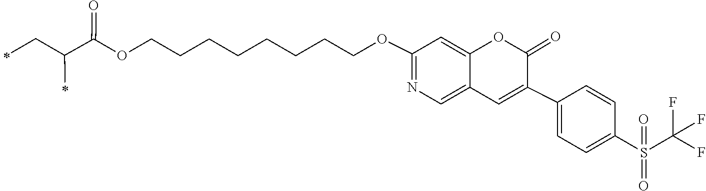 | M⁰-083 |
| 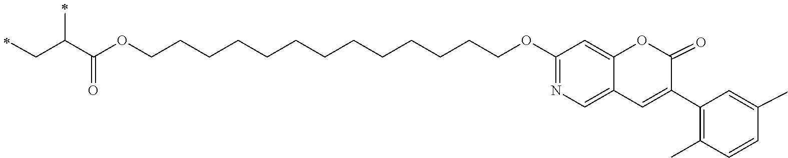 | M⁰-084 |
| 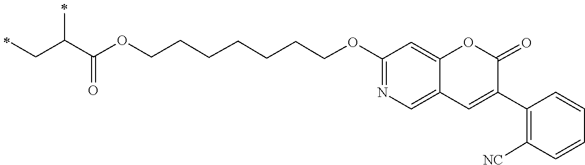 | M⁰-085 |
| 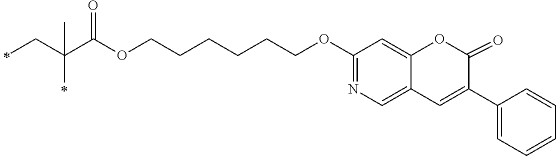 | M⁰-086 |
| 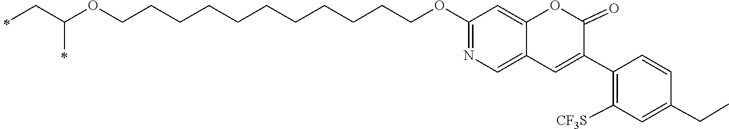 | M⁰-087 |
| 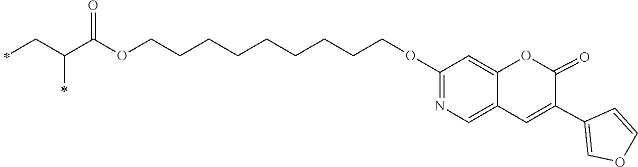 | M⁰-088 |
| 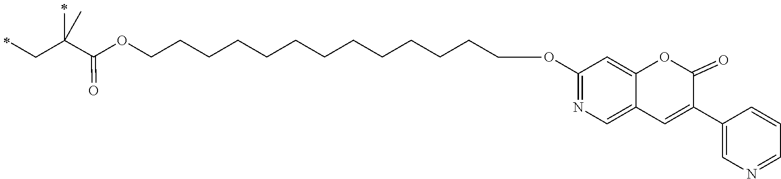 | M⁰-089 |
| 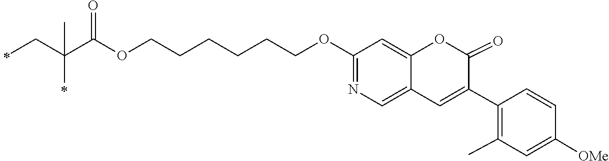 | M⁰-090 |
| 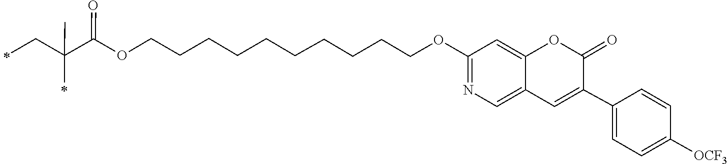 | M⁰-091 |
| 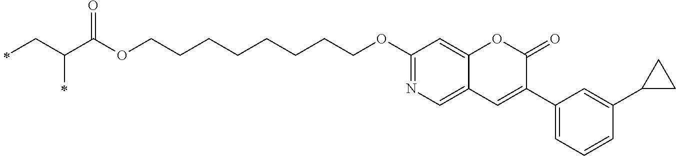 | M⁰-092 |
| 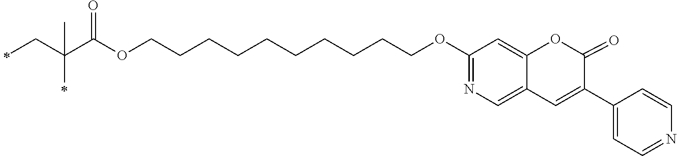 | M⁰-093 |
| 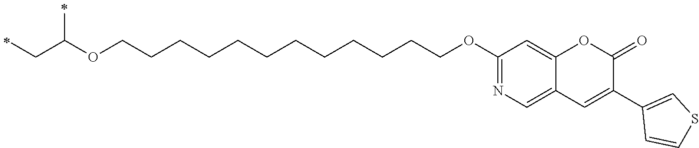 | M⁰-094 |
| 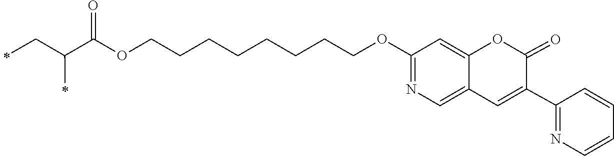 | M⁰-095 |
| 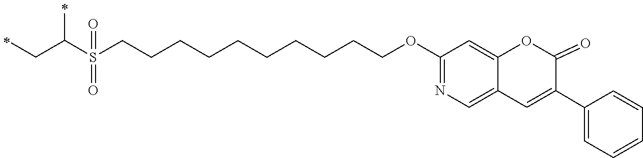 | M⁰-096 |
| 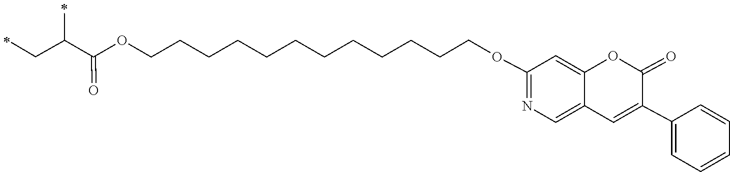 | M⁰-097 |
| 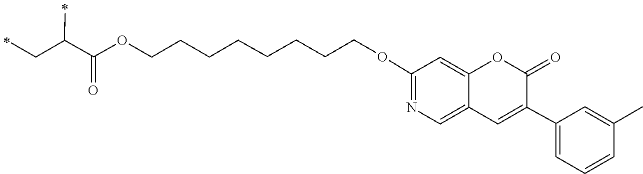 | M⁰-098 |
| 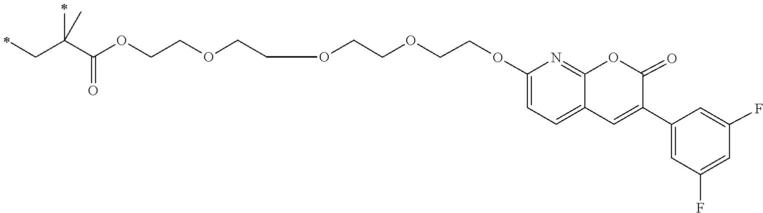 | M⁰-099 |
| 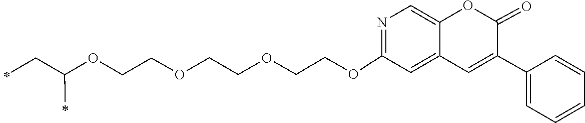 | M⁰-100 |
| 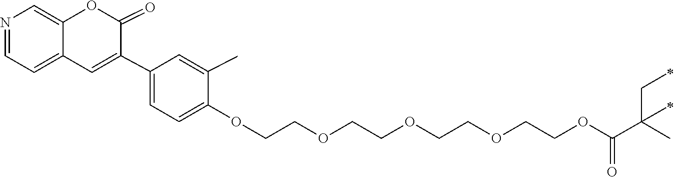 | M⁰-101 |
| 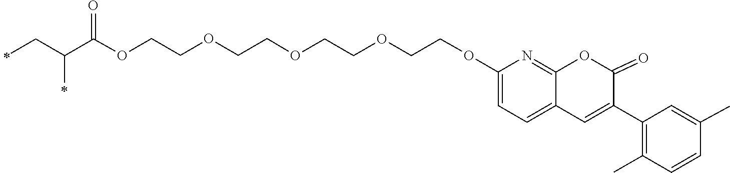 | M⁰-102 |
| 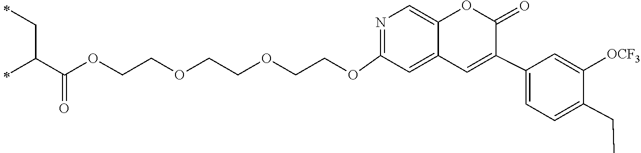 | M⁰-103 |
| 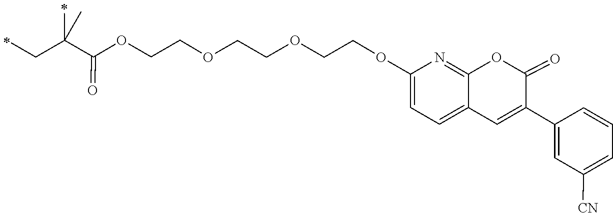 | M⁰-104 |
| 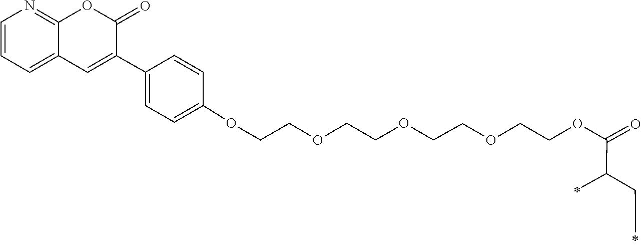 | M⁰-105 |
| 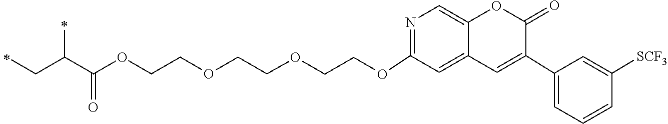 | M⁰-106 |
| 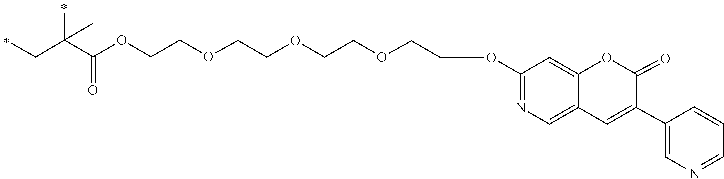 | M⁰-107 |
| 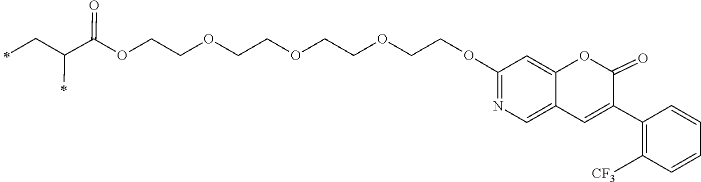 | M⁰-108 |
| 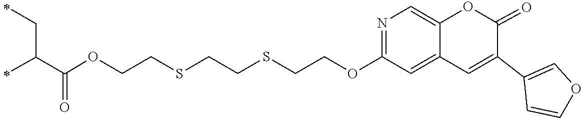 | M⁰-109 |
| 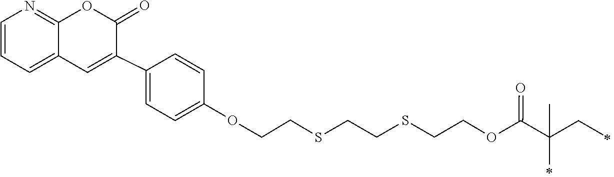 | M⁰-110 |
| 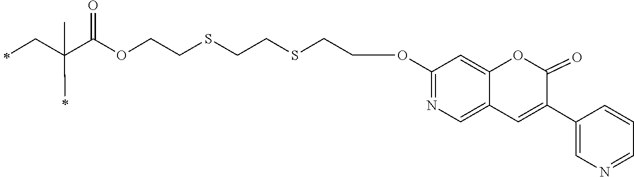 | M⁰-111 |
| 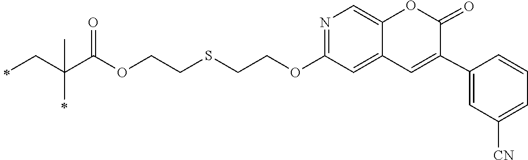 | M⁰-112 |
| 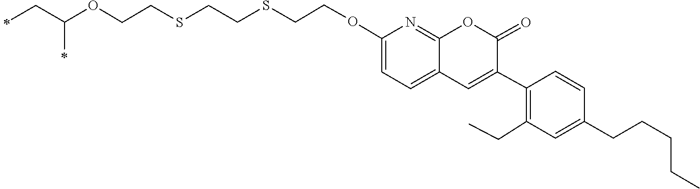 | M⁰-113 |
| 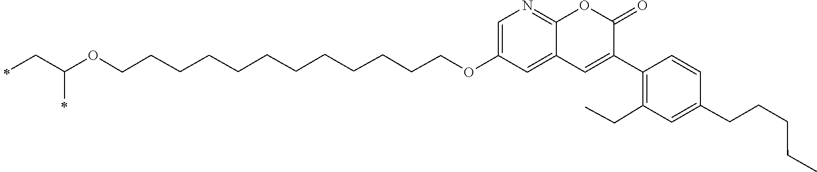 | M⁰-114 |
| 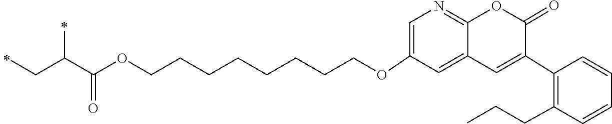 | M⁰-115 |
| 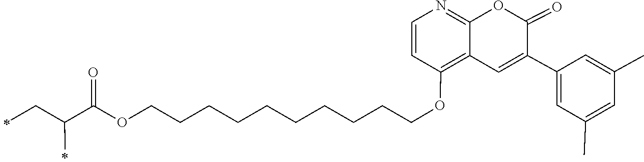 | M⁰-116 |
| 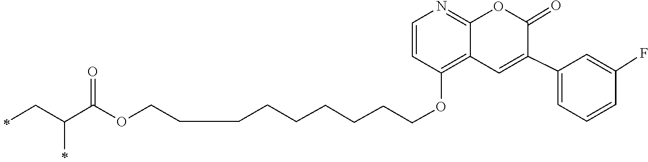 | M⁰-117 |
| 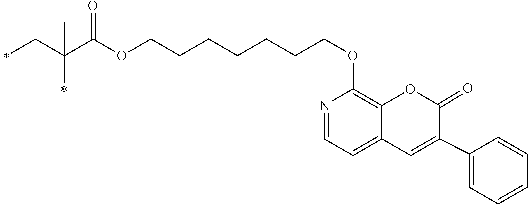 | M⁰-118 |
| 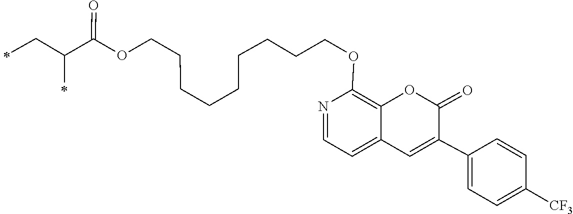 | M⁰-119 |
| 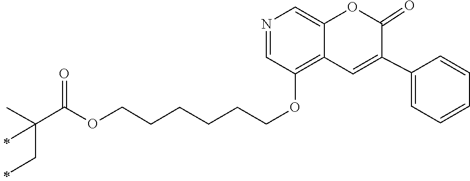 | M⁰-120 |
| 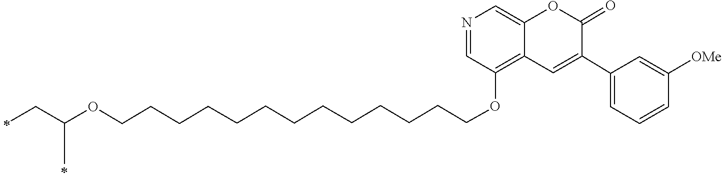 | M⁰-121 |
| 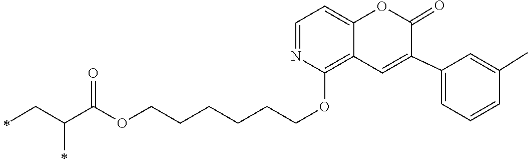 | M⁰-122 |
| 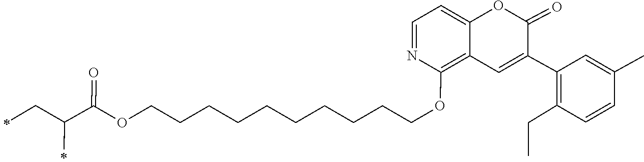 | M⁰-123 |
| 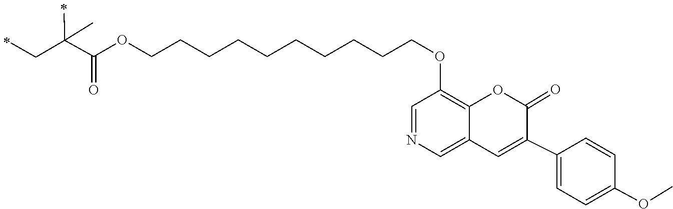 | M⁰-124 |
| 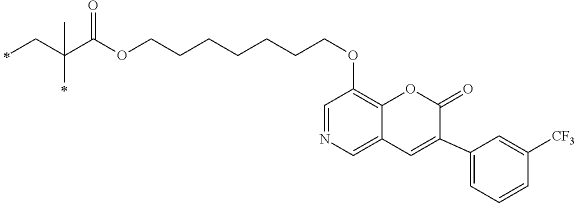 | M⁰-125 |
| 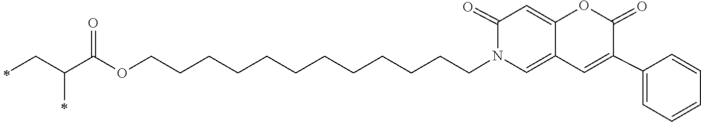 | M⁰-126 |
| 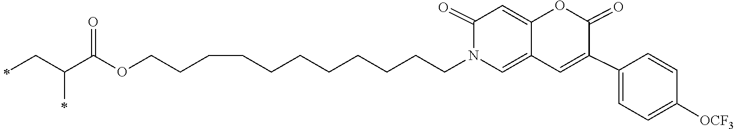 | M⁰-127 |
| 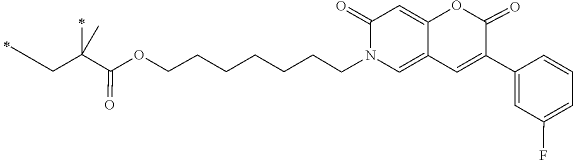 | M⁰-128 |
| 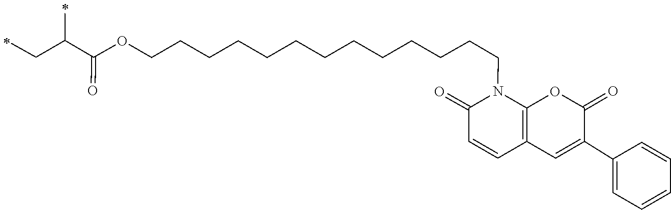 | M⁰-129 |
| 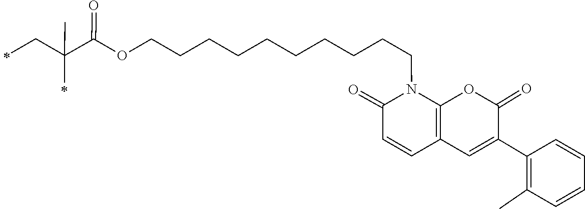 | M⁰-130 |
| 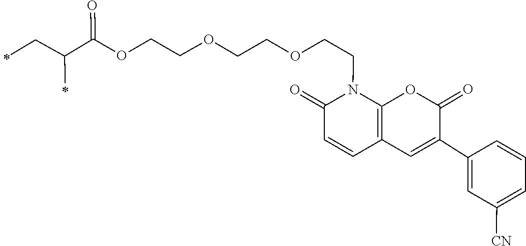 | M⁰-131 |
| 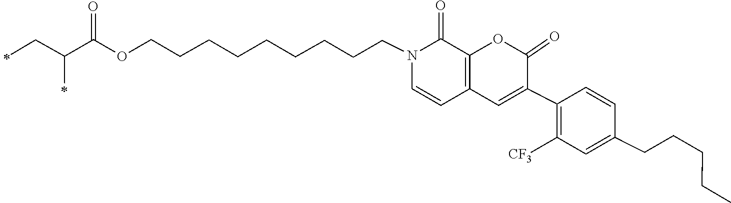 | M⁰-132 |
| 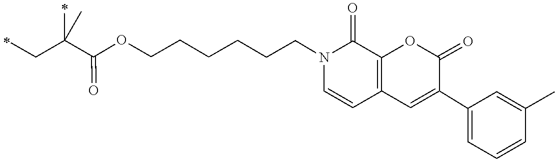 | M⁰-133 |
| 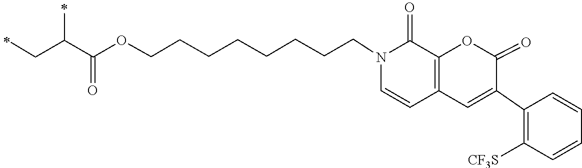 | M⁰-134 |
| 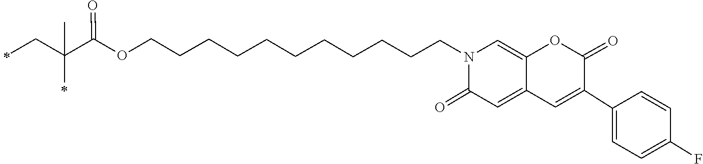 | M⁰-135 |
| 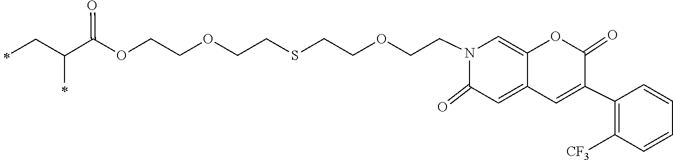 | M⁰-136 |
| 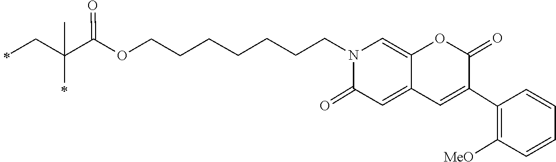 | M⁰-137 |
| 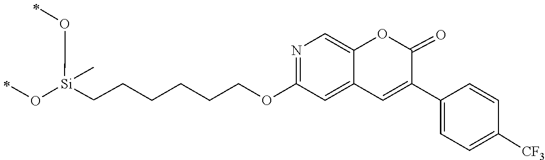 | M⁰-138 |
| 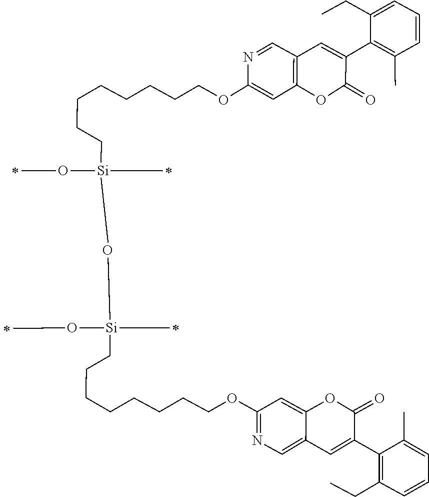 | M⁰-139 |
| 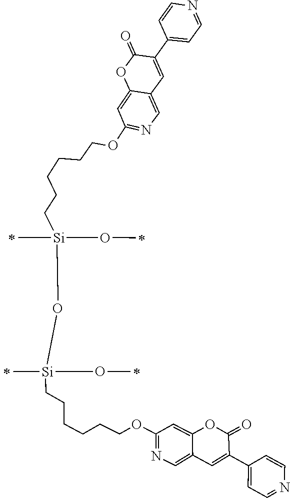 | M⁰-140 |
| 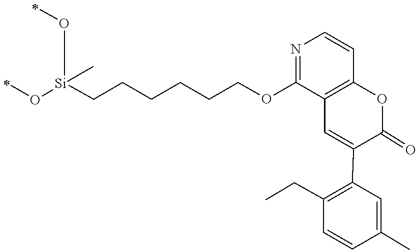 | M⁰-141 |
| 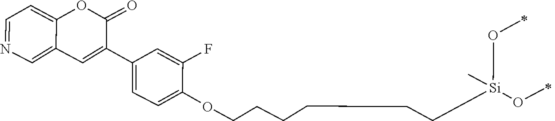 | M⁰-142 |
| 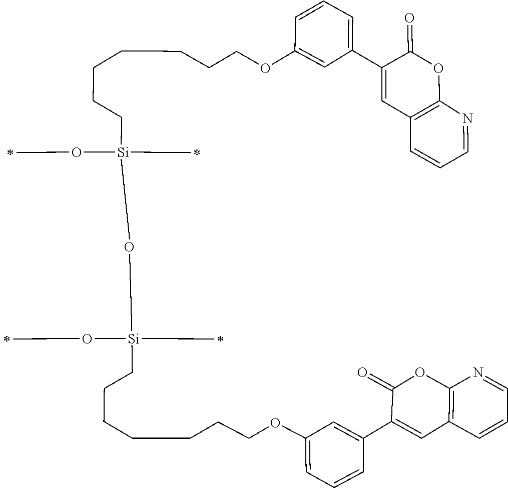 | M⁰-143 |
| 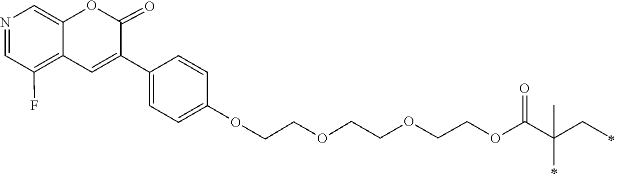 | M⁰-144 |
| 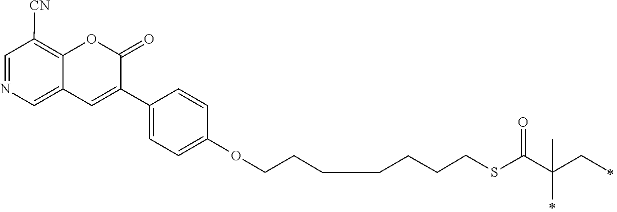 | M⁰-145 |
| 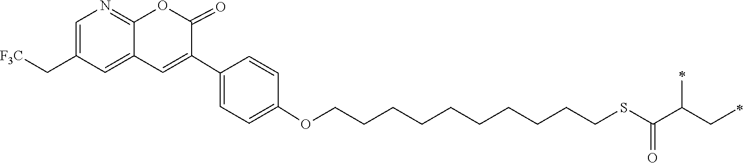 | M⁰-146 |
| 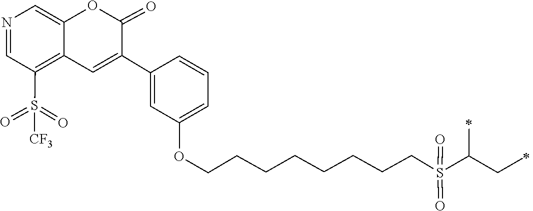 | M⁰-147 |
| 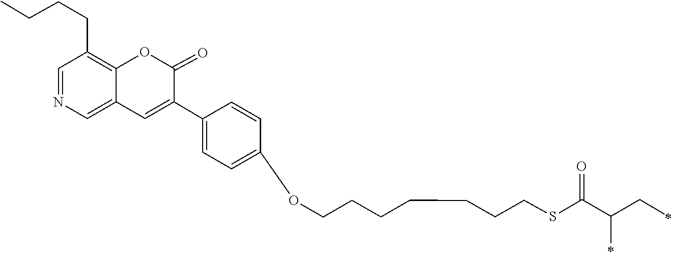 | M⁰-148 |
| 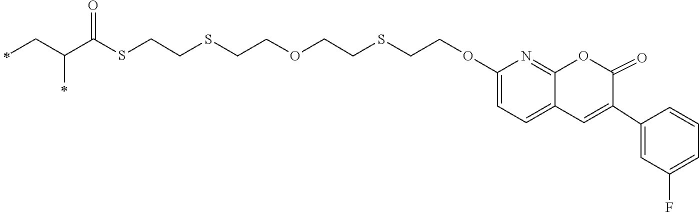 | M⁰-149 |
| 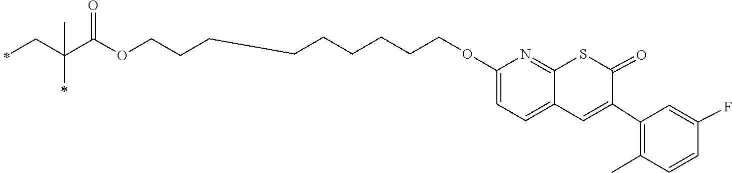 | M⁰-150 |
| 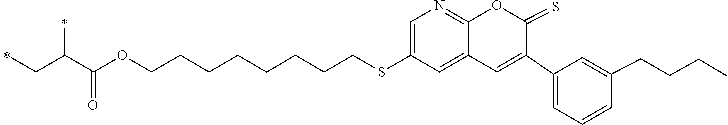 | M⁰-151 |
| 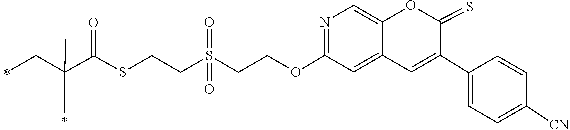 | M⁰-152 |
| 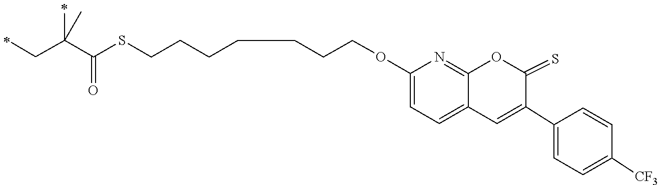 | M⁰-153 |
| 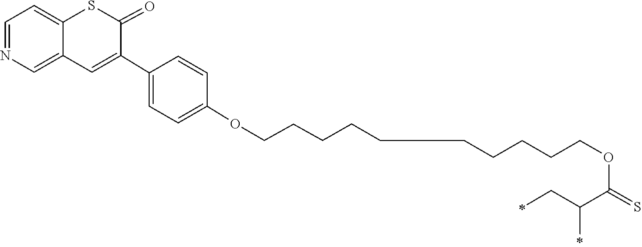 | M⁰-154 |
| 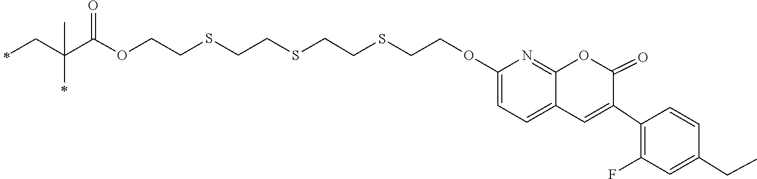 | M⁰-155 |
| 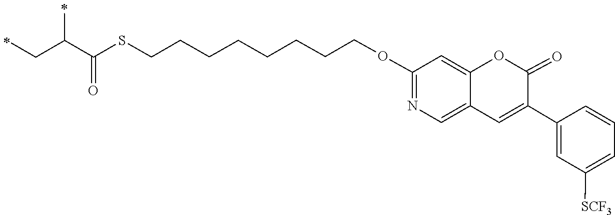 | M⁰-156 |
| 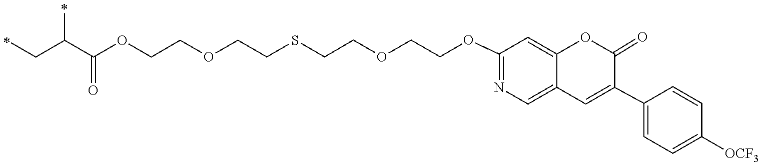 | M⁰-157 |
| 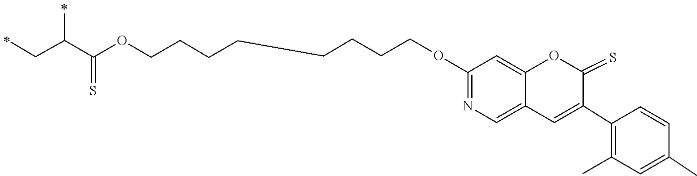 | M⁰-158 |
| 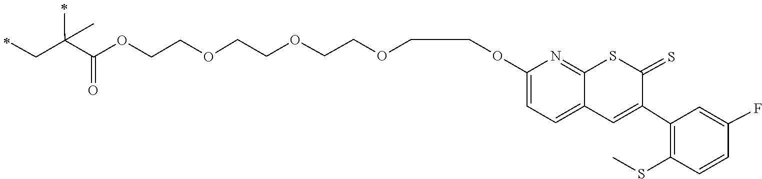 | M⁰-159 |
| 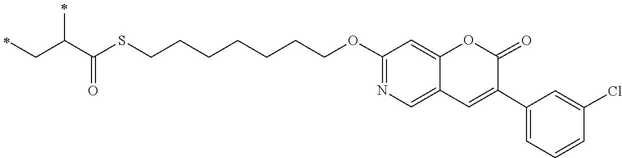 | M⁰-160 |
| 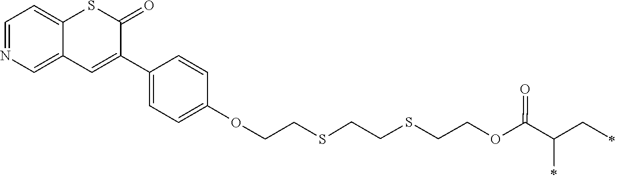 | M⁰-161 |
| 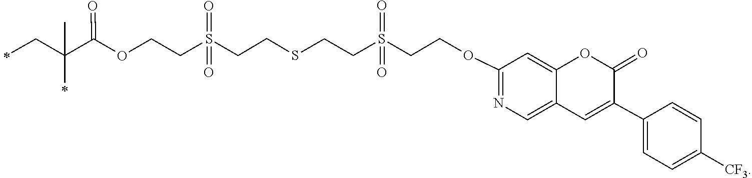 | M⁰-162 |

Preferably a copolymer according to the invention as described before or preferably described before comprises the one or more constitutional units M° as described before with substituents as described before or preferably described before in a molar ratio m1 and the one or more constitutional units M² in a molar ratio m2, wherein the ratio m1:m2 is at least 0.01 and at most 100.

Particularly preferably, such copolymer is comprised in the ophthalmic device or the precursor article for manufacturing an ophthalmic device according to the invention.

The oligomers, polymers or copolymers, preferably polymers or polymers, according to the invention as described before or preferably described may be cross-linked. Particularly preferably, such polymer or copolymer is comprised in the ophthalmic device or the precursor article for manufacturing an ophthalmic device according to the invention.

The oligomers or polymers of the present invention may be made by any suitable method. It is, however, preferred that the present oligomers, polymers and copolymers are made by radical polymerization, wherein the polymerization reaction is started by means of a suitable radical polymerization initiator. For the purposes of the present invention the type of radical polymerization initiator is not particularly limited and may be any suitable radical generating compound. Such compounds are well known to the skilled person. Suitable polymerization initiators may be selected from thermal initiators or photoinitiators, i.e. compounds that generate radicals by exposure to heat or irradiation with light of a suitable wavelength. Examples of suitable thermal polymerization initiators may be selected from the groups of compounds comprising one or more peroxide groups, i.e. compounds comprising a group —O—O—, and/or compounds comprising one or more azo groups, i.e. compounds comprising a group —N≡N—.

Suitable polymerization initiators comprising one or more peroxide groups may, for example, be selected from the groups consisting of t-butyl(peroxy-2-ethyl-hexanoate), di-(tert-butylcyclohexyl)peroxydicarbonate and benzoylperoxide.

Suitable polymerization initiators comprising one or more azo groups may, for example, be selected from the group consisting of 1,1′-azobis(cyclohexancarbonitrile) and 2,2′azobis(cyclohexanecarbonitrile) (AIBN).

Suitable examples of a photoinitiator are dimethylaminobenzoate/camphorquinone, diphenyl(2,4,6-trimethylbenzoyl)phosphine oxide (TPO) or phenylbis(2,4,6-trimethylbenzoyl)phosphine oxide (BAPO).

If a photoinitiator is used as polymerization initiator, it is preferred that the wavelength required to decompose said photoinitiator is different from the wavelength needed to irradiate the compound of the present application so as to change its optical properties.

Preferably, the radical initiators are used in an amount of at least 0.0001 eq and of at most 0.1 eq of the main monomer. Such radical initiators could be thermal initiators, e.g. azobisisobutyronitrile (AIBN) or photochemical initiators like dimethylaminobenzoate/camphorquinone, diphenyl(2,4,6-trimethylbenzoyl)phosphine oxide (TPO) or phenylbis(2,4,6-trimethylbenzoyl)phosphine oxide (BAPO).

The present invention is also directed to a composition for polymerization. Depending upon the intended use such composition as described or preferably described before may comprise further different components. Such further components may, for example, be selected from the group consisting of UV absorbers, antioxidants and cross-linkers. Cross-linkers may also be referred to as crosslinking agents.

The present invention is also directed to a composition for polymerization comprising at least one compound of formulae (I), (I #), (I′), (I′-a), (I′-b), (I′-c), (I′-d), (I′-e), (I′-f), (I′-g), (I′-h), (I′-i), or (I″) or compounds (A-001) to (A-162) as described or preferably described before and/or an oligomer or polymer as described before or preferably described before but having at least one reactive group left for polymerization and/or a crosslinking agent and/or a UV absorber and/or a radical initiator and optionally further monomers different from compounds of formulae (I), (I #), (I′), (I′-a), (I′-b), (I′-c), (I′-d), (I′-e), (I′-f), (I′-g), (I′-h), (I′-i) or (I″) or the compounds (A-001) to (A-162).

A composition comprising at least one compound of formulae (I), (I #), (I′), (I′-a), (I′-b), (I′-c), (I′-d), (I′-e), (I′-f), (I′-g), (I′-h), (I′-i) or (I″) or compounds (A-001) to (A-162) as described or preferably described before and an oligomer or polymer according to the invention as described before is primarily used for the synthesis of block copolymers with the condition that the oligomer or polymer has at least one reactive group left which may react with the monomers.

The compositions may include or comprise, essentially consist of or consist of the said requisite or optional constituents. All compounds or components which can be used in the compositions are either known and commercially available or can by synthesized by known processes or as described herein.

The components of the composition according to the invention are combined in such amounts that at least 2 wt % to 100 wt %, preferably 3 wt % to 70 wt %, particularly preferably 4 wt % to 51 wt %, very particularly preferably 5 wt % to 45 wt % of photoactive chromophores of polymerized formulae (I), (I #), (I′), (I′-a), (I′-b), (I′-c), (I′-d), (I′-e), (I′-f), (I′-g), (I′-h), (I′-i) or (I″) are comprised in the resulting oligomers, polymers or copolymers according to the invention.

The components of the composition according to the invention are combined in such amounts that at least 2 wt % to 100 wt %, preferably 3 wt % to 70 wt %, particularly preferably 4 wt % to 51 wt %, very particularly preferably 5 wt % to 45 wt % of photoactive chromophores of polymerized formulae (I), (I #), (I′), (I′-a), (I′-b), (I′-c), (I′-d), (I′-e), (I′-f), (I′-g), (I′-h), (I′-i) or (I″) are comprised in the resulting oligomers, polymers or copolymers building the material of the ophthalmic device or the precursor article for manufacturing an ophthalmic device according to the invention.

The UV absorber that may be used in the present composition is not particularly limited and can easily be selected from those generally known to the skilled person. Generally suitable UV absorbers are characterized by being unsaturated compounds, preferably compounds comprising one or more selected from group consisting of olefinic groups, aryl groups and heteroaryl groups; these groups may be present in any combination.

Suitable UV-absorber for use in the present composition may, for example, be selected from those comprising a group selected from benzotriazole, benzophenone and triazine. Suitable UV-absorbers are, for example, disclosed in U.S. Pat. Nos. 5,290,892; 5,331,073 and 5,693,095.

Suitable UV-absorber are 2-(3-(t-butyl)-4-hydroxy-5-(5-methoxy-2-benzotriazolyl)phenoxy)ethyl methacrylate, 3-(3-(t-butyl)-4-hydroxy-5-(5-methoxy-2-benzotriazolyl)phenoxy)propyl methacrylate, 3-(3-t-Butyl-5-(5-chlorobenzotriazol-2-yl)-4-hydroxyphenyl)propyl methacrylate 3-(3-(tert-Butyl)-4-hydroxy-5-(5-methoxy-2H-benzo[d][1,2,3]triazol-2-yl)phenoxy)propylmethacrylat, 2-(2-Hydroxy-5-vinylphenyl)-2H-benzotriazol, Allyl-2-hydroxybenzophenon, 2-Allyl-6-(2H-benzotriazol-2-yl)-p-cresol, 4-Methacryloxy-2-hydroxybenzophenon, 2-(2′-Hydroxy-3′-methallyl-5′-methylphenyl)benzotriazol, 2-Hydroxy-4-methacryloyloxybenzophenon, 4-Acryloylethoxy-2-hydroxybenzophenon, 2-[3-(2H-Benzotriazol-2-yl)-4-hydroxyphenyl]ethylmethacrylat, 2-(2′-Hydroxy-5′-methacrylamidophenyl)-5-methoxybenzotriazol, 2-(2′-Hydroxy-5′-methacrylamidophenyl)-5-chlorobenzotriazol, 2-(2′-Hydroxy-5′-methacryloxypropylphenyl)benzotriazol, 2-(2′-Hydroxy-5′-methacryloylpropyl-3′-tert-butyl-phenyl)-5-methoxy-2H-benzotriazol, 2-(3-(tert-Butyl)-4-hydroxy-5-(5-methoxy-2H-benzo[d][1,2,3]triazol-2-yl)phenoxy)ethylmethacrylat, 2-[3′-tert-Butyl-2′-hydroxy-5′-(3″-methacryloyloxypropyl)phenyl]-5-chlorbenzotriazol, 2-{2′-Hydroxy-3′-tert-butyl-5′-[3′-methacryloyloxypropoxy]phenyl}-5-methoxy-2H-benzotriazol, 2-[3′tert-Butyl-5′-(3″-dimethylvinylsilylpropoxy)-2′-hydroxyphenyl]-5-methoxybenzotriazol, 2-(tert-Butyl)-6-(5-chloro-2H-benzo[d][1,2,3]triazol-2-yl)-4-vinylphenol, 2-(2H-1,2,3-benzotriazol-2-yl)-4-methyl-6-(2-methylprop-2-enyl)phenol, 2-(3-acetyl-2-aminophenoxy)ethyl methacrylat, 2-(4-benzoyl-3-hydroxyphenoxy)ethyl acrylat or a combination of this compounds.

Preferred UV-Absorber are selected from the group of 2-[3′-2′H-benzotriazol-2′-yl)-4′-hydroxyphenyl]ethyl methacrylate (BTPEM), 2-(3-(t-butyl)-4-hydroxy-5-(5-methoxy-2-benzotriazolyl)phenoxy)ethyl methacrylate, 3-(3-(t-butyl)-4-hydroxy-5-(5-methoxy-2-benzotriazolyl)phenoxy)propyl methacrylate, 3-(3-t-Butyl-5-(5-chlorobenzotriazol-2-yl)-4-hydroxyphenyl)propyl methacrylate which may be polymerized together with the monomers as described or preferably described before.

Suitable cross-linker may be used to impart elastomeric properties to the present composition and the ophthalmic devices or precursor articles produced therewith. Typically any suitable di- or tri-functional monomer may be used as crosslinker. Such monomers are generally well known to the skilled person and may be selected from the group of poly(ethylene glycol) diacrylate, poly(ethyleneglycol) dimethacrylate, ethyleneglycoldimethacrylate (EGDMA), ethyleneglycoldiacrylate, 1,3-propanedioldiacrylat, 1,6-hexanedioldiacrylate, 1,8-octanedioldiacrylate, 1,11-undecandioldiacrylate, 1,12-dodecyldiacrylate, 1,15-pentadecandioldiacrylate, 1,16-hexadecanedioldiacrylate, 1,18-octadecanedioldiacrylate, 1,3-propanedioldimethacrylate, 1,6-hexanedioldimethacrylate, 1,8-octanedioldimethacrylate, 1,11-undecandioldimethacrylate, 1,12-dodecyldimethacrylate, 1,15-pentadecanedioldimethacrylate, 1,16-hexadecanedioldimethacrylate, 1,18-octadecanedioldimethacrylate.

Preferred cross-linker may be selected from the following group of compounds

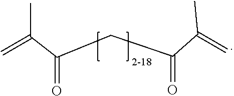

Ethylene glycol dimethacrylate (EGDMA) is particularly preferred.

Suitable antioxidants are phenyl acrylate derivatives bearing a hindered phenol moiety. A preferred antioxidant is

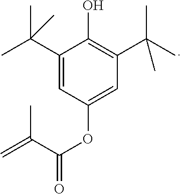 [SMILES: C=C(C)C(=O)Oc1cc(C(C)(C)C)c(O)c(C(C)(C)C)c1]

The compounds of formulae (I), (I #), (I′), (I′-a), (I′-b), (I′-c), (I′-d), (I′-e), (I′-f), (I′-g), (I′-h), (I′-i) or (I″) or the compounds (A-001) to (A-162) according to the invention as described or preferably described before and their oligomers, polymers or copolymers as described before or preferably described before comprising one or more constitutional units M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) or one or more constitutional units (M⁰-001) to (M⁰-162) as described before or preferably described before are particularly well suited for use in optically active devices e.g. ophthalmic devices as described before.

The compounds of formulae (I), (I #), (I′), (I′-a), (I′-b), (I′-c), (I′-d), (I′-e), (I′-f), (I′-g), (I′-h), (I′-i) or (I″) or the compounds (A-001) to (A-162) according to the invention as described or preferably described before and their oligomers, polymers or copolymers as described before or preferably described before comprising one or more constitutional units M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) or one or more constitutional units (M⁰-001) to (M⁰-162) as described before or preferably described before are particularly sensitive to two-photon or multiphoton absorption. Hence the ophthalmic device and the precursor article for manufacturing the ophthalmic device are sensitive to two-photon or multiphoton absorption.

The system for two-photon or multi-photon irradiating of the ophthalmic device according to the invention, preferably of an intraocular lens preferably arranged within an eye of a patient is not restricted. Some examples are described below.

Hence the present invention is also directed to precursor articles for manufacturing an ophthalmic device wherein said precursor article is a blank which may be transformed into optically active ophthalmic devices comprising at least one oligomer, polymer or copolymer as described before or preferably described before comprising one or more constitutional units M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) or one or more constitutional units (M⁰-001) to (M⁰-162) as described before or preferably described before.

Preferred ophthalmic devices are optically active ophthalmic devices. Examples of such ophthalmic devices or eye-implants include lenses, keratoprostheses, and corneal inlays or rings. More preferably, said ophthalmic device or eye-implant is a lens article. Most preferably, such ophthalmic device is a lens. The type of lens is not restricted and may comprise a contact lens or an intraocular lens. Most preferably, such ophthalmic device is an intraocular lens, which may, for example, be a posterior chamber intraocular lens or an anterior chamber intraocular lens.

A blank of this invention may be produced as a step in the manufacturing process used to create an ophthalmic device as described before, preferably an intraocular lens. For example, without limitation, a manufacturing process may include the steps of polymer synthesis, polymer sheet casting, blank cutting, optic lathe cutting, optic milling, haptic milling or attachment, polishing, solvent extraction, sterilization and packaging while the term polymer is used as described before or preferably described before.

The present ophthalmic devices or the precursor articles for manufacturing an ophthalmic device according to the invention as described before or preferably described before may be formed by a process comprising the steps of
providing a composition comprising at least one compound of formulae (I), (I #), (I′), (I′-a), (I′-b), (I′-c), (I′-d), (I′-e), (I′-f), (I′-g), (I′-h), (I′-i) or (I″) or the compounds (A-001) to (A-162) as described herein or preferably described herein and/or an oligomer or polymer as described herein or preferably described herein but having at least one reactive group left for polymerization and optionally further monomers different from compounds of formulae (I), (I #), (I′), (I′-a), (I′-b), (I′-c), (I′-d), (I′-e), (I′-f), (I′-g), (I′-h), (I′-i) or (I″) or the compounds (A-001) to (A-162) as described herein or preferably described herein and/or crosslinking agents and/or UV absorbers and/or radical initiators; and
subsequently forming the ophthalmic device or the precursor article of said composition.

Intraocular lenses in accordance with the present invention are believed to show particularly advantageous properties in that they are flexible enough so as to be rolled or folded and consequently requiring a much smaller incision for them to be inserted into the eye. It is believed that this will allow for improved healing of the eye, particularly in respect to the time for the eye to heal.

The type of intraocular lens is not limited in any way. It may, for example, be a pseudo-phakic intraocular lens or a phakic intraocular lens. The former type replaces the eye's natural, crystalline lens, usually to replace a cataractous lens that has been removed. The latter type is used to supplement an existing lens and functions as a permanent corrective lens, which is implanted in the anterior or posterior chamber to correct refractive errors of the eye. It may, for example, comprise one or more optic and one or more haptic components, wherein the one or more optic components serve as lens and the one or more haptic components are attached to the one or more optic components and hold the one or more optic components in place in the eye. The present intraocular lens may be of a one-piece design or of multi-piece design, depending on whether the one or more optic components and the one or more haptic components are formed from a single piece of material (one-piece design) or are made separately and then combined (multi-piece design). The present intraocular lens is also designed in such a way that it allows to be, for example, rolled up or folded small enough so that it fits through an incision in the eye, said incision being as small as possible, for example, at most 3 mm in length.

Additionally, intraocular lenses in accordance with the present invention allow for the non-invasive adjustment of the optical properties, particularly the polarizability or the refractive power, after implantation of the lens into the eye, thus reducing the need for post-surgery vision aids or reducing or totally avoiding follow-up surgery.

In order to change the optical properties and particularly the polarizability or refractive power of the ophthalmic device according to the invention e.g. an intraocular lens it is exposed to irradiation having a wavelength of at least 200 nm and of at most 1500 nm. Said irradiation is not limited and may be a based on a single-photon or two- or multi-photon process.

Hence, the present invention is also directed to a process of changing the optical properties of an ophthalmic device or a precursor article for manufacturing an ophthalmic device as defined or preferably defined herein, said process comprising the steps of
providing an ophthalmic device or a precursor article for manufacturing an ophthalmic device as defined herein; and
subsequently exposing said ophthalmic device or said precursor article to irradiation having a wavelength of at least 200 nm and at most 1500 nm.

Preferably, said irradiation has a wavelength of at least 250 nm or 300 nm, more preferably of at least 350 nm, even more preferably of at least 400 nm, still even more preferably of at least 450 nm, and most preferably of at least 500 nm. Preferably, said irradiation has a wavelength of at most 1400 nm or 1300 nm or 1200 nm or 1100 nm or 1000 nm, more preferably of at most 950 nm or 900 nm, even more preferably of at most 850 nm, still even more preferably of at most 800 nm and most preferably of at most 750 nm.

Hence, the present invention is also directed to an ophthalmic device or a precursor article for manufacturing an ophthalmic device obtainable by said irradiation process as described before or preferably described before or below.

Alternatively, you may describe the change of refractive power as a modification of the index of refraction of said ophthalmic device as described before or preferably described before. Alternatively, you may describe the change of refractive power as a modification of the index of refraction of said intraocular lens as described before or preferably described before. Irradiation within the focal volume results in refractive optical structures characterized by a change in refractive index relative to the index of refraction of the bulk of said ophthalmic device or alternatively the non-irradiated portion of said ophthalmic device. Irradiation within the focal volume results in refractive optical structures characterized by a change in refractive index relative to the index of refraction of the bulk of said ophthalmic device or intraocular lens or alternatively the non-irradiated portion of said ophthalmic device or intraocular lens. The change in polarizability or refractive index can in other words be used to form patterned desired refractive structures in the optical ophthalmic device as described or preferably described before, preferably in the intraocular lens as described or preferably described before.

Hence, the present invention is also directed to an ophthalmic device obtainable by said irradiation process as described before or preferably described before and below having refractive optical structures characterized by a change in refractive index relative to the index of refraction of the bulk of said ophthalmic device or alternatively the non-irradiated portion of said ophthalmic device.

It is preferred to provide refractive structures that exhibit a change in refractive index, and exhibit little or no scattering loss in such a way that ablation or removal of the optical ophthalmic device, preferably the intraocular lens article is not observed in the irradiated region.

In such processes, the irradiated regions of the ophthalmic device as described before or preferably described before can take the form of two- or three-dimensional, area or volume filled refractive structures that can provide spherical, aspherical, toroidal, or cylindrical correction. In fact, any optical structure can be formed to yield power correction in both physical directions. Moreover, the optical structures can be stacked vertically or written in separate planes in the ophthalmic device as described before or preferably described before to act as a single lens element.

The invention is therefore further related to a method for locally adjusting a polarizability and/or a refractive index of an ophthalmic device according to the invention preferably an intraocular lens preferably arranged within an eye of a patient. The method relates in particular to fabrication of optical profiles by adjusting polarizability through two- or multi-photon processes in a non-destructive manner. Said two- or multi-photon processes allow for different optical profiles compared to single-photon processes and can be advantageously used for the manufacture of the ophthalmic devices according to the invention containing optical profiles.

The system to be used for said two- or multi-photon process advantageously allows for post-operative and non-invasive adjustment of optical properties/profiles of an implanted intraocular lens (IOL) to remove visual impairments such as refractive errors. Furthermore, when manufacturing the ophthalmic device according to the invention, the system advantageously allows for a gentle preparation of the ophthalmic device so as to in particular allow for refractive structures that can provide spherical, aspherical, toroidal, or cylindrical correction and/or maintaining flexibility of the ophthalmic device once preparation of the ophthalmic device is completed. The polarizability of the ophthalmic device is modified based on a two-photon (or generally multi-photon) process which allows adjustment of optical properties/profiles of said ophthalmic device or which allows adjustment of optical properties in different planes of the ophthalmic device. Furthermore, the modification of polarizability based on a two-photon or multi-photon process allows for improved maintaining of flexibility of the ophthalmic device when treated with wavelength of 400 nm to 590 nm.

Therefore, the invention further relates to a process for adjusting a polarizability of an ophthalmic device according to the invention based on a two- or multi-photon absorption process, the process comprising the steps of:

providing said ophthalmic device as described before or preferably described before; and

adjusting the polarizability of said ophthalmic device through irradiation of said ophthalmic device by using a system,

said system comprising:
one or more irradiation sources for two-photon or multi-photon irradiating a said ophthalmic device with an irradiation beam focused with an optic and of a first wavelength and/or a second wavelength different from the first wavelength,
a scanner coupled to the one or more irradiation sources and configured to scan a said irradiation beam across said ophthalmic device, and
an input unit coupled to the one or more irradiation sources and the scanner, wherein the input unit is configured to input data for treating said ophthalmic device by scanning a said irradiation beam across said ophthalmic device based on the input data, and
   wherein the first wavelength is between 600 nm and 800 nm for locally decreasing, based on said treating of said ophthalmic device, a polarizability of said ophthalmic device, and wherein the second wavelength is between 400 nm and 590 nm for locally increasing, based on said treating of said ophthalmic device, the polarizability of said ophthalmic device and thereupon changing the polymeric optical material of said ophthalmic device, preferably with significant differences in the UV/Vis spectrum with respect to the non-irradiated polymeric optical material of the ophthalmic device.

Ultraviolet-visible spectroscopy or ultraviolet-visible spectrophotometry (UV-Vis or UV/Vis) is known to a person skilled in the art. It refers to absorption spectroscopy or reflectance spectroscopy in part of the ultraviolet and the full, adjacent visible spectral regions. Suitable UV/Vis spectrometers are commercially available. The choice of the UV/Vis spectrometer is not critical for the comparison of the UV/Vis spectrum of the initial ophthalmic device and the UV/Vis spectrum of said irradiated ophthalmic device to be made according to the present invention. As long as both measurements are made under comparable conditions so that the results can be compared which is known to the person skilled in the art. A suitable spectrometer is the UV/Vis spectrometer Lambda 900 from Perkin Elmer.

The polarizability may hereby be locally changed particularly precisely.

The invention is furthermore related to a method for correcting vision in a patient by modifying the refractive index of an intraocular lens within the eye of said patient comprising
identifying and measuring the degree of vision correction of the patient;
determining the position and type of refractive structures to be written into said intraocular lens to correct the patient's vision; and
subsequently exposing said intraocular lens to two-photon or multi-photon irradiation having a wavelength between 600 nm and 800 nm to locally decrease the polarizability of the intraocular lens or exposing said intraocular lens or

subsequently exposing said intraocular lens to two-photon or multi-photon irradiation having a wavelength between 400 nm and 590 nm to locally increase the polarizability of the intraocular lens.

In the present application, input data are all kinds of data used for creating the treatment plan which is defined as the translation of the ophthalmic need into control commands for the writing process of the ophthalmic device according to the invention. During the writing process the optical pattern is written by irradiation in said ophthalmic device.

The term “control commands” refers to commands directly controlling the process of writing as defined before. A control command may control e.g. the movement of the scanner.

The term “scanner” used within the description is not part of the input unit according to the invention. The “scanner” as described herein is a component of the system to be used in the process for adjusting a polarizability of an ophthalmic device according to the invention which controls the movement of the irradiation beam.

The ophthalmic need refers to the desired optical profile which has to be created in the ophthalmic device through the system as described.

The optical profile is the needed change defined by the surgeon according to the patient's examination results before or after the ophthalmic device, preferably the intraocular lens is implanted; for example but not limiting to a spherical full diopter change, a toric profile, an EDOF profile or a bi-, tri- or multifocal profile. Alternatively, the optical profile is the optical property adjustment of the ophthalmic device.

The optical pattern is the necessary change of polarizability resulting in change of refractive index in every voxel of the ophthalmic device.

The term “optic” as used herein as a part of the system to be used in the process for adjusting a polarizability of an ophthalmic device according to the invention includes all optical equipment necessary to control the spatial distribution of the irradiation source (focus) on the ophthalmic device. Critical parameters of the focus include the lateral focus size (or beam waist) and the focus length (or Rayleigh range). The optic comprises all elements along the optical beam path that determine the focus, such as beam expanders, aperture stops, shutter and in particular the focusing optics, such as a microscope objective or a single aspherical lens.

Multi-photon excitation occurs only in the vicinity of the focal point and preferably by employing ultra-short laser pulses. The average power is limited by the sample damage threshold such threshold being part of the input data as defined before.

Criteria for the selection and optimization of system parameters: One ultimate purpose is to generate localized refractive-index modification of IOLs post-implantation as prescribed by the physician to improve the visual acuity of the patient. A crucial criterion for the procedure of refractive-index modification is the total treatment time required to obtain the desired result. It is generally recognized that such procedure should not take more than a few minutes in order to be recognized as viable. The systems capable of localized refractive-index modifications of the state of the art do not include an approach to obtain practical treatment times for IOL applications.

Discussion of System Tradeoffs and Limitations:

For a practical high-performing system capable of adjusting ophthalmic devices in general or specifically IOLs after implantation it is recognized that its subcomponents have to be treated as a system and must therefore be optimized jointly as many interdependencies and tradeoffs between the subcomponents exist. The subcomponents include the irradiation source, the optic, the scanner and treatment plan.

A key requirement of any system/parameter optimization is to stay within safe limits for the ophthalmic device material in case of the treatment or the material and the eye with its components (e.g. retina) in case of the treatment of an ophthalmic device being an IOL. Such requirements build the basis for input data as described before. In particular, two main damage mechanisms for radiation from an irradiation source, preferably a pulsed laser source can be distinguished: Single-pulse damage (dielectric breakdown and avalanche breakdown), and thermal damage, where the temperature of the lens material and/or the eye is heated up subsequently for repeated pulses to the same volume. For example: the average power of a pulsed irradiation source relates to the heating and therefore to the potential damage of the lens material and/or the eye. Therefore, while keeping the average power of the irradiation source below the threshold of overheating the lens material and/or eye, pulse energy and pulse repetition rate are inversely related product of pulse energy and number of pulses per second (=inverse of repetition rate) is equal to the average power.

Average power is defined as pulse energy multiplied by number of pulses per second) and is characterized by Watt (W).

Irradiance is equal to flux density) (W/cm²).

Radiant exposure is equal to fluence) (J/cm²).

One overall objective is to minimize the treatment time for an IOL adjustment after implantation. In theory, higher and higher pulse energies with more frequent pulses (=higher repetition rate) could be applied, however, above an average power of typically 1 Watt, overheating starts creating unsafe conditions for IOL material and retina. Therefore, in order to stay within safe operating limits, while completing a treatment of the full IOL volume in a few minutes, one can define a preferred radiant exposure. The preferred radiant exposure is ≤5 kJ/cm², particular preferably <1 kJ/cm² and very particular preferably <0.3 kJ/cm². This described radiant exposure applies additionally to the processes and methods according to the invention as further described below.

For the case a treatment plan is too extensive and would exceed the limits of laser safety concerning overheating, it is possible to interrupt the treatment to allow a cool down of all by the treatment affected ophthalmic device material and tissues. After the cool-down the locating system can compare the treated voxel in the ophthalmic device with the optical pattern and the treatment can be continued.

The process of adjustment of optical properties/profiles of said ophthalmic device through the system and with requirements as described before will be done according to a treatment plan as described before. According to a treatment plan, profiles for e.g. toric, spheric, multifocal or EDOF (extended depth of focus) can be written into the ophthalmic device according to the invention. An algorithm may be utilized to write in profiles for e.g. toric, spheric, multifocal or EDOF (extended depth of focus) profiles.

By combining the information of the desired optical profile together with the input data, the needed optical pattern and the control commands for irradiation source, optics and scanner of the system as described before can be calculated. Further input data are for example lens data as such as the needed laser-energy for a certain refractive index change per voxel of said ophthalmic device material, and further patient data as the exact position and orientation of the ophthalmic device in the patient's eye being part of the treatment plan data.

The control commands can be updated and modified during the writing process by in-process input data such as temperature data of the patient's eye by e.g. IR-temperature measurements, in-process positioning data of the irradiation beam, the ophthalmic device or the eye acquired for example by OCT (optical coherence tomography) and/or refractive data acquired from Scheimpflug images.

In a further embodiment of input data, the input data comprises lens data of said ophthalmic device preferably of said intraocular lens and/or treatment plan data relating to a treatment plan for said treating of said ophthalmic device. For example, the lens data may comprise data relating to one or more of the polarizability and/or refractive index of the ophthalmic device as a function of the location of a respective volume or part of the ophthalmic device, shape, diopter, cylinder and sphere and/or its individual aberrations in said dimensions. The polarizability may therefore be increased or decreased at a particular location or volume in one or more planes of the ophthalmic device depending on the current polarizability (or refractive index) and the polarizability (or refractive index) to be obtained via the treatment.

The treatment plan calculation may, in some examples, generate control commands resulting in one or more of treatment plan data comprising: scan strategy control command data of a scan strategy (for example a scanning pattern and/or a scanning sequence and/or a scanning speed and/or a scanning duration of the scanning pattern and/or a scanning duration of the scanning sequence and/or a pulse duration of a pulse of the irradiation beam of the first and/or second wavelength (for example, nanosecond or picosecond or femtosecond pulses) and/or an irradiation beam profile of the irradiation beam of the first and/or second wavelength and/or a radiation (photon) density and/or a radiation intensity and/or a radiation power and/or radiation wavelength) for said scanning of the irradiation beam of the first and/or second wavelength across the ophthalmic device, in-process input data such as temperature data of a current and/or predicted temperature of the ophthalmic device during said exposure, refractive index/polarizability data of a refractive index/polarizability of the ophthalmic device to be obtained based on said exposure, the refractive index/polarizability to be obtained in particular relating to a mapping of the refractive index/polarizability to be obtained to a specific location/coordinates of the ophthalmic device, rhexis dimension data of a dimension of a rhexis, and input data such as eye data relating to a dimension and/or a shape of the eye of the patient, positioning data relating to a position and/or orientation of the ophthalmic device relative to the eye, and registration data relating to an identification of the patient and/or the specific eye of the patient.

Preferably, the scan strategy control command data of a scan strategy are a scanning pattern and/or a scanning speed and/or a pulse duration of a pulse and/or radiation intensity as described further below.

The parameters of the irradiation beam(s) may then be adjusted according to the lens data and/or the treatment plan data as defined herein in order to precisely (locally) change the polarizability/refractive index of the ophthalmic device, where desired.

Preferably, the parameters of the irradiation beam(s) are adjusted according to the lens data and/or the treatment plan data as described before or preferably described herein.

The skilled artisan is well aware in this regard that optimum irradiation focus conditions are reached when the depth-of-field (Rayleigh range) of the irradiation beam is matched to the desired thickness of the optical structure to be written into the ophthalmic device.

The skilled artisan is well aware in this regard that optimum irradiation focus conditions are reached when the depth-of-field (Rayleigh range) of the irradiation beam is matched adapted to the local thickness of the ophthalmic device.

In a further embodiment, the lens data comprises data relating to a radiation absorption property (for example an absorption and/or light attenuation coefficient, which may be dependent from the wavelength of light) of a said ophthalmic device, and wherein the system is configured to adjust the first wavelength and/or the second wave-length for said ophthalmic device to locally change the polarizability based on a multi-photon absorption process. For example, based on the material used for the ophthalmic device, a particular wavelength or wavelength ranges may be input for a precise local change of the polarizability of the ophthalmic device.

The one or more irradiation sources as part of the system to be used in the process for adjusting a polarizability of an ophthalmic device according to the invention may comprise one or more pulsed lasers which may be utilized to generate nano-second pulses, preferably pico-second pulses and more preferably femto-second pulses. Preferably, one irradiation source is used. Particular preferably, the one or more irradiation sources comprise one or more pulsed lasers which are used to generate femto-second pulses. Particular preferably, one pulsed laser is used to generate femto-second pulses is used as irradiation for the system according to the invention or for the processes and methods according to the invention.

In one embodiment, the one or more irradiation sources comprise a laser which is tunable to emit a laser beam having the first and second wavelengths, respectively. This may be particularly advantageous as a single laser may be used to (locally) increase or decrease the polarizability/refractive index of the ophthalmic device or intraocular lens, as desired.

Different pulsed laser types are suitable for said irradiation sources within the system to be used in the process for adjusting a polarizability of an ophthalmic device according to the invention. MHz laser as well as kHz laser are suitable and have their particular merits. While a MHz laser system, for example, operates at lower pulse energy, the focused laser spots can be kept at μm-scale (<1 μm to several μm) and thus be used for precise local index modifications in all three dimensions, for example, to generate diffractive structures. A preferred MHz-irradiation source is an 80 MHz laser with a pulse energy ranging from 0.1 to 10 nJ.

A kHz-laser on the other hand operates at higher pulse energy of typically 0.1 to 10 μJ and thus requires a larger spot size of, for example, 10 to 100 μm in order to not damage the lens material. A larger laser spot size, however, implies a large depth of field (=long Rayleigh range) that can be equal to or even exceed the thickness of the ophthalmic device material. For such long Rayleigh ranges, it might not be possible to modify the refractive index layer by layer in the IOL but only uniformly along a line around the focus. A preferred kHz-irradiation source is a laser with a repetition rate of 100 to 500 kHz.

The average power of the irradiation source as described before or preferably described before is preferably between 300 and 600 mW, particular preferably between 400 and 500 mW.

The irradiation source as part of the system to be used in the process for adjusting a polarizability of an ophthalmic device according to the invention preferably comprises a tunable laser that can provide a variable wavelength in the range of approximately 680-1080 nm, such as a Ti:Sapphire laser (for example Chameleon Ultra II by Coherent, Santa Clara, Calif., USA). The system may also comprise an optical parametric oscillator (for example frequency doubled Chameleon Compact OPO-Vis by Coherent, Santa Clara, Calif., USA).

The irradiation source as part of the system to be used in the process for adjusting a polarizability of an ophthalmic device according to the invention particularly preferably comprises a femtosecond pump laser along with an optical parametric amplifier. Said pump laser emits irradiation >10 Watt average power at 1030 nm in <350 fs pulses with a repetition rate of 0.1 to 700 kHz. The radiation of said pump laser is directed to an optical parametric amplifier, where the pump laser output is frequency-doubled and optically mixed, resulting in a final tunable output in a wavelength range of 600 nm to 800 nm. A preferred repetition rate is between 50 and 600 kHz. A particular preferred repetition rate is between 100 and 500 kHz.

The irradiation source as part of the system to be used in the process for adjusting a polarizability of an ophthalmic device according to the invention particularly preferably comprises a femto-second pump laser >10 Watt average power at 1030 nm in combination with an optical parametric amplifier, which is emitting irradiation pulses <350 fs at a repetition rate of 1 to 700 kHz. The radiation of said pump laser is directed to an optical parametric amplifier with one or multiple second-harmonic stages, resulting in a final optical output in a wavelength range of 400 nm to 590 nm. A preferred repetition rate is between 50 and 600 kHz. A particular preferred repetition rate is between 100 and 500 kHz.

The laser types as described before or preferably described before generate a collimated optical beam of a few millimeter in diameter, which is then directed to optics and scanner. The optical beam quality (characterized through the beam quality factor or beam propagation factor) is ideally between 1.0 and 1.5, more ideally between 1.0 and 1.3. According to DIN EN ISO 11146, the optical beam quality is given in the dimension of M².

The first wavelength of the irradiation beam within the system to be used in the process for adjusting a polarizability of an ophthalmic device according to the invention is between 600 nm and 800 nm, preferably between 650 nm and 750 nm, more preferably between 670 nm and 720 nm, more preferably between 680 and 710, in order to (locally) decrease the polarizability (and hence the refractive index) of the IOL.

The second wavelength of the irradiation beam within the system to be used in the process for adjusting a polarizability of an ophthalmic device according to the invention is between 400 nm and 590 nm, preferably between 500 nm and 580 nm, more preferably between 530 nm and 570 nm, in order to (locally) increase the polarizability (and hence the refractive index) of the IOL.

The polarizability may hereby be locally changed particularly precisely.

Optics within the system to be used in the process for adjusting a polarizability of an ophthalmic device according to the invention: The main function of the optics is to focus the irradiation beam, which is emitted from the irradiation source and controlled by the scanner, onto the ophthalmic device. Key considerations as described before are spot size and depth of focus in order to minimize treatment time while staying within limits given by laser safety requirements and material damage as described before as part of common input data. The most important characteristics of the optic is given by its numerical aperture (NA), along with its effective focal length (EFL) and the diameter of the irradiation beam at the entry aperture of the focusing optics. Additionally, all optical elements within the system to be used in the process for adjusting a polarizability of an ophthalmic device according to the invention should be selected for diffraction- or near-diffraction-limited properties, in order to not substantially degrade the optical beam quality.

Different ophthalmic needs will require different spot sizes as the spot size determines the obtainable spatial resolution. Ideally, the spot size is between 1 and 100 μm, more ideally between 50 and 100 μm in order to minimize treatment time while also keeping the potential for material damage low.

Scanner within the system to be used in the process for adjusting a polarizability of an ophthalmic device according to the invention: The scanner to be used within the system to be used in the process for adjusting a polarizability of an ophthalmic device according to the invention may comprise a Galvano-scanner, a piezo scanner, a rotational scanner or an acousto optic modulator or it may be digital such as a spatial light modulator, a digital micromirror device or stereolithography apparatus. Preferably, the scanner as part of the inventive system according to the description is selected from a Galvano-scanner, a piezo scanner, a rotational scanner, an acousto optic modulator, a spatial light modulator, a digital micromirror device or stereolithography apparatus. A preferred Galvano-scanner is a single Pivot-Point-Scanner.

Preferably, the scanner is configured to operate at a scanning speed of more than 50 mm/s. This may allow for keeping the treatment time short. As a general rule, the treatment time should not exceed several minutes and is preferably less than 10 minutes, preferably less than 5 minutes, particularly preferably less than 3 minutes per treatment session.

The treatment area may be defined as the ophthalmic devices volume and size. Typically, the optic of said ophthalmic device or intraocular lens is 5 mm to 7 mm in diameter and typically between 0.2 mm and 2.0 mm thick.

The optimum radiation exposure is <1 kJ/cm² and more ideally <0.3 kJ/cm² to keep the overall irradiation exposure low and treatment time short, while addressing the full volume of the ophthalmic device.

Particularly preferably, random scan patterns or interleaved scanning lines are used to spread out the irradiation energy of the irradiation beam.

The scanning can be performed with three modes. In the bottom-up scanning, the laser may travel from spot-to-spot with a specific dwell time on each spot (“bottom-up, spot-to-spot”). Alternatively, in the bottom-up scanning, the laser may dwell on spots that overlap with one another (“bottom-up, spot overlay”). Alternatively, the laser can travel with a fixed velocity without dwelling on any spots (“fly by, constant velocity”).

In one embodiment of a scan pattern, the IOL is scanned with the irradiation source as described before or preferably described before by shining through the pupil. The IOL, contained in the capsular bag at the time of scanning, is previously inserted through an incision in the cornea using conventional operation procedures. In this embodiment, the full volume of the IOL is scanned and the scanning is performed in a bottom-up manner (i.e. parts of the IOL which are further away from the cornea are scanned first), this way the optical profile is created in order to avoid unnecessary changes in refractive index in the light path.

As described before a key consideration when selecting the scanning program is to minimize local heating of the ophthalmic device and/or the eye of the patient and therefore various variables are used in the scanning program. Taking into account anatomical features such as Rhexis and pupil size as well as optical features such as numerical aperture and laser pulse characteristics, a laser program is created with a specific scanning speed and sequence. The relation between lens coordinate and eye coordinate system are, in this example, automatically taken into account.

The parameters for the scanning program and/or treatment plan are preferably the first and second wavelengths, the scanning speed and sequence, the positioning (e.g. in Cartesian coordinates) of the lens relative to the eye, the scan strategy, the refractive index change which is to be obtained (optical pattern), the numerical aperture of the objective, the Rhexis, the optical diameter (in some examples approximately 6 mm) of the pupil and/or the lens, the pulse duration (shape, intensity and x-y positioning) of the laser beam, laser safety when operating the laser, and centration with respect to the positioning of the lens and the eye.

The photons generated in the laser are in one embodiment of the system to be used in the process for adjusting a polarizability of an ophthalmic device according to the invention preferably guided through mirrors (e.g. as optic 1) to a e.g. a beam expander, which prepares the beam for the subsequent scanner and focusing optic. After passing through the beam expander, the photons are directed toward the scanner (e.g. Galvano-scanner or piezo scanner or rotational scanner or acousto optic modulator or digitally with a spatial light modulator or digital micromirror device or stereolithography apparatus).

After having gone through the scanner, the laser beam travels through another optic such as a divider mirror. In this embodiment, the divider mirror splits up the beam into the main imaging beam for the ophthalmic device irradiation and a beam for monitoring beam properties as well as for positioning feedback. After the divider mirror, the optical beam is focused onto the ophthalmic device by imaging group or focusing optic. In one embodiment, the imaging group comprise a microscope objective to obtain high numerical apertures (for μm-level spatial resolution) or low-NA optics to allow higher pulse energy of μJ-level.

The system as described before or preferably described before may further comprise a microscope objective coupled to the scanner for focusing, by the microscope objective, a said irradiation beam onto said ophthalmic device, wherein the microscope objective has a numerical aperture of between 0.1 and 0.8, preferably between 0.2 and 0.5, and more preferably between 0.2 and 0.4. Providing a microscope objective having such numerical aperture may allow for high irradiation beam quality in particular in terms of focusing and resolution characteristics of the beam used for treating the intraocular lens.

The microscope objective comprises of a typical lens configuration to allow for e.g. correction of chromatic aberration. The microscope objective is preferably linked to an eye interface system, typically a suction system that keeps the eye of the patient in a fixed position as further described below.

In a further embodiment of an objective to be used within the system as described before, the objective is an Olympus LUCPLFLN objective in order to focus the irradiation beam onto the ophthalmic device.

An alternative focusing optic/imaging group is configured with a single aspherical lens with an effective focal length preferably within 50 to 150 mm and a numerical aperture of preferably 0.025 to 0.1.

The system as described before or preferably described before may further comprise a positioning system for determining a position of a said focus of said irradiation beam within a said eye of a said patient, wherein the positioning system is coupled to the scanner and wherein the scanning, by the scanner, of said irradiation beam across said intraocular lens is based on the position of said focus of said irradiation beam within the eye.

The positioning system may comprise a locating system such as an optical coherence tomography system, a confocal microscope or a Scheimpflug camera. The positioning system may be directly or indirectly coupled to the scanner. In some examples in which a confocal microscope is used, the confocal microscope may be directly coupled to the scanner.

The locating system as described before is used to provide topographic data of the eye to the positioning system for determination of the position of the laser focus in dependence of the eye and said intraocular lens.

For confocal microscopy, a partially transparent mirror is used to allow for video imaging.

The system as described before or preferably described before is preferably configured further to determine a location and/or orientation of said intraocular lens relative to the eye and the outlet of the irradiation beam, and wherein the scanning, by the scanner, of said irradiation beam across said intraocular lens is based on the location and/or orientation of said intraocular lens relative to the eye. This may be particularly advantageous since the position of the intraocular lens may not be centered relative to the eye, which misalignment may be taken into account when treating the intraocular lens with the irradiation beam(s).

With respect to the position of the IOL, at least 2 coordinate systems may be considered relevant: coordinates-system of the eye and coordinates-system of the lens within the eye, as both may not be centered with respect to each other.

With respect to the position of the IOL, at least 2 coordinate systems may be considered relevant: x,y,z coordinates of the eye and x,y,z coordinates of the lens within the eye, as both may not be centered with respect to each other.

In one embodiment, the locating system creates input data. These input data contain for example data concerning lens position and/or orientation of the ophthalmic device within the eye and relative to the laser beam outlet, and/or an optical power mapping of the eye and/or the ophthalmic device. These data are used for the calculation of the optical pattern or a continuation of a treatment.

Additionally, it is possible that the locating system creates input data during the writing process. These in-process input data contain for example data concerning lens position and/or orientation of the ophthalmic device within the eye and relative to the laser beam outlet, and/or an optical power mapping of the eye and/or the ophthalmic device. These data are used for in-process modification of the control commands used to generate the optical pattern.

The system as described before or preferably described before may further comprise a temperature management unit coupled to one or both of (i) the one or more irradiation sources and (ii) the scanner, wherein the temperature management unit is configured to determine, based on an irradiation beam property of a said irradiation beam and an ophthalmic device property of a said ophthalmic device, a temperature of a part of said ophthalmic device during said treating of said ophthalmic device by said scanning, and wherein the system is configured to control, based on said determination of the temperature, one or both of (i) the one or more irradiation sources and (ii) the scanner. This may allow for ensuring that the eye and/or the ophthalmic device may not be detrimentally affected based on the treatment with an irradiation beam.

Additionally, the temperature management unit is preferably configured to predict said temperature during said treating of said ophthalmic device, and wherein said input data comprises the predicted temperature. This may allow for taking preventative measures to ensure that the eye and/or the ophthalmic device may not be detrimentally affected based on the treatment with an irradiation beam.

Alternatively, the temperature management unit is an infrared camera logging the temperature of the eye and correlating the measured data with common data bearing calibration data to calculate the real temperature in the eye.

In another embodiment, the temperature dependence of refractive index is used for temperature controlling. In these examples, the system comprises a refractive power mapping device. Based on the deviation of the measured refractive power map and the progress of the writing predicted refractive power map, temperatures in the lens can be calculated in process.

In another embodiment, the temperature dependence of the emission spectrum is used for temperature controlling. In these examples, the system comprises a UV-Vis spectrometer. Based on the deviation of the measured emission peak wavelength and/or peak width, temperatures in the focal spot can be calculated in process.

The system as described before or preferably described before may further comprise an eye interface system configured to keep a said eye of a said patient in a fixed position. The eye interface system may comprise a suction system for fixing the position of the eye of the patient during treatment.

The patient may be “docked” to the system in a lie flat or upright position.

The system as described before or preferably described before may further comprise a wireless or wired receiver and/or transceiver for one or more of (i) sending control commands to the one or more irradiation sources, (ii) sending control commands to the scanner, and (iii) inputting the control command data needed for creating the optical pattern into the scanner.

The one or more irradiation sources and/or the scanner may therefore be controlled remotely. Additionally or alternatively, the data relating to one or both of the lens data and the treatment plan data may be stored externally from the system and may be provided to the system as and when desired.

In some examples, it may be preferable to provide a wired receiver or transceiver at least for controlling the one or more irradiation sources and/or for controlling the scanner in order to reduce (or avoid) any delay when sending a control signal to the one or more irradiation sources and/or the scanner

In another example, the receiver/transceiver sends treatment plan data and lens data to a central computing unit which calculates the optical pattern and sends this as input data back to the receiver which provides it to the system.

The system as described before or preferably described before may further comprise a device for locally measuring the refractive power of said ophthalmic device during said treating of the ophthalmic device. Adjustments to one or more of the irradiation source(s), the scanner and the input data may hereby be made during the treatment process.

The system as described before or preferably described before may further comprise a refractometer for locally measuring the refractive index of said ophthalmic device during said treating of the ophthalmic device. Adjustments to one or more of the irradiation source(s), the scanner and the input data may hereby be made during the treatment process.

Further components of the system providing the photons are optionally a cover in which all the equipment is built in, a power unit to provide the system and all sub-systems with sufficient energy, and sub-systems like a suction system and/or chiller.

In addition to the above mentioned components, controller, firmware and a graphics user interface (GUI) as well as treatment algorithms may be provided. To connect to the system, connectivity may be established via Bluetooth, Wi-Fi or other ports like RS-232.

The invention further relates to a method for locally adjusting a polarizability of an intraocular lens according to the invention arranged within an eye of a patient, wherein the treatment plan data comprises one or more of:
scan strategy control command data of a scan strategy (for example a scanning pattern and/or a scanning sequence and/or a scanning speed and/or a scanning duration of the scanning pattern and/or a scanning duration of the scanning sequence and/or a pulse duration of a pulse of the irradiation beam of the first and/or second wavelength and/or an irradiation beam profile of a said irradiation beam and/or a radiation (photon) density and/or a radiation intensity and/or a radiation power and/or radiation wavelength) for said scanning of a said irradiation beam across the intraocular lens,
temperature data of a current and/or predicted temperature of the intraocular lens during said exposure,
refractive index data of a refractive index of the intraocular lens to be obtained based on said exposure, the refractive index to be obtained in particular relating to a mapping of the refractive index to be obtained to a specific location/coordinates of the intraocular lens,
rhexis dimension data of a dimension of a rhexis,
eye data relating to a dimension and/or a shape of the eye of the patient,
positioning data relating to a position and/or orientation of the intraocular lens relative to the eye, and
registration data relating to an identification of the patient and/or the specific eye of the patient.

The invention further relates to a method for locally adjusting a polarizability of an intraocular lens according to the invention arranged within an eye of a patient, wherein said exposing of the intraocular lens to a said irradiation beam comprises exposing a first volume of the intraocular lens prior to exposing a second volume of the intraocular lens, wherein the first volume is further away from the cornea of the eye of the patient than the second volume.

In the above-stated clauses, an initial step of the methods may be to provide a said intraocular lens.

In examples, in which said exposing of the intraocular lens to a said irradiation beam comprises exposing a first volume and/or plane and/or location of the intraocular lens prior to exposing a second volume and/or plane and/or location of the intraocular lens, wherein the first volume and/or plane and/or location is further away from the cornea of the eye of the patient than the second volume and/or plane and/or location, volumes and/or planes and/or locations irradiated at later time points in the irradiation sequence may be closer to the cornea than volumes and/or planes and/or locations irradiated at earlier time points. A said volume may hereby relate to one or more planes of the intraocular lens.

The invention is furthermore related to a method for correcting vision in a patient by modifying the refractive index of an intraocular lens according to the invention within the eye of said patient comprising
identifying and measuring the degree of vision correction of the patient;
determining the position and type of refractive structures to be written into said intraocular lens to correct the patient's vision; and
subsequently exposing said intraocular lens to two-photon or multi-photon irradiation having a wavelength between 600 nm and 800 nm to locally decrease the polarizability of the intraocular lens and/or
subsequently exposing said intraocular lens to two-photon or multi-photon irradiation having a wavelength between 400 nm and 590 nm to locally increase the polarizability of the intraocular lens, preferably by using the system and/or the process as described before for exposing said intraocular lens to said irradiation.

As outlined above, the change of polarizability results in a change of refractive index.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive.

This is especially true of preferred features of the present invention. Equally, features of non-essential combinations may be used separately (and not in combination).

It should also be pointed out that many of the features, and especially those of the preferred embodiments of the present invention, are themselves inventive and should not be regarded merely as some of the embodiments of the present invention. For these features, independent protection may be sought in addition to or as an alternative to any currently claimed invention.

The technical teaching disclosed with the present invention may be abstracted and combined with other examples.

No doubt many other effective alternatives will occur to the skilled person. It will be understood that the invention is not limited to the described embodiments and encompasses modifications apparent to those skilled in the art and lying within the scope of the claims appended hereto.

### EXAMPLES

The following examples are intended to show the advantages of the present compounds in a non-limiting way.

Unless indicated otherwise, all syntheses are carried out under an inert atmosphere using dried (i.e. water-free) solvents. Solvents and reagents are purchased from commercial suppliers.

DCM is used to denote dichloromethane. DMF is used to denote dimethylformamide. EE or EtOAc is used to denote ethyl acetate. THF is used to denote tetrahydrofuran. RT means room temperature.

Copolymer-properties can be investigated on blanks, prepared by bulk polymerization of the monomers. Co-monomers, cross-linkers and initiators therefore can be purchased from commercial sources. All chemicals are of highest purity available and can be used as received.

### Synthesis of Precursor Materials

### Example 1

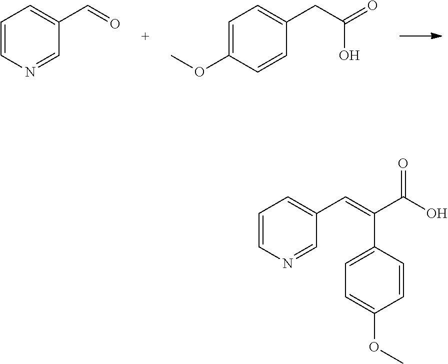 [SMILES: O=Cc1cccnc1 ; COc1ccc(CC(=O)O)cc1 ; COc1ccc(/C(=C\c2cccnc2)C(=O)O)cc1]

4-Methoxyphenylacetic acid (30.000 mmol; 1.00 eq.; 5.087 g) is suspended with acetic anhydride (302.400 mmol; 10.08 eq.; 30.872 g; 28.585 ml). Then pyridin-3-carbaldehyde (30.00 mmol; 1.00 eq.; 3.246 g; 2.847 ml) and triethylamine (30.00 mmol; 1.00 eq.; 3.036 g; 4.181 ml) are added. The reaction mixture is heated up to 120° C. overnight. Water is added at 110° C., the mixture is stirred for half an hour before cooling down to RT. Volatiles are removed in vacuo. The crude mixture is purified by column chromatography using dichlormethane/methanol as an eluent. 3.3 g (E)-2-(4-methoxy-phenyl)-3-pyridin-3-yl-acrylic acid is isolated as beige solid (45% yield of theory).

¹H NMR (500 MHz, DMSO-d₆) δ 12.78 (br s, 1H), 8.39 (dd, J=4.8, 1.6 Hz, 1H), 8.32 (d, J=2.2 Hz, 1H), 7.72 (s, 1H), 7.35 (dt, J=8.0, 1.8 Hz, 1H), 7.23 (dd, J=8.0, 4.8 Hz, 1H), 7.09 (d, J=8.7 Hz, 2H), 6.94 (d, J=8.8 Hz, 2H), 3.78 (s, 3H).

Analogously, other derivatives are prepared in the same manner: R1 means reactant, R2 means reactant 2, [P] means product.

| No. | | | Yield [%] |
|---|---|---|---|
| 1a | R1 | 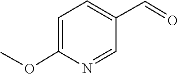 | |
| | R2 | 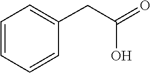 | |
| | [P] | 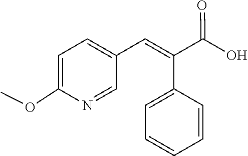 [SMILES: COc1ccc(/C=C(/C(=O)O)c2ccccc2)cn1] | 34 |

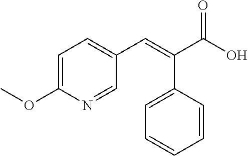 [SMILES: COc1ccc(/C=C(/C(=O)O)c2ccccc2)cn1]

¹H NMR (500 MHz, DMSO-d₆) δ 12.65 (s, 1H), 8.01 (d, J=2.4 Hz, 1H), 7.73 (s, 1H), 7.44-7.34 (m, 3H), 7.22-7.15 (m, 2H), 7.11 (dd, J=8.8, 2.5 Hz, 1H), 6.61 (d, J=8.7 Hz, 1H), 3.80 (s, 3H).

### Example 2

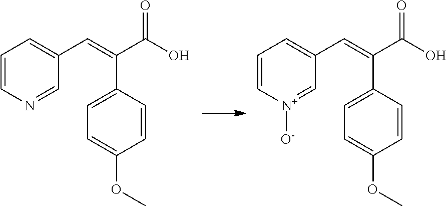 [SMILES: COc1ccc(/C(=C\c2cccnc2)C(=O)O)cc1 ; COc1ccc(/C(=C\c2ccc[n+]([O-])c2)C(=O)O)cc1]

(E)-2-(4-Methoxy-phenyl)-3-pyridin-3-yl-acrylic acid (11.61 mmol; 1.00 eq.; 2.964 g) is suspended in dry dichloromethane (952.125 mmol; 82.0 eq.; 60.80 ml). Then, the suspension is cooled with an ice-bath and meta-chlorperoxybenzoic acid (13.93 mmol; 1.20 eq.; 3.123 g) is added. The reaction mixture is warmed to RT while stirring overnight. The suspension is filtrated and the collected solid is washed twice with diethyl ether and dried in vacuo. 2.83 g (E)-2-(4-methoxy-phenyl)-3-(1-oxy-pyridin-3-yl)-acrylic acid is isolated as colourless solid (90% yield of theory).

¹H NMR (500 MHz, DMSO-d₆) δ 12.96 (s, 1H), 8.05 (d, J=7.0 Hz, 1H), 7.92 (s, 1H), 7.60 (s, 1H), 7.25 (dd, J=7.9, 6.6 Hz, 1H), 7.11 (d, J=8.7 Hz, 2H), 7.02-6.85 (m, 3H), 3.78 (s, 3H).

Analogously, other derivatives are prepared in the same manner:

| No. | Reactant | Product | Yield [%] |
|---|---|---|---|
| 2a | 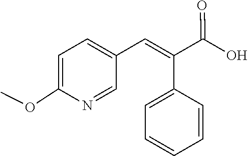 [SMILES: COc1ccc(/C=C(/C(=O)O)c2ccccc2)cn1] | 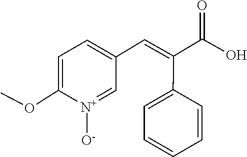 [SMILES: COc1ccc(/C=C(/C(=O)O)c2ccccc2)c[n+]1[O-]] | 22 |

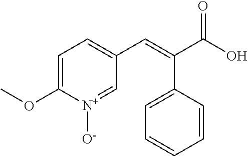 [SMILES: COc1ccc(/C=C(/C(=O)O)c2ccccc2)c[n+]1[O-]]

¹H NMR (500 MHz, DMSO-d₆) δ 12.86 (s, 1H), 7.93 (d, J=2.0 Hz, 1H), 7.63 (s, 1H), 7.44-7.38 (m, 3H), 7.21-7.19 (m, 2H), 7.04 (d, J=8.8 Hz, 1H), 6.86 (dd, J=8.8, 2.1 Hz, 1H), 3.90 (s, 3H).

### Example 3

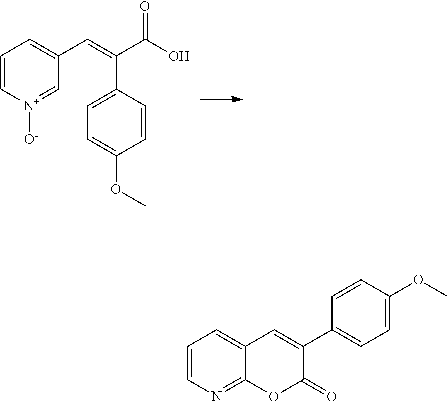 [SMILES: COc1ccc(/C(=C\c2ccc[n+]([O-])c2)C(=O)O)cc1 ; COc1ccc(-c2cc3cccnc3oc2=O)cc1]

(E)-2-(4-Methoxyphenyl)-3-(1-oxy-pyridin-3-yl)-acrylic acid (9.37 mmol; 1.00 eq.; 2.54 g) is suspended in acetic anhydride (618.72 mmol; 66.00 eq.; 63.164 g; 58.49 ml). Then sodium carbonate (28.12 mmol; 3.00 eq.; 3.89 g) und distilled water (65.62 mmol; 7.00 eq.; 1.182 g; 1.182 ml) are added. The evolution of gas is visible and the reaction mixture is stirred at 130° C. overnight. During cooling to RT the reaction mixture solidifies and distilled water is added. The suspension is filtrated and washed several times with more water. The solid is dried in vacuo. 2.1 g of 3-(4-methoxy-phenyl)-pyrano[2,3-b]pyridin-2-one is isolated as a grey-beige solid (89% yield of theory).

¹H NMR (500 MHz, Chloroform-d) δ 8.51 (dd, J=4.8, 1.8 Hz, 1H), 7.91 (dd, J=7.6, 1.8 Hz, 1H), 7.74 (s, 1H), 7.69 (d, J=8.8 Hz, 2H), 7.31 (dd, J=7.5, 4.8 Hz, 1H), 6.98 (d, J=8.8 Hz, 2H), 3.86 (s, 2H).

Analogously, other derivatives are prepared in the same manner:

| No. | Reactant | Product | Yield [%] |
|---|---|---|---|
| 3a | 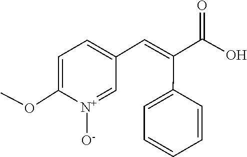 [SMILES: COc1ccc(/C=C(/C(=O)O)c2ccccc2)c[n+]1[O-]] | 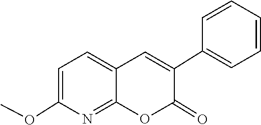 [SMILES: COc1ccc2cc(-c3ccccc3)c(=O)oc2n1] | 3 |

### Example 4

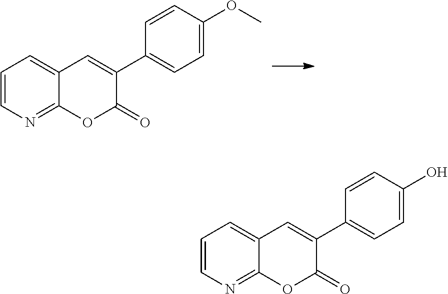 [SMILES: COc1ccc(-c2cc3cccnc3oc2=O)cc1 ; O=c1oc2ncccc2cc1-c1ccc(O)cc1]

3-(4-Methoxy-phenyl)-pyrano[2,3-b]pyridin-2-one (7.42 mmol; 1.00 eq.; 1.88 g) is suspended in 50 ml dry dichloromethane and the solution is cooled with an ice-bath. Then, borontribromide (8.91 mmol; 1.20 eq.; 2.23 g; 0.85 ml), dissolved in 10 ml DCM, is added dropwise. The solution is warmed overnight to RT and the reaction mixture is quenched with water. The suspension is filtrated and the solid is washed with water several times. The crude solid is recrystallized using acetone and ethanol. 1.2 g of 3-(4-hydroxy-phenyl)-pyrano[2,3-b]pyridin-2-one is isolated as a light-grey solid (68% yield of theory).

¹H NMR (500 MHz, DMSO-d₆) δ 9.79 (s, 1H), 8.49 (dd, J=4.8, 1.9 Hz, 1H), 8.23 (dd, J=7.6, 1.9 Hz, 1H), 8.18 (s, 1H), 7.60 (d, J=8.7 Hz, 2H), 7.46 (dd, J=7.6, 4.8 Hz, 1H), 6.86 (d, J=8.7 Hz, 2H).

### Example 5

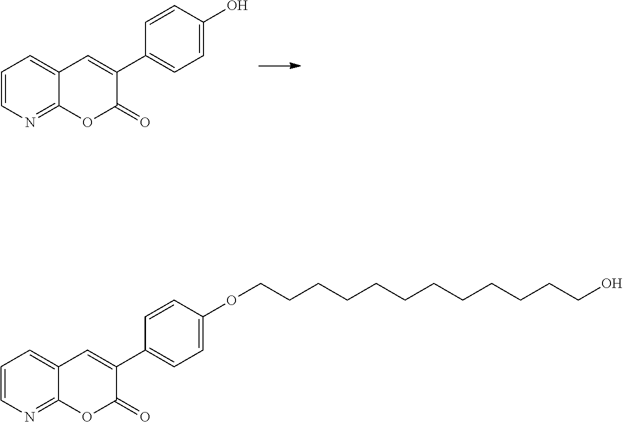 [SMILES: O=c1oc2ncccc2cc1-c1ccc(O)cc1 ; O=c1oc2ncccc2cc1-c1ccc(OCCCCCCCCCCCCO)cc1]

3-(4-Hydroxy-phenyl)-pyran[2,3-b]pyridin-2-one (1.2 g, 5.02 mmol, 1.00 eq.) is refluxed with potassium carbonate (2.81 g, 20.07 mmol, 4.00 eq.) and 12-bromo-dodecan-1-ol (1.43 g, 5.27 mmol, 1.05 mmol) in acetone (40 ml) for minimum 2 d. The suspension is filtered, and the solvent of the filtrate is evaporated. The crude residue is recrystallized from acetone and n-butanol yielding 0.685 g of 3-[4-(12-Hydroxy-dodecyloxy)-phenyl]-pyran[2,3-b]pyridin-2-on (32% yield of theory).

¹H NMR (500 MHz, DMSO-d₆) δ 8.50 (dd, J=4.8, 1.8 Hz, 1H), 8.24 (d, J=6.1 Hz, 2H), 7.70 (d, J=8.8 Hz, 2H), 7.48 (dd, J=7.5, 4.8 Hz, 1H), 7.03 (d, J=8.8 Hz, 2H), 4.30 (t, J=5.1 Hz, 1H), 4.02 (t, J=6.5 Hz, 2H), 3.36 (td, J=6.5, 4.9 Hz, 2H), 1.73 (p, J=6.6 Hz, 2H), 1.41 (dt, J=12.1, 5.6 Hz, 5H), 1.34-1.22 (m, 17H).

### Preparation of Compounds According to the Invention

### Example 6.1

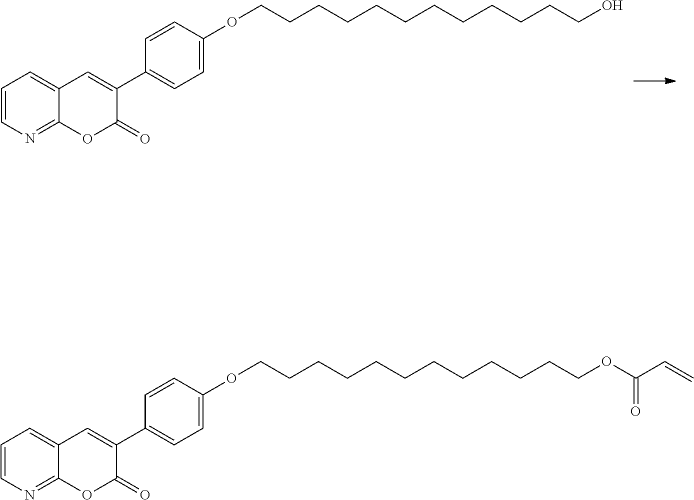 [SMILES: O=c1oc2ncccc2cc1-c1ccc(OCCCCCCCCCCCCO)cc1 ; C=CC(=O)OCCCCCCCCCCCCOc1ccc(-c2cc3cccnc3oc2=O)cc1]

3-[4-(12-Hydroxy-dodecyloxy)-phenyl]-pyran[2,3-b]pyridin-2-one (0.67 g, 1.58 mmol, 1.00 eq.) is dissolved in dry THF (60 ml) and triethylamine (0.88 ml, 6.33 mmol, 4.00 eq.) is added. Then acryloyl chloride (0.161 ml, 1.9 mmol, 1.20 eq.) is added at 0° C. and stirred at room temperature until completion of the reaction. The reaction is quenched with 2-propanol (0.25 ml), the suspension is filtered, and the solvent of the filtrate is removed. The crude product is cleaned by column chromatography using CHCl₃/MeOH as eluent. The synthesis yield 0.375 g acrylic acid 12-[4-(2-oxo-2H-pyrano[2,3-b]pyridin-3-yl)-phenoxy]-dodecyl ester (49% of theory).

¹H NMR (500 MHz, Chloroform-d) δ 8.50 (dd, J=4.8, 1.8 Hz, 1H), 7.91 (dd, J=7.6, 1.8 Hz, 1H), 7.73 (s, 1H), 7.68 (d, J=8.8 Hz, 2H), 7.30 (dd, J=7.6, 4.8 Hz, 1H), 6.97 (d, J=8.8 Hz, 2H), 6.39 (dd, J=17.3, 1.4 Hz, 1H), 6.12 (dd, J=17.3, 10.4 Hz, 1H), 5.81 (dd, J=10.4, 1.4 Hz, 1H), 4.15 (t, J=6.8 Hz, 2H), 4.01 (t, J=6.6 Hz, 2H), 1.80 (dt, J=14.5, 6.6 Hz, 2H), 1.67 (p, J=6.8 Hz, 2H), 1.46 (q, J=7.5 Hz, 2H), 1.39-1.25 (m, 17H).

m. p. (DSC): 109° C.

Absorption maximum (UV-Vis): 347 nm

Analogously, other derivatives are prepared in the same manner:

| No. | Reactant | |
|---|---|---|
| 6a | 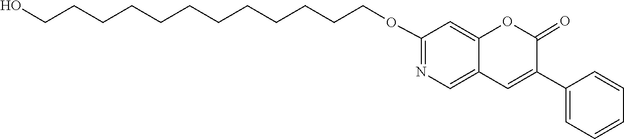 [SMILES: O=c1oc2cc(OCCCCCCCCCCCCO)ncc2cc1-c1ccccc1] | |
| 6b | 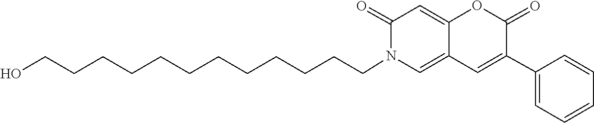 [SMILES: O=c1oc2cc(=O)n(CCCCCCCCCCCCO)cc2cc1-c1ccccc1] | |
| | | Yield |
| No. | Product | [%] |
| 6a | 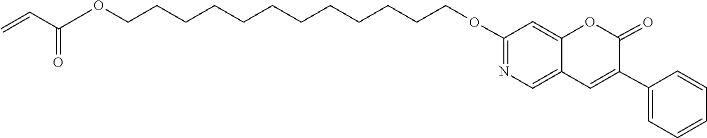 [SMILES: C=CC(=O)OCCCCCCCCCCCCOc1cc2oc(=O)c(-c3ccccc3)cc2cn1] | 82 |
| 6b | 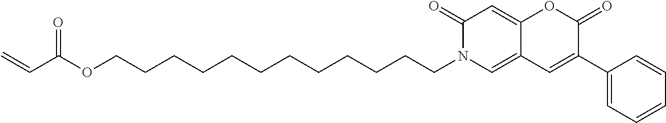 [SMILES: C=CC(=O)OCCCCCCCCCCCCn1cc2cc(-c3ccccc3)c(=O)oc2cc1=O] | 93 |

Compound 6a:

¹H NMR (500 MHz, DMSO-d₆) δ 8.61 (s, 1H), 8.24 (d, J=0.7 Hz, 1H), 7.70-7.67 (m, 2H), 7.48-7.44 (m, 2H), 7.43-7.40 (m, 1H), 6.83 (s, 1H), 6.30 (dd, J=17.3, 1.6 Hz, 1H), 6.16 (dd, J=17.3, 10.3 Hz, 1H), 5.92 (dd, J=10.3, 1.6 Hz, 1H), 4.34 (t, J=6.6 Hz, 2H), 4.08 (t, J=6.7 Hz, 2H), 1.73 (p, J=6.8 Hz, 2H), 1.59 (p, J=6.7 Hz, 2H), 1.40 (p, J=6.9 Hz, 2H), 1.35-1.21 (m, 16H).

m.p. (DSC): 88.2° C.

Absorption maximum (UV-Vis): 328 nm

Compound 6b:

¹H NMR (500 MHz, Chloroform-d) δ 7.68 (s, 1H), 7.60-7.56 (m, 2H), 7.48 (s, 1H), 7.45-7.37 (m, 3H), 6.39 (dd, J=17.3, 1.5 Hz, 1H), 6.35 (s, 1H), 6.11 (dd, J=17.3, 10.4 Hz, 1H), 5.81 (dd, J=10.4, 1.5 Hz, 1H), 4.14 (t, J=6.7 Hz, 2H), 4.04-3.97 (m, 2H), 1.78 (p, J=7.4 Hz, 2H), 1.69-1.62 (m, 2H), 1.38-1.24 (m, 17H).

m.p. (DSC): 87.3° C.

Absorption maximum (UV-Vis): 359 nm

### Example 6.2

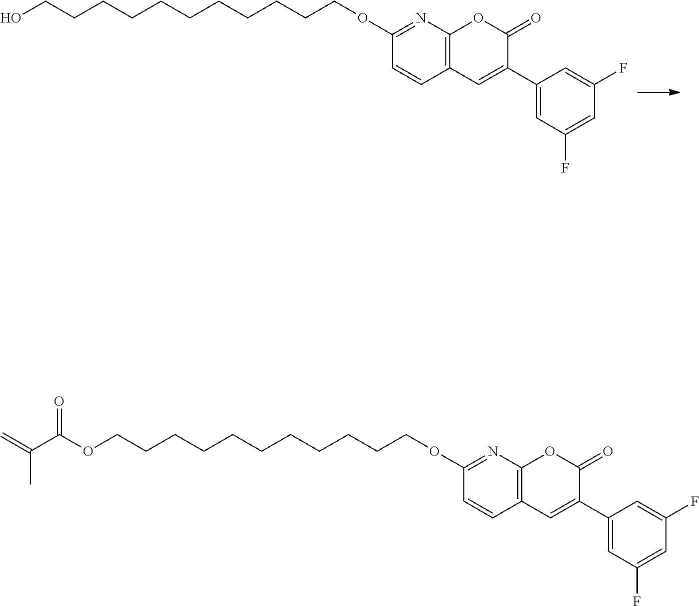 [SMILES: O=c1oc2nc(OCCCCCCCCCCCO)ccc2cc1-c1cc(F)cc(F)c1 ; C=C(C)C(=O)OCCCCCCCCCCCOc1ccc2cc(-c3cc(F)cc(F)c3)c(=O)oc2n1]

3-(3,5-Difluorophenyl)-7-[(11-hydroxyundecyl)oxy]-2H-pyrano[2,3-b]pyridin-2-one (0.15 g, 0.348 mmol, 1.00 eq.) is dissolved in DCM (10 mL) and triethylamine (193 μL, 1.392 mmol, 4.00 eq.). 4-(Dimethylamino)pyridine (9 mg, 0.07 mmol, 0.20 eq.) and methacrylic anhydride (30 μL, 0.418 mmol, 1.20 eq.) are added at 0° C. After stirring at RT for one day further methacrylic anhydride is added (66.2 μL, 0.189 mmol, 0.60 eq). The reaction is stirred for one additional day until completion of the reaction. The reaction mixture is concentrated in vacuo and subjected to column chromatography using cyclohexane/chloroform as an eluent. The synthesis yield 0.108 g 11-{[3-(3,5-difluorophenyl)-2-oxo-2H-pyrano[2,3-b]pyridin-7-yl]oxy}undecyl 2-methylprop-2-enoate (0.21 mmol, 66% of theory).

¹H NMR (500 MHz, DMSO-d₆) δ 8.45 (s, 1H), 8.13 (d, J=8.4 Hz, 1H), 7.54-7.48 (m, 2H), 7.31 (tt, J=9.3, 2.4 Hz, 1H), 6.92 (d, J=8.4 Hz, 1H), 6.00 (d, J=1.5 Hz, 1H), 5.65 (t, J=1.7 Hz, 1H), 4.35 (t, J=6.6 Hz, 2H), 4.07 (t, J=6.6 Hz, 2H), 1.87 (s, 3H), 1.75 (p, J=6.8 Hz, 2H), 1.60 (p, J=6.8 Hz, 2H), 1.44-1.37 (m, 2H), 1.35-1.22 (m, 12H).

¹⁹F NMR (470 MHz, DMSO-d₆) 5-110.0.

m. p. (DSC): 102° C.

Absorption maximum (UV-Vis): 347 nm

### Synthesis of Precursor Materials

### Example 7

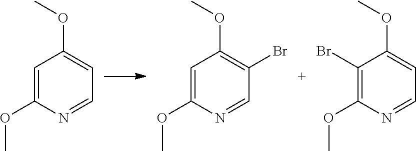

2,4-Dimethoxypyridine (15.00 g; 103.48 mmol; 1.00 eq.) is dissolved in dry acetonitrile (140.53 ml; 2.69 mol; 26.00 eq.) and N-bromosuccinimide is added (18.60 g; 103.48 mmol; 1.00 eq.). The reaction mixture is refluxed overnight until completion of the reaction. The solvent is removed under reduced pressure and the crude product is purified via column chromatography using heptane/ethyl acetate as eluent. 16.65 g of 5-bromo-2,4-dimethoxypyridine is isolated (74% yield of theory) together with 4.42 g of 3-bromo-2,4-dimethoxypyridine as side-product (20% yield of theory).

¹H NMR (500 MHz, DMSO-d₆) δ 8.15 (s, 1H), 6.53 (s, 1H), 3.89 (s, 4H), 3.83 (s, 3H).

Analogously, other derivatives are prepared in the same manner:

| | | | Yield |
|---|---|---|---|
| No. | Reactant | Product | [%] |
| 7a | 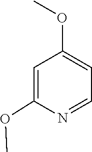 | 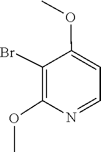 | 20 |
| 7b | 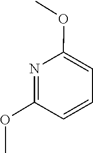 | 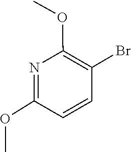 | 80 |
| 7c | 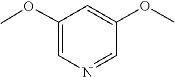 | 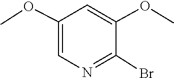 | 74 |
| 7d | 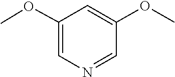 | 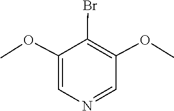 | 15 |

Compound 7a:

¹H NMR (500 MHz, Chloroform-d) δ 7.99 (dd, J=5.7, 2.1 Hz, 1H), 6.52 (dd, J=5.8, 2.0 Hz, 1H), 4.00 (d, J=1.9 Hz, 3H), 3.94 (d, J=2.1 Hz, 3H).

Compound 7c:

¹H NMR (500 MHz, Chloroform-d) δ 7.69 (dd, J=2.4 Hz), 6.73 (dd, J=2.4 Hz), 3.89 (s, 3H), 3.87 (s, 3H).

Compound 7d:

¹H NMR (500 MHz, Chloroform-d) δ 6.73 (s, 2H), 3.94 (s, 6H).

### Example 8

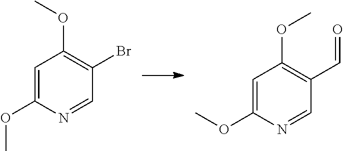

A 2M solution of n-butyllithium in hexane (10 ml, 20 mmol, 1.0 eq.) is added from a syringe over 1 min to a cooled (0° C.) and stirred mixture of a 2M solution of iso-propyl magnesium chloride in THF (5 ml, 10 mmol, 0.5 eq.) and anhydrous THF (52 ml) in a Schlenk flask under argon. The mixture is stirred for 5 min to give a yellow solution that is cooled to −2 to 0° C. A solution of 5-bromo-2,4-dimethoxypyridine (4.361 g, 20 mmol, 1.0 eq.) in 15 ml anhydrous THF is added from a syringe and the resulting solution is stirred for 45 min at −2 to 0° C. Then N,N-dimethylformamide (4.65 ml, 3.0 eq.) is added and the mixture is continuously stirred for 30 min at 0° C. and then for 45-60 min at r.t. Saturated aqueous NH₄Cl is added and the aqueous layer is separated and then extracted with EtOAc. The combined organic layers are dried over MgSO₄, filtered, concentrated in vacuo, and purified by recrystallization using a mixture of methanol and distilled water to give 2.57 g 4,6-dimethoxy-pyridine-3-carbaldehyde as a yellow crystalline solid (78% yield of theory).

¹H NMR (500 MHz, Chloroform-d) δ 10.23 (s, 1H), 8.54 (s, 1H), 6.22 (s, 1H), 4.00 (s, 3H), 3.93 (s, 3H).

Analogously, other derivatives are prepared in the same manner:

| | | | Yield |
|---|---|---|---|
| No. | Reactant | Product | [%] |
| 8a | 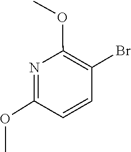 | 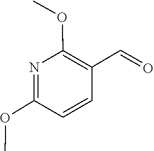 | 73 |
| 8b | 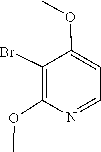 | 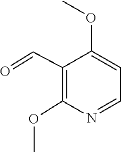 | 92 |
| 8c | 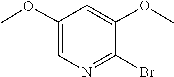 | 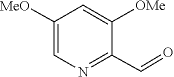 [SMILES: COc1cnc(C=O)c(OC)c1] | 34 |

Compound 8b:

¹H NMR (500 MHz, Chloroform-d) δ 10.42 (s, 1H), 8.19 (d, J=6.0 Hz, 1H), 6.58 (d, J=6.0 Hz, 1H), 4.02 (s, 3H), 3.95 (s, 4H).

Compound 8c:

¹H NMR (500 MHz, Chloroform-d) δ 10.20 (s, 1H), 8.09 (d, J=2.3 Hz, 1H), 6.81 (d, J=2.3 Hz, 1H), 3.97 (s, 4H), 3.96 (s, 4H).

### Example 9

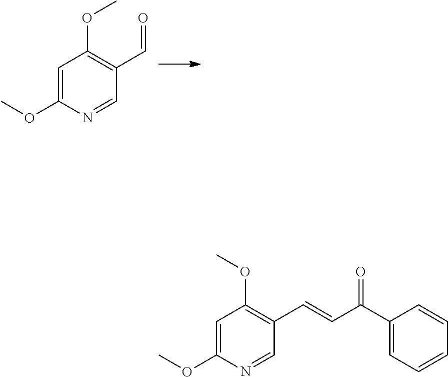 [SMILES: COc1cc(OC)c(C=O)cn1 ; COc1cc(OC)c(/C=C/C(=O)c2ccccc2)cn1]

To a solution of phenacyltriphenylphosphoniumbromide (21.71 g; 42.35 mmol; 1.20 eq.) and triethylamine (5.91 ml; 42.35 mmol; 1.20 eq.) in anhydrous tetrahydrofuran (143.62 ml; 50.00 eq.) is added 4,6-dimethoxypyridine-3-carbaldehyde (5.90 g; 35.30 mmol; 1.00 eq.) at 0° C. The solution is slowly warmed to RT and then refluxed until completion. Saturated aqueous NH₄Cl is added and the aqueous layer is separated and then extracted with EtOAc. The combined organic layers are dried over MgSO₄, filtered, concentrated in vacuo and purified via column chromatography on silica gel using heptane/ethyl acetate. 7.2 g of (E)-3-(4,6-dimethoxypyridin-3-yl)-1-phenylprop-2-en-1-one is isolated as a red solid (76% yield of theory).

¹H NMR (500 MHz, Chloroform-d) δ 8.29 (s, 1H), 8.02-7.99 (m, 2H), 7.85 (d, J=15.8 Hz, 1H), 7.69 (d, J=15.8 Hz, 1H), 7.60-7.55 (m, 1H), 7.50 (dd, J=8.2, 6.8 Hz, 2H), 6.26 (s, 1H), 3.97 (s, 3H), 3.95 (s, 3H).

Analogously, other derivatives are prepared in the same manner; R1 and R2 are starting materials; [P] is the product of the reaction:

| No. | | | Yield [%] |
|---|---|---|---|
| 9a | R1 | 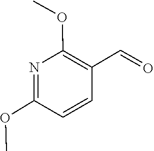 | |
| | R2 | 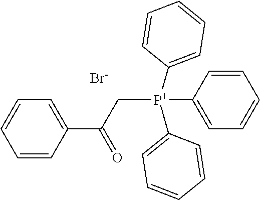 [SMILES: O=C(C[P+](c1ccccc1)(c1ccccc1)c1ccccc1)c1ccccc1 ; [Br-]] | |
| | [P] | 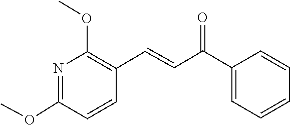 [SMILES: COc1ccc(/C=C/C(=O)c2ccccc2)c(OC)n1] | 78 |

[P] in example 9a:

¹H NMR (500 MHz, Chloroform-d) δ 8.03-7.98 (m, 2H), 7.92 (d, J=15.7 Hz, 1H), 7.79 (d, J=8.2 Hz, 1H), 7.58-7.54 (m, 2H), 7.49 (dd, J=8.3, 6.9 Hz, 2H), 6.37 (d, J=8.2 Hz, 1H), 4.06 (s, 3H), 3.97 (s, 3H).

### Example 10

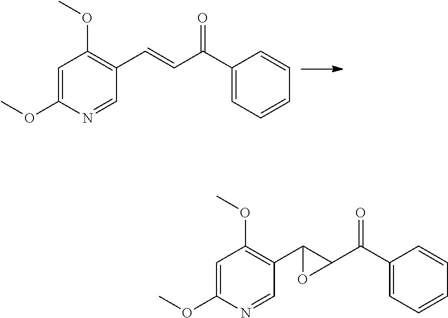 [SMILES: COc1cc(OC)c(/C=C/C(=O)c2ccccc2)cn1 ; COc1cc(OC)c(C2OC2C(=O)c2ccccc2)cn1]

(E)-3-(4,6-Dimethoxypyridin-3-yl)-1-phenylprop-2-en-1-one (7.76 g; 28.82 mmol; 1.00 eq.) is dissolved in methanol (46.75 ml; 40.00 eq.) and tetrahydrofuran (46.69 ml; 20.00 eq.) and cooled to 0° C. Sodium hydroxide pellets (1.15 g; 28.82 mmol; 1.00 eq.) dissolved in methanol are added followed by the addition of hydrogen peroxide (11.67 ml; 115.26 mmol; 4.00 eq.; 30 wt. % aqueous solution). After 0.5 h 0.25 ml THF is added to dissolve the precipitate. The reaction mixture is stirred for 3 h at 0° C., then at RT overnight. The mixture is diluted with saturated NaHCO₃ and water. The aqueous phase is extracted with tert-butyl methylether three times. The organic phase is washed with sat. NaCl and dried over MgSO₄. The crude product is purified via dissolving in hot THF followed by the addition of heptane. The formed precipitate is filtered and washed with heptane yielding 5.13 g 5-(3-benzoyloxiran-2-yl)-2,4-dimethoxypyridine (63% yield of theory).

¹H NMR (500 MHz, Chloroform-d) δ 8.07-8.03 (m, 2H), 7.99 (d, J=0.7 Hz, 1H), 7.65-7.60 (m, 1H), 7.53-7.48 (m, 2H), 6.22 (s, 1H), 4.30 (d, J=2.0 Hz, 1H), 4.22 (dd, J=2.0, 0.7 Hz, 1H), 3.94 (s, 3H), 3.84 (s, 3H).

### Example 11

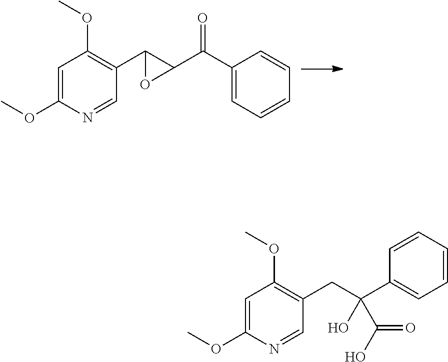 [SMILES: COc1cc(OC)c(C2OC2C(=O)c2ccccc2)cn1 ; COc1cc(OC)c(CC(O)(C(=O)O)c2ccccc2)cn1]

5-(3-Benzoyloxiran-2-yl)-2,4-dimethoxypyridine (5.13 g; 17.98 mmol; 1.00 eq.) is placed in a flask and ethanol (52.49 ml; 50.00 eq.) (saturated with sodium hydroxide) is added. The mixture is refluxed for 2 h. Then the solvent is removed under reduced pressure and water is added to the residue. The aqueous phase is extracted with tert-butyl methylether. Then the aqueous phase is acidified with 2M HCl to pH 2 followed by the dilution of an equal amount with saturated NaCl solution. Afterwards the solution is extracted 10 times with THF. The combined organic layers are dried over MgSO₄, filtered, concentrated in vacuo and purified via column chromatography on silica gel using dichloromethane/methanol/AcOH as the eluent. 3.9 g of 3-(4,6-dimethoxypyridin-3-yl)-2-hydroxy-2-phenylpropanoic acid is isolated (72% yield of theory).

¹H NMR (500 MHz, DMSO-d₆) δ 12.13 (br s, 1H), 7.90 (s, 1H), 7.47-7.4fd5 (m, 2H), 7.29 (dd, J=8.4, 6.7 Hz, 2H), 7.25-7.22 (m, 1H), 6.57 (s, 1H), 5.88 (br s, 1H), 3.97 (s, 3H), 3.72 (s, 3H), 3.28 (dd, J=14.5 Hz, 2H).

### Example 12

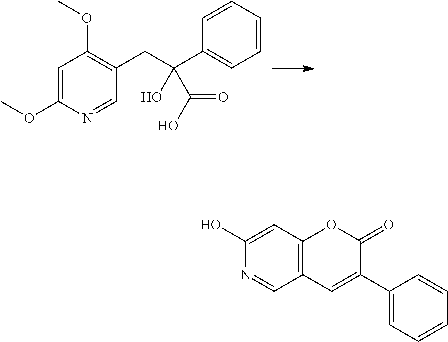 [SMILES: COc1cc(OC)c(CC(O)(C(=O)O)c2ccccc2)cn1 ; O=c1oc2cc(O)ncc2cc1-c1ccccc1]

3-(4,6-Dimethoxypyridin-3-yl)-2-hydroxy-2-phenylpropanoic acid (100.00 mg; 0.33 mmol; 1.00 eq.) is refluxed in a mixture of hydriodic acid (0.92 ml; 6.97 mmol; 21.14 eq.; 57 wt. %) and acetic acid (4.56 ml; 79.79 mmol; 242.00 eq.) for 1 h and is poured afterwards on ice. The aqueous phase is extracted four times with DCM and the combined organic phases are dried over MgSO₄. The crude product is purified via column chromatography on silica gel using DCM/methanol yielding 17 mg 7-hydroxy-3-phenyl-2H-pyrano[3,2-c]pyridin-2-one (22% yield of theory).

¹H NMR (500 MHz, DMSO-d₆) δ 8.13 (s, 1H), 7.98 (s, 1H), 7.60 (dd, J=7.3, 1.7 Hz, 2H), 7.43 (dd, J=8.3, 6.6 Hz, 2H), 7.40-7.36 (m, 1H), 6.11 (s, 1H).

### Example 13

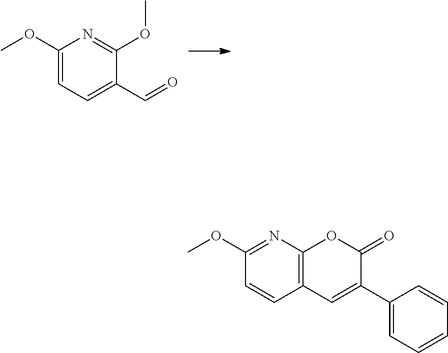 [SMILES: COc1ccc(C=O)c(OC)n1 ; COc1ccc2cc(-c3ccccc3)c(=O)oc2n1]

2,6-Dimethoxypyridine-3-carboxaldehyde (1.73 g; 10.35 mmol; 1.00 eq.) is refluxed together with potassium acetate (0.772 g; 7.87 mmol; 0.76 eq.), phenylacetic acid (1.42 g; 10.35 mmol; 1.00 eq.) and acetic anhydride (14.26 ml; 13.5 eq.) at 130° C. overnight. After cooling to RT, water is added and the mixture is stirred for 30 min. The precipitate is filtrated and recrystallized from 2-propanol. 0.435 g of 7-methoxy-3-phenyl-2H-pyrano[2,3-b]pyridin-2-one is isolated (19% yield of theory).

¹H NMR (500 MHz, DMSO-d₆) δ 8.27 (s, 1H), 8.15 (d, J=8.4 Hz, 1H), 7.73-7.68 (m, 2H), 7.49-7.44 (m, 2H), 7.43-7.38 (m, 1H), 6.92 (d, J=8.3 Hz, 1H), 3.96 (s, 3H).

Analogously, other derivatives are prepared in the same manner; R1 and R2 are starting materials; [P] is the product of the reaction:

| No. | | | Yield [%] |
|---|---|---|---|
| 13a | R1 | 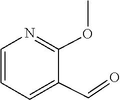 | |
| | R2 | 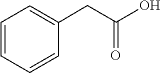 | |
| | [P] | 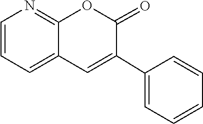 [SMILES: O=c1oc2ncccc2cc1-c1ccccc1] | 39 |
| 13b | R1 | 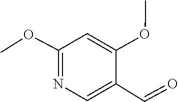 | |
| | R2 | 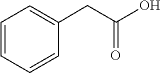 | |
| | [P] | 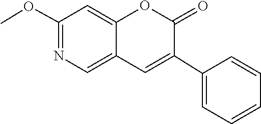 [SMILES: COc1cc2oc(=O)c(-c3ccccc3)cc2cn1] | 40 |
| 13c | R1 | 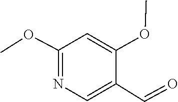 | |
| | R2 | 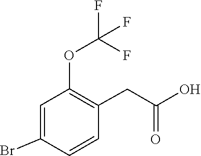 [SMILES: O=C(O)Cc1ccc(Br)cc1OC(F)(F)F] | |
| | [P] | 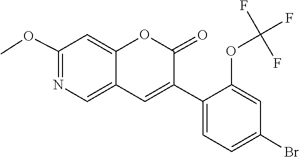 [SMILES: COc1cc2oc(=O)c(-c3ccc(Br)cc3OC(F)(F)F)cc2cn1] | 36 |
| 13d | R1 | 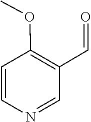 | |
| | R2 | 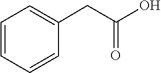 | |
| | [P] | 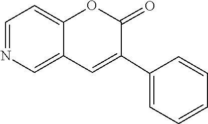 [SMILES: O=c1oc2ccncc2cc1-c1ccccc1] | 44 |
| 13e | R1 | 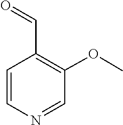 | |
| | R2 | 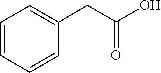 | |
| | [P] | 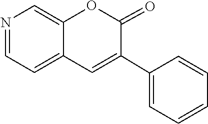 [SMILES: O=c1oc2cnccc2cc1-c1ccccc1] | <10 |
| 13f | R1 | 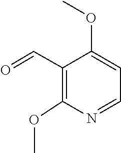 | |
| | R2 | 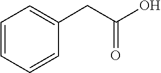 | |
| | [P1] | 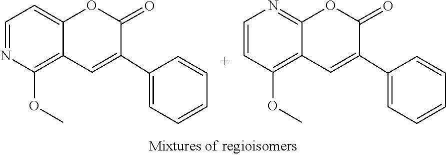 | 36 |
| 13g | R1 | 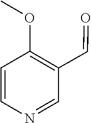 | |
| | R2 | 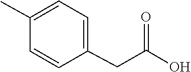 | |
| | [P] | 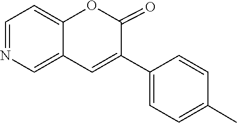 [SMILES: Cc1ccc(-c2cc3cnccc3oc2=O)cc1] | 57 |
| 13h | R1 | 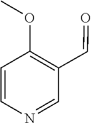 | |
| | R2 | 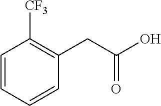 [SMILES: O=C(O)Cc1ccccc1C(F)(F)F] | |
| | [P] | 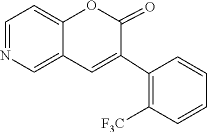 [SMILES: O=c1oc2ccncc2cc1-c1ccccc1C(F)(F)F] | <10 |
| 13i | R1 | 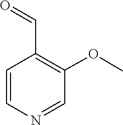 | |
| | R2 | 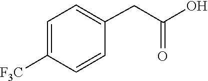 [SMILES: O=C(O)Cc1ccc(C(F)(F)F)cc1] | |
| | [P] | 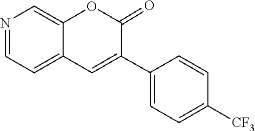 [SMILES: O=c1oc2cnccc2cc1-c1ccc(C(F)(F)F)cc1] | <10 |
| 13j | R1 | 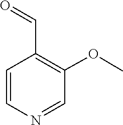 | |
| | R2 | 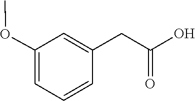 | |
| | [P] | 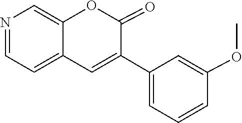 [SMILES: COc1cccc(-c2cc3ccncc3oc2=O)c1] | <10 |
| 13k | R1 | 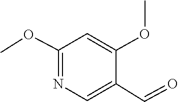 | |
| | R2 | 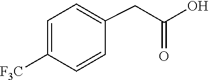 [SMILES: O=C(O)Cc1ccc(C(F)(F)F)cc1] | |
| | [P] | 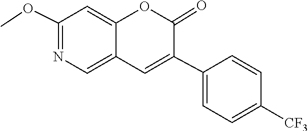 [SMILES: COc1cc2oc(=O)c(-c3ccc(C(F)(F)F)cc3)cc2cn1] | 39 |
| 13l | R1 | 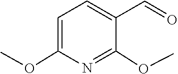 | |
| | R2 | 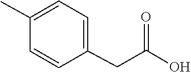 | |
| | [P] | 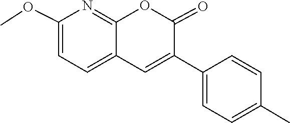 | <10 |
| 13m | R1 | 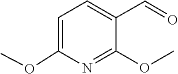 | |
| | R2 | 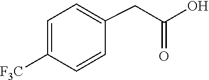 [SMILES: O=C(O)Cc1ccc(C(F)(F)F)cc1] | |
| | [P] | 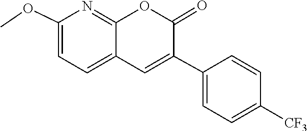 [SMILES: COc1ccc2cc(-c3ccc(C(F)(F)F)cc3)c(=O)oc2n1] | 33 |
| 13n | R1 | 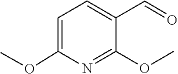 | |
| | R2 | 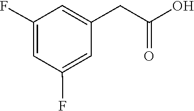 [SMILES: O=C(O)Cc1cc(F)cc(F)c1] | |
| | [P] | 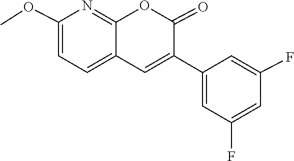 [SMILES: COc1ccc2cc(-c3cc(F)cc(F)c3)c(=O)oc2n1] | 32 |
| 13o | R1 | 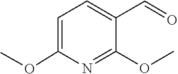 | |
| | R2 | 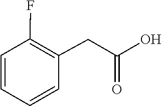 [SMILES: O=C(O)Cc1ccccc1F] | |
| | [P] | 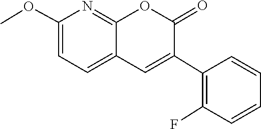 [SMILES: COc1ccc2cc(-c3ccccc3F)c(=O)oc2n1] | 23 |
| 13p | R1 | 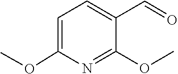 | |
| | R2 | 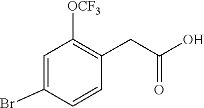 [SMILES: O=C(O)Cc1ccc(Br)cc1OC(F)(F)F] | |
| | [P] | 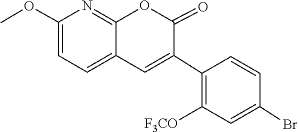 [SMILES: COc1ccc2cc(-c3ccc(Br)cc3OC(F)(F)F)c(=O)oc2n1] | 32 |
| 13q | R1 | 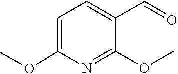 | |
| | R2 | 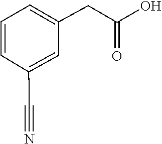 [SMILES: N#Cc1cccc(CC(=O)O)c1] | |
| | [P] | 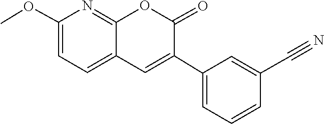 [SMILES: COc1ccc2cc(-c3cccc(C#N)c3)c(=O)oc2n1] | 34 |

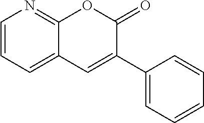 [SMILES: O=c1oc2ncccc2cc1-c1ccccc1]

¹H NMR (500 MHz, DMSO-d6) δ 8.54 (dd, J=4.8, 1.8 Hz, 1H), 8.31 (s, 1H), 8.27 (dd, J=7.6, 1.9 Hz, 1H), 7.77-7.66 (m, 2H), 7.55-7.40 (in, 4H).

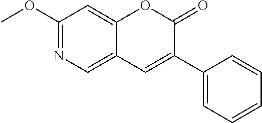 [SMILES: COc1cc2oc(=O)c(-c3ccccc3)cc2cn1]

¹H NMR (500 MHz, Chloroform-d) δ 8.41 (s, 1H), 7.79 (d, J=0.8 Hz, 1H), 7.71-7.61 (m, 2H), 7.47-7.38 (m, 3H), 6.65 (s, 1H), 4.03 (s, 3H).

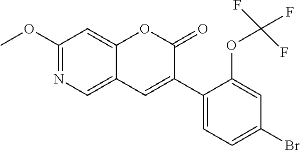 [SMILES: COc1cc2oc(=O)c(-c3ccc(Br)cc3OC(F)(F)F)cc2cn1]

¹H NMR (500 MHz, DMSO-d₆) δ 8.66 (s, 1H), 8.20 (s, 1H), 7.79-7.71 (m, 2H), 7.57 (d, J=8.7 Hz, 1H), 6.92 (s, 1H), 3.96 (s, 3H).

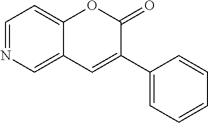 [SMILES: O=c1oc2ccncc2cc1-c1ccccc1]

¹H NMR (500 MHz, DMSO-d₆) 8.98 (s, 1H), 8.67 (d, J=5.7 Hz, 1H), 8.33 (s, 1H), 7.75-7.70 (m, 2H), 7.51-7.43 (m, 4H).

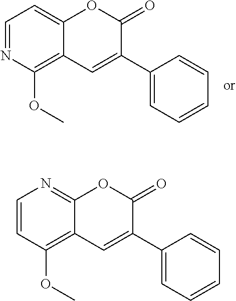 [SMILES: COc1nccc2oc(=O)c(-c3ccccc3)cc12 ; COc1ccnc2oc(=O)c(-c3ccccc3)cc12]

¹H NMR (500 MHz, DMSO-d₆) δ 8.13-8.10 (m, 1H), 7.97 (d, J=5.8 Hz, 1H), 7.52 (s, 1H), 7.19 (dd, J=5.3, 1.9 Hz, 3H), 6.98-6.96 (m, 2H), 6.63 (d, J=5.9 Hz, 1H), 3.56 (s, 5H), 3.51 (s, 5H).

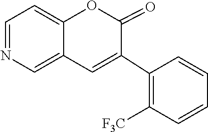 [SMILES: O=c1oc2ccncc2cc1-c1ccccc1C(F)(F)F]

¹H NMR (500 MHz, DMSO-d₆) δ 8.97 (s, 1H), 8.73 (d, J=5.7 Hz, 1H), 8.17 (s, 1H), 7.88 (dd, J=8.0, 1.3 Hz, 1H), 7.81-7.78 (m, 1H), 7.71 (tt, J=7.7, 1.1 Hz, 1H), 7.60 (d, J=7.5 Hz, 1H), 7.54 (d, J=5.7 Hz, 1H).

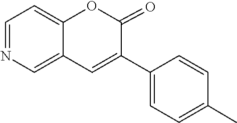 [SMILES: Cc1ccc(-c2cc3cnccc3oc2=O)cc1]

¹H NMR (500 MHz, DMSO-d₆) δ 8.97 (s, 1H), 8.66 (d, J=5.7 Hz, 1H), 8.30 (s, 1H), 7.64 (d, J=8.2 Hz, 2H), 7.47 (d, J=5.7 Hz, 1H), 7.30 (d, J=7.9 Hz, 2H), 2.36 (s, 3H).

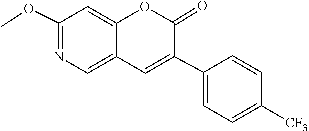 [SMILES: COc1cc2oc(=O)c(-c3ccc(C(F)(F)F)cc3)cc2cn1]

¹H NMR (500 MHz, DMSO-d₆) δ 8.67 (s, 1H), 8.39 (s, 1H), 7.73 (d, J=8.2 Hz, 2H), 7.40 (d, J=8.0 Hz, 2H), 6.90 (s, 1H), 3.97 (s, 3H).

¹⁹F NMR (470 MHz, DMSO-d₆) δ −60.9.

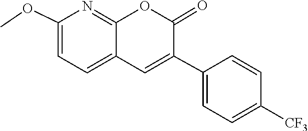 [SMILES: COc1ccc2cc(-c3ccc(C(F)(F)F)cc3)c(=O)oc2n1]

¹H NMR (500 MHz, DMSO-d₆) δ 8.41 (s, 1H), 7.95 (d, J=8.1 Hz, 1H), 7.84 (d, J=8.3 Hz, 1H), 7.78 (d, J=8.0 Hz, 2H), 7.39 (d, J=7.9 Hz, 2H), 3.95 (s, 3H).

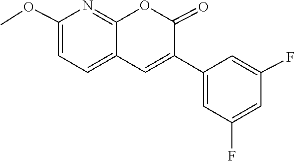 [SMILES: COc1ccc2cc(-c3cc(F)cc(F)c3)c(=O)oc2n1]

¹H NMR (500 MHz, DMSO-d₆) δ 8.46 (s, 1H), 8.14 (d, J=8.4 Hz, 1H), 7.51 (d, 2H, J=9.0 Hz), 7.31 (tt, J=9.3, 2.4 Hz, 1H), 6.95 (d, J=8.4 Hz, 1H), 3.97 (s, 3H).

¹⁹F NMR (470 MHz, DMSO-d₆) δ −110.0.

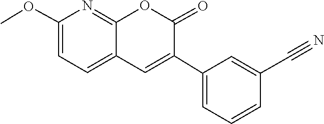 [SMILES: COc1ccc2cc(-c3cccc(C#N)c3)c(=O)oc2n1]

¹H NMR (500 MHz, DMSO-d₆) δ 8.43 (s, 1H), 8.17 (t, J=1.7 Hz, 1H), 8.15 (d, J=8.4 Hz, 1H), 8.10-8.06 (m, 1H), 7.89 (dt, J=7.8, 1.3 Hz, 1H), 7.69 (t, J=7.9 Hz, 1H), 6.95 (d, J=8.4 Hz, 1H), 3.97 (s, 3H).

### Example 14

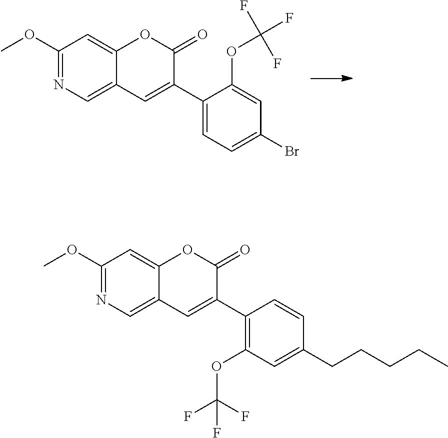 [SMILES: COc1cc2oc(=O)c(-c3ccc(Br)cc3OC(F)(F)F)cc2cn1 ; CCCCCc1ccc(-c2cc3cnc(OC)cc3oc2=O)c(OC(F)(F)F)c1]

0.446 g (1.7 mmol) of 3-[4-Bromo-2-(trifluoromethoxy)phenyl]-7-methoxy-2H-pyrano[3,2-c]pyridin-2-one), 0.152 g (1.29 mmol; 1.2 equiv.) of n-pentylboronic acid and 0.524 g (2.27 mmol; 2.1 equiv.) of tri-potassium phosphate trihydrate are dissolved in 3 ml of toluene and degassed three times. 9.92 mg (0.043 mmol; 0.04 equiv.) of Palladium(II) acetate and 35.92 mg (0.086 mmol; 0.08 equiv.) of 2-dicyclohexylphosphino-2′,6′-dimethoxy-1,1′-biphenyl are added. The reaction mixture is subsequently stirred at 90° C. for 2 h under a protective-gas atmosphere. The cooled solution is diluted with 2-methyl tetrahydrofuran and water and the phases are separated. The aqueous phase is extracted three times with 2-methyl tetrahydrofuran and the organic phase is dried over MgSO₄ and evaporated. The crude product is purified by column chromatography on silica gel. 0.26 g of 7-methoxy-3-[4-pentyl-2-(trifluoromethoxy)phenyl]-2H-pyrano[3,2-c]pyridin-2-one is isolated (60% of theory).

¹H NMR (500 MHz, Chloroform-d) δ 8.38 (s, 1H), 7.71 (d, J=0.8 Hz, 1H), 7.38 (d, J=7.7 Hz, 1H), 7.19-7.16 (m, 2H), 6.66 (s, 1H), 4.03 (s, 3H), 2.69-2.64 (m, 2H), 1.65 (dq, J=9.6, 7.3 Hz, 2H), 1.35 (tt, J=6.7, 2.4 Hz, 4H), 0.92-0.90 (m, 3H).

Analogously, other derivatives are prepared in the same manner; R1 and R2 are starting materials; [P] is the product of the reaction:

| | | | Yield |
|---|---|---|---|
| No. | | | [%] |
| 14A | R1 | 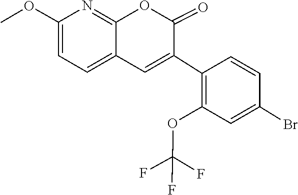 [SMILES: COc1ccc2cc(-c3ccc(Br)cc3OC(F)(F)F)c(=O)oc2n1] | |
| | R2 | 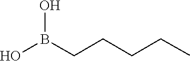 | |
| | [P] | 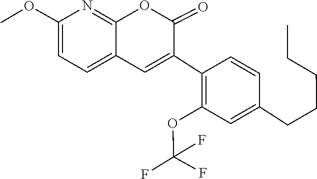 [SMILES: CCCCCc1ccc(-c2cc3ccc(OC)nc3oc2=O)c(OC(F)(F)F)c1] | 89 |

### Example 15

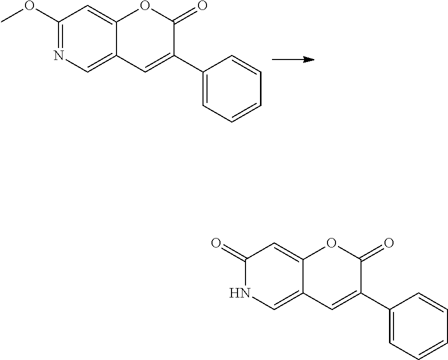 [SMILES: COc1cc2oc(=O)c(-c3ccccc3)cc2cn1 ; O=c1cc2oc(=O)c(-c3ccccc3)cc2c[nH]1]

To a solution of 7-methoxy-3-phenyl-2H-pyrano[3,2-c]pyridin-2-one (0.5 g; 1.974 mmol) in 4 ml anhydrous N,N-dimethylformamide is added anhydrous lithium chloride (0.418 g; 9.87 mmol; 5.0 equiv.) and para-toluenesulfonic acid monohydrate (1.88 g; 9.87 mmol; 5.0 equiv.). The reaction mixture is stirred at 180° C. for 2 h. Water is added at RT and a precipitate is formed, filtrated and the filtrated is again extracted three times with DCM. The combined organic solids are dried in vacuo yielding 0.376 g of 3-phenyl-2H,6H,7H-pyrano[3,2-c]pyridine-2,7-dione (80% yield of theory).

¹H NMR (500 MHz, DMSO-d₆) δ 12.34 (br s, 1H), 8.13 (s, 1H), 7.98 (s, 1H), 7.63-7.57 (m, 2H), 7.46-7.41 (m, 2H), 7.41-7.36 (m, 1H), 6.13 (s, 1H).

Analogously, other derivatives are prepared in the same manner:

| | | | Yield |
|---|---|---|---|
| No. | Reactant | Product | [%] |
| 15a | 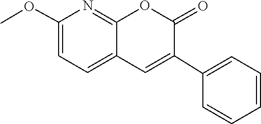 [SMILES: COc1ccc2cc(-c3ccccc3)c(=O)oc2n1] | 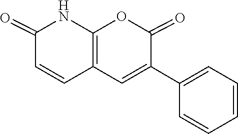 [SMILES: O=c1ccc2cc(-c3ccccc3)c(=O)oc2[nH]1] | 85 |
| 15b | 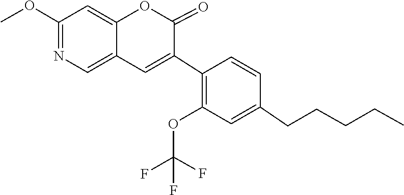 [SMILES: CCCCCc1ccc(-c2cc3cnc(OC)cc3oc2=O)c(OC(F)(F)F)c1] | 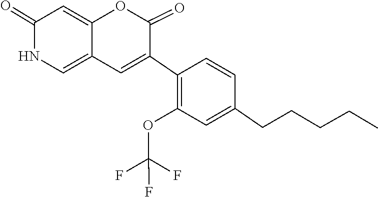 [SMILES: CCCCCc1ccc(-c2cc3c[nH]c(=O)cc3oc2=O)c(OC(F)(F)F)c1] | 80 |
| 15c | 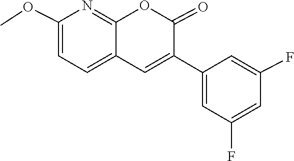 [SMILES: COc1ccc2cc(-c3cc(F)cc(F)c3)c(=O)oc2n1] | 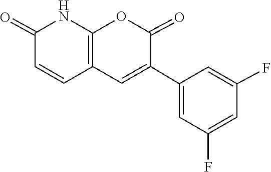 [SMILES: O=c1ccc2cc(-c3cc(F)cc(F)c3)c(=O)oc2[nH]1] | 89 |
| 15d | 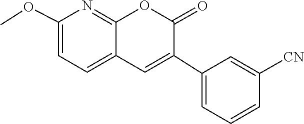 [SMILES: COc1ccc2cc(-c3cccc(C#N)c3)c(=O)oc2n1] | 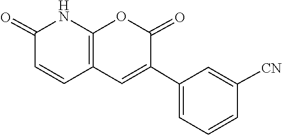 [SMILES: N#Cc1cccc(-c2cc3ccc(=O)[nH]c3oc2=O)c1] | 90 |

Compound 15b:

¹H NMR (500 MHz, DMSO-d₆) δ 12.40 (s, 1H), 8.16 (s, 1H), 7.85 (s, 1H), 7.41 (d, J=7.8 Hz, 1H), 7.30 (dd, J=7.9, 1.6 Hz, 1H), 7.25 (d, J=2.2 Hz, 1H), 6.16 (s, 1H), 2.66 (t, J=7.7 Hz, 2H), 1.60 (p, J=7.5 Hz, 2H), 1.30 (ddq, J=16.4, 7.3, 4.5, 2.6 Hz, 4H), 0.86 (t, J=6.9 Hz, 3H).

Compound 15c:

¹H NMR (500 MHz, DMSO-d₆) δ 12.34 (br s, 1H), 8.40 (s, 1H), 8.04 (d, J=8.4 Hz, 1H), 7.52-7.47 (m, 2H), 7.29 (tt, J=9.3, 2.4 Hz, 1H), 6.70 (br s, 1H).

Compound 15d:

¹H NMR (500 MHz, DMSO-d₆) δ 12.28 (br s, 1H), 8.38 (s, 1H), 8.15 (t, J=1.7 Hz, 2H), 8.08-8.03 (m, 1H), 7.86 (dt, J=7.7, 1.4 Hz, 1H), 7.68 (t, J=7.8 Hz, 1H) 6.71 (br s, 1H).

### Example 16

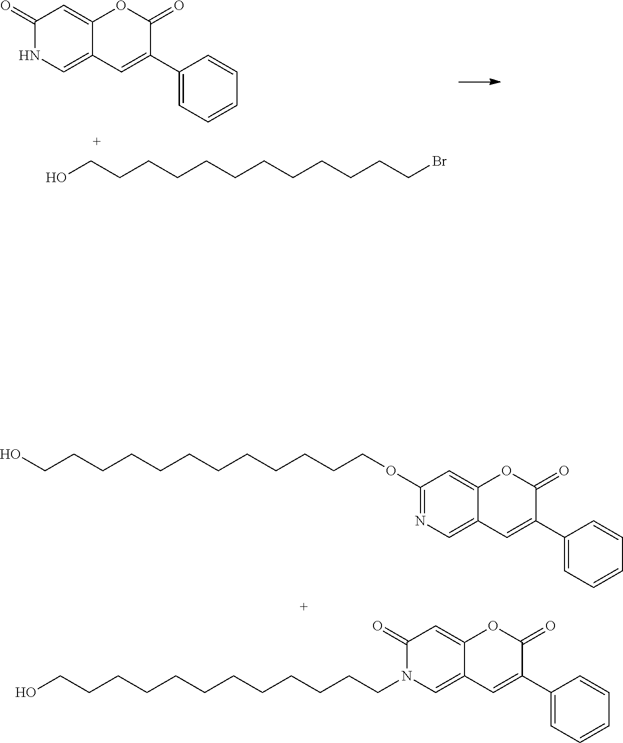

3-Phenyl-2H,6H,7H-pyrano[3,2-c]pyridine-2,7-dione (2.0 g, 8.36 mmol, 1.00 eq.) is refluxed with potassium carbonate (4.67 g, 33.00 mmol, 4.00 eq.) and 12-bromo-dodecan-1-ol (2.337 g, 8.79 mmol, 1.05 mmol) in N,N-dimethylformamide (35 ml) for minimum 2 d. The suspension is filtered, and the solvent of the filtrate is evaporated. The crude residue is purified via column chromatography using DCM/methanol and/or heptane/ethyl acetate as an eluent. 0.628 g 7-[(12-hydroxydodecyl)oxy]-3-phenyl-2H-pyrano[3,2-c]pyridin-2-one (18% yield of theory) is isolated together with 1.95 g of 6-(12-hydroxydodecyl)-3-phenyl-2H,6H,7H-pyrano[3,2-c]pyridine-2,7-dione (55% yield of theory) as a byproduct.

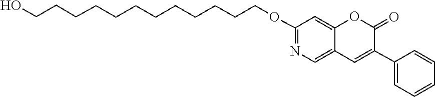 [SMILES: O=c1oc2cc(OCCCCCCCCCCCCO)ncc2cc1-c1ccccc1]

¹H NMR (500 MHz, DMSO-d₆) δ 8.61 (s, 1H), 8.24 (s, 1H), 7.69 (d, J=7.9 Hz, 2H), 7.48-7.40 (m, 3H), 6.82 (s, 1H), 4.35-4.30 (m, 3H), 3.38-3.34 (m, 2H), 1.73 (p, J=6.8 Hz, 2H), 1.39 (dq, J=12.5, 6.3, 5.0 Hz, 4H), 1.34-1.21 (m, 14H).

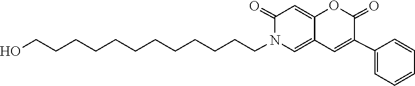 [SMILES: O=c1oc2cc(=O)n(CCCCCCCCCCCCO)cc2cc1-c1ccccc1]

¹H NMR (500 MHz, DMSO-d₆) δ 8.44 (s, 1H), 7.91 (s, 1H), 7.61-7.59 (m, 2H), 7.46-7.43 (m, 2H), 7.40-7.37 (m, 1H), 6.21 (s, 1H), 4.31 (t, J=5.1 Hz, 1H), 3.97 (t, J=7.3 Hz, 2H), 3.38-3.34 (m, 2H), 1.67 (q, J=7.1 Hz, 2H), 1.38 (p, J=6.8 Hz, 2H), 1.25 (d, J=21.5 Hz, 16H).

Analogously, other derivatives are prepared in the same manner; R1 and R2 are starting materials; [P] is the product of the reaction:

| | | | Yield |
|---|---|---|---|
| No. | | | [%] |
| 16a | R1 | 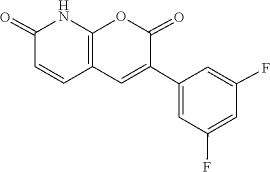 [SMILES: O=c1ccc2cc(-c3cc(F)cc(F)c3)c(=O)oc2[nH]1] | |
| | R2 | 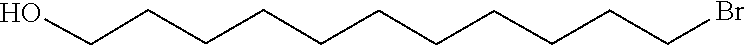 [SMILES: OCCCCCCCCCCCBr] | |
| | [P] | 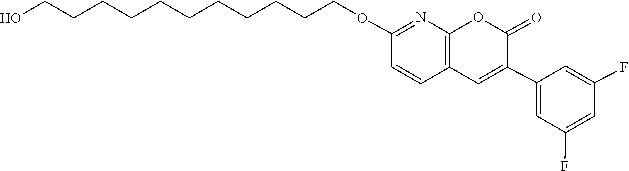 [SMILES: O=c1oc2nc(OCCCCCCCCCCCO)ccc2cc1-c1cc(F)cc(F)c1] | 24 |
| 16b | R1 | 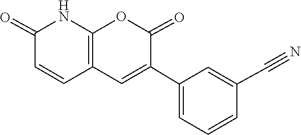 [SMILES: N#Cc1cccc(-c2cc3ccc(=O)[nH]c3oc2=O)c1] | |
| | R2 | 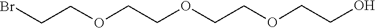 [SMILES: OCCOCCOCCOCCBr] | |
| | [P] | 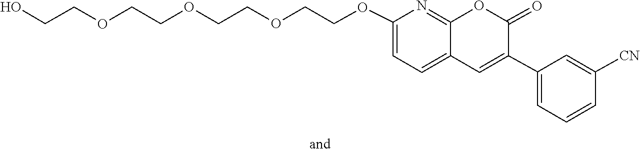 [SMILES: N#Cc1cccc(-c2cc3ccc(OCCOCCOCCOCCO)nc3oc2=O)c1] | 19 |
| | | 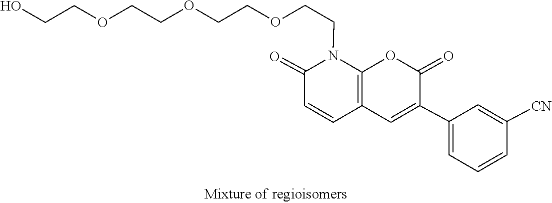 [SMILES: N#Cc1cccc(-c2cc3ccc(=O)n(CCOCCOCCOCCO)c3oc2=O)c1] | |

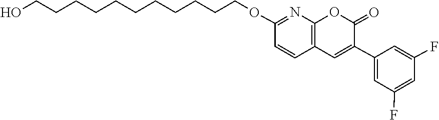 [SMILES: O=c1oc2nc(OCCCCCCCCCCCO)ccc2cc1-c1cc(F)cc(F)c1]

¹H NMR (500 MHz, DMSO-d₆) δ 8.45 (s, 1H), 8.12 (d, J=8.4 Hz, 1H), 7.51 (dd, J=9.0, 2.2 Hz, 2H), 7.31 (tt, J=9.3, 2.4 Hz, 1H), 6.92 (d, J=8.4 Hz, 1H), 4.35 (t, J=6.6 Hz, 2H), 4.31 (t, J=5.1 Hz, 1H), 3.36 (td, J=6.6, 5.2 Hz, 2H), 1.75 (p, J=6.8 Hz, 2H), 1.39 (p, J=7.2, 6.3 Hz, 4H), 1.34-1.21 (m, 12H).

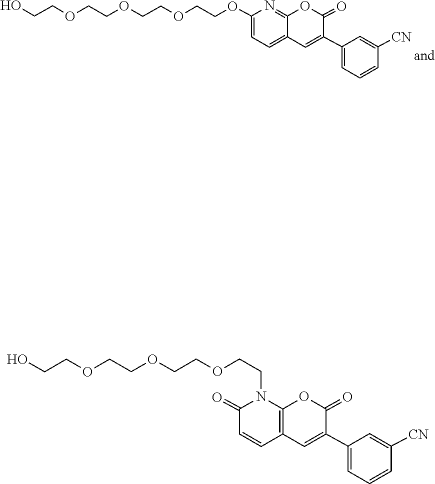 [SMILES: N#Cc1cccc(-c2cc3ccc(OCCOCCOCCOCCO)nc3oc2=O)c1 ; N#Cc1cccc(-c2cc3ccc(=O)n(CCOCCOCCOCCO)c3oc2=O)c1]

¹H NMR (500 MHz, DMSO-d₆) δ 7.97 (s, 1H), 7.85-7.79 (m, 3H), 7.72 (d, J=1.7 Hz, 1H), 7.61 (t, J=7.9 Hz, 1H), 7.53-7.49 (m, 1H), 4.59-4.54 (m, 5H), 4.48-4.43 (m, 2H), 4.36-4.33 (m, 2H), 4.32-4.27 (m, 2H), 3.76 (t, J=4.8 Hz, 2H), 3.41 (dt, J=10.5, 5.3 Hz, 4H).

### Example 17

Characterization of compounds/monomers according to the invention through melting points and compared to a reference material Ref-[1] as disclosed in M. Schraub et al, European Polymer Journal 51 (2014) 21-27 describes the photochemistry of 3-phenyl-coumarin containing polymethacrylates.

| Compound | m.p. [° C.] |
|---|---|
| 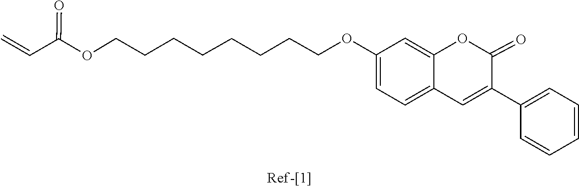 [SMILES: C=CC(=O)OCCCCCCCCOc1ccc2cc(-c3ccccc3)c(=O)oc2c1] | 100 |
| 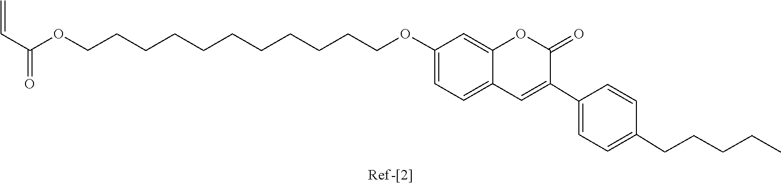 [SMILES: C=CC(=O)OCCCCCCCCCCCOc1ccc2cc(-c3ccc(CCCCC)cc3)c(=O)oc2c1] | 82-83.5 |
| 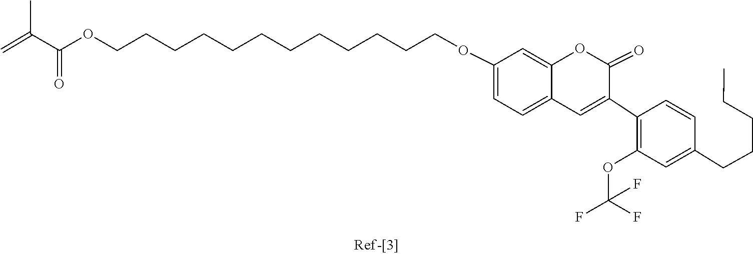 [SMILES: C=C(C)C(=O)OCCCCCCCCCCCCOc1ccc2cc(-c3ccc(CCCCC)cc3OC(F)(F)F)c(=O)oc2c1] | 64.6-68.5 |
| 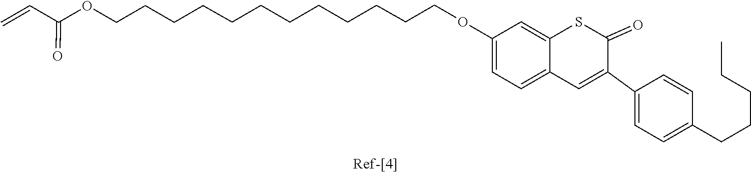 [SMILES: C=CC(=O)OCCCCCCCCCCCCOc1ccc2cc(-c3ccc(CCCCC)cc3)c(=O)sc2c1] | 57.5-59.5 |
| 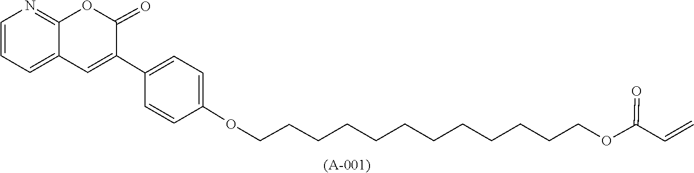 | 109 |
| 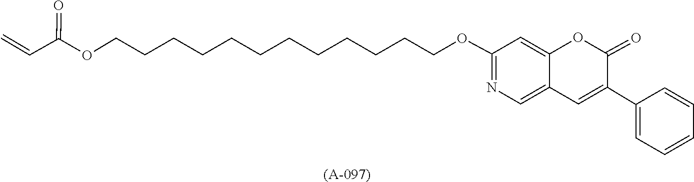 | 88 |
| 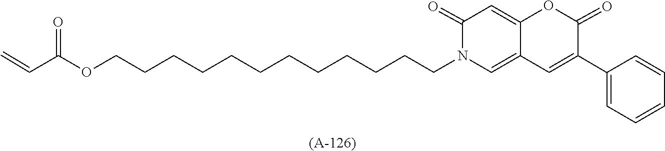 | 87 |
| 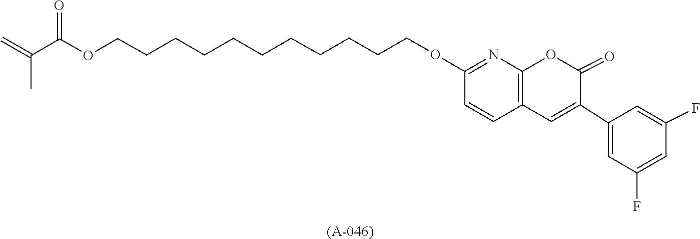 | 102 |

Example of Application:

### Example 18—General Polymerization Procedure to Produce Bulk Copolymer

For production of bulk polymer blanks, the monomers are melted under vacuum and with amounts and further components as indicated in table 3 below.

The compositions shown in table 3 are formulated in the same way as described in the following mixing all compounds together while stirring. A heating bath for the stirring is used, where necessary. These formulations are also the basis for the copolymers compared in table 5.

TABLE 3
Compositions-Amount of components is given in mol-%, the
amount of the respective chosen radical initiator adds to 100 mol-%:

| | Monomer | | | | | |
|---|---|---|---|---|---|---|
| | (photo- | | | | | |
| | active | | | | | |
| Exam- | chromo- | mol % | mol % | mol % | mol % | mol % |
| ple | phore) | monomer | n-BuAc | EGDMA | HEMA | UV-Abs. |
| 18-1 | Ref-[1] | 18.4 | 65.8 | 0.9 | 14.1 | 0.6 |
| 18-2 | Ref-[2] | 18.6 | 77.2 | 2.5 | — | — |
| 18-3 | Ref-[3] | 25.2 | 50.4 | 2.1^{a} | 21.2 | 0.4 |
| 18-4 | Ref-[4] | 27.8 | 66.7^{b} | 4.5^{c} | — | — |
| 18-5 | A-001 | 22.5 | 70.4 | 5.4 | — | 0.6 |
| 18-6 | A-097 | 22.5 | 70.4 | 5.4 | — | 0.6 |
| 18-7 | A-126 | 22.5 | 70.4 | 5.4 | — | 0.6 |
| 18-8 | A-046 | 50.5 | 42.4^{a} | 5.1 | — | — |

n-BuAc = n-butylacrylate;
EGDMA = Ethylene glycol dimethacrylate;
HEMA = Hydroxyethylmethacrylate;
EtMAc = Ethylmethacrylate.
^{a}Octadecane-1,18-diyl diacrylate is used in instead of EGDMA.
^{b}8-methylnonyl acrylate is used instead of n-butylacrylate.
^{c}Poly(ethylene glycol) diacrylate (Mₙ 250) is used instead of EGDMA.

Exemplarily, a composition of 12-({2-oxo-3-phenyl-2H-pyrano[3,2-c]pyridin-7-yl}oxy)dodecyl prop-2-enoate (A-097) (0.2 g, 0.42 mmol, 22.5 mol %), n-butyl acrylate (0.17 g, 1.31 mmol, 70.4 mol %) and ethylene glycol dimethacrylate (0.02 g, 0.10 mmol, 5.4 mol %) (EGDMA) as crosslinker is well mixed under stirring using gentle heat and degassed by three freeze pump-thaw cycles. Appropriate amounts (0.02-0.12 equiv.) of a radical initiator (e. g. 1,1′-(3,3,5-trimethylcyclohexylidene)bis[2-(1,1-dimethylethyl)-peroxide [Luperox® 231] or 2-[(E)-2-(1-cyano-1-methylethyl)diazen-1-yl]-2-methylpropanenitrile) are added.

Two glass plates are coated with a polyethylene sheet and a 1 mm thick cell is created between the polyethylene sheets using a silicone rubber gasket. The coated faces of the glass sheets are clipped together using spring clips with a syringe needle being placed between the gasket and the polyethylene sheets. The cavity is then filled with the above formulation or with formulations as indicated further in table 3 through the needle using a gastight syringe. Once the cavity is filled the syringe needle is removed, a final clip is used to seal the mould and the assembly is placed in an oven.

The polymerization temperature is between 60° C. and 180° C. and the individual polymerization conditions are chosen for the respective initiators, which can be extracted for the skilled person from the formulation mixture. The moulds are allowed to cool to room temperature before the polymer plate is removed from the mould.

Refractive index change is induced by irradiation at 340-365 nm. The refractive indices (n) of the blanks at 590 nm are measured on Schmidt+Haensch AR12 before and after irradiation. The following table shows the refractive indice after irradiation as well as the change in refractive index (max. Δn).

The phase transition temperatures are determined with a TA Instruments Q2000 differential scanning calorimeter during heating in the second heating run with 20 K/min from −100° C. to 200° C. in a hermetic aluminium pans.

TABLE 4
Results after irradiation and
refractive index change:

| Example | T_{g} [° C.] | n_{D}, _{35° C.} | Δn |
|---|---|---|---|
| 18-1 | 8.0 | 1.536 | 0.013 |
| 18-2 | −5.09 | 1.525 | 0.011 |
| 18-3 | 9.0 | 1.523 | 0.009 |
| 18-4 | −1.6 | 1.523 | 0.006 |
| 18-5 | 22.3 | 1.572 | 0.024 |
| 18-6 | 17.0 | 1.560 | 0.035 |
| 18-7 | 24.0 | 1.537 | 0.015 |
| 18-8 | 37.6 | 1.524 | 0.019 |

The results of the application examples 18-5 to 18-8 show a significant refractive index change after irradiation.

Moreover, the addition of the nitrogen within the photoactive chromophore according to the invention has a significant impact on the optical properties over prior art compounds. Homopolymers of representative monomers of the prior art (Ref-[1], Ref-[2], Ref-[3] and Ref-[4]) and the monomers A-001, A-097, A-126 and A-046 are prepared and compared to explain this effect.

Ref-[2] is a monomer as disclosed in WO2017032442 (compound (M-1)).

Ref-[3] is a monomer as disclosed in WO2017032442 (compound (M-42)).

Ref-[4] is a monomer as disclosed in WO2017032442 (compound (M-53)).

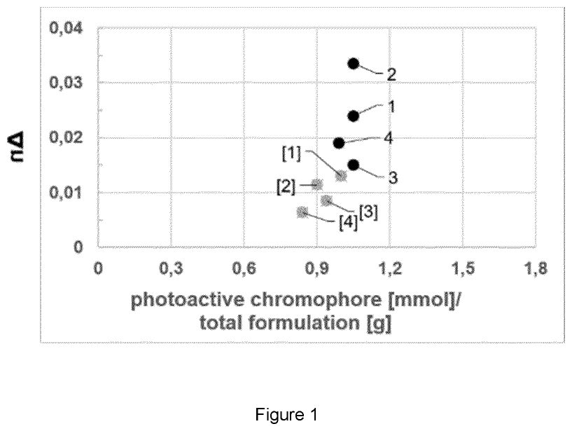

This effect and the direct comparison can be seen from the data in Table 5 and is visualized in FIG. 1.

TABLE 5
Comparison of copolymers of prior art compounds Ref-[1], Ref-[2], Ref-[3] and Ref-[4] with copolymers of representative A-001, A-097, A-126
and A-046 resulting from the described formulations of table 3 with regard to chromophore concentration and the observed refractive index change:

| | | | mmol |
|---|---|---|---|
| | | | photo- |
| | | | active |
| | | | chromo- |
| mono- | Copolymer comprising the monomeric | Δn | phere/ |
| mer | photoactive chromophore | max | g^{a} |
| Ref- [1] | 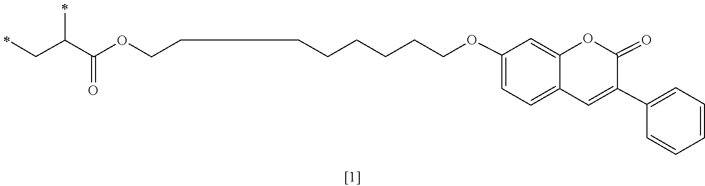 | 0.013 | 1.0 |
| Ref- [2] | 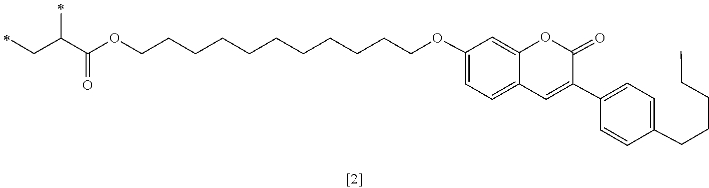 | 0.011 | 0.9 |
| Ref- [3] | 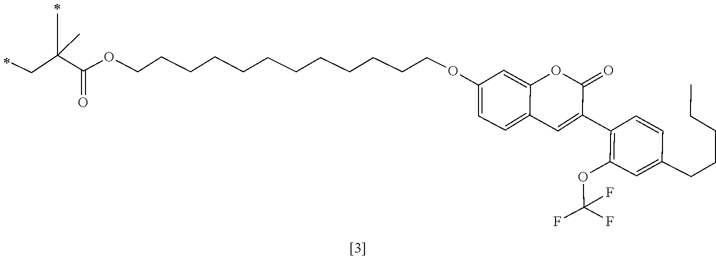 | 0.009 | 0.94 |
| Ref- [4] | 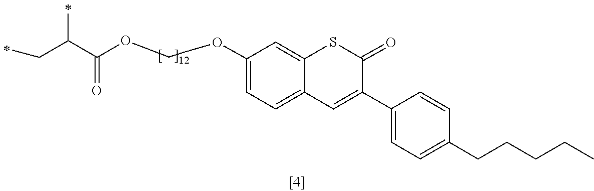 | 0.006 | 0.84 |
| A-001 | 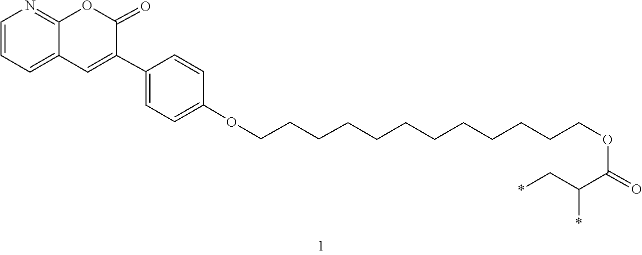 [SMILES: *CC(*)C(=O)OCCCCCCCCCCCCOc1ccc(-c2cc3cccnc3oc2=O)cc1] | 0.024 | 1.05 |
| A-097 | 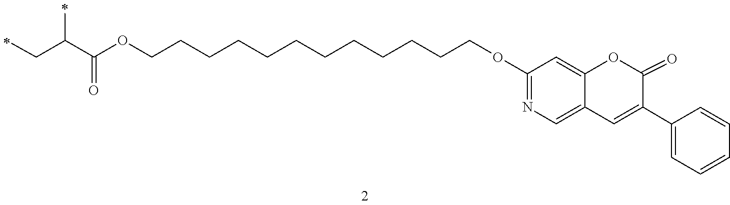 | 0.035 | 1.05 |
| A-126 | 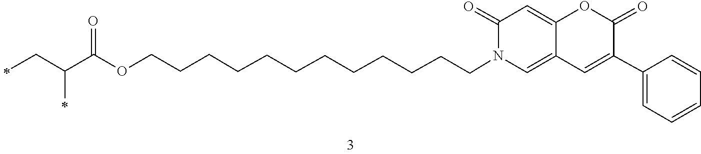 | 0.015 | 1.05 |
| A-046 | 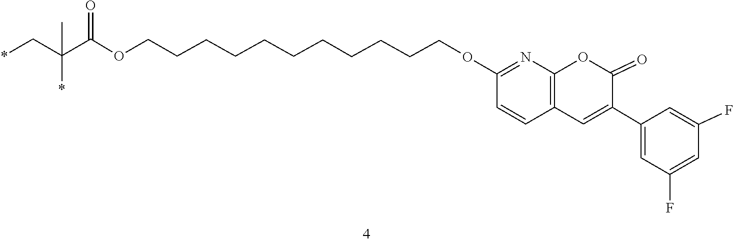 [SMILES: *CC(*)(C)C(=O)OCCCCCCCCCCCOc1ccc2cc(-c3cc(F)cc(F)c3)c(=O)oc2n1] | 0.019 | 0.99 |

^{a}means ratio photoactive chromophore (in mmol) per total amount of formulation mixture (in gram).

The ratio photoactive chromophore (in mmol) per total amount of formulation mixture (in gram) is calculated as follows: The compositions according to table 3 consist of various components as described. The weight in grams of all said components in each formulation are summed up. Then the amount of the individual photoactive chromophore in mmol in each formulation (amount of Ref-[1], Ref-[2], Ref-[3], Ref-[4], A-001, A-097, A-126 or A-046) is divided by the prior calculated sum for said formulation comprising said individual photoactive chromophore. The quotient of this mathematic operation is the above mentioned ratio photoactive chromophore (in mmol) per total amount of formulation mixture (in gram).

The advantage as described before of compounds as described in Table 5 is visualized in FIG. 1 where the refractive index change Δn is plotted versus the amount of mmol photoactive chromophore per total amount of the formulation in grams. FIG. 1 shows that the examples according to the invention show a higher refractive index change and partially increased higher refractive starting indices and the total value of refractive index change per mmol photoactive chromophore is much higher compared to the reference materials used.

1. An ophthalmic device or a precursor article for manufacturing an ophthalmic device comprising at least one polymerized compound of formula (I) 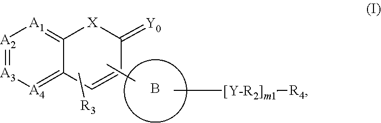
wherein
the divalent group 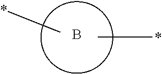
is selected from the group of formulae (B-1), (B-2), (B-3), (B-4) or (B-5) 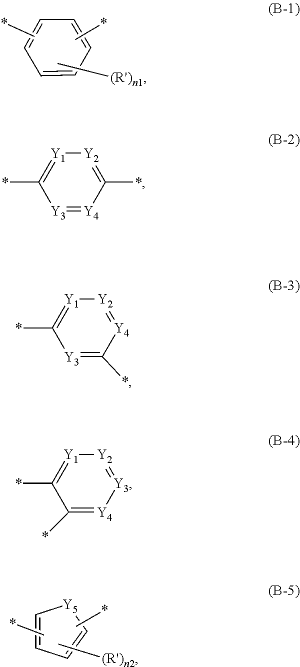
the asterisk * indicates the linkage to the remainder of formula (I);
Y₁, Y₂, Y₃, Y₄ are each independently of each other CR′ or N, provided that only one of Y₁, Y₂, Y₃ and Y₄ is N and the others are CR′;
Y₅ is O, S or NR_{B};
R_{B} is at each occurrence independently selected from a linear or branched alkyl group having 1 to 10 C atoms or a linear or branched partially or fully fluorinated alkyl group having 1 to 10 C atoms;
X is O or S;
Y₀ is O or S;
A₁, A₂, A₃, A₄ are each independently of each other N, CR″ or C—Y—R₂—R₁, provided that in case m1 is 1 only one of A₁, A₂, A₃ and A₄ is N and the others are CR″ and provided that in case m1 is 0, only one of A₁, A₂, A₃ and A₄ is N, only one of A₁, A₂, A₃ and A₄ is C—Y—R₂—R₁ and the others are each independently CR″; or
in case of m1 is 0, adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″;
Y is independently of each other O, S, SO₂, or a bond;
m1 is 0 or 1;
n1 is 4;
n2 is 2;
R′ is at each occurrence independently selected from the group consisting of H, F, SF₅, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated, partially or completely halogenated thioalkyl group having 1 to 20 C atoms;
R″ is at each occurrence independently selected from the group consisting of H, F, Cl, Br, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated and partially or completely halogenated thioalkyl group having 1 to 20 C atoms;
R₁ is a trialkoxysilyl group or a dialkoxyalkylsilyl group where the alkyl and/or alkoxy groups are each independently linear or branched having 1 to 6 C atoms, or a silyl group of formula (1), (2) or (3) or a polymerizable group of formula (4), 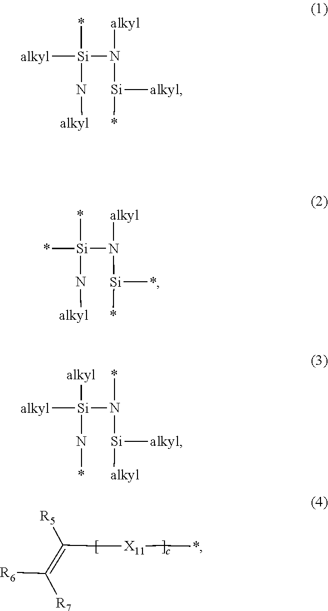
where alkyl means at each occurrence independently of each other a linear or branched alkyl group having 1 to 6 C atoms and the asterisk “*” denotes at each occurrence independently of each other a linkage to the linker —R₂—, —R₂—Y or [Y—R₂—]ₘ₁; and wherein
X₁₁ is selected from the group consisting of O, S, O—SO₂, SO₂—O, C(═O), OC(═O), C(═O)O, S(C═O) and (C═O)S,
R₅, R₆, R₇ are at each occurrence independently of each other selected from the group consisting of H, F, a linear or branched, non-fluorinated, partially or completely fluorinated alkyl group having 1 to 20 C atoms and aryl with 6 to 14 C atoms and
c is 0 or 1;
—R₂— is —(C(R)₂)ₒ—, or —(C(R)₂)ₚ—X₈—(C(R)₂)_{q}—(X₉)ₛ—(C(R)₂)ᵣ—(X₁₀)ₜ—(C(R)₂)ᵤ—;
R is at each occurrence independently selected from the group consisting of H, F, a linear or branched alkyl group having 1 to 4 C atoms or a linear or branched partially or fully fluorinated alkyl group having 1 to 4 C atoms;
o is selected from the group consisting of 0 to 20,
X₈, X₉, X₁₀ are at each occurrence independently O, S, SO₂, or NR₀,
s, t is 0 or 1,
p, q are at each occurrence independently selected from the group consisting of 1 to 10,
r, u are at each occurrence independently selected from the group consisting of 0 to 10, wherein the overall number of atoms for —(C(R)₂)ₚ—X₈—(C(R)₂)_{q}—(X₉)ₛ—(C(R)₂)ᵣ—(X₁₀)ₜ—(C(R)₂)ᵤ— is up to 20 atoms,
R₀ is at each occurrence independently selected from the group consisting of a linear or branched alkyl group having 1 to 4 C atoms and a linear or branched partially or fully fluorinated alkyl group having 1 to 4 C atoms;
R₃ is H, F, Cl, Br, CN or a linear or branched, non-halogenated, partially or completely halogenated alkyl group having 1 to 20 C atoms;
R₄ is R′ in case m1 is 0 and
R₄ is R₁ in case m1 is 1.

2. The ophthalmic device or the precursor article for manufacturing an ophthalmic device according to claim 1 wherein in polymerized compounds of formula (I) m1 is 0 said compounds being of formula (I′) 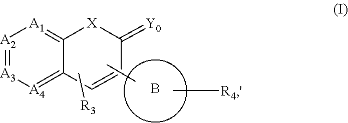
wherein R₁, —R₂—, Y, R₃, X, Y₀, R′, R″ and 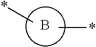
have a meaning as indicated in claim 1 and
A₁, A₂, A₃, A₄ are each independently of each other N, CR″ or C—Y—R₂—R₁, provided that only one of A₁, A₂, A₃ and A₄ is N, only one of A₁, A₂, A₃ and A₄ is C—Y—R₂—R₁ and the others are each independently CR″; or
adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″; and
R₄ is H or R′.

3. The ophthalmic device or the precursor article for manufacturing an ophthalmic device according to claim 1 wherein in polymerized compounds of formula (I) and m1 is 1 said compounds being of formula (I″) 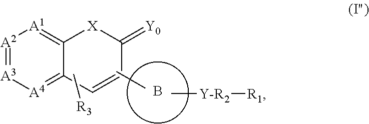
wherein R₁, —R₂—, Y, R₃, X, Y₀, R′, R″ and 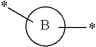
have a meaning as indicated in claim 1 and
A₁, A₂, A₃, A₄ are each independently of each other N or CR″ provided that only one of A₁, A₂, A₃ and A₄ is N and the others are CR″.

4. The ophthalmic device or the precursor article for manufacturing an ophthalmic device according to one or more of claims 1 to 3 comprising an oligomer, polymer or copolymer comprising a constitutional unit M⁰ based on formulae (I), (I′) or (I″) where R₁ on each occurrence is polymerized, R₁ thus forms the regioregular, alternated, regiorandom, statistical, block or random oligomer or polymer backbone or is part of the copolymer backbone.

5. The ophthalmic device or the precursor article for manufacturing an ophthalmic device according to one or more of claims 1 to 4 where said polymerized group R₁ is of formulae (1-p), (2-p), (3-p) or (4-p) 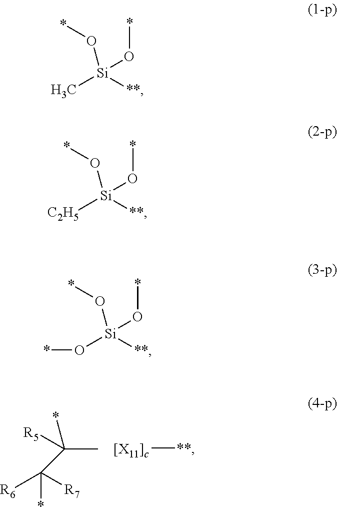
where the asterisk “*” within formulae (1-p) to (4-p) denotes a linkage to the adjacent repeating unit in the polymer chain or oligomer chain or to a terminal end group, the asterisk “**” within formulae (1-p) to (4-p) denotes the linkage to the remainder of formulae (I), (I′) or (I″) and R₅, R₆, R₇, X₁₁ and c has a meaning according to claim 1.

6. The ophthalmic device or the precursor article for manufacturing an ophthalmic device according to one or more of claims 1 to 5 wherein the constitutional unit M⁰ is of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″), 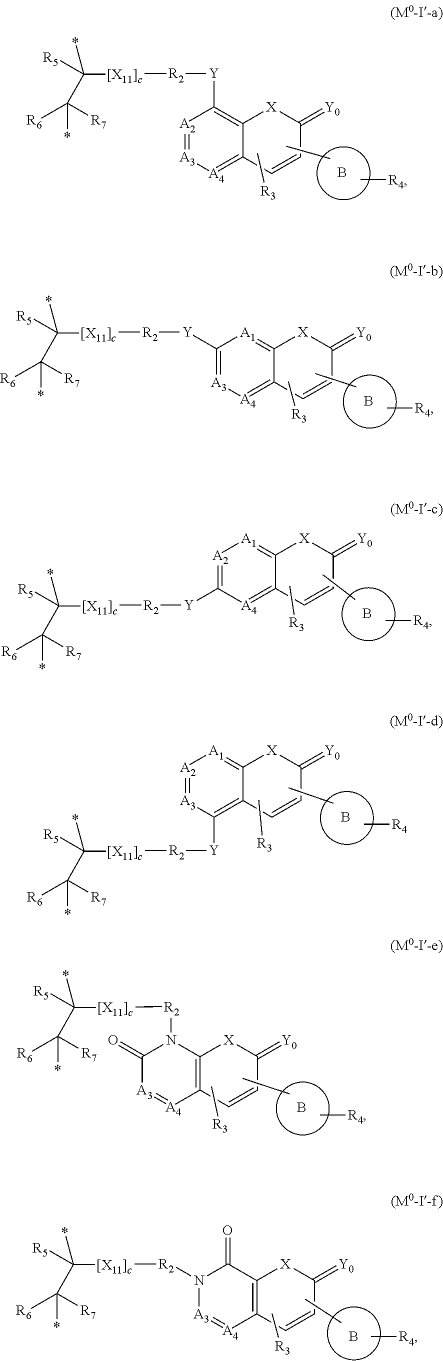 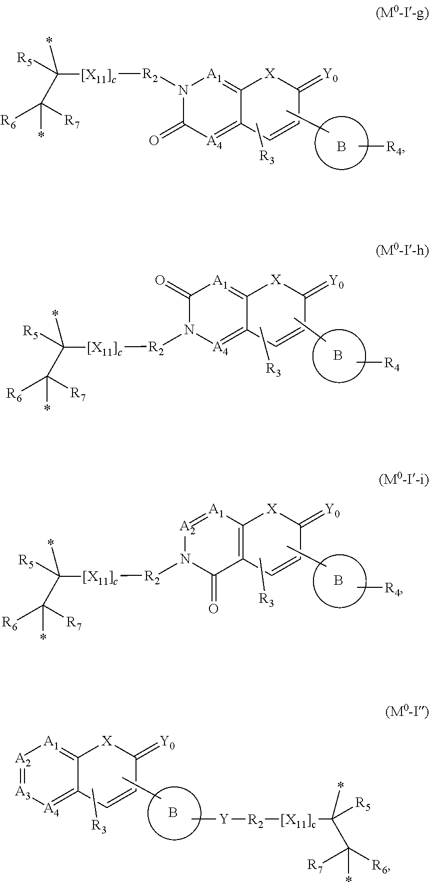
where R₁, —R₂—, X, Y₀, Y, A₁, A₂, A₃, A₄, R₃, R₄, R₅, R₆, R₇, X₁₁, c and 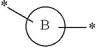
have a meaning according to claim 1 and R₄ is R′ and where the asterisk “*” denotes at each occurrence a linkage to the adjacent repeating unit in the polymer chain or oligomer chain or to a terminal end group.

7. The ophthalmic device or the precursor article for manufacturing an ophthalmic device according to one or more of claims 1 to 6 comprising beside of the at least one polymerized compound of formulae (I), (I′) or (I″) or the constitutional unit M⁰ of formulae (M⁰-I′-a), (M⁰-I′-b), (M⁰-I′-c), (M⁰-I′-d), (M⁰-I′-e), (M⁰-I′-f), (M⁰-I′-g), (M⁰-I′-h), (M⁰-I′-i) or (M⁰-I″) at least one further polymerized monomer selected from the group consisting of styrene, ethoxyethyl methacrylate (EOEMA), methyl methacrylate (MMA), methyl acrylate, n-alkyl acrylates (the n-alkyl group comprising 2-20 C-atoms), n-alkyl methacrylates (the n-alkyl group comprising 2-20 C-atoms), i-alkyl acrylates (the i-alkyl group comprising 3-20 C-atoms), i-alkyl methacrylates (the i-alkyl group comprising 3-20 C-atoms), ethoxyethoxy ethylacrylate (EEEA), 2-hydroxyethyl methacrylate (HEMA), tetrahydrofuryl methacrylate (THFMA), glycidylmethacrylate (GMA), 16-hydroxyhexadecyl acrylate, 16-hydroxyhexadecyl methacrylate, 18-hydroxyoctadecyl acrylate, 18-hydroxyoctadecyl methacrylate, 2-phenoxyethyl acrylate (EGPEA), heptafluorobutyl acrylate, heptafluorobutyl methacrylate, hexafluorobutyl acrylate, hexafluorobutyl methacrylate, hexafluoroisopropyl acrylate, hexafluoroisopropyle methacrylate, octafluoropentyl acrylate, octafluoropentyl methacrylate, petanfluoropropyl acrylate, pentafluoropropyl methacrylate, tetrafluoropropyl methacrylate, trifluoroethyl acrylate, trifluoroethyl methacrylate, Bisphenol A diacrylate-1 EO/Phenol (BPADA), 2-[3′-2′H-benzotriazol-2′-yl)-4′-hydroxyphenyl]ethyl methacrylate (BTPEM) or ehtyleneglycoldimethacrylate.

8. The ophthalmic device or the precursor article for manufacturing an ophthalmic device according to claim 7 wherein the at least one further polymerized monomer is selected from methyl methacrylate, 2-hydroxyethyl methacrylate, 2-phenoxyethyl acrylate, ethoxyethoxy ethylacrylate, 8-methylnonyl methacrylate, n-butyl methacrylate, 2-ethyl hexylmethacrylate or a mixture thereof.

9. The ophthalmic device or the precursor article for manufacturing an ophthalmic device according to one or more of claims 1 to 8 wherein —R₂— is at each occurrence independently —(C(R)₂)ₒ— and R and o have a meaning as indicated in claim 1.

10. The ophthalmic device or the precursor article for manufacturing an ophthalmic device according to one or more of claims 1 to 9 wherein X is O and Y₀ is O.

11. The ophthalmic device or the precursor article for manufacturing an ophthalmic device according to one or more of claims 1 to 10 wherein polymerized R₁ is at each occurrence independently derived from an acryl or methacryl radical.

12. The precursor article for manufacturing an ophthalmic device according to one or more of claims 1 to 11 wherein said precursor article is a blank which may be transformed into an eye implant, preferably an intraocular lens.

13. Process of forming an ophthalmic device or a precursor article for manufacturing an ophthalmic device according to one or more of claims 1 to 12, said process comprising the steps of
providing a composition comprising at least one compound of formulae (I), (I′) or (I″) as described in one or more of claims 1 to 3, 9 and 10 and/or an oligomer or polymer as described in one or more of claims 4 to 8 and 11 but having at least one reactive group left for polymerization and optionally further monomers different from compounds of formulae (I), (I′) or (I″) and/or crosslinking agents and/or UV absorbers and/or radical initiators,
subsequently forming the ophthalmic device or precursor article of said composition.

14. Process of changing the optical properties of an ophthalmic device or a precursor article for manufacturing an ophthalmic device according to one or more of claims 1 to 12, said process comprising the steps of
providing an ophthalmic device or a precursor article according to claim 13, and
subsequently exposing said ophthalmic device or said precursor article to irradiation having a wavelength of at least 200 nm and at most 1500 nm.

15. Ophthalmic device or precursor article for manufacturing an ophthalmic device obtainable by the process according to claim 14.

16. Oligomer, polymer or copolymer comprising at least one polymerized compound of formula (I) as described in claim 1.

17. Polymer according to claim 16 comprising beside of the polymerized compounds of formula (I) at least one further polymerized monomer selected from the group consisting of styrene, ethoxyethyl methacrylate (EOEMA), methyl methacrylate (MMA), methyl acrylate, n-alkyl acrylates (the n-alkyl group comprising 2-20 C-atoms), n-alkyl methacrylates (the n-alkyl group comprising 2-20 C-atoms), i-alkyl acrylates (the i-alkyl group comprising 3-20 C-atoms), i-alkyl methacrylates (the i-alkyl group comprising 3-20 C-atoms), ethoxyethoxy ethylacrylate (EEEA), 2-hydroxyethyl methacrylate (HEMA), tetrahydrofuryl methacrylate (THFMA), glycidylmethacrylate (GMA), 16-hydroxyhexadecyl acrylate, 16-hydroxyhexadecyl methacrylate, 18-hydroxyoctadecyl acrylate, 18-hydroxyoctadecyl methacrylate, 2-phenoxyethyl acrylate (EGPEA), heptafluorobutyl acrylate, heptafluorobutyl methacrylate, hexafluorobutyl acrylate, hexafluorobutyl methacrylate, hexafluoroisopropyl acrylate, hexafluoroisopropyle methacrylate, octafluoropentyl acrylate, octafluoropentyl methacrylate, petanfluoropropyl acrylate, pentafluoropropyl methacrylate, tetrafluoropropyl methacrylate, trifluoroethyl acrylate, trifluoroethyl methacrylate, Bisphenol A diacrylate-1 EO/Phenol (BPADA), 2-[3′-2′H-benzotriazol-2′-yl)-4′-hydroxyphenyl]ethyl methacrylate (BTPEM) or ehtyleneglycoldimethacrylate.

18. Composition for polymerization comprising at least one compound of formulae (I), (I′) or (I″) as described in one or more of claims 1 to 3, 9 and 10 and/or an oligomer or polymer according to claim 16 or 17 having at least one reactive group left for polymerization and/or a crosslinking agent and/or a UV absorber and/or a radical initiator and optionally further monomers different from compounds of formulae (I), (I′) or (I″).

19. Compounds of formula (I) 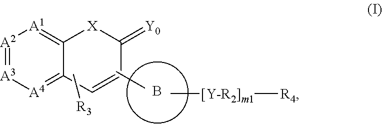
wherein
the divalent group 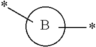
is selected from the group of formulae (B-1), (B-2), (B-3), (B-4) or (B-5) 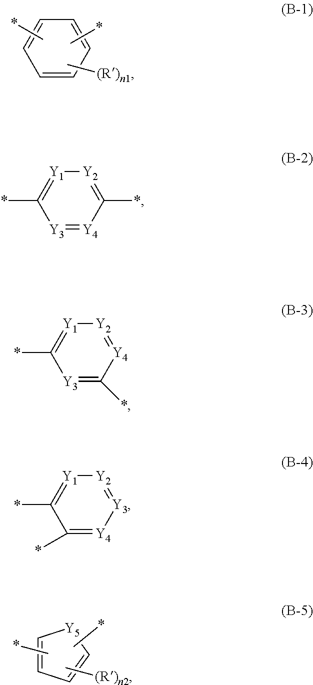
the asterisk * indicates the linkage to the remainder of formula (I);
Y₁, Y₂, Y₃, Y₄ are each independently of each other CR′ or N, provided that only one of Y₁, Y₂, Y₃ and Y₄ is N and the others are CR′;
Y₅ is O, S or NR_{B};
R_{B} is at each occurrence independently selected from a linear or branched alkyl group having 1 to 10 C atoms or a linear or branched partially or fully fluorinated alkyl group having 1 to 10 C atoms;
A₁, A₂, A₃, A₄ are each independently of each other N, CR″ or C—Y—R₂—R₁, provided that in case m1 is 1 only one of A₁, A₂ and A₃ is N and the others and A₄ are CR″ and provided that in case m1 is 0, only one of A₁, A₂ and A₃ is N, only one of A₁, A₂, A₃ and A₄ is C—Y—R₂—R₁ and the others are each independently CR″; or
in case of m1 is 0, adjacent A₁-A₂, A₂-A₃ or A₃-A₄ are each independently of each other —N(R₂—R₁)—C(═O)— or —C(═O)—N(R₂—R₁)— and the remaining A₃, A₄, A₁ and A₂ are each independently of each other CR″;
Y is independently of each other O, S, SO₂, or a bond;
m1 is 0 or 1;
n1 is 4;
n2 is 2;
R′ is at each occurrence independently selected from the group consisting of H, F, SF₅, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated, partially or completely halogenated thioalkyl group having 1 to 20 C atoms;
R″ is at each occurrence independently selected from the group consisting of H, F, Cl, Br, CN, SO₂CF₃, a linear or branched, non-halogenated, partially or completely halogenated alkyl group having 1 to 20 C atoms, a non-halogenated, partially or completely halogenated cycloalkyl group having 3 to 6 C atoms, a linear or branched, non-halogenated, partially or completely halogenated alkoxy group having 1 to 20 C atoms and a linear or branched, non-halogenated and partially or completely halogenated thioalkyl group having 1 to 20 C atoms;
R₁ is a trialkoxysilyl group or a dialkoxyalkylsilyl group where the alkyl and/or alkoxy groups are each independently linear or branched having 1 to 6 C atoms, or a silyl group of formula (1), (2) or (3) or a polymerizable group of formula (4), 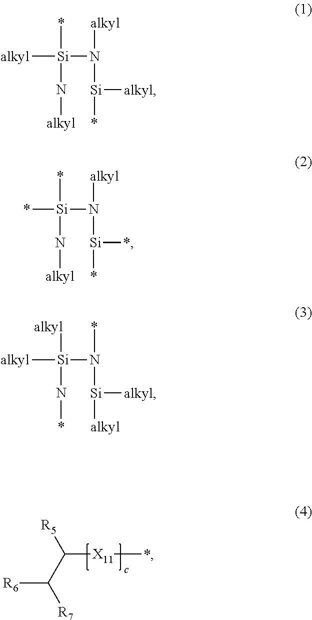
where alkyl means at each occurrence independently of each other a linear or branched alkyl group having 1 to 6 C atoms and the asterisk “*” denotes at each occurrence independently of each other a linkage to the linker —R₂—, —R₂—Y or [Y—R₂—]ₘ₁; and wherein
X₁₁ is selected from the group consisting of O, S, O—SO₂, SO₂—O, C(═O), OC(═O), C(═O)O, S(C═O) and (C═O)S,
R₅, R₆, R₇ are at each occurrence independently of each other selected from the group consisting of H, F, a linear or branched, non-fluorinated, partially or completely fluorinated alkyl group having 1 to 20 C atoms and aryl with 6 to 14 C atoms and
c is 0 or 1;
—R₂— is —(C(R)₂)ₒ—, or —(C(R)₂)ₚ—X₈—(C(R)₂)_{q}—(X₉)ₛ—(C(R)₂)ᵣ—(X₁₀)ₜ—(C(R)₂)ᵤ—;
R is at each occurrence independently selected from the group consisting of H, F, a linear or branched alkyl group having 1 to 4 C atoms or a linear or branched partially or fully fluorinated alkyl group having 1 to 4 C atoms;
o is 0 to 20,
X₈, X₉, X₁₀ are at each occurrence independently O, S, SO₂, or NR₀,
s, t is 0 or 1,
p, q are at each occurrence independently selected from the group consisting of 1 to 10,
r, u are at each occurrence independently selected from the group consisting of 0 to 10, wherein the overall number of atoms for —(C(R)₂)ₚ—X₈—(C(R)₂)_{q}—(X₉)ₛ—(C(R)₂)ᵣ—(X₁₀)ₜ—(C(R)₂)ᵤ— is up to 20 atoms,
R₀ is at each occurrence independently selected from the group consisting of a linear or branched alkyl group having 1 to 4 C atoms and a linear or branched partially or fully fluorinated alkyl group having 1 to 4 C atoms;
R₃ is H, F, Cl, Br, CN or a linear or branched, non-halogenated, partially or completely halogenated alkyl group having 1 to 20 C atoms;
R₄ is R′ in case m1 is 0;
R₄ is R₁ in case m1 is 1 and
provided that in case of m1 is 0, Y is O or S, A₂ is C—Y—R₂—R₁,
the divalent group 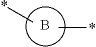
is in position 3 and is selected from formulae (B-3) and (B-4) where Y₄ within formula (B-3) is N and where Y₃ in formula (B-4) is N, c is 1;
provided that in case of m1 is 1, A₂ is CR″ and R″ is a linear or branched, non-halogenated, partially or completely halogenated alkoxy group having 1 to 20 C atoms and a linear or branched, non-halogenated and partially or completely halogenated thioalkyl group having 1 to 20 C atoms,
the divalent group 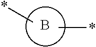
is in position 3 and is selected from formula (B-3), Y₂ within formula (B-3) is CR′ and R′ is H, Y₄ within formula (B-3) is N, and Y is a bond, O or S, c is 1;
provided that in case of m1 is 1, Y is a bond, c is 0, R₃ is Cl and
the divalent group 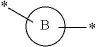
is in position 3, o is 5 to 20;
provided that in case of m1 is 0, Y is a bond or O, c is 0, R₃ is Cl and
the divalent group 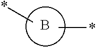
is in position 3, o is 7 to 20;
provided that in case of m1 is 0, X is O, Y₀ is O and Y is a bond, c is 1 and X₁₁ is O, S, O—SO₂, SO₂—O, OC(═O), C(═O)O, S(C═O) and (C═O)S;
provided that in case of m1 is 1, X is O and Y₀ is O, o is 5 to 20;
provided that in case of m1 is 1, c is 0, Y is a bond or O and
the divalent group 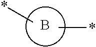
is in position 3, o is 11 to 20;
provided that in case of m1 is 1, A₁ or A₃ is N, Y is a bond, c is 0,
the divalent group 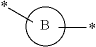
is in position 3, o is 7 to 20 and
provided that in case of m1 is 0, X is O, Y₀ is O, Y is O or S, c is 0, A₂ is C—Y—R₂—R₁, A₃ is Br, A₁ is N,
the divalent group 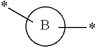
is in position 4 and is selected from formulae (B-1), (B-3) and (B-4), o is 2 to 20.
